(19) United States
(12) Patent Application Publication
(10) Pub. No.: US 2026/0130985 A1
(43) Pub. Date: May 14, 2026

(54) NUCLEIC ACID-BASED UNIVERSAL VACCINE AND METHODS OF USE THEREOF

(71) Applicant: ADVANCED RNA VACCINE (ARV) TECHNOLOGIES, INC., North Potomac, MD (US)

(72) Inventors: Huabin ZHU, Evans, GA (US); Yangiun QIN, Potomac, MD (US); Renhuan XU, North Potomac, MD (US)

(21) Appl. No.: 19/114,594

(22) PCT Filed: Sep. 25, 2023
(86) PCT No.: PCT/US2023/075052

§ 371 Date: Mar. 24, 2025

Related U.S. Application Data
(60) Provisional application No. 63/376,908, filed on Sep. 23, 2022.

(51) Int. Cl. A61K 39/215; A61K 39/00; A61K 39/275; A61P 31/14; C12N 15/11

(52) U.S. Cl. CPC A61K 39/215; A61K 39/275; A61P 31/14; C12N 15/11; A61K 2039/53; A61K 2039/54; A61K 2039/55555; A61K 2039/572; A61K 2039/575; A61K 2039/70; C12N 2710/16634; C12N 2710/24034; C12N 2770/20034

(57) ABSTRACT

Described herein are compositions including a nucleic acid sequence (e.g., mRNA) encoding an infection agent antigenic polypeptide and a nucleic acid sequence (e.g., mRNA) encoding at least one universal T-cell epitope (UTE), as well as compositions including a nucleic acid sequence (e.g., mRNA) encoding an infection agent antigenic polypeptide and at least one universal T-cell epitope, and methods for using the compositions.

### CROSS-REFERENCE TO RELATED APPLICATION

This application claims the benefit of U.S. Provisional Application No. 63/376,908, filed on Sep. 23, 2022, the contents of which is hereby incorporated in its entirety.

### REFERENCE TO A SEQUENCE LISTING

The Sequence Listing submitted Sep. 25, 2023 as a XML file named “11538-003WO1_Sequence_Listing.xml,” created on Sep. 22, 2022, and having a size of 1,353,272 bytes is hereby incorporated by reference pursuant to 37 C.F.R. § 1.834.

### BACKGROUND

Vaccine-induced neutralizing antibodies are considered the gold standard for preventing pathogens from entering or causing diseases. However, many pathogens have developed mechanisms to evade antibody protection, such as some RNA viruses (e.g., SARS-CoV-2), which frequently mutate viral structural proteins, resulting in diminished protective efficacy over time. Therefore, there is a need to develop a universal vaccine that ensures broad efficacy against multiple strains or different variants of one pathogen.

To date, the importance of T cell immunity in vaccination may have been relatively underestimated. T cells, particularly cytotoxic CD8 T cells, play a central role in the control of infections, including the prevention of severe COVID-19 and HSV-1/2-associated diseases. These cytotoxic T cells can kill any infected cell by recognizing foreign peptides presented on the cell surface bound to MHC class I molecules (MHC I). If the pathogen-derived peptides are selected from conserved regions of different strains or variants, then the activated T cells will be capable of providing a broad spectrum of protection. Thus, a vaccine platform capable of inducing sufficiently high-quality antibodies and memory T cells would greatly increase the efficacy of protection, especially against infections that cannot be effectively controlled by antibodies alone.

Accordingly, there is a need for a platform for generating universal vaccines.

### SUMMARY

Described herein are compositions, nanoparticles, and/or vaccines including a nucleic acid sequence for example, DNA or RNA (e.g., mRNA) encoding an infection agent antigenic polypeptide and a nucleic acid encoding at least one universal T-cell epitope, as well as, compositions, nanoparticles, and/or vaccines including a nucleic acid sequence encoding an infection agent antigenic polypeptide and at least one universal T-cell epitope. The universal T-cell epitopes (UTE) described herein are epitopes highly conserved across various strains/variants/serotypes of a virus. In some embodiments, the compositions, nanoparticles, and/or vaccines can further include pharmaceutically acceptable carriers.

In some embodiments, the compositions including a nucleic acid sequence (e.g., mRNA) encoding an infection agent antigenic polypeptide and a nucleic acid sequence encoding a universal T-cell epitope can elicit an antibody response in a subject. In some embodiments, the compositions including a nucleic acid sequence encoding the infection agent antigenic polypeptide and a nucleic acid sequence encoding a universal T-cell epitope can elicit both an antibody response and a T-cell response in a subject. In some embodiments, the compositions including a nucleic acid sequence encoding the infection agent antigenic polypeptide and a nucleic acid sequence encoding a universal T-cell epitope can elicit a broad T-cell response in a subject. A broad T-cell response as described herein refers to a T-cell response against at least one universal T-cell epitope in order for a composition or vaccine to protect against different variants of the same virus.

In some embodiments, the compositions including a nucleic acid sequence (e.g., mRNA) encoding the infection agent antigenic polypeptide and a universal T-cell epitope can elicit an antibody response in a subject. In some embodiments, the compositions including a nucleic acid sequence encoding the infection agent antigenic polypeptide and a universal T-cell epitope can elicit both an antibody response and a T-cell response in a subject. In some embodiments, the compositions including a nucleic acid sequence encoding the infection agent antigenic polypeptide and the universal T-cell epitopes can elicit a broad T-cell response in a subject. For example, in some embodiments, the nucleic acid sequence encoding an infection agent antigenic polypeptide and at least one universal T-cell epitope may be recognized by the immune system of a subject to elicit a CD4+ T cell response or/and CD8+ T cell response.

In some embodiments, the universal T-cell epitope(s) can provide greater protection against infection agent variants.

In some embodiments, the nucleic acid sequence can encode an infection agent antigenic polypeptide. In some embodiments, the infection agent can be a virus. For example, negative-sense, single-stranded RNA virus of the family Paramyxoviridae such as human Metapneumovirus (hMPV), parainfluenza viruses (PIV), respiratory syncytial virus (RSV), measles virus (MeV), varicella-zoster, influenza virus (e.g., influenza A and B), herpes simplex virus 1 (HSV1), herpes simplex virus 2 (HSV2), poxvirus (e.g., smallpox, monkeypox), HIV, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, coronaviruses (e.g., MERS-COV, SARS-COV, SARS-COV2, HCoV-OC43. HCOV-229E, HCOV-NL63, HCOV-NL, HCOV-NH, HCOV-HKU1), African swine fever (ASF), foot-and-mouth disease virus (FMDV), feline herpesvirus-1/feline viral rhinotracheitis, canine distemper, feline coronavirus (FCOV) or any combination thereof. In some embodiments, the infection agent can be monkeypox, herpes simplex virus 1 (HSV1), herpes simplex virus 2 (HSV2), SARS-COV-2, or any combination thereof.

Described herein are methods of treating, inhibiting, reducing, ameliorating, and/or preventing a viral infection caused by an infection agent in a subject comprising administering to the subject a composition described herein, a nanoparticle described herein, or a vaccine described herein.

In some embodiments, also described are methods of activating T cells, B cells, or any combination thereof, stimulating the proliferation of T cells, B cells, or any combination thereof, eliciting an immune response in a subject to an infection agent, and/or enhancing an immune response generated by a nucleic acid-based vaccine including introducing a nucleic acid sequence encoding at least one universal T-cell epitope into the nucleic acid-based vaccine. In some embodiments, the methods can include administering to the subject a composition described herein, a nanoparticle described herein, or a vaccine described herein.

In some embodiments, the nucleic acid can include DNA, RNA, any combination thereof (e.g., plasmid DNA, minicircle DNA, minimalistic, immunologically defined gene expression (MIDGE) and Doggybone, messenger RNA (mRNA), circle (cirRNA), self-amplifying-RNA (saRNA, also refer as SAM), or DNA launched SAM (DLSAM)). The nucleic acid can encode a bicistronic or multi-cistronic construct, for example, a DNA comprising at least two antigens.

In some embodiments, the subject can be a human. In some embodiments, the subject can be non-human vertebrate.

### BRIEF DESCRIPTION OF THE DRAWINGS

The accompanying figures, which are incorporated in and constitute a part of this specification, illustrate several aspects described below.

FIGS. 1A-1H Designation and validation of PanCoVAX mRNA. (1A) scheme of PanCoVAX mRNAs. (1B) IVT mRNA of DVS, OVS and MTE. (1C-1G) expression level of mRNA DVS and OVS in 293T cells are determined by Western blot (1C) Flow Cytometry (1D-1E) DVS (1D) and OVS (1E) and Immunostaining (1F-1G) DVS (1F) and OVS (1G). (1H) expression level of mRNA MTE in 293 T cells.

FIGS. 2A-2C Physiochemical characterization of lipid nanoparticle formulation. (2A) Size and polydispersity index (PDI) of ARV-L002 (“ARV-T1”) and ARV-L001 (“SM102”) LNP formulations with DVS or DVS/MTE. (2B) Surface charge (zeta potential) and mRNA encapsulation efficiency of ARV-L001 and ARV-L002 LNP formulation with DVS or DVS/MTE. (2C) Western blot analysis showing in vitro expression of spike protein after transfecting cells with 2.5 ug mRNA over time in 293 T cells.

FIGS. 3A-3F ELISA and pseudovirus neutralization assay. (3A) immunization scheme: BALB/c mice were immunized at week 0 and 3. Serum samples were collected on day 14 and 35 post primary immunization. Convalescent serum derived from COVID positive human serum was used as control. Spike specific antibody response total IgG (3B) and (3C) and neutralizing antibodies (3D-3F) delta pseudovirus (3D), omicron pseudovirus (3E) and omicron-BA2 pseudovirus (3F) were evaluated with serum by ELISA and pseudovirus neutralization assay.

FIGS. 4A-4D T cell response detection by Elispot assay. ELISPOT as described in the M&M, 3×10⁵ splenocytes were used to measure the Ag-specific T cell response upon stimulation of Spike (4A, 4B) and MTE (4C, 4D) overlapping peptides (synthesized by Genscript and JPT, respectively) at 1 μg/ml.

FIGS. 5A-5C Dosing study. BALB/c mice were immunized twice at week 0 and 3 with 0.01 ug, 0.lug and 1 μg mRNA formulated with either SM102 or ARV-T1 LNP. Serum samples were collected on day 14 (5A) and 35 (5B) post primary immunization. Spike specific antibody response total IgG (5A and 5B) and neutralizing antibodies (5C) on day 35 were evaluated with serum by ELISA and pseudovirus neutralization assay.

FIGS. 6A-6C. mRNA encoding MTE, OVS or MTE/OVS efficiently protected hACE2 transgenic mice from SARS-COV-2 Delta variant infection. (6A) scheme of immunization and challenge schedule, hACE-transgenic mice (K18) were immunized twice with ARV TI formulated LNP containing mRNA of MTE, OVS and MTE/OVS, 14 days after the last immunization, mice were infected with a lethal dose of SARS-COV-2 Delta Variant. (6B) and (6C), after infection, the animal body weight (6B) and survival (6C) were observed on daily basis. (6D) virus titers were determined by qRT-PCR from lungs at 4 days post-infection.

FIGS. 7A-7E T-cell response detection by intracellular staining. Splenocytes were isolated and stimulated with different peptides pool of spike or MTE, After 6h, intracellular cytokine staining was performed and analyzed by flow cytometry (7A). (7B) Spike-specific CD4 T cell response. (7C) Spike-specific CD8 T cell response. (7D) MTE-specific CD4 T cell response. (7E) MTE-specific CD8 T cell response.

FIGS. 8A-8F show in vitro transfection efficiency of GFP mRNA (1 μg/mL) using LNP deliver into BHK cells after 24 hours. (8A) Representative fluorescent images of BHK cells after transfection. (8B) Transfection efficacy analysis of GFP expression using flow cytometer. MFI denotes Mean Fluorescent Intensity. (8C-8F) Characterization of LNP (T1)-mRNA GFP, (8C) size and PDI. (8D) S potential and encapsulation efficacy, (8E) CryoEM LNP image, (8F) LNP size based on CryoEM image. FIG. 9 Biodistribution and delivered cell type of LNP (T1) in vivo. (9A) shows in vivo transfection efficiency of luciferase-expressing mRNA in vivo. LNPs were formulated with indicated ionizable lipids and 1 μg of formulated Luciferase-expressing mRNA were injected intramuscularly. After administration, the luciferase expression was determined by whole body bioluminescence imaging using an IVIS Spectrum in vivo imaging system at 6, 24, 48, and 72 hours, respectively. (9B) Biodistribution of LNP (T1)-mRNA luciferase in vivo. (9C-9D), delivery cell type of LNP (T1)-mRNA Cre in Ai14 mice by I.M. and I.V.

FIG. 10 Antigen-specific T cell responses were evaluated by Elispot. Data were presented as Mean±SD. Statistical comparisons were analyzed using by one-way ANOVA with Tukey's multiple comparison test. *p<0.05, **p<0.01.

FIG. 11 Synthetizations of HSV mRNA vaccine in vitro. HSV gB, gD, gH, gL, UL19, and MTE mRNAs were synthesized with T7 RNA polymerase in vitro transcription and run on 0.8% MOPS agarose gel.

FIGS. 12A-12F validation of HSV mRNA vaccine in vitro. (12A-12C) HSV mRNA gB (12A), gD (12B) and gL (12C) were transfected into 293T cells with mRNA transfection kit and detected by western blot. (12D) HSV mRNA MTE-His was transfected into 293T cells with mRNA transfection kit. After 48h, MTE-His protein were concentrated from cell lysis and detected by western blot. (12E-12F) HSV mRNA gH-HA (12E) and UL-19 (12F) were transfected into 293T cells with mRNA transfection kit and detected by flow cytometry.

FIGS. 13A-13B Physiochemical characterization of lipid nanoparticle formulation. (13A) Size and polydispersity index (PDI) of LNP formulations with indicated mRNA using ARV-L002 (ARV-T1). (13B) encapsulation efficiency of mRNA vaccine of ARV-L002 LNP formulation with indicated mRNA.

FIGS. 14A-14G HSV mRNA vaccine elicits robust T cell responses and strong antibody responses in BALB/c mice. (14A) BALB/c mice were intramuscularly immunized on D0, D21, and D42 with 5 μg of LNP formulated mRNA vaccines (gD-wt, gD, gB/gD, gB/gD+gH/gL and gH/gL+MTE/UL19). Mouse serum were collected on D14, D35, and D56, respectively. Mouse spleens were collected on D56. (14B) gD specific IgG were detected by ELISA from serum samples. (14C) Neutralization assays were performed with HSV-2 MS strain in Vero cells by counting plaque. (14D-14G) Splenocytes were isolated from mouse spleen and performed for ELISPOT assay (14D-14E) or flow cytometry intracellular staining (14F-14G) with stimulation of a UL-19 peptides pool (14E-14F) or gD peptides pool (14D and 14G).

FIGS. 15A-15D HSV mRNA vaccines protect mice from HSV-2 challenge. (15A-15C) 50% lethal dose (LD50) of HSV2 MS in BALB/c. (15A) Female BALB/c mice were injected with 2 mg medroxyprogesterone on D-7 and -3 and challenged intravaginally with different PFU of HSV-2. Mouse body weight and survival were recorded after the challenge. (15B) Mouse survival curve after challenge. (15C) The value of LD50 of HSV-2 MS strain in BALB/c mice. (15D), Female BALB/c mice were intramuscularly immunized on D0, D21 with 5 μg of LNP formulated mRNA vaccines (gB/gD, gB/gD+MTE/UL19, gB/gD+gH/gL+MTE/UL19 and gB/gD+gH/gL+gC/gE+MTE/UL19). Mouse serum samples were collected on D14, D28. Mice were injected with 2 mg medroxyprogesterone on D35 and 39 and challenged intravaginally with 10+PFU HSV2 MS strain or HSV-1 HF on D42. Vaginal cultures were collected on D44 and 46 to determine the copy number of HSV2 or HSV-1. Mouse genital disease, body weight and survival were recorded until to D56.

FIG. 16A Synthetization and formulation of FIPV mRNA vaccine in. Linearization of DNA plasmids with BspQI before in vitro transcription. 1, 2 indicated before and after digestion respectively.

FIGS. 17A-17B Validation of FIPV mRNA vaccine in vitro. (17A) FIPV Spike mRNAs (Fcov-I-S-2P-HA, Fcov-I-S-4P-HA and Fcov-I-S-2P2Cb-HA) were synthesized with T7 RNA polymerase in vitro transcription and run on 0.8% MOPS agarose gel. (17B) FIPV Spike mRNAs were transfected into 293T cells with mRNA transfection kit and detected by western blot.

FIGS. 18A-18C mRNA with human derived UTR can be efficiently expressed in feline cells. eGFP mRNAs franking with human derived UTRs were transfected into feline cell line FCWF-4cu and human cell line 293T cells with mRNA transfection kit. The expression levels of eGFP were detected by Flow cytometry (18A-18B) and fluorescence microscope (18C).

FIGS. 19A-19B Production of Fcov-I SI antigen and antibody. (19A) Recombinant Fcov-I S1 subunit His tag protein was produced in vitro and detected with His antibody. (19B) Rabbits were immunized 4 doses with mRNA formulated with ARV-T1 LNP expressing FCov-I full length S protein. Serum samples were collected 3 weeks after immunizations and performed ELISA assay for S1 antibody titers with recombinant Fcov-I S1-His protein.

FIGS. 20A-20D FIPV mRNA vaccine elicits robust T cell responses and strong antibody responses in BALB/c mice. (20A) BALB/c mice were intramuscularly immunized on DO and D21 with 2 μg of LNP formulated mRNA vaccines (Fcov-I-S-wt, Fcov-I-S-2P, Fcov-I-S-2P2Cb, Fcov-I-S-4P and Fcov-II-RBD-MN). Mouse serum samples were collected on D14 and D35. Mouse spleens were collected on D35. (20B) SI specific IgG were detected by ELISA from serum samples. (20C-20D) Splenocytes were isolated from mouse spleens and performed for ELISPOT assay with stimulation of a S1 protein (20C) or N peptides pool (20D).

FIG. 21 FIPV mRNA vaccine protects cat from Fcov-I and Fcov-II challenge. Cats were intramuscularly immunized on DO and D21 with 10 ug of LNP formulated mRNA vaccines (Fcov-I-S-2P, Fcov-II-RBD-MN and Fcov-I-S-2P/Fcov-II-RBD-MN). Cat sera were collected on D14 and D28. Cats were challenged with Fcov-I and Fcov-II virus on D35. Cats body weight and survival were recorded until D49.

### DETAILED DESCRIPTION

The present disclosure provides compositions including a nucleic acid sequence, for example an mRNA, encoding an infection agent antigenic polypeptide and a nucleic acid sequence encoding at least one universal T-cell epitope (UTE), as well as, compositions including a nucleic acid sequence encoding an infection agent antigenic polypeptide and at least one T-cell epitope. In some embodiments, the composition can be a nanoparticle (e.g., lipid nanoparticle), or a vaccine. The present disclosure also provides methods of using the compositions for delivering a nucleic acid sequence (e.g., mRNA) described herein to a subject.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this invention belongs. The following definitions are provided for the full understanding of terms used in this specification.

### Definitions

### General Definitions

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. A range may be construed to include the start and the end of the range. For example, a range of 10% to 20% (i.e., range of 10%-20%) can includes 10% and also includes 20%, and includes percentages in between 10% and 20%, unless explicitly stated otherwise herein.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

“Administration” to a subject includes any route of introducing or delivering to a subject an agent. Administration can be carried out by any suitable route, including oral, topical, transcutaneous, transdermal, intra-joint, intra-arteriole, intradermal, intraventricular, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used herein, the term “controlled-release” or “controlled-release drug delivery” or “extended release” refers to release or administration of a drug from a given dosage form in a controlled fashion in order to achieve the desired pharmacokinetic profile in vivo. An aspect of “controlled” drug delivery is the ability to manipulate the formulation and/or dosage form in order to establish the desired kinetics of drug release.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers. In some embodiments, the polynucleotide is composed of nucleotide monomers of generally greater than 100 nucleotides in length and up to about 8,000 or more nucleotides in length.

Nucleic acids may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a-D-ribo configuration, a-LNA having an a-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

In some embodiments, polynucleotides of the present disclosure function as messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The skilled artisan will appreciate that, except where otherwise noted, polynucleotide sequences set forth in the instant application will recite “T”'s in a representative DNA sequence but where the sequence represents RNA (e.g., mRNA), the “T”s would be substituted for “U”s. Thus, any of the RNA polynucleotides encoded by a DNA identified by a particular sequence identification number may also comprise the corresponding RNA (e.g., mRNA) sequence encoded by the DNA, where each “T” of the DNA sequence is substituted with “U.”

The basic components of an mRNA molecule typically include at least one coding region, a 5′ untranslated region (UTR), a 3′ UTR, a 5′ cap and a poly-A tail. Polynucleotides of the present disclosure may function as mRNA but can be distinguished from wild-type mRNA in their functional and/or structural design features, which serve to overcome existing problems of effective polypeptide expression using nucleic-acid based therapeutics.

In some embodiments, a RNA polynucleotide an of RNA (e.g., mRNA) vaccine encodes 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9 or 9-10 antigenic polypeptides. In some embodiments, a nucleic acid sequence (e.g., mRNA) encodes at least 10, 20, 30, 40, 50, 60, 70, 80, 90 or 100 antigenic polypeptides. In some embodiments, a nucleic acid sequence (e.g., mRNA) encodes at least 100 or at least 200 antigenic polypeptides. In some embodiments, a RNA polynucleotide of an encodes 1-10, 5-15, 10-20, 15-25, 20-30, 25-35, 30-40, 35-45, 40-50, 1-50, 1-100, 2-50 or 2-100 antigenic polypeptides.

Polynucleotides of the present disclosure, in some embodiments, are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art-non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments, a codon optimized sequence shares less than 95% sequence identity, less than 90% sequence identity, less than 85% sequence identity, less than 80% sequence identity, or less than 75% sequence identity to a naturally-occurring or wild-type sequence (e.g., a naturally-occurring or wild-type mRNA sequence encoding a polypeptide or protein of interest (e.g., an antigenic protein or antigenic polypeptide)).

In some embodiments, a codon-optimized sequence shares between 65% and 85% (e.g., between about 67% and about 85%, or between about 67% and about 80%) sequence identity to a naturally-occurring sequence or a wild-type sequence (e.g., a naturally-occurring or wild-type mRNA sequence encoding a polypeptide or protein of interest (e.g., an antigenic protein or polypeptide)). In some embodiments, a codon-optimized sequence shares between 65% and 75%, or about 80% sequence identity to a naturally-occurring sequence or wild-type sequence (e.g., a naturally-occurring or wild-type mRNA sequence encoding a polypeptide or protein of interest (e.g., an antigenic protein or polypeptide)).

In some embodiments a codon-optimized RNA (e.g., mRNA) may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

The term “polypeptide” refers to a compound made up of a single chain of D- or L-amino acids or a mixture of D- and L-amino acids joined by peptide bonds.

In some embodiments, a polypeptide is longer than 25 amino acids and shorter than 50 amino acids. The term “antigenic polypeptide” includes full length polypeptides/proteins as well as immunogenic fragments thereof (immunogenic fragments capable of inducing an immune response to an infection agent). Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer, or tetramer. Polypeptides may also comprise single chain or multichain polypeptides such as antibodies or insulin and may be associated or linked. Most commonly, disulfide linkages are found in multichain polypeptides. The term polypeptide may also apply to amino acid polymers in which at least one amino acid residue is an artificial chemical analogue of a corresponding naturally-occurring amino acid.

A “polypeptide variant” is a molecule that differs in its amino acid sequence relative to a native sequence or a reference sequence. Amino acid sequence variants may possess substitutions, deletions, insertions, or a combination of any two or three of the foregoing, at certain positions within the amino acid sequence, as compared to a native sequence or a reference sequence. Ordinarily, variants possess at least 50% identity to a native sequence or a reference sequence. In some embodiments, variants share at least 80% identity or at least 90% identity with a native sequence or a reference sequence.

In some embodiments “variant mimics” are provided. A “variant mimic” contains at least one amino acid that would mimic an activated sequence. For example, glutamate may serve as a mimic for phosphoro-threonine and/or phosphoro-serine. Alternatively, variant mimics may result in deactivation or in an inactivated product containing the mimic. For example, phenylalanine may act as an inactivating substitution for tyrosine, or alanine may act as an inactivating substitution for serine.

“Orthologs” refers to genes in different species that evolved from a common ancestral gene by speciation. Normally, orthologs retain the same function in the course of evolution. Identification of orthologs is important for reliable prediction of gene function in newly sequenced genomes.

“Analogs” is meant to include polypeptide variants that differ by one or more amino acid alterations, for example, substitutions, additions or deletions of amino acid residues that still maintain one or more of the properties of the parent or starting polypeptide.

The present disclosure provides several types of compositions that are polynucleotide or polypeptide based, including variants and derivatives. These include, for example, substitutional, insertional, deletion and covalent variants and derivatives. The term “derivative” is synonymous with the term “variant” and generally refers to a molecule that has been modified and/or changed in any way relative to a reference molecule or a starting molecule.

As such, polynucleotides encoding peptides or polypeptides containing substitutions, insertions and/or additions, deletions and covalent modifications with respect to reference sequences, in particular the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal residues or N-terminal residues) alternatively may be deleted depending on the use of the sequence, as for example, expression of the sequence as part of a larger sequence that is soluble, or linked to a solid support.

“Substitutional variants” when referring to polypeptides are those that have at least one amino acid residue in a native or starting sequence removed and a different amino acid inserted in its place at the same position. Substitutions may be single, where only one amino acid in the molecule has been substituted, or they may be multiple, where two or more (e.g., 3, 4 or 5) amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Features” when referring to polypeptide or polynucleotide are defined as distinct amino acid sequence-based or nucleotide-based components of a molecule respectively. Features of the polypeptides encoded by the polynucleotides include surface manifestations, local conformational shape, folds, loops, half-loops, domains, half-domains, sites, termini and any combination(s) thereof.

As used herein when referring to polypeptides the term “domain” refers to a motif of a polypeptide having one or more identifiable structural or functional characteristics or properties (e.g., binding capacity, serving as a site for protein-protein interactions).

As used herein when referring to polypeptides the terms “site” as it pertains to amino acid based embodiments is used synonymously with “amino acid residue” and “amino acid side chain.” As used herein when referring to polynucleotides the terms “site” as it pertains to nucleotide based embodiments is used synonymously with “nucleotide.” A site represents a position within a peptide or polypeptide or polynucleotide that may be modified, manipulated, altered, derivatized or varied within the polypeptide-based or polynucleotide-based molecules.

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein having a length of 10, 20, 30, 40, 50, 60, 70, 80, 90, 100 or longer than 100 amino acids. In another example, any protein that includes a stretch of 20, 30, 40, 50, or 100 (contiguous) amino acids that are 40%, 50%, 60%, 70%, 80%, 90%, 95%, or 100% identical to any of the sequences described herein can be utilized in accordance with the disclosure. In some embodiments, a polypeptide includes 2, 3, 4, 5, 6, 7, 8, 9, 10, or more mutations as shown in any of the sequences provided herein or referenced herein. In another example, any protein that includes a stretch of 20, 30, 40, 50, or 100 amino acids that are greater than 80%, 90%, 95%, or 100% identical to any of the sequences described herein, wherein the protein has a stretch of 5, 10, 15, 20, 25, or 30 amino acids that are less than 80%, 75%, 70%, 65% to 60% identical to any of the sequences described herein can be utilized in accordance with the disclosure.

Polypeptide or polynucleotide molecules of the present disclosure may share a certain degree of sequence similarity or identity with the reference molecules (e.g., reference polypeptides or reference polynucleotides), for example, with art-described molecules (e.g., engineered or designed molecules or wild-type molecules). The term “identity,” as known in the art, refers to a relationship between the sequences of two or more polypeptides or polynucleotides, as determined by comparing the sequences. In the art, identity also means the degree of sequence relatedness between two sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. Identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al. (1997). “Gapped BLAST and PSI-BLAST: a new generation of protein database search programs,” Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently, a Fast Optimal Global Sequence Alignment Algorithm (FOGSAA) was developed that purportedly produces global alignment of nucleotide and protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g. between nucleic acid molecules (e.g. DNA molecules and/or RNA molecules) and/or between polypeptide molecules. Polymeric molecules (e.g. nucleic acid molecules (e.g. DNA molecules and/or RNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one.

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press. New York, 1988; Biocomputing: Informatics and Genome Projects, Smith, D. W., ed., Academic Press, New York. 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12 (1), 387 (1984)), BLASTP, BLASTN, and FASTA Altschul, S. F. et al., J. Molec. Biol., 215, 403 (1990)).

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures, resulting in the separation of the double strand into two single strands by breaking the hydrogen bonds between the strands.

The term “target” refers to a molecule that has an affinity for a given probe. Targets may be naturally-occurring or man-made molecules. Also, they can be employed in their unaltered state or as aggregates with other species.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)|, the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8 (1), p. 466-472, 1988); SV40 enhancer; and the intron sequence between exons 2 and 3 of rabbit β-globin (Proc. Natl. Acad. Sci. USA., Vol. 78 (3), p. 1527-31, 1981). It is appreciated by those skilled in the art that the design of the expression vector can depend on such factors as the choice of the host cell to be transformed, the level of expression desired, etc.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

A nucleic acid sequence is “heterologous” to a second nucleic acid sequence if it originates from a foreign species, or, if from the same species, is modified by human action from its original form. For example, a heterologous promoter (or heterologous 5′ untranslated region (5′UTR)) operably linked to a coding sequence refers to a coding sequence from a species different from that from which the promoter was derived, or, if from the same species, a coding sequence which is different from naturally occurring allelic variants (for example, the 5′UTR or 3′UTR from a different gene is operably linked to a nucleic acid encoding for a co-stimulatory molecule).

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or antigen binding fragment thereof” or “antibody or fragments thereof” encompasses chimeric antibodies and hybrid antibodies, with dual or multiple antigen or epitope specificities, and fragments, such as F(ab′)2, Fab′, Fab, Fv, sFv, scFv and the like, including hybrid fragments. Thus, fragments of the antibodies that retain the ability to bind their specific antigens are provided. For example, fragments of antibodies which maintain binding activity are included within the meaning of the term “antibody or antigen binding fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or antigen binding fragment thereof” are conjugates of antibody fragments and antigen binding proteins (single chain antibodies). Also included within the meaning of “antibody or antigen binding fragment thereof” are immunoglobulin single variable domains, such as for example a nanobody.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

As used herein, “immune effector cells” refers to cells capable of binding an antigen or a peptide and which mediate an immune response. These cells include, but are not limited to, T cells (include CD4+ and CD8+ T cells), B cells, monocytes, macrophages, NK cells and cytotoxic T lymphocytes (CTLs).

### Chemical Definitions

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OZ¹ where Z¹ is alkyl as defined above.

The term “alkenyl” as used herein is a hydrocarbon group of from 2 to 24 carbon atoms with a structural formula containing at least one carbon-carbon double bond. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl group can be substituted or unsubstituted. The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” or “CO” is a short hand notation for C═O.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C (O) Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” as used herein refers to the fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “phosphonyl” is used herein to refer to the phospho-oxo group represented by the formula —P(O)(OZ¹)₂, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “thiol” as used herein is represented by the formula —SH.

The term “thio” as used herein is represented by the formula —S—.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxyl group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, articles, and methods, examples of which are illustrated in the accompanying Examples and Figures.

### Compositions

Described herein are compositions including a nucleic acid sequence (e.g., mRNA) encoding an infection agent antigenic polypeptide and a nucleic acid sequence (e.g., mRNA) encoding at least one universal T-cell epitope, as well as, compositions including a nucleic acid sequence (e.g., mRNA) encoding an infection agent antigenic polypeptide and at least one universal T-cell epitope.

The compositions described herein, can include multiple nucleic acid sequences (e.g., mRNA), each encoding a single infection agent antigenic polypeptide and multiple nucleic acid sequences (e.g., mRNA), each encoding a single universal T-cell epitope, as well as compositions including a single nucleic acid sequence (e.g., mRNA) encoding one or more infection agent antigenic polypeptide and a single nucleic acid sequence (e.g., mRNA) encoding one or more universal T-cell epitope(s).

The compositions described herein, can include multiple nucleic acid sequences (e.g., mRNA) each encoding a single infection agent antigenic polypeptide and a single universal T-cell epitope, as well as, compositions including a single nucleic acid sequence (e.g., mRNA) encoding one or more infection agent antigenic polypeptide and one or more universal T-cell epitope(s).

Thus, a composition including a nucleic acid sequence encoding an infection agent antigenic polypeptide and at least one universal T-cell epitope, encompasses compositions that include a nucleic acid sequence encoding a first infection agent antigenic polypeptide, a second infection agent antigenic polypeptide, a first universal T-cell epitope, and a second universal T-cell epitope. In some embodiments, a composition including a nucleic acid sequence encoding an infection agent antigenic polypeptide and a nucleic acid sequence encoding at least one universal T-cell epitope, encompasses compositions that include a first nucleic acid sequence encoding a first infection agent antigenic polypeptide, a second nucleic acid sequence encoding a second infection agent antigenic polypeptide, a third nucleic acid sequence encoding a first universal T-cell epitope, and a fourth nucleic acid sequence encoding a second universal T-cell epitope.

The compositions described herein, in some embodiments, include 2-10 (e.g., 2, 3, 4, 5, 6, 7, 8, 9 or 10), or more, nucleic acid sequence (e.g. mRNA) having an open reading frame, each of which encodes a different infection agent antigenic polypeptide (or a single nucleic acid sequence (e.g. mRNA) encoding 2-10, or more, different infection agent antigenic polypeptides). The compositions described herein, in some embodiments, include 2-10 (e.g., 2, 3, 4, 5, 6, 7, 8, 9 or 10), or more, nucleic acid sequence (e.g. mRNA) having an open reading frame, each of which encodes a different universal T-cell epitope (or a single nucleic acid sequence (e.g. mRNA) encoding 2-10, or more, different universal T-cell epitopes).

In some embodiments, the nucleic acid sequence (e.g. mRNA) encoding the infection agent antigenic polypeptide can elicit an antibody response in a subject. In some embodiments, the nucleic acid sequence (e.g. mRNA) encoding the infection agent antigenic polypeptide can elicit a broad T-cell response in a subject. In some embodiments, the nucleic acid sequence (e.g. mRNA) encoding the infection agent antigenic polypeptide can elicit both an antibody response and a T-cell response in a subject.

In some embodiments, the nucleic acid sequence (e.g. mRNA) encoding the infection agent antigenic polypeptide can elicit a cellular immune response, a humoral immune response, or a combination thereof. In some embodiments, the nucleic acid sequence (e.g. mRNA) encoding the infection agent antigenic polypeptide can elicit a cellular immune response, a humoral immune response, or a combination thereof, without risking the possibility of insertional mutagenesis. In some embodiments, the universal T-cell epitope can provide broader protection against infection agent variants.

In some embodiments, the compositions can further include a pharmaceutically acceptable carrier. In some aspects, disclosed herein is a pharmaceutical composition can include a nucleic acid sequence (e.g. mRNA) encoding an infection agent antigenic polypeptide, a nucleic acid sequence (e.g. mRNA) encoding at least one universal T-cell epitope, and a pharmaceutically acceptable carrier. In some aspects, disclosed herein is a pharmaceutical composition including an nucleic acid sequence (e.g. mRNA) encoding an infection agent antigenic polypeptide and at least one T-cell epitope; and a pharmaceutically acceptable carrier. In some embodiments, the composition can be a nanoparticle, a lipid nanoparticle dispersion, a liposomal formulation, a lipid emulsion, vaccine, vector, or any combination thereof.

In some embodiments, the nucleic acid sequence can include a DNA, RNA, any combination thereof (e.g., plasmid DNA, minicircle DNA, minimalistic, immunologically defined gene expression (MIDGE) and Doggybone, messenger RNA (mRNA), circle (cirRNA), self-amplifying-RNA (saRNA, also refer as SAM), or DNA launched SAM (DLSAM)). In some embodiments, the compositions can include a plasmid DNA(s) encoding an infection agent antigenic polypeptide and a universal T-cell epitope.

### Signal Peptides

In some embodiments, antigenic polypeptides encoded by nucleic acid sequence (e.g., mRNA) comprise a signal peptide. In some embodiments, T-cell epitope encoded by nucleic acid sequence (e.g., mRNA) comprise a signal peptide. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. Signal peptides generally include three regions: an N-terminal region of differing length, which usually comprises positively charged amino acids; a hydrophobic region; and a short carboxy-terminal peptide region. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane. The signal peptide, however, is not responsible for the final destination of the mature protein. Secretory proteins devoid of additional address tags in their sequence are by default secreted to the external environment. During recent years, a more advanced view of signal peptides has evolved, showing that the functions and immunodominance of certain signal peptides are much more versatile than previously anticipated.

The compositions described herein may include, for example, nucleic acid sequence (e.g., mRNA) encoding an artificial signal peptide, wherein the signal peptide coding sequence is operably linked to and is in frame with the coding sequence of the antigenic polypeptide, at least one universal T-cell epitope, or any combination thereof. Thus, composition of the present disclosure, in some embodiments, produce an antigenic polypeptide, at least one universal T-cell epitope, or any combination thereof comprising an antigenic polypeptide fused to a signal peptide. In some embodiments, a signal peptide is fused to the N-terminus of the antigenic polypeptide, at least one universal T-cell epitope, or any combination thereof. In some embodiments, a signal peptide is fused to the C-terminus of the antigenic polypeptide, at least one universal T-cell epitope, or any combination thereof.

In some embodiments, the signal peptide fused to the antigenic polypeptide, at least one universal T-cell epitope, or any combination thereof is an artificial signal peptide. In some embodiments, an artificial signal peptide fused to the antigenic polypeptide encoded by the composition is obtained from an immunoglobulin protein, e.g., an IgE signal peptide or an IgG signal peptide. In some embodiments, a signal peptide fused to the antigenic polypeptide, at least one universal T-cell epitope, or any combination thereof encoded by composition is an Ig heavy chain epsilon-1 signal peptide (IgE HC SP) having the sequence of: MDWTWILFLVAAATRVHS (SEQ ID NO: 16). In some embodiments, a signal peptide fused to the antigenic polypeptide, at least one universal T-cell epitope, or any combination thereof encoded by the composition is an IgGk chain V-III region HAH signal peptide (IgGk SP) having the sequence of METPAQLLFLLLLWLPDTTG (SEQ ID NO: 15). In some embodiments, the signal peptide is selected from: Japanese encephalitis PRM signal sequence (MLGSNSGQRVVFTILLLLVAPAYS: SEQ ID NO: 17), VSVg protein signal sequence (MKCLLYLAFLFIGVNCA; SEQ ID NO: 18) and Japanese encephalitis JEV signal sequence (MWLVSLAIVTACAGA; SEQ ID NO: 19).

In some embodiments, the antigenic polypeptide encoded by a composition comprises an amino acid sequence identified by any one of SEQ ID NO: 5-8, 12-13, 24-34, 47-50 or 54-56 fused to a signal peptide identified by any one of SEQ ID NO: 15-19. The examples disclosed herein are not meant to be limiting and any signal peptide that is known in the art to facilitate targeting of a protein to ER for processing and/or targeting of a protein to the cell membrane may be used in accordance with the present disclosure.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

A signal peptide is typically cleaved from the nascent polypeptide at the cleavage junction during ER processing. The mature antigenic polypeptide produce by a composition of the present disclosure typically does not comprise a signal peptide.

### Chemical Modifications

In some embodiments, antigenic polypeptides encoded by nucleic acid sequence (e.g., mRNA) comprise at least one chemical modification. In some embodiments, T-cell epitope encoded by nucleic acid sequence (e.g., mRNA) comprise at least one chemical modification.

The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population. Generally, these terms do not refer to the ribonucleotide modifications in naturally occurring 5′-terminal mRNA cap moieties. With respect to a polypeptide, the term “modification” refers to a modification relative to the canonical set 20 amino acids. Polypeptides, as provided herein, are also considered “modified” of they contain amino acid substitutions, insertions or a combination of substitutions and insertions.

Nucleic acids (e.g., RNA polynucleotides, such as mRNA polynucleotides), in some embodiments, comprise various (more than one) different modifications. In some embodiments, a particular region of a polynucleotide contains one, two or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Modifications of polynucleotides include, without limitation, those described herein. Nucleic acid sequences (e.g., RNA polynucleotides, such as mRNA polynucleotides) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

Nucleic acids (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

The present disclosure provides for modified nucleosides and nucleotides of a nucleic acid (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphdioester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

Modifications of nucleic acids (e.g., RNA polynucleotides, such as mRNA polynucleotides) that are useful in the composition of the present disclosure include, but are not limited to the following: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladeno sine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6,N6 (dimethyl)adenine; N6-cis-hydroxy-isopentenyl-adenosine; a-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP: 2′-Deoxy-2′-a-aminoadenosine TP; 2′-Deoxy-2′-a-azidoadenosine TP; 6 (alkyl)adenine; 6 (methyl)adenine: 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adeno sine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP: 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP: 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP: 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP: 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP: 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine: 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; a-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine: 3 (methyl)cytosine; 3-(alkyl)cytosine; 3-(deaza) 5 (aza)cytosine; 3-(methyl) cytidine: 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine: 5-(propynyl)cytosine; 5-(trifluoromethyl)cytosine; 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl) cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl) cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O)-methylguanosine; 2′-0)-ribosylguanosine (phosphate); 2′-O-methylguanosine: 2′-O-ribosylguanosine (phosphate); 7-aminomethyl-7-deazaguanosine; 7-cyano-7-deazaguanosine; Archaeosine; Methylwyo sine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine: 7-deaza-guanosine; 8-oxo-guanosine; N1-methyl-guanosine; a-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N(methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguano sine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine: 5-Carbamoylmethyluridine TP: 5-methoxycarbonylmethyl-2′-O-methyluridine; 5-methoxycarbonylmethyl-2-thiouridine; 5-methoxycarbonylmethyluridine; 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; a-thio-uridine; 1 (aminoalkylamino-carbonylethylenyl)-2 (thio)-pseudouracil; 1 (aminoalkylaminocarbonylethylenyl)-2,4-(dithio)pseudouracil; 1 (aminoalkylaminocarbonylethylenyl)-4 (thio)pseudouracil; 1 (aminoalkylaminocarbonylethylenyl)-pseudouracil; 1 (aminocarbonylethylenyl)-2 (thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2 (thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-Methyl-3-(3-amino-3-carboxypropyl)pseudouridine TP; 1-Methyl-3-(3-amino-3-carboxypropyl)pseudo-UTP; 1-Methyl-pseudo-UTP; 2 (thio)pseudouracil; 2′ deoxy uridine; 2′ fluorouridine; 2-(thio)uracil; 2,4-(dithio)psuedouracil; 2′ methyl, 2′amino, 2′azido, 2′fluro-guanosine; 2′-Amino-2′-deoxy-UTP: 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouracil; 4-(thio)pseudouracil; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-4 (thio)uracil; 5 (methylaminomethyl)-2,4 (dithio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2 (thio)uracil; 5-(methyl) 2,4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4 (dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (±) 1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1 (4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP: 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouridine TP; 1-{3-[2-(2-Aminoethoxy)-ethoxy]-propionyl}pseudouridine TP; 1-Acetylpseudouridine TP; 1-Alkyl-6-(1-propynyl)-pseudo-UTP: 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP: 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP: 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP: 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP: 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2 (2-ethoxy)-ethoxy}-ethoxy]-ethoxy)-ethoxy}]propionic acid; Pseudouridine TP 1-[3-{2-(2-[2-ethoxy]-ethoxy)-ethoxy}]propionic acid: Pseudouridine TP 1-[3-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino) purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino) purine; 2,4,5-(trimethyl)phenyl; 2′ methyl, 2′amino, 2′azido, 2′fluro-cytidine; 2′ methyl, 2′amino, 2′azido, 2′fluro-adenine; 2′methyl, 2′amino, 2′azido, 2′fluro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl) isocarbostyrilyl; 3-(methyl) isocarbostyrilyl; 4-(fluoro)-6-(methyl)benzimidazole; 4-(methyl)benzimidazole; 4-(methyl) indolyl: 4,6-(dimethyl) indolyl; 5 nitroindole; 5 substituted pyrimidines; 5-(methyl) isocarbostyrilyl; 5-nitroindole; 6-(aza)pyrimidine; 6-(azo) thymine; 6-(methyl)-7-(aza) indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza) indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl) isocarbostyrilyl; 7-(propynyl) isocarbostyrilyl, propynyl-7-(aza) indolyl; 7-deaza-inosinyl; 7-substituted 1-(aza)-2-(thio)-3-(aza)-phenoxazin-1-yl; 7-substituted 1.3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza) indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP: Formycin A TP; Formycin B TP; Pyrrolosine TP; 2′-OH-ara-adenosine TP; 2′-OH-ara-cytidine TP: 2′-OH-ara-uridine TP; 2′-OH-ara-guanosine TP; 5-(2-carbomethoxyvinyl)uridine TP; and N6-(19-Amino-pentaoxanonadecyl)adenosine TP.

In some embodiments, nucleic acids (e.g., RNA polynucleotides, such as mRNA polynucleotides) include a combination of at least two (e.g., 2, 3, 4 or more) of the aforementioned modified nucleobases.

In some embodiments, modified nucleobases in nucleic acids (e.g., RNA polynucleotides, such as mRNA polynucleotides) are selected from the group consisting of pseudouridine (ψ), N1-methylpseudouridine (mψ¹), N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, nucleic acids (e.g., RNA polynucleotides, such as mRNA polynucleotides) include a combination of at least two (e.g., 2, 3, 4 or more) of the aforementioned modified nucleobases.

In some embodiments, modified nucleobases in nucleic acids (e.g., RNA polynucleotides, such as mRNA polynucleotides) are selected from the group consisting of 1-methyl-pseudouridine (mψ¹), 5-methoxy-uridine (mo⁵U), 5-methyl-cytidine (m⁵C), pseudouridine (ψ), α-thio-guanosine and α-thio-adenosine. In some embodiments, nucleic acid includes a combination of at least two (e.g., 2, 3, 4 or more) of the aforementioned modified nucleobases.

In some embodiments, nucleic acids (e.g., RNA polynucleotides, such as mRNA polynucleotides) comprise pseudouridine (ψ) and 5-methyl-cytidine (m⁵C). In some embodiments, nucleic acids (e.g., RNA polynucleotides, such as mRNA polynucleotides) comprise 1-methyl-pseudouridine (mψ¹). In some embodiments, nucleic acids (e.g., RNA polynucleotides, such as mRNA polynucleotides) comprise 1-methyl-pseudouridine (mψ¹) and 5-methyl-cytidine (m⁵C). In some embodiments, nucleic acids (e.g., RNA polynucleotides, such as mRNA polynucleotides) comprise 2-thiouridine (s²U). In some embodiments, nucleic acids (e.g., RNA polynucleotides, such as mRNA polynucleotides) comprise 2-thiouridine and 5-methyl-cytidine (m⁵C). In some embodiments, nucleic acids (e.g., RNA polynucleotides, such as mRNA polynucleotides) comprise methoxy-uridine (mo⁵U). In some embodiments, nucleic acids (e.g., RNA polynucleotides, such as mRNA polynucleotides) comprise 5-methoxy-uridine (mo⁵U) and 5-methyl-cytidine (m⁵C). In some embodiments, nucleic acids (e.g., RNA polynucleotides, such as mRNA polynucleotides) comprise 2′-O-methyl uridine. In some embodiments, nucleic acids (e.g., RNA polynucleotides, such as mRNA polynucleotides) comprise 2′-O-methyl uridine and 5-methyl-cytidine (m⁵C). In some embodiments, nucleic acids (e.g., RNA polynucleotides, such as mRNA polynucleotides) comprise N6-methyl-adenosine (m⁶A). In some embodiments, nucleic acids (e.g., RNA polynucleotides, such as mRNA polynucleotides) comprise N6-methyl-adenosine (m⁶A) and 5-methyl-cytidine (m⁵C).

In some embodiments, nucleic acids (e.g., RNA polynucleotides, such as mRNA polynucleotides) are uniformly modified (e.g., fully modified, modified throughout the entire sequence) for a particular modification. For example, a nucleic acids can be uniformly modified with 5-methyl-cytidine (m⁵C), meaning that all cytosine residues in the mRNA sequence are replaced with 5-methyl-cytidine (m⁵C). Similarly, a nucleic acid can be uniformly modified for any type of nucleoside residue present in the sequence by replacement with a modified residue such as those set forth above.

Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleobase is a modified uridine. Exemplary nucleobases and in some embodiments, a modified nucleobase is a modified cytosine. nucleosides having a modified uridine include 5-cyano uridine, and 4′-thio uridine.

In some embodiments, a modified nucleobase is a modified adenine. Exemplary nucleobases and nucleosides having a modified adenine include 7-deaza-adenine, 1-methyl-adenosine (m1A), 2-methyl-adenine (m2A), and N6-methyl-adenosine (m⁶A).

In some embodiments, a modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine.

The nucleic acids described herein may be partially or fully modified along the entire length of the molecule. For example, one or more or all or a given type of nucleotide (e.g., purine or pyrimidine, or any one or more or all of A, G, U, C) may be uniformly modified in a polynucleotide of the disclosure, or in a given predetermined sequence region thereof (e.g., in the mRNA including or excluding the polyA tail). In some embodiments, all nucleotides X in a polynucleotide of the present disclosure (or in a given sequence region thereof) are modified nucleotides, wherein X may any one of nucleotides A. G, U, C, or any one of the combinations A+G, A+U, A+C, G+U, G+C, U+C, A+G+U, A+G+C, G+U+C or A+G+C.

The nucleic acids may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). Any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acid may contain a modified pyrimidine such as a modified uracil or cytosine. In some embodiments, at least 5%, at least 10%, at least 25%, at least 50%, at least 80%, at least 90% or 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the polynucleotide is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

Thus, in some embodiments, the compositions comprise a 5′UTR element, an optionally codon optimized open reading frame, and a 3′UTR element, a poly(A) sequence and/or a polyadenylation signal wherein the RNA is not chemically modified.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridineor 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm³U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm³se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (tm⁵U), 1-taurinomethyl-pseudouridine, 5-taurinomethyl-2-thio-uridine (tm⁵s²U), 1-taurinomethyl-4-thio-pseudouridine, 5-methyl-uridine (m5U, i.e., having the nucleobase deoxythymine), 1-methyl-pseudouridine (mψ¹), 5-methyl-2-thio-uridine (m5s2U), 1-methyl-4-thio-pseudouridine (m¹s⁴y), 4-thio-1-methyl-pseudouridine, 3-methyl-pseudouridine (m3v), 2-thio-1-methyl-pseudouridine, 1-methyl-1-deaza-pseudouridine, 2-thio-1-methyl-1-deaza-pseudouridine, dihydrouridine (D), dihydropseudouridine, 5.6-dihydrouridine, 5-methyldihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ w), 5-(isopentenylaminomethyl)uridine (inm⁵U), 5-(isopentenylaminomethyl)-2-thio-uridine (inm⁵s²U), a-thio-uridine. 2′-O-methyl-uridine (Urn), 5,2′-O-dimethyl-uridine (m5Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m3Um). and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl)uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formylcytidine (f⁵C), N4-methyl-cytidine (m4C), 5-methyl-cytidine (m⁵C), 5-halo-cytidine (e.g., 5-iodo-cytidine), 5-hydroxymethyl-cytidine (hm5° C.), 1-methyl-pseudoisocytidine, pyrrolo-cytidine, pyrrolo-pseudoisocytidine, 2-thio-cytidine (s2C). 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), a-thio-cytidine, 2′-O-methyl-cytidine (Cm), 5,2′-O-dimethylcytidine (m⁵Cm), N4-acetyl-2′-O-methyl-cytidine (ac⁴Cm), N4,2′-O-dimethylcytidine (m⁴Cm), 5-formyl-2′-O-methyl-cytidine (f⁵Cm), N4,N4,2′-O-trimethyl-cytidine (m₂⁴Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include 2-amino-purine, 2,6-diaminopurine. 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms² m⁶A), N6-isopentenyl-adenosine (i⁶A). 2-methylthio-N6-isopentenyl-adenosine (ms²i⁶A), N6-(cis-hydroxyisopentenyl)adenosine (i⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6,N6-dimethyl-adenosine (m⁶t⁶A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac6A), 7-methyl-adenine, 2-methylthio-adenine, 2-methoxy-adenine, a-thio-adenosine, 2′-O-methyl-adenosine (Am), N6,2′-O-dimethyl-adenosine (m6Am), N6,N6,2′-O-trimethyl-adenosine (ms₂⁶Am), 1,2′-O-dimethyl-adenosine (m1Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include inosine (I), 1-methyl-inosine (m¹I), wyosine (imG), methylwyosine (mimG), 4-demethyl-wyosine (imG-14), isowyosine (imG2), wybutosine (yW), peroxywybutosine (o_{2y}W), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G±), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m₂²G), N2,7-dimethyl-guano sine (m^{2,7}G), N2,N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, a-thio-guanosine, 2′-O-methyl-guanosine (Gm), N2-methyl-2′-O-methyl-guanosine (m²Gm), N2,N2-dimethyl-2′-O-methyl-guanosine (m₂²Gm), 1-methyl-2′-O-methyl-guanosine (m¹Gm), N2,7-dimethyl-2′-O-methyl-guanosine (m2.7Gm), 2′-O-methyl-inosine (Im), 1,2′-O-dimethyl-inosine (m¹Im), 2′-O-ribosylguanosine (phosphate) (Gr(p)). 1-thio-guanosine. 06-methyl-guanosine, 2′-F-ara-guanosine, and 2′-F-guanosine.

### N-Linked Glycosylation Site Mutants

N-linked glycans of viral proteins play important roles in modulating the immune response. Glycans can be important for maintaining the appropriate antigenic conformations, shielding potential neutralization epitopes, and may alter the proteolytic susceptibility of proteins. Some viruses have putative N-linked glycosylation sites. Deletion or modification of an N-linked glycosylation site may enhance the immune response. Thus, the present disclosure provides, in some embodiments, compositions comprising nucleic acids (e.g., mRNA) encoding antigenic polypeptides that comprise a deletion or modification at one or more N-linked glycosylation sites.

In Vitro Transcription of RNA (e.g., mRNA)

Compositions of the present disclosure comprise at least one RNA polynucleotide, such as a mRNA (e.g., modified mRNA). mRNA, for example, is transcribed in vitro from template DNA, referred to as an “in vitro transcription template.” In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

A “5′ untranslated region” (5′UTR) refers to a region of an mRNA that is directly upstream (i.e., 5′) from the start codon (i.e., the first codon of an mRNA transcript translated by a ribosome) that does not encode a polypeptide.

A “3′ untranslated region” (3′UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, export of the mRNA from the nucleus and translation.

In some embodiments, a polynucleotide includes 200 to 3,000 nucleotides. For example, a polynucleotide may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides.

### Flagellin Adjuvants

Flagellin is an approximately 500 amino acid monomeric protein that polymerizes to form the flagella associated with bacterial motion. Flagellin is expressed by a variety of flagellated bacteria (Salmonella typhimurium for example) as well as non-flagellated bacteria (such as Escherichia coli). Sensing of flagellin by cells of the innate immune system (dendritic cells, macrophages, etc.) is mediated by the Toll-like receptor 5 (TLR5) as well as by Nod-like receptors (NLRs) Ipaf and Naip5. TLRs and NLRs have been identified as playing a role in the activation of innate immune response and adaptive immune response. As such, flagellin provides an adjuvant effect in a vaccine.

The nucleotide and amino acid sequences encoding known flagellin polypeptides are publicly available in the NCBI GenBank database. The flagellin sequences from S. Typhimurium, H. Pylori, V. Cholera, S. marcescens, S. flexneri, T. Pallidum, L. pneumophila, B. burgdorferei, C. difficile, R. meliloti, A. tumefaciens, R. lupini, B. clarridgeiae, P. Mirabilis, B. subtilus, L. monocytogenes, P. aeruginosa, and E. coli, among others are known.

A flagellin polypeptide. as used herein, refers to a full length flagellin protein, immunogenic fragments thereof, and peptides having at least 50% sequence identify to a flagellin protein or immunogenic fragments thereof. Exemplary flagellin proteins include flagellin from Salmonella Entry typhi (UniPro number: Q56086), Salmonella typhimurium (A0A0C9DG09). Salmonella enteritidis (A0A0C9BAB7), and Salmonella choleraesuis (Q6V2X8), and SEQ ID NO: 54-56. In some embodiments, the flagellin polypeptide has at least 60%, 70%, 75%, 80%, 90%, 95%, 97%, 98%, or 99% sequence identify to a flagellin protein or immunogenic fragments thereof.

In some embodiments, the flagellin polypeptide is an immunogenic fragment. An immunogenic fragment is a portion of a flagellin protein that provokes an immune response. In some embodiments, the immune response is a TLR5 immune response. An example of an immunogenic fragment is a flagellin protein in which all or a portion of a hinge region has been deleted or replaced with other amino acids. For example, an antigenic polypeptide may be inserted in the hinge region. Hinge regions are the hypervariable regions of a flagellin. Hinge regions of a flagellin are also referred to as “D3 domain or region, “propeller domain or region,” “hypervariable domain or region” and “variable domain or region.” “At least a portion of a hinge region,” as used herein, refers to any part of the hinge region of the flagellin, or the entirety of the hinge region. In other embodiments an immunogenic fragment of flagellin is a 20, 25, 30, 35, or 40 amino acid C-terminal fragment of flagellin.

The flagellin monomer is formed by domains DO through D3. DO and D1, which form the stem, are composed of tandem long alpha helices and are highly conserved among different bacteria. The DI domain includes several stretches of amino acids that are useful for TLR5 activation. The entire DI domain or one or more of the active regions within the domain are immunogenic fragments of flagellin. Examples of immunogenic regions within the DI domain include residues 88-114 and residues 411-431 (in Salmonella typhimurium FliC flagellin. Within the 13 amino acids in the 88-100 region, at least 6 substitutions are permitted between Salmonella flagellin and other flagellins that still preserve TLR5 activation. Thus, immunogenic fragments of flagellin include flagellin like sequences that activate TLR5 and contain a 13 amino acid motif that is 53% or more identical to the Salmonella sequence in 88-100 of FliC (LQRVRELAVQSAN; SEQ ID NO: 84).

In some embodiments, the composition includes a nucleic acid sequence that encodes a fusion protein of flagellin and one or more antigenic polypeptides. A “fusion protein” as used herein, refers to a linking of two components of the construct. In some embodiments, a carboxy-terminus of the antigenic polypeptide is fused or linked to an amino terminus of the flagellin polypeptide. In other embodiments, an amino-terminus of the antigenic polypeptide is fused or linked to a carboxy-terminus of the flagellin polypeptide. The fusion protein may include, for example, one, two, three, four, five, six or more flagellin polypeptides linked to one, two, three, four, five, six or more antigenic polypeptides. When two or more flagellin polypeptides and/or two or more antigenic polypeptides are linked such a construct may be referred to as a “multimer.”

Each of the components of a fusion protein may be directly linked to one another or they may be connected through a linker. For instance, the linker may be an amino acid linker. The amino acid linker encoded for by the composition to link the components of the fusion protein may include, for instance, at least one member selected from the group consisting of a lysine residue, a glutamic acid residue, a serine residue and an arginine residue. In some embodiments the linker is 1-30, 1-25, 1-25, 5-10, 5, 15, or 5-20 amino acids in length.

In other embodiments, the composition includes at least three separate nucleic acid sequences, one encoding one or more antigenic polypeptides, one encoding one or more universal T-cell epitope, and the other encoding the flagellin polypeptide. The at least three nucleic acid sequences may be co-formulated in a carrier such as a lipid nanoparticle.

### Stabilizing Elements

Naturally-occurring eukaryotic mRNA molecules have been found to contain stabilizing elements, including, but not limited to untranslated regions (UTR) at their 5′-end (5′UTR) and/or at their 3′-end (3′UTR), in addition to other structural features, such as a 5′-cap structure or a 3′-poly(A) tail. Both the 5′UTR and the 3′UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing. The 3′-poly(A) tail is typically a stretch of adenine nucleotides added to the 3′-end of the transcribed mRNA. It can comprise up to about 400 adenine nucleotides. In some embodiments the length of the 3′-poly(A) tail may be an essential element with respect to the stability of the individual mRNA.

In some embodiments the nucleic acid sequence (e.g., mRNA) may include one or more stabilizing elements. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, the nucleic acid sequence (e.g., mRNA) include a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, B-Galactosidase, EGFP), or a marker or selection protein (e.g. alpha-Globin, Galactokinase and Xanthine: guanine phosphoribosyl transferase (GPT)).

In some embodiments, the combination of a poly(A) sequence or polyadenylation signal and at least one histone stem-loop, even though both represent alternative mechanisms in nature, acts synergistically to increase the protein expression beyond the level observed with either of the individual elements. It has been found that the synergistic effect of the combination of poly(A) and at least one histone stem-loop does not depend on the order of the elements or the length of the poly(A) sequence.

In some embodiments, the nucleic acid sequence (e.g., mRNA) does not comprise a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. Ideally, the inventive nucleic acid does not include an intron.

In some embodiments, the nucleic acid sequence (e.g., mRNA) may or may not contain an enhancer and/or promoter sequence, which may be modified or unmodified or which may be activated or inactivated. In some embodiments, the histone stem-loop is generally derived from histone genes, and includes an intramolecular base pairing of two neighbored partially or entirely reverse complementary sequences separated by a spacer, including (e.g., consisting of) a short sequence, which forms the loop of the structure. The unpaired loop region is typically unable to base pair with either of the stem loop elements. It occurs more often in RNA, as is a key component of many RNA secondary structures, but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In other embodiments the nucleic acid sequence (e.g., mRNA) may have one or more AU-rich sequences removed. These sequences, sometimes referred to as AURES are destabilizing sequences found in the 3′UTR. The AURES may be removed from the nucleic acid sequence (e.g., mRNA). Alternatively, the AURES may remain in the nucleic acid sequence (e.g., mRNA).

### Delivery Methods

Suitable nucleic acid delivery vehicles are well known in the art and can include, but are not limited to lipid-based (e.g., a liposome formulation, lipoplexes, or lipid nanoparticles (LNP)), viral-based, or physical methods such as injection, microinjection, electroporation, ultrasound, gene gun, hydrodynamic applications, or any combination thereof.

### Nanoparticles

Described herein are nanoparticles including a nucleic acid sequence (e.g., mRNA) encoding an infection agent antigenic polypeptide and a nucleic acid sequence (e.g., mRNA) encoding at least one universal T-cell epitope, as well as, nanoparticles including a nucleic acid sequence (e.g., mRNA) encoding an infection agent antigenic polypeptide and at least one universal T-cell epitope.

The nanoparticles described herein, can include multiple nucleic acid sequence (e.g., mRNA) each encoding a single infection agent antigenic polypeptide and multiple nucleic acid sequences (e.g., mRNA) each encoding a single universal T-cell epitope, as well as, nanoparticles including a single nucleic acid sequence (e.g., mRNA) encoding one or more infection agent antigenic polypeptide and a single nucleic acid sequence (e.g., mRNA) polynucleotide encoding one or more universal T-cell epitope.

The nanoparticles described herein, can include multiple nucleic acid sequence (e.g., mRNA) each encoding a single infection agent antigenic polypeptide and a single universal T-cell epitope, as well as, nanoparticles including a single nucleic acid sequence (e.g., mRNA) polynucleotides encoding one or more infection agent antigenic polypeptide and one or more universal T-cell epitope.

The nanoparticles described herein, in some embodiments, include 2-10 (e.g., 2, 3, 4, 5, 6, 7, 8, 9 or 10), or more, nucleic acid sequences having an open reading frame, each of which encodes a different infection agent antigenic polypeptide (or a single nucleic acid sequence encoding 2-10, or more, different infection agent antigenic polypeptides). The nanoparticles described herein, in some embodiments, include 2-10 (e.g., 2, 3, 4, 5, 6, 7, 8, 9 or 10), or more, nucleic acid sequence having an open reading frame, each of which encodes a different universal T-cell epitope (or a single nucleic acid sequence encoding 2-10, or more, different universal T-cell epitopes).

In some embodiments, the nanoparticle can be a lipid nanoparticle. In some embodiments, the nanoparticles can be a lipid-polycation complex, referred to as a cationic lipid nanoparticle. As a non-limiting example, the polycation may include a cationic peptide or a polypeptide such as, but not limited to, polylysine, polyornithine and/or polyarginine. In some embodiments, the lipid nanoparticle can include a non-cationic lipid such as, but not limited to, cholesterol or dioleoyl phosphatidylethanolamine (DOPE).

A lipid nanoparticle formulation may be influenced by, but not limited to, the selection of the cationic lipid component, the degree of cationic lipid saturation, the nature of the PEGylation, ratio of all components and biophysical parameters such as size. In one example by Semple et al. (Nature Biotech. 2010 28:172-176), the lipid nanoparticle can further include 57% cationic lipid, 7% dipalmitoylphosphatidylcholine, 34% cholesterol, and 1.5% PEG-c-DMA. As another example, changing the composition of the cationic lipid can more effectively deliver siRNA to various antigen presenting cells (Basha et al. Mol Ther. 2011 19:2186-2200).

In some embodiments, lipid nanoparticle formulations may comprise 35 to 45% cationic lipid, 40% to 50% cationic lipid, 50% to 60% cationic lipid and/or 55% to 65% cationic lipid. In some embodiments, the ratio of lipid to nucleic acid (e.g., mRNA) in lipid nanoparticles may be 5:1 to 20:1, 10:1 to 25:1, 15:1 to 30:1 and/or at least 30:1.

Lipid nanoparticle formulations typically comprise a lipid, in particular, an ionizable cationic lipid, and further comprise a neutral lipid, a sterol and a molecule capable of reducing particle aggregation, for example a PEG or PEG-modified lipid.

In some embodiments, a lipid nanoparticle formulation comprises at least one ionizable lipid, cationic lipid, vitamin-based lipid, or any combination thereof; a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of 20-60% cationic lipid: 5-25% neutral lipid: 25-55% sterol; 0.5-15% PEG-lipid.

In some embodiments, a lipid nanoparticle formulation includes 25% to 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy) heptadecanedioate (L319), e.g., 35 to 65%, 45 to 65%, 60%, 57.5%, 50% or 40% on a molar basis.

In some embodiments, lipid nanoparticle formulations include 25-75% of a cationic lipid, 0.5-15% of the neutral lipid, 5-50% of the sterol, and 0.5-20% of the PEG or PEG-modified lipid on a molar basis.

In some embodiments, lipid nanoparticle formulations include 35-65% of a cationic lipid, 3-12% of the neutral lipid, 15-45% of the sterol, and 0.5-10% of the PEG or PEG-modified lipid on a molar basis.

In some embodiments, lipid nanoparticle formulations include 45-65% of a cationic lipid, 5-10% of the neutral lipid, 25-40% of the sterol, and 0.5-10% of the PEG or PEG-modified lipid on a molar basis.

In some embodiments, lipid nanoparticle formulations include 60% of a cationic lipid, 7.5% of the neutral lipid, 31% of the sterol, and 1.5% of the PEG or PEG-modified lipid on a molar basis.

In some embodiments, lipid nanoparticle formulations include 50% of a cationic lipid, 10% of the neutral lipid, 38.5% of the sterol, and 1.5% of the PEG or PEG-modified lipid on a molar basis.

In some embodiments, lipid nanoparticle formulations include 50% of a cationic lipid, 10% of the neutral lipid, 35% of the sterol, 4.5% or 5% of the PEG or PEG-modified lipid, and 0.5% of the targeting lipid on a molar basis.

In some embodiments, lipid nanoparticle formulations include 40% of a cationic lipid, 15% of the neutral lipid, 40% of the sterol, and 5% of the PEG or PEG-modified lipid on a molar basis.

In some embodiments, lipid nanoparticle formulations include 57.2% of a cationic lipid, 7.1% of the neutral lipid, 34.3% of the sterol, and 1.4% of the PEG or PEG-modified lipid on a molar basis.

In some embodiments, lipid nanoparticle formulations include 57.5% of a cationic lipid selected from the PEG lipid is PEG-cDMA (PEG-CDMA is further discussed in Reyes et al. (J. Controlled Release, 107, 276-287 (2005), the contents of which are herein incorporated by reference in their entirety), 7.5% of the neutral lipid, 31.5% of the sterol, and 3.5% of the PEG or PEG-modified lipid on a molar basis.

In some embodiments, lipid nanoparticle formulations including a lipid mixture in molar ratios of 20-70% cationic lipid: 5-45% neutral lipid: 20-55% cholesterol: 0.5-15% PEG-modified lipid. In some embodiments, lipid nanoparticle formulations including a lipid mixture in a molar ratio of 20-60% cationic lipid: 5-25% neutral lipid: 25-55% cholesterol: 0.5-15% PEG-modified lipid.

In some embodiments, the molar lipid ratio is 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1/34.3/1.4 (mol % cationic lipid/neutral lipid, e.g., DPPC/Chol/PEG-modified lipid, e.g., PEG-cDMA), 40/15/40/5 (mol % cationic lipid/neutral lipid, e.g., DSPC/Chol/PEG-modified lipid, e.g., PEG-DMG). 50/10/35/4.5/0.5 (mol % cationic lipid/neutral lipid, e.g., DSPC/Chol/PEG-modified lipid, e.g., PEG-DSG), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-cDMA) or 52/13/30/5 (mol % cationic lipid/neutral lipid, e.g., DSPC/Chol/PEG-modified lipid, e.g., PEG-DMG or PEG-cDMA).

Non-limiting examples of lipid nanoparticle compositions and methods of making them are described, for example, in Semple et al. (2010) Nat. Biotechnol. 28:172-176; Jayarama et al. (2012), Angew. Chem. Int. Ed., 51:8529-8533; and Maier et al. (2013) Molecular Therapy 21, 1570-1578 (the contents of each of which are incorporated herein by reference in their entirety).

In some embodiments, lipid nanoparticle formulations may comprise a cationic lipid, a PEG lipid and a structural lipid and optionally comprise a non-cationic lipid. As a non-limiting example, a lipid nanoparticle may comprise 40-60% of cationic lipid, 5-15% of a non-cationic lipid, 1-2% of a PEG lipid and 30-50% of a structural lipid. As another non-limiting example, the lipid nanoparticle may comprise 50% cationic lipid, 10% non-cationic lipid, 1.5% PEG lipid and 38.5% structural lipid. As yet another non-limiting example, a lipid nanoparticle may comprise 55% cationic lipid, 10% non-cationic lipid, 2.5% PEG lipid and 32.5% structural lipid.

In some embodiments, the lipid nanoparticle formulations described herein may be 4 component lipid nanoparticles. The lipid nanoparticle may comprise a cationic lipid, a non-cationic lipid, a PEG lipid and a structural lipid. As a non-limiting example, the lipid nanoparticle may comprise 40-60% of cationic lipid, 5-15% of a non-cationic lipid, 1-2% of a PEG lipid and 30-50% of a structural lipid. As another non-limiting example, the lipid nanoparticle may comprise 50% cationic lipid, 10% non-cationic lipid, 1.5% PEG lipid and 38.5% structural lipid. As yet another non-limiting example, the lipid nanoparticle may comprise 55% cationic lipid, 10% non-cationic lipid, 2.5% PEG lipid and 32.5% structural lipid.

In some embodiments, the lipid nanoparticle formulations described herein may comprise a cationic lipid, a non-cationic lipid, a PEG lipid and a structural lipid. As a non-limiting example, the lipid nanoparticle comprises 50% of the cationic lipid DLin-KC2-DMA, 10% of the non-cationic lipid DSPC, 1.5% of the PEG lipid PEG-DOMG and 38.5% of the structural lipid cholesterol. As a non-limiting example, the lipid nanoparticle comprises 50% of the cationic lipid DLin-MC3-DMA, 10% of the non-cationic lipid DSPC, 1.5% of the PEG lipid PEG-DOMG and 38.5% of the structural lipid cholesterol. As a non-limiting example, the lipid nanoparticle comprises 50% of the cationic lipid DLin-MC3-DMA, 10% of the non-cationic lipid DSPC, 1.5% of the PEG lipid PEG-DMG and 38.5% of the structural lipid cholesterol. As yet another non-limiting example, the lipid nanoparticle comprises 55% of the cationic lipid L319, 10% of the non-cationic lipid DSPC. 2.5% of the PEG lipid PEG-DMG and 32.5% of the structural lipid cholesterol.

In some embodiments, the nanoparticles may be such as those described in U.S. Pat. No. 10,933,127, the contents of which is herein incorporated by reference in its entirety. In some embodiments, the nanoparticles may be such as those described in U.S. Pat. No. 10,933,127, for example, nanoparticles including a compound according to Formula (I), (Ia), (II), (IIa), (IIb), (IIc), (IId), or (IIe) described from column 101 to column 187.

In some embodiments, lipid nanoparticles can include 20% to 80% of an ionizable lipid, cationic lipid, vitamin based derivative lipid, or any combination thereof; greater than 0% to 5% polyethylene glycol-lipid; greater than 0% to 20% helper lipids; 20% to 80% sterol; a nucleic acid sequence encoding an infection agent antigenic polypeptide; and a nucleic acid sequence encoding at least one T-cell epitope encapsulated in the nanoparticle.

In some embodiments, lipid nanoparticles can include 20% to 80% of an ionizable lipid, cationic lipid, a vitamin based derivative lipid, or any combination thereof; greater than 0% to 5% polyethylene glycol-lipid; greater than 0% to 20% helper lipids; 20% to 80% sterol; and a nucleic acid sequence encoding an infection agent antigenic polypeptide and at least one T-cell epitope encapsulated in the nanoparticle.

In one embodiment, the nanoparticle can include from 40% to 60% of an ionizable lipid, cationic lipid, a vitamin based derivative lipid, or any combination thereof; from 1% to 2% polyethylene glycol-lipid; from 8% to 12% helper lipids; from 35% to 40% sterol; a nucleic acid sequence encoding an infection agent antigenic polypeptide; and a nucleic acid sequence encoding at least one T-cell epitope encapsulated in the nanoparticle.

In one embodiment, the nanoparticle can include from 40% to 60% of an ionizable lipid, cationic lipid, a vitamin based derivative lipid, or any combination thereof; from 1% to 2% polyethylene glycol-lipid; from 8% to 12% helper lipids; from 35% to 40% sterol; and a nucleic acid sequence encoding an infection agent antigenic polypeptide and at least one T-cell epitope encapsulated in the nanoparticle.

In some embodiments, a nanoparticle (e.g., a lipid nanoparticle) has a mean diameter of 10-500 nm, 20-400 nm, 30-300 nm, 40-200 nm. In some embodiments, a nanoparticle (e.g., a lipid nanoparticle) has a mean diameter of 50-150 nm, 50-200 nm, 80-100 nm or 80-200 nm.

The lipid nanoparticles described herein may be made in a sterile environment.

In some embodiments, the LNP formulation may be formulated in a nanoparticle such as a nucleic acid-lipid particle. As a non-limiting example, the lipid particle may comprise one or more active agents or therapeutic agents; one or more cationic lipids comprising from about 50 mol % to about 85 mol % of the total lipid present in the particle; one or more non-cationic lipids comprising from about 13 mol % to about 49.5 mol % of the total lipid present in the particle; and one or more conjugated lipids that inhibit aggregation of particles comprising from about 0.5 mol % to about 2 mol % of the total lipid present in the particle.

The nanoparticle formulations may comprise a phosphate conjugate. The phosphate conjugate may increase in vivo circulation times and/or increase the targeted delivery of the nanoparticle. As a non-limiting example, the phosphate conjugates may include a compound of any one of the formulas described in International Application No. WO2013033438, the contents of which are herein incorporated by reference in its entirety.

The nanoparticle formulation may comprise a polymer conjugate. The polymer conjugate may be a water soluble conjugate. The polymer conjugate may have a structure as described in U.S. patent application No. 20130059360, the contents of which are herein incorporated by reference in its entirety. In some embodiments, polymer conjugates with the polynucleotides of the present disclosure may be made using the methods and/or segmented polymeric reagents described in U.S. patent application No. 20130072709, the contents of which are herein incorporated by reference in its entirety. In some embodiments, the polymer conjugate may have pendant side groups comprising ring moieties such as, but not limited to, the polymer conjugates described in U.S. Patent Publication No. US20130196948, the contents which are herein incorporated by reference in its entirety.

The nanoparticle formulations may comprise a conjugate to enhance the delivery of nanoparticles of the present disclosure in a subject. Further, the conjugate may inhibit phagocytic clearance of the nanoparticles in a subject. In one aspect, the conjugate may be a “self”′ peptide designed from the human membrane protein CD47 (e.g., the “self” particles described by Rodriguez et al. (Science 2013 339, 971-975), herein incorporated by reference in its entirety). As shown by Rodriguez et al., the self peptides delayed macrophage-mediated clearance of nanoparticles which enhanced delivery of the nanoparticles. In another aspect, the conjugate may be the membrane protein CD47 (e.g., see Rodriguez et al. Science 2013 339, 971-975, herein incorporated by reference in its entirety). Rodriguez et al. showed that, similarly to “self” peptides, CD47 can increase the circulating particle ratio in a subject as compared to scrambled peptides and PEG coated nanoparticles.

In some embodiments, the nanoparticles can include a conjugate to enhance the delivery of the nanoparticles of the present disclosure in a subject. The conjugate may be the CD47 membrane or the conjugate may be derived from the CD47 membrane protein, such as the “self” peptide described previously. In some embodiments, the nanoparticle may comprise PEG and a conjugate of CD47 or a derivative thereof. In some embodiments, the nanoparticle may comprise both the “self” peptide described above and the membrane protein CD47.

In some embodiments, a “self” peptide and/or CD47 protein may be conjugated to a virus-like particle or pseudovirion, as described herein for delivery of the nucleic acid sequence (e.g., mRNA) of the present disclosure.

In some embodiments, compositions comprising the polynucleotides of the present disclosure and a conjugate that may have a degradable linkage. Non-limiting examples of conjugates include an aromatic moiety comprising an ionizable hydrogen atom, a spacer moiety, and a water-soluble polymer. As a non-limiting example, pharmaceutical compositions comprising a conjugate with a degradable linkage and methods for delivering such pharmaceutical compositions are described in U.S. Patent Publication No. US20130184443, the contents of which are herein incorporated by reference in their entirety.

### Neutral Lipid

In some embodiments, a lipid nanoparticle formulation includes 0.5% to 15% on a molar basis of the neutral lipid, e.g., 3 to 12%, 5 to 10% or 15%, 10%, or 7.5% on a molar basis. Examples of neutral lipids include, without limitation, DSPC, POPC, DPPC, DOPE and SM. In some embodiments, the formulation includes 5% to 50% on a molar basis of the sterol (e.g., 15 to 45%, 20 to 40%, 40%, 38.5%, 35%, or 31% on a molar basis. A non-limiting example of a sterol is cholesterol. In some embodiments, a lipid nanoparticle formulation includes 0.5% to 20% on a molar basis of the PEG or PEG-modified lipid (e.g., 0.5 to 10%, 0.5 to 5%, 1.5%, 0.5%, 1.5%, 3.5%, or 5% on a molar basis. In some embodiments, a PEG or PEG modified lipid comprises a PEG molecule of an average molecular weight of 2,000 Da. In some embodiments, a PEG or PEG modified lipid comprises a PEG molecule of an average molecular weight of less than 2,000, for example around 1,500 Da, around 1,000 Da, or around 500 Da. Non-limiting examples of PEG-modified lipids include PEG-distearoyl glycerol (PEG-DMG) (also referred herein as PEG-C14 or C14-PEG), PEG-CDMA (further discussed in Reyes et al. J. Controlled Release, 107, 276-287 (2005) the contents of which are herein incorporated by reference in their entirety).

### Zwitterionic Lipids

In some embodiments, the composition may be encapsulated in, linked to and/or associated with zwitterionic lipids. Non-limiting examples of zwitterionic lipids and methods of using zwitterionic lipids are described in U.S. Patent Publication No. US20130216607, the contents of which are herein incorporated by reference in their entirety. In some aspects, the zwitterionic lipids may be used in the liposomes and lipid nanoparticles described herein.

### Cationic Lipids

Suitable cationic lipids may include, but are not limited to, 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9, 12-dien-1-yloxy]-2-[(octyloxy)methyl]propan-1-ol (Compound 3 in US20130150625); and 2-(dimethylamino)-3-[(9Z, 12Z)-octadeca-9, 12-dien-1-yloxy]-2-{[(9Z,12Z)-octadeca-9,12-dien-1-yloxy]methyl}propan-1-ol (Compound 4 ir US20130150625); or any pharmaceutically acceptable salt or stereoisomer thereof. As a non-limiting example, the cationic lipid may be selected from (20Z,23Z)-N,N-dimethylnonacosa-20,23-dien-10-amine, (17Z,20Z)-N,N-dimemylhexacosa-17,20-dien-9-amine, (1Z,19Z)-N5N-dimethylpentacosa-16, 19-dien-8-amine, (13Z,16Z)-N,N-dimethyldocosa-13,16-dien-5-amine, (12Z, 15Z)-N,N-dimethylhenicosa-12,15-dien-4-amine, (14Z,17Z)-N,N-dimethyltricosa-14,17-dien-6-amine, (15Z,18Z)-N,N-dimethyltetracosa-15,18-dien-7-amine, (18Z,21Z)-N,N-dimethylheptacosa-18,21-dien-10-amine, (15Z,18Z)-N,N-dimethyltetracosa-15,18-dien-5-amine, (14Z,17Z)-N,N-dimethyltricosa-14,17-dien-4-amine, (19Z,22Z)-N,N-dimeihyloctacosa-19,22-dien-9-amine, (18Z,21 Z)-N,N-dimethylheptacosa-18,21-dien-8 amine, (17Z,20Z)-N,N-dimethylhexacosa-17,20-dien-7-amine, (16Z, 19Z)-N,N-dimethylpentacosa-16,19-dien-6-amine, (22Z,25Z)-N,N-dimethylhentriaconta-22,25-dien-10-amine, (21 Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-(20Z,23Z)-N-ethyl-N-methylnonacosa-20,23-dien-10-dimethylheptacosan-10-amine, amine, 1-[(11Z, 14Z)-1-nonylicosa-11,14-dien-1-yl]pyrrolidine, (20Z)-N,N-dimethylheptacos-20-en-10-amine, (15Z)-N,N-dimethyl eptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimethylhentriacont-22-en-10-amine, (16Z)-N,N-dimethylpentacos-16-en-8-amine, (12Z,15Z)-N,N-dimethyl-2-nonylhenicosa-12,15-dien-1-amine, (13Z,16Z)-N,N-dimethyl-3-nonyldocosa-13,16-dien-1 amine, N,N-dimethyl-1-[(1S,2R)-2-octylcyclopropylJeptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-](1S,2R)-2-octylcyclopropyl]henicosan-10-amine, N,N-dimethyl-1-[(1S,2S)-2-{[(1R,2R)-2-pentylcyclopropyl]methyl}cyclopropyl]nonadecan-10-amine,N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-RIS,2R)-2-octylcyclopropyllpentadecan-8-amine, R-N,N-dimethyl-1-[(9Z, 12Z)-octadeca-9, 12-dien-1-yloxy]-3-(octyloxy) propan-2-amine, S-N,N-dimethyl-1-[(9Z, 12Z)-octadeca-9, 12-dien-1-yloxy]-3-(octyloxy) propan-2-amine, 1-{2-[(9Z,12Z)-octadeca-9, 12-dien-1-yloxy]-1-[(octyloxy)methyl]ethyl}pyrrolidine, (2S)-N,N-dimethyl-1-[(9Z, 12Z)-octadeca-9,12-dien-1-yloxy]-3-[(5Z)-oct-5-en-1-yloxy]propan-2-amine, 1-{2-[(9Z, 12Z)-octadeca-9, 12-dien-1-yloxy]-1-[(octyloxy)methyl]ethyl}azetidine, (2S)-1-(hexyloxy)-N,N-dimethyl-3-R9Z,12Z)-octadeca-9,12-dien-1-yloxylpropan-2-amine, (2S)-1-(heptyloxy)-N,N-dimethyl-3-R9Z,12Z)-octadeca-9,12-dien-1-yloxylpropan-2-amine, N,N-dimethyl-1-(nonyloxy)-3-R9Z,12Z)-octadeca-9,12-dien-1-yloxylpropan-2-amine, N,N-dimethyl-1-[(9Z)-octadec-9-en-1-yloxy]-3-(octyloxy) propan-2-amine; (2S)-N,N-dimethyl-1-[(6Z,9Z,12Z)-octadeca-6,9, 12-trien-1-yloxy]-3-(octyloxy) propan-2-amine, (2S)-1-[(11Z,14Z)-icosa-11,14-dien-1-yloxy]-N,N-dimethyl-3-(pentyloxy) propan-2-amine, (2S)-1-(hexyloxy)-3-[(11Z,14Z)-icosa-11,14-dien-1-yloxy]-N,N-dimethylpropan-2-amine, 1-[(11Z, 14Z)-icosa-11,14-dien-1-yloxy]-N,N-dimethyl-3-(octyloxy) propan-2-amine, 1-[(13Z, 16Z)-docosa-13,16-dien-1-yloxy]-N,N-dimethyl-3-(octyloxy) propan-2-amine, (2S)-1-[(13Z, 16Z)-docosa-13, 16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy) propan-2-amine, 1-[(9Z)-hexadec-9-en-1-yloxy]-N,N-dimethyl-3-(octyloxy) propan-2-amine, (2R)-N,N-dimethyl-H (1-metoyloctyl)oxy]-3-[(9Z, 12Z)-octadeca-9, 12-dien-1-yloxy]propan-2-amine, (2R)-1-[(3,7-dimethyloctyl)oxy]-N,N-dimethyl-3-R9Z,12Z)-octadeca-9,12-dien-1-yloxylpropan-2-amine, N,N-dimethyl-1-(octyloxy)-3-({8-R1S,25)-2-{[(1R,2R)-2-pentylcyclopropyl]methyl}cyclopropyl]octyl}oxy) propan-2-amine, N,N-dimethyl-1-1 [8-(2-oclylcyclopropyl) octyl]oxy}-3-(octyloxy) propan-2-amine and (11E,20Z,23Z)-N,N-dimethylnonacosa-11,20,2-trien-10-amine or a pharmaceutically acceptable salt or stereoisomer thereof. In some embodiments, the lipid may be a cationic lipid such as, but not limited to, DLin-DMA, DLin-D-DMA. DLin-MC3-DMA, DLin-KC2-DMA, DODMA and amino alcohol lipids. The amino alcohol cationic lipid may be the lipids described in and/or made by the methods described in U.S. Patent Publication No. US20130150625, herein incorporated by reference in its entirety. In some embodiments, the nanoparticles described herein may include an amine cationic lipid such as those described in International Patent Application No. WO2013059496, the contents of which are herein incorporated by reference in their entirety. In some embodiments, the cationic lipids may have an amino-amine or an amino-amide moiety.

In some embodiments, the cationic lipid may be a low molecular weight cationic lipid such as those described in U.S. patent application No. 20130090372, the contents of which are herein incorporated by reference in their entirety.

### Ionizable Lipid

Exemplary ionizable lipids are described in the US patent publications Nos. U.S. 2016/0311759, U.S. 2015/0376115, U.S. 2016/0151284, U.S. 2017/0210697, U.S. 2015/0140070, U.S. 2013/0178541, U.S. 2013/0303587, U.S. 2015/0141678, U.S. 2015/0239926, U.S. 2016/0376224, U.S. 2017/0119904, U.S. 2012/0149894, U.S. 2015/0057373, U.S. 2013/0090372, U.S. 2013/0274523, U.S. 2013/0274504, U.S. 2013/0274504, U.S. 2009/0023673, U.S. 2012/0128760, U.S. 2010/03241240, U.S. 2014/0200257. U.S. 2015/0203446, U.S. 2018/0005363, U.S. 2014/0308304, U.S. 2013/0338210, U.S. 2012/0101148, U.S. 2012/0027796, U.S. 2012/0058144, U.S. 2013/0323269. U.S. 2011/0117125, U.S. 2011/0256175, U.S. 2012/0202871, U.S. 2011/0076335, U.S. 2006/0083780, U.S. 2013/0123338, U.S. 2015/0064242, U.S. 2006/0051405, U.S. 2013/0065939, U.S. 2006/0008910, U.S. 2003/0022649, U.S. 2010/0130588, U.S. 2013/0116307, U.S. 2010/0062967, U.S. 2013/0202684, U.S. 2014/0141070, U.S. 2014/0255472, U.S. 2014/0039032, U.S. 2018/0028664, U.S. 2016/0317458, U.S. 2013/0195920, U.S. 2022/0062175, U.S. 2021/0121411, U.S. 2022/0009878, U.S. 2022/0040325, U.S. 2012/61657480, U.S. 2016/0074514, U.S. 2013/0330401, U.S. 2019/0185410, U.S. 2012/61617468, U.S. 2019/0032087, U.S. 2015/62184188, U.S. 2019/0127318, U.S. 2021/0002813, U.S. 2020/0345641, U.S. 2014/61944336, U.S. 2012/61657480, U.S. 2021/0059953, U.S. 2022/0162521, U.S. 2022/0235377, U.S. 2018/0085474, U.S. 2018/0000953, U.S. 2020/0129445, U.S. 2021/0145982, U.S. 2021/0378980, U.S. 2020/0254086, U.S. 2021/0346306, and U.S. 2018/0000953, the contents of all of which are incorporated herein by reference in their entirety.

In some embodiments, the nanoparticle comprises an ionizable lipid in a molar ratio of from 0% to 80%. In some embodiments, the ionizable lipid can be present in a molar ratio of at least 0%, (e.g., at least 5%, at least 10%, at least 20%, at least 30%, at least 40%, at least 50%, at least 60%, at least 70%, or at least 80%). In some embodiments, the ionizable lipid can be present in a molar ratio of 80% or less, (e.g., 80% or less, 70% or less, 60% or less, 50% or less, 40% or less, 30% or less, 20% or less, 10% or less, 5% or less, 1% or less, or 0.5% or less).

The ionizable lipid can be present in a molar ratio ranging from any of the minimum values described above to any of the maximum values described above. For example, in some embodiments, the ionizable lipid can be present in a molar ratio of from 0% to 80% (e.g., from greater than 0% to 80%, from greater than 0% to 70%, from greater than 0% to 60%, from greater than 0% to 50%, from greater than 0% to 40%, from greater than 0% to 30%, from greater than 0% to 20%, from greater than 0% to 10%, from greater than 0% to 5%, from greater than 0% to 1%, from greater than 0% to 0.5%, from 1% to 30%, from 1% to 20%, from 1% to 10%, from 1% to 5%, from 5% to 30%, from 5% to 20%, from 5% to 10%, from 10% to 30%, from 10% to 20%, from 20% to 30%, from 20% to 40%, or from 30% to 40%).

### Helper Lipid

In some embodiments, the nanoparticle comprises a helper lipid. In some embodiments, the helper lipid can be a non-cationic lipid. In some embodiments, the non-cationic lipid can include, but is not limited to, 1,2-dioleoyl-sn-glycero-3-phosphoethanolamine (DOPE), 1-palmitoyl-2-oleoyl-sn-glycero-3-phosphoethanolamine (POPE), 1,2-distearoyl-sn-glycero-3-phosphocholine (DSPC), 1-stearoyl-2-oleoyl-sn-glycero-3-phosphoethanolamine (SOPE), DPPC (1,2-dipalmitoyl-sn-glycero-3-phosphocholine), 1,2-dioleyl-sn-glycero-3-phosphotidylcholine (DOPC), 1,2-dipalmitoyl-sn-glycero-3-phosphoethanolamine (DPPE), 1,2-dimyristoyl-sn-glycero-3-phosphoethanolamine (DMPE), 1,2-dioleoyl-5/7-glycero-3-phospho-(l′-rac-glycerol) (DOPG), or combinations thereof. In one embodiment, the non-cationic lipid is 1,2-dioleoyl-sn-glycero-3-phosphoethanolamine (DOPE). In one embodiment, the non-cationic lipid is 1-palmitoyl-2-oleoyl-sn-glycero-3-phosphoethanolamine (POPE), In one embodiment, the non-cationic lipid is 1,2-distearoyl-sn-glycero-3-phosphocholine (DSPC). In one embodiment, the non-cationic lipid is 1-stearoyl-2-oleoyl-sn-glycero-3-phosphoethanolamine (SOPE). While several non-cationic lipids are described here, additional non-cationic lipids can be used in combination with the compounds disclosed herein.

In some embodiments, the nanoparticle comprises a helper lipid in a molar ratio of from 0% to 20%. In some embodiments, the nanoparticle comprises a polyethylene glycol-lipid in a molar ratio of about 0%, 0.25%, 0.5%, 0.75%, 1%, 1.5%, 2%, 3%, 4%, 5%, 10%, 15%, or 20%.

### Polyethylene Glycol-Lipid

In some embodiments, the nanoparticle includes a polyethylene glycol-lipid (PEG-lipid). PEG-lipid is incorporated to form a hydrophilic outer layer and stabilize the particles. Nonlimiting examples of polyethylene glycol-lipids include PEG-modified lipids such as PEG-modified phosphatidylethanolamines, PEG-modified phosphatidic acids, PEG-modified ceramides, PEG-modified dialkylamines. PEG-modified diacylglycerols, and PEG-modified dialkylglycerols. Representative polyethylene glycol-lipids include DMG-PEG, DLPE-PEGs, DMPE-PEGs, DPPC-PEGs, and DSPE-PEGs. In one embodiment, the polyethylene glycol-lipid is 1,2-dimyristoyl-sn-glycerol, methoxypolyethylene glycol (DMG-PEG). In one embodiment, the polyethylene glycol-lipid is 1,2-dimyristoyl-sn-glycerol, methoxypolyethylene glycol-2000 (DMG-PEG2000). DMG-PEGXXXX means 1,2-dimyristoyl-sn-glycerol, methoxypolyethylene glycol-XXXX, wherein XXXX signifies the molecular weight of the polyethylene glycol moiety, e.g. DMG-PEG2000 or DMG-PEG5000.

In some embodiments, the nanoparticle comprises a polyethylene glycol-lipid in a molar ratio of from 0% to 5%. In some embodiments, the nanoparticle comprises a polyethylene glycol-lipid in a molar ratio of about 0%, 0.25%, 0.5%, 0.75%, 1%, 1.5%, 2%, 3%, 4%, or 5%. In one embodiment, the nanoparticle comprises a polyethylene glycol-lipid in a molar ratio of 0.75%.

In some embodiments, the ratio of PEG in the lipid nanoparticle formulations may be increased or decreased and/or the carbon chain length of the PEG lipid may be modified from C14 to C18 to alter the pharmacokinetics and/or biodistribution of the lipid nanoparticle formulations. As a non-limiting example, lipid nanoparticle formulations may contain 0.5% to 3.0%, 1.0% to 3.5%, 1.5% to 4.0%, 2.0% to 4.5%, 2.5% to 5.0% and/or 3.0% to 5.0% of the lipid molar ratio of PEG-c-DOMG (R-3-[(ω-methoxy-poly(ethyleneglycol) 2000) carbamoyl)]-1,2-dimyristyloxypropyl-3-amine) (also referred to herein as PEG-DOMG) as compared to the cationic lipid, DSPC and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol).

In some embodiments, the LNP formulations may contain PEG-c-DOMG at 3% lipid molar ratio. In some embodiments, the LNP formulations may contain PEG-c-DOMG at 1.5% lipid molar ratio.

In some embodiments, the pharmaceutical compositions may include at least one of the PEGylated lipids described in International Publication No. WO2012099755, the contents of which are herein incorporated by reference in their entirety.

In some embodiments, the LNP formulation may contain PEG-DMG 2000 (1,2-dimyristoyl-sn-glycero-3-phophoethanolamine-N-[methoxy (polyethylene glycol)-2000). In some embodiments, the LNP formulation may contain PEG-DMG 2000, a cationic lipid known in the art and at least one other component. In some embodiments, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example the LNP formulation may contain PEG-DMG 2000, DLin-DMA, DSPC and cholesterol in a molar ratio of 2:40:10:48 (see e.g., Geall et al., Nonviral delivery of self-amplifying RNA (e.g., mRNA) vaccines, PNAS 2012; PMID: 22908294, the contents of each of which are herein incorporated by reference in their entirety).

### Sterol

In some embodiments, the nanoparticle includes a sterol. Sterols are well known to those skilled in the art and generally refers to those compounds having a perhydrocyclopentanophenanthrene ring system and having one or more OH substituents. Examples of sterols include, but are not limited to, cholesterol, campesterol, ergosterol, sitosterol, and the like.

In some embodiments, the sterol is selected from a cholesterol-based lipid. In some embodiments, the one or more cholesterol-based lipids are selected from cholesterol, PEGylated cholesterol, DC-Choi (N,N-dimethyl-N-ethylcarboxamidocholesterol), 1,4-bis(3-N-oleylamino-propyl) piperazine, or combinations thereof.

The sterol can be used to tune the particle permeability and fluidity base on its function in cell membranes. In one embodiment, the sterol is cholesterol.

In some embodiments, the nanoparticle comprises a sterol in a molar ratio of from 20% to 80%. In some embodiments, the nanoparticle comprises a sterol in a molar ratio of 25%, 30%, 35%, 40%, 45%, or 50%. In one embodiment, the nanoparticle comprises a sterol in a molar ratio of 40%.

The nanoparticle formulations may be a carbohydrate nanoparticle comprising a carbohydrate carrier and a nucleic acid sequence (e.g., mRNA) described herein. As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121; the contents of which are herein incorporated by reference in their entirety).

Nanoparticle formulations of the present disclosure may be coated with a surfactant or polymer in order to improve the delivery of the particle. In some embodiments, the nanoparticle may be coated with a hydrophilic coating such as, but not limited to, PEG coatings and/or coatings that have a neutral surface charge. The hydrophilic coatings may help to deliver nanoparticles with larger payloads such as, but not limited to, RNA (e.g., mRNA) within the central nervous system. As a non-limiting example nanoparticles comprising a hydrophilic coating and methods of making such nanoparticles are described in U.S. Patent Publication No. US20130183244, the contents of which are herein incorporated by reference in their entirety.

In some embodiments, the lipid nanoparticles of the present disclosure may be hydrophilic polymer particles. Non-limiting examples of hydrophilic polymer particles and methods of making hydrophilic polymer particles are described in U.S. Patent Publication No. US20130210991, the contents of which are herein incorporated by reference in their entirety.

In some embodiments, the lipid nanoparticles of the present disclosure may be hydrophobic polymer particles.

In some embodiments, an immune response may be elicited by delivering a lipid nanoparticle which may include a nanospecies, a polymer and an immunogen. (U.S. Publication No. 20120189700 and International Publication No. WO2012099805; each of which is herein incorporated by reference in their entirety). The polymer may encapsulate the nanospecies or partially encapsulate the nanospecies. The immunogen may be a recombinant protein, a modified RNA and/or a polynucleotide described herein. In some embodiments, the lipid nanoparticle may be formulated for use in a vaccine such as, but not limited to, against a pathogen.

Lipid nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosa tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. PNAS 2007 104 (5): 1482-487; Lai et al. Adv Drug Deliv Rev. 2009 61 (2): 158-171; each of which is herein incorporated by reference in their entirety). The transport of nanoparticles may be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier may be made as described in U.S. Pat. No. 8,241,670 or International Patent Publication No. WO2013110028, the contents of each of which are herein incorporated by reference in its entirety.

The lipid nanoparticle engineered to penetrate mucus may comprise a polymeric material (i.e. a polymeric core) and/or a polymer-vitamin conjugate and/or a tri-block co-polymer. The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates. The polymeric material may be biodegradable and/or biocompatible. Non-limiting examples of biocompatible polymers are described in International Patent Publication No. WO2013116804, the contents of which are herein incorporated by reference in their entirety. The polymeric material may additionally be irradiated. As a non-limiting example, the polymeric material may be gamma irradiated (see e.g., International App. No. WO201282165, herein incorporated by reference in its entirety). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho) esters, poly(butyric acid), poly(valeric acid), poly(lactide-co-caprolactone), PEG-PLGA-PEG and trimethylene carbonate, polyvinylpyrrolidone. The lipid nanoparticle may be coated or associated with a co-polymer such as, but not limited to, a block co-polymer (such as a branched polyether-polyamide block copolymer described in International Publication No. WO2013012476, herein incorporated by reference in its entirety), and (poly(ethylene glycol))-(poly(propylene oxide))-(poly(ethylene glycol))triblock copolymer (see e.g., U.S. Publication 20120121718 and U.S. Publication 20100003337 and U.S. Pat. No. 8,263,665, the contents of each of which is herein incorporated by reference in their entirety). The co-polymer may be a polymer that is generally regarded as safe (GRAS) and the formation of the lipid nanoparticle may be in such a way that no new chemical entities are created. For example, the lipid nanoparticle may comprise poloxamers coating PLGA nanoparticles without forming new chemical entities which are still able to rapidly penetrate human mucus (Yang et al. Angew. Chem. Int. Ed. 2011 50:2597-2600; the contents of which are herein incorporated by reference in their entirety). A non-limiting scalable method to produce nanoparticles which can penetrate human mucus is described by Xu et al. (see, e.g., J Control Release 2013, 170 (2): 279-86; the contents of which are herein incorporated by reference in their entirety).

The vitamin of the polymer-vitamin conjugate may be vitamin E. The vitamin portion of the conjugate may be substituted with other suitable components such as, but not limited to, vitamin A, vitamin E, other vitamins, cholesterol, a hydrophobic moiety, or a hydrophobic component of other surfactants (e.g., sterol chains, fatty acids, hydrocarbon chains and alkylene oxide chains).

The lipid nanoparticle engineered to penetrate mucus may include surface altering agents such as, but not limited to, polynucleotides, anionic proteins (e.g., bovine serum albumin), surfactants (e.g., cationic surfactants such as for example dimethyldioctadecylammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4 dornase alfa, neltenexine, erdosteine) and various DNases including rhDNase. The surface altering agent may be embedded or enmeshed in the particle's surface or disposed (e.g., by coating, adsorption, covalent linkage, or other process) on the surface of the lipid nanoparticle. (see e.g., U.S. Publication 20100215580 and U.S. Publication 20080166414 and US20130164343; the contents of each of which are herein incorporated by reference in their entirety).

In some embodiments, the mucus penetrating lipid nanoparticles may comprise at least one polynucleotide described herein. The polynucleotide may be encapsulated in the lipid nanoparticle and/or disposed on the surface of the particle. The polynucleotide may be covalently coupled to the lipid nanoparticle. Formulations of mucus penetrating lipid nanoparticles may comprise a plurality of nanoparticles.

In some embodiments, the compositions can be formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In some embodiments, the lipid nanoparticle may be a self-assembly lipid-polymer nanoparticle (see Zhang et al., ACS Nano, 2008, 2 (8), pp 1696-1702; the contents of which are herein incorporated by reference in their entirety). As a non-limiting example, the SLN may be the SLN described in International Patent Publication No. WO2013105101, the contents of which are herein incorporated by reference in their entirety. As another non-limiting example, the SLN may be made by the methods or processes described in International Patent Publication No. WO2013105101, the contents of which are herein incorporated by reference in their entirety.

In some embodiments, the compositions of the present disclosure may be encapsulated in a nanoparticle. Nanoparticles may be formulated by methods described herein and known in the art such as, but not limited to, International Pub Nos. WO2010005740, WO2010030763, WO2010005721, WO2010005723, WO2012054923, U.S. Publication Nos. US20110262491, US20100104645, US20100087337, US20100068285, US20110274759, US20100068286, US20120288541, US20130123351 and US20130230567 and U.S. Pat. Nos. 8,206,747, 8,293,276, 8,318,208 and 8,318,211; the contents of each of which are herein incorporated by reference in their entirety. In some embodiments, polymer nanoparticles may be identified by the methods described in US Pub No. US20120140790, the contents of which are herein incorporated by reference in their entirety.

In some embodiments, the nanoparticle may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and nucleic acid sequence of the present disclosure (see International Pub No. 2010075072 and US Pub No. US20100216804, US20110217377 and US20120201859, the contents of each of which are incorporated herein by reference in their entirety). In another non-limiting example, the sustained release formulation may comprise agents which permit persistent bioavailability such as, but not limited to, crystals, macromolecular gels and/or particulate suspensions (see U.S. Patent Publication No US20130150295, the contents of each of which are incorporated herein by reference in their entirety).

In some embodiments, the nanoparticles of the present disclosure may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters. poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In some embodiments, the nanoparticle comprises a diblock copolymer. In some embodiments, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine. poly(ethylene imine), poly(serine ester), poly(L-lactide-co-L-lysine), poly(4-hydroxy-L-proline ester) or combinations thereof. In yet another embodiment, the diblock copolymer may be a high-X diblock copolymer such as those described in International Patent Publication No. WO2013120052, the contents of which are incorporated herein by reference in their entirety.

As a non-limiting example, the nanoparticle comprises a PLGA-PEG block copolymer (see U.S. Publication No. US20120004293 and U.S. Pat. No. 8,236,330, each of which is herein incorporated by reference in their entirety). In another non-limiting example, the therapeutic nanoparticle is a stealth nanoparticle comprising a diblock copolymer of PEG and PLA or PEG and PLGA (see U.S. Pat. No. 8,246,968 and International Publication No. WO2012166923, the contents of each of which are herein incorporated by reference in their entirety). In yet another non-limiting example, the nanoparticle is a stealth nanoparticle or a target-specific stealth nanoparticle as described in U.S. Patent Publication No. US20130172406, the contents of which are herein incorporated by reference in their entirety.

In some embodiments, the nanoparticle may comprise a multiblock copolymer (see e.g., U.S. Pat. Nos. 8,263,665 and 8.287,910 and U.S. Patent Pub. No. US20130195987, the contents of each of which are herein incorporated by reference in their entirety).

In yet another non-limiting example, the lipid nanoparticle comprises the block copolymer PEG-PLGA-PEG (see e.g., the thermosensitive hydrogel (PEG-PLGA-PEG) was used as a TGF-beta1 gene delivery vehicle in Lee et al. Thermosensitive Hydrogel as a Tgf-β1 Gene Delivery Vehicle Enhances Diabetic Wound Healing. Pharmaceutical Research, 2003 20(12):1995-2000; as a controlled gene delivery system in Li et al. Controlled Gene Delivery System Based on Thermosensitive Biodegradable Hydrogel. Pharmaceutical Research 2003 20(6):884-888; and Chang et al., Non-ionic amphiphilic biodegradable PEG-PLGA-PEG copolymer enhances gene delivery efficiency in rat skeletal muscle. J Controlled Release. 2007 118:245-253, the contents of each of which are herein incorporated by reference in their entirety). The nucleic acid (e.g., mRNA) vaccines of the present disclosure may be formulated in lipid nanoparticles comprising the PEG-PLGA-PEG block copolymer.

In some embodiments, the nanoparticle may comprise a multiblock copolymer (see e.g., U.S. Pat. Nos. 8,263,665 and 8,287,910 and U.S. Patent Pub. No. US20130195987, the contents of each of which are herein incorporated by reference in their entirety).

In some embodiments, the block copolymers described herein may be included in a polyion complex comprising a non-polymeric micelle and the block copolymer. (see e.g., U.S. Publication No. 20120076836, the contents of which are herein incorporated by reference in their entirety).

In some embodiments, the nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In some embodiments, the nanoparticles may comprise at least one poly(vinyl ester) polymer. The poly(vinyl ester) polymer may be a copolymer such as a random copolymer. As a non-limiting example, the random copolymer may have a structure such as those described in International Application No. WO2013032829 or U.S. Patent Publication No US20130121954, the contents of each of which are herein incorporated by reference in their entirety. In some embodiments, the poly(vinyl ester) polymers may be conjugated to the polynucleotides described herein.

In some embodiments, the nanoparticle may include at least one diblock copolymer. The diblock copolymer may be, but it not limited to, a poly(lactic) acid-poly(ethylene)glycol copolymer (see, e.g., International Patent Publication No. WO2013044219, the contents of which are herein incorporated by reference in their entirety). As a non-limiting example, the nanoparticle may be used to treat cancer (see International publication No. WO2013044219, the contents of which are herein incorporated by reference in their entirety).

In some embodiments, the nanoparticles may include at least one cationic polymer described herein and/or known in the art.

In some embodiments, the nanoparticles may include at least one amine-containing polymer such as, but not limited to polylysine, polyethylene imine, poly(amidoamine) dendrimers, poly(beta-amino esters) (see, e.g., U.S. Pat. No. 8,287,849, the contents of which are herein incorporated by reference in their entirety) and combinations thereof.

In some embodiments, the nanoparticles may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In some embodiments, the degradable polyesters may include a PEG conjugation to form a PEGylated polymer.

In some embodiments, the compositions may be formulated in colloid nanocarriers as described in U.S. Patent Publication No. US20130197100, the contents of which are herein incorporated by reference in their entirety.

In some embodiments, the nanoparticle may be optimized for oral administration. The nanoparticle may comprise at least one cationic biopolymer such as, but not limited to, chitosan or a derivative thereof. As a non-limiting example, the nanoparticle may be formulated by the methods described in U.S. Publication No. 20120282343, the contents of which are herein incorporated by reference in their entirety.

In some embodiments, LNPs comprise the lipid KL52 (an amino-lipid disclosed in U.S. Application Publication No. 2012/0295832, the contents of which are herein incorporated by reference in their entirety. Activity and/or safety (as measured by examining one or more of ALT/AST, white blood cell count and cytokine induction, for example) of LNP administration may be improved by incorporation of such lipids. LNPs comprising KL52 may be administered intravenously and/or in one or more doses. In some embodiments, administration of LNPs comprising KL52 results in equal or improved mRNA and/or protein expression as compared to LNPs comprising MC3.

In some embodiments, the lipid nanoparticle may be a limit size lipid nanoparticle described in International Patent Publication No. WO2013059922, the contents of which are herein incorporated by reference in their entirety. The limit size lipid nanoparticle may comprise a lipid bilayer surrounding an aqueous core or a hydrophobic core; where the lipid bilayer may comprise a phospholipid such as, but not limited to, diacylphosphatidylcholine, a diacylphosphatidylethanolamine, a ceramide, a sphingomyelin, a dihydrosphingomyelin, a cephalin, a cerebroside, a C8-C20 fatty acid diacylphophatidylcholine, and 1-palmitoyl-2-oleoyl phosphatidylcholine (POPC). In some embodiments, the limit size lipid nanoparticle may comprise a polyethylene glycol-lipid such as, but not limited to, DLPE-PEG, DMPE-PEG, DPPC-PEG and DSPE-PEG.

In some embodiments, the compositions may be formulated in a nanoparticle comprising an inner core comprising a non-cellular material and an outer surface comprising a cellular membrane. The cellular membrane may be derived from a cell or a membrane derived from a virus. As a non-limiting example, the nanoparticle may be made by the methods described in International Patent Publication No. WO2013052167, the contents of which are herein incorporated by reference in their entirety. As another non-limiting example, the nanoparticle described in International Patent Publication No. WO2013052167, the contents of which are herein incorporated by reference in their entirety, may be used to deliver the compositions described herein.

In some embodiments, the compositions may be formulated in porous nanoparticle-supported lipid bilayers (protocells). Protocells are described in International Patent Publication No. WO2013056132, the contents of which are herein incorporated by reference in their entirety.

In some embodiments, the compositions described herein may be formulated in polymeric nanoparticles as described in or made by the methods described in U.S. Pat. Nos. 8,420,123 and 8,518,963 and European Patent No. EP2073848B1, the contents of each of which are herein incorporated by reference in their entirety. As a non-limiting example, the polymeric nanoparticle may have a high glass transition temperature such as the nanoparticles described in or nanoparticles made by the methods described in U.S. Pat. No. 8,518,963, the contents of which are herein incorporated by reference in their entirety. As another non-limiting example, the polymer nanoparticle for oral and parenteral formulations may be made by the methods described in European Patent No. EP2073848B1, the contents of which are herein incorporated by reference in their entirety.

In some embodiments, the compositions described herein may be formulated in nanoparticles used in imaging. The nanoparticles may be liposome nanoparticles such as those described in U.S. Patent Publication No US20130129636, herein incorporated by reference in its entirety. As a non-limiting example, the liposome may comprise gadolinium (III) 2-{4,7-bis-carboxymethyl-10-[(N,N-distearylamidomethyl-N′-amido-methyl]-1,4,7,10-tetra-azacyclododec-1-yl}-acetic acid and a neutral, fully saturated phospholipid component (see, e.g., U.S. Patent Publication No US20130129636, the contents of which are herein incorporated by reference in their entirety).

In some embodiments, the nanoparticles which may be used in the present disclosure are formed by the methods described in U.S. Patent Application No. US20130130348, the contents of which are herein incorporated by reference in their entirety.

In some embodiments, the compositions of the present disclosure may be formulated in a swellable nanoparticle. The swellable nanoparticle may be, but is not limited to, those described in U.S. Pat. No. 8,440,231, the contents of which are herein incorporated by reference in their entirety. As a non-limiting embodiment, the swellable nanoparticle may be used for delivery of the RNA (e.g., mRNA) vaccines of the present disclosure to the pulmonary system (see, e.g., U.S. Pat. No. 8,440,231, the contents of which are herein incorporated by reference in their entirety).

The compositions of the present disclosure may be formulated in polyanhydride nanoparticles such as, but not limited to, those described in U.S. Pat. No. 8,449,916, the contents of which are herein incorporated by reference in their entirety.

The nanoparticles and microparticles of the present disclosure may be geometrically engineered to modulate macrophage and/or the immune response. In some embodiments, the geometrically engineered particles may have varied shapes, sizes and/or surface charges in order to incorporated the polynucleotides of the present disclosure for targeted delivery such as, but not limited to, pulmonary delivery (see, e.g., International Publication No WO2013082111, the contents of which are herein incorporated by reference in their entirety). Other physical features the geometrically engineering particles may have include, but are not limited to, fenestrations, angled arms, asymmetry and surface roughness, charge which can alter the interactions with cells and tissues. As a non-limiting example, nanoparticles of the present disclosure may be made by the methods described in International Publication No WO2013082111, the contents of which are herein incorporated by reference in their entirety.

In some embodiments, the nanoparticles of the present disclosure may be water soluble nanoparticles such as, but not limited to, those described in International Publication No. WO2013090601, the contents of which are herein incorporated by reference in their entirety. The nanoparticles may be inorganic nanoparticles which have a compact and zwitterionic ligand in order to exhibit good water solubility. The nanoparticles may also have small hydrodynamic diameters (HD), stability with respect to time, pH, and salinity and a low level of non-specific protein binding.

In some embodiments, the nanoparticles of the present disclosure may be developed by the methods described in U.S. Patent Publication No. US20130172406, the contents of which are herein incorporated by reference in their entirety.

In some embodiments, the nanoparticles of the present disclosure are stealth nanoparticles or target-specific stealth nanoparticles such as, but not limited to, those described in U.S. Patent Publication No. US20130172406, the contents of which are herein incorporated by reference in their entirety. The nanoparticles of the present disclosure may be made by the methods described in U.S. Patent Publication No. US20130172406, the contents of which are herein incorporated by reference in their entirety.

In some embodiments, the stealth or target-specific stealth nanoparticles may comprise a polymeric matrix. The polymeric matrix may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates or combinations thereof.

In some embodiments, the nanoparticle may be a nanoparticle-nucleic acid hybrid structure having a high-density nucleic acid layer. As a non-limiting example, the nanoparticle-nucleic acid hybrid structure may made by the methods described in U.S. Patent Publication No. US20130171646, the contents of which are herein incorporated by reference in their entirety. The nanoparticle may comprise a nucleic acid such as, but not limited to, polynucleotides described herein and/or known in the art.

At least one of the nanoparticles of the present disclosure may be embedded in the core a nanostructure or coated with a low density porous 3-D structure or coating which is capable of carrying or associating with at least one payload within or on the surface of the nanostructure. Non-limiting examples of the nanostructures comprising at least one nanoparticle are described in International Patent Publication No. WO2013123523, the contents of which are herein incorporated by reference in their entirety.

In some embodiments, compositions may be delivered using smaller LNPs. Such particles may comprise a diameter from below 0.1 μm up to 100 nm such as, but not limited to, less than 0.1 μm, less than 1.0 μm, less than 5 μm, less than 10 μm, less than 15 μm, less than 20 μm, less than 25 μm, less than 30 μm, less than 35 μm, less than 40 μm, less than 50 μm, less than 55 μm, less than 60 μm, less than 65 μm, less than 70 μm, less than 75 μm, less than 80 μm, less than 85 μm, less than 90 μm, less than 95 μm, less than 100 μm, less than 125 μm, less than 150 μm, less than 175 μm, less than 200 μm, less than 225 μm, less than 250 μm, less than 275 μm, less than 300 μm, less than 325 μm, less than 350 μm, less than 375 μm, less than 400 μm, less than 425 μm, less than 450 μm, less than 475 μm, less than 500 μm, less than 525 μm, less than 550 μm, less than 575 μm, less than 600 μm, less than 625 μm, less than 650 μm, less than 675 μm, less than 700 μm, less than 725 μm, less than 750 μm, less than 775 μm, less than 800 μm, less than 825 μm, less than 850 μm, less than 875 μm, less than 900 μm, less than 925 μm, less than 950 μm, less than 975 μm, or less than 1000 μm.

In some embodiments, compositions may be delivered using smaller LNPs, which may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nm, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm. from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm.

In some embodiments, such LNPs are synthesized using methods comprising microfluidic mixers. Examples of microfluidic mixers may include, but are not limited to, a slit interdigital micromixer including, but not limited to those manufactured by Microinnova (Allerheiligen bei Wildon, Austria) and/or a staggered herringbone micromixer (SHM) (Zhigaltsev, I. V. et al., Bottom-up design and synthesis of limit size lipid nanoparticle systems with aqueous and triglyceride cores using millisecond microfluidic mixing have been published (Langmuir. 2012. 28:3633-40; Belliveau, N. M. et al., Microfluidic synthesis of highly potent limit-size lipid nanoparticles for in vivo delivery of siRNA. Molecular Therapy-Nucleic Acids. 2012. 1: e37; Chen, D. et al., Rapid discovery of potent siRNA-containing lipid nanoparticles enabled by controlled microfluidic formulation. J Am Chem Soc. 2012. 134 (16): 6948-51, the contents of each of which are herein incorporated by reference in their entirety). In some embodiments, methods of LNP generation comprising SHM, further comprise the mixing of at least two input streams wherein mixing occurs by microstructure-induced chaotic advection (MICA). According to this method, fluid streams flow through channels present in a herringbone pattern causing rotational flow and folding the fluids around each other. This method may also comprise a surface for fluid mixing wherein the surface changes orientations during fluid cycling. Methods of generating LNPs using SHM include those disclosed in U.S. Application Publication Nos. 2004/0262223 and 2012/0276209, the contents of each of which are herein incorporated by reference in their entirety.

In some embodiments, the compositions of the present disclosure may be formulated in lipid nanoparticles created using a micromixer such as, but not limited to, a Slit Interdigital Microstructured Mixer (SIMM-V2) or a Standard Slit Interdigital Micro Mixer (SSIMM) or Caterpillar (CPMM) or Impinging-jet (IJMM) from the Institut für Mikrotechnik Mainz GmbH, Mainz Germany).

In some embodiments, the compositions of the present disclosure may be formulated in lipid nanoparticles created using microfluidic technology (see, e.g., Whitesides, George M. The Origins and the Future of Microfluidics. Nature, 2006 442:368-373; and Abraham et al. Chaotic Mixer for Microchannels. Science, 2002 295:647-651; each of which is herein incorporated by reference in its entirety). As a non-limiting example, controlled microfluidic formulation includes a passive method for mixing streams of steady pressure-driven flows in micro channels at a low Reynolds number (see, e.g., Abraham et al. Chaotic Mixer for Microchannels. Science, 2002 295:647-651, the contents of which are herein incorporated by reference in their entirety).

In some embodiments, the compositions of the present disclosure may be formulated in lipid nanoparticles created using a micromixer chip such as, but not limited to, those from Harvard Apparatus (Holliston, Mass.) or Dolomite Microfluidics (Royston, UK). A micromixer chip can be used for rapid mixing of two or more fluid streams with a split and recombine mechanism.

In some embodiments, the compositions of the disclosure may be formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles may have a diameter from about 10 to 500 nm.

In some embodiments, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

Described herein are also composition including an effective amount of a nanoparticle described herein and a pharmaceutically acceptable carrier. In some aspects, the pharmaceutical compositions can include a pharmaceutically acceptable carrier and a nanoparticle including a nucleic acid sequence (e.g., mRNA) encoding an infection agent antigenic polypeptide and a nucleic acid sequence (e.g., mRNA) encoding at least one universal T-cell epitope, as well as, compositions including a nucleic acid sequence (e.g., mRNA) encoding an infection agent antigenic polypeptide and at least one universal T-cell epitope. In some aspects, the pharmaceutical composition can include a pharmaceutically acceptable carrier and a lipid nanoparticle including an nucleic acid sequence (e.g., mRNA) encoding an infection agent antigenic polypeptide and a nucleic acid sequence (e.g., mRNA) encoding at least one universal T-cell epitope, as well as compositions including a nucleic acid sequence (e.g., mRNA) encoding an infection agent antigenic polypeptide and at least one universal T-cell epitope.

Liposomes, lipoplexes, or lipid nanoparticles may be used to improve the efficacy of polynucleotides directed protein production as these formulations may be able to increase cell transfection by the RNA (e.g., mRNA) polynucleotide; and/or increase the translation of encoded protein. One such example involves the use of lipid encapsulation to enable the effective systemic delivery of polyplex plasmid DNA (Heyes et al., Mol Ther. 2007 15:713-720; the contents of which are incorporated herein by reference in their entirety). The liposomes, lipoplexes, or lipid nanoparticles may also be used to increase the stability of the polynucleotide.

Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In some embodiments, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.).

In some embodiments, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104; all of which are incorporated herein in their entireties). The original manufacture method by Wheeler et al. was a detergent dialysis method, which was later improved by Jeffs et al. and is referred to as the spontaneous vesicle formation method. The liposome formulations are composed of 3 to 4 lipid components in addition to the polynucleotide. As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may contain. but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In some embodiments, liposome formulations may comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In some embodiments, formulations may comprise a percentage of cholesterol selected from the group consisting of 28.5%, 31.5%, 33.5%, 36.5%, 37.0%, 38.5%, 39.0% and 43.5%. In some embodiments, formulations may comprise from about 5.0% to about 10.0% DSPC and/or from about 7.0% to about 15.0% DSPC.

In some embodiments, the compositions may be formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5 (12) 1708-1713); herein incorporated by reference in its entirety) and hyaluronan-coated liposomes (Quiet Therapeutics, Israel).

In some embodiments, the compositions may be formulated in a lipid vesicle, which may have crosslinks between functionalized lipid bilayers.

In some embodiments, the compositions may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine. In some embodiments, the compositions may be formulated in a lipid-polycation complex, which may further include a non-cationic lipid such as, but not limited to, cholesterol or dioleoyl phosphatidylethanolamine (DOPE).

In some embodiments, the composition can be formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, Mass.), and polyethylenimine (PEI) or protamine-based targeted and non-targeted delivery of nucleic acids acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293 Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15: Song et al., Nature Biotechnol. 2005, 23:709-717; Peer et al., Proc Natl Acad Sci USA. 2007 6; 104:4095-4100; deFougerolles Hum Gene Ther. 2008 19:125-132, the contents of each of which are incorporated herein by reference in their entirety).

In some embodiments, such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133, the contents of each of which are incorporated herein by reference in their entirety). One example of passive targeting of formulations to liver cells includes the DLin-DMA, DLin-KC2-DMA and DLin-MC3-DMA-based lipid nanoparticle formulations, which have been shown to bind to apolipoprotein E and promote binding and uptake of these formulations into hepatocytes in vivo (Akinc et al. Mol Ther. 2010 18:1357-1364, the contents of which are incorporated herein by reference in their entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133, the contents of each of which are incorporated herein by reference in their entirety).

In some embodiments, the composition of the present disclosure can be formulated for controlled release and/or targeted delivery. As used herein, “controlled release” refers to a pharmaceutical composition or compound release profile that conforms to a particular pattern of release to effect a therapeutic outcome. In some embodiments, the compositions may be encapsulated into a delivery agent described herein and/or known in the art for controlled release and/or targeted delivery. As used herein, the term “encapsulate” means to enclose, surround or encase. As it relates to the formulation of the compounds of the disclosure, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the disclosure may be enclosed, surrounded or encased within the delivery agent. “Partially encapsulation” means that less than 10, 10, 20, 30, 40 50 or less of the pharmaceutical composition or compound of the disclosure may be enclosed, surrounded or encased within the delivery agent. Advantageously, encapsulation may be determined by measuring the escape or the activity of the pharmaceutical composition or compound of the disclosure using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the disclosure are encapsulated in the delivery agent.

In some embodiments, the controlled release formulation may include, but is not limited to, tri-block co-polymers. As a non-limiting example, the formulation may include two different types of tri-block co-polymers (International Pub. No. WO2012131104 and

WO2012131106, the contents of each of which are incorporated herein by reference in their entirety).

In some embodiments, the compositions may be encapsulated into a lipid nanoparticle or a rapidly eliminated lipid nanoparticle and the lipid nanoparticles or a rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

In some embodiments, the compositions described herein formulated for controlled release and/or targeted delivery may also include at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In some embodiments, the degradable polyesters may include a PEG conjugation to form a PEGylated polymer.

In some embodiments, the compositions described herein formulated for controlled release and/or targeted delivery may also include at least one PEG and/or PEG related polymer derivatives as described in U.S. Pat. No. 8,404,222, the contents of which are incorporated herein by reference in their entirety.

In some embodiments, the compositions described herein formulated for controlled release delivery may be the controlled release polymer system described in US20130130348, the contents of which are incorporated herein by reference in their entirety.

In some embodiments, the synthetic nanocarriers may be formulated for targeted release. In some embodiments, the synthetic nanocarrier is formulated to release the polynucleotides at a specified pH and/or after a desired time interval. As a non-limiting example, the synthetic nanoparticle may be formulated to release the RNA (e.g., mRNA) vaccines after 24 hours and/or at a pH of 4.5 (see International Publication Nos. WO2010138193 and WO2010138194 and US Pub Nos. US20110020388 and US20110027217, each of which is herein incorporated by reference in their entireties).

In some embodiments, the synthetic nanocarriers may be formulated for controlled and/or sustained release of the nucleic acids described herein. As a non-limiting example, the synthetic nanocarriers for sustained release may be formulated by methods known in the art, described herein and/or as described in International Pub No. WO2010138192 and US Pub No. 20100303850, each of which is herein incorporated by reference in their entirety.

In some embodiments, the synthetic nanocarrier may be formulated for use as a vaccine. In some embodiments, the synthetic nanocarrier may encapsulate at least one polynucleotide which encode at least one antigen. As a non-limiting example, the synthetic nanocarrier may include at least one antigen and an excipient for a vaccine dosage form (see International Publication No. WO2011150264 and U.S. Publication No. US20110293723, the contents of each of which are herein incorporated by reference in their entirety). As another non-limiting example, a vaccine dosage form may include at least two synthetic nanocarriers with the same or different antigens and an excipient (see International Publication No. WO2011150249 and U.S. Publication No. US20110293701, the contents of each of which are herein incorporated by reference in their entirety). The vaccine dosage form may be selected by methods described herein, known in the art and/or described in International Publication No. WO2011150258 and U.S. Publication No. US20120027806, the contents of each of which are herein incorporated by reference in their entirety).

In some embodiments, the synthetic nanocarrier may encapsulate at least one polynucleotide that encodes a peptide, fragment or region from a virus. As a non-limiting example, the synthetic nanocarrier may include, but is not limited to, any of the nanocarriers described in International Publication No. WO2012024621, WO201202629, WO2012024632 and U.S. Publication No. US20120064110, US20120058153 and US20120058154, the contents of each of which are herein incorporated by reference in their entirety.

In some embodiments, the compositions may be formulated in colloid nanocarriers as described in U.S. Patent Publication No. US20130197100, the contents of which are herein incorporated by reference in their entirety.

In some embodiments, the compositions of the disclosure may be formulated for delivery using the drug encapsulating microspheres described in International Patent Publication No. WO2013063468 or U.S. Pat. No. 8,440,614, the contents of each of which are herein incorporated by reference in their entirety. The microspheres may comprise a compound of the formula (I), (II), (III), (IV), (V) or (VI) as described in International Patent Publication No. WO2013063468, the contents of which are herein incorporated by reference in their entirety. In some embodiments, the amino acid, peptide, polypeptide, lipids (APPL) are useful in delivering the RNA (e.g., mRNA) polynucleotides of the disclosure to cells (see International Patent Publication No. WO2013063468, the contents of which are herein incorporated by reference in their entirety).

In some embodiments, the compositions may be delivered, localized and/or concentrated in a specific location using the delivery methods described in International Patent Publication No. WO2013063530, the contents of which are herein incorporated by reference in their entirety. As a non-limiting example, a subject may be administered an empty polymeric particle prior to, simultaneously with or after delivering the nucleic acid (e.g., mRNA) compositions to the subject. The empty polymeric particle undergoes a change in volume once in contact with the subject and becomes lodged, embedded, immobilized or entrapped at a specific location in the subject.

In some embodiments, the compositions may be formulated in an active substance release system (see, e.g., U.S. Patent Publication No. US20130102545, the contents of which are herein incorporated by reference in their entirety). The active substance release system may comprise 1) at least one nanoparticle bonded to an oligonucleotide inhibitor strand which is hybridized with a catalytically active nucleic acid and 2) a compound bonded to at least one substrate molecule bonded to a therapeutically active substance (e.g., polynucleotides described herein), where the therapeutically active substance is released by the cleavage of the substrate molecule by the catalytically active nucleic acid.

In some embodiments the compositions may be associated with a cationic or polycationic compounds, including protamine, nucleoline, spermine or spermidine, or other cationic peptides or proteins, such as poly-L-lysine (PLL), polyarginine, basic polypeptides, cell penetrating peptides (CPPs), including HIV-binding peptides, HIV-1 Tat (HIV), Tat-derived peptides, Penetratin, VP22 derived or analog peptides, Pestivirus Ems, HSV, VP22 (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides (particularly from Drosophila antennapedia), pAntp, plsl, FGF, Lactoferrin, Transportan, Buforin-2, Bac715-24, SynB, SynB (1), pVEC, hCT-derived peptides, SAP, histones, cationic polysaccharides, for example chitosan, polybrene, cationic polymers, e.g. polyethyleneimine (PEI), cationic lipids, e.g. DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS: Dioctadecylamidoglicylspermin, DIMRI: Dimyristooxypropyl dimethyl hydroxyethyl ammonium bromide, DOTAP: dioleoyloxy-3-(trimethylammonio) propane, DC-6-14: O,O-ditetradecanoyl-N-.alpha.-trimethylammonioacetyl) diethanolamine chloride, CLIP 1: rac-[(2,3-dioctadecyloxypropyl) (2-hydroxyethyl)]-dimethylammonium chloride, CLIP6: rac-[2 (2,3-dihexadecyloxypropyloxymethyloxy)ethyl]-trimethylammonium, CLIP9: rac-[2 (2,3-dihexadecyloxypropyloxysuccinyloxy)ethyl]-trimethylammonium, oligofectamine, or cationic or polycationic polymers, e.g. modified polyaminoacids, such as beta-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified amidoamines such as pAMAM (poly(amidoamine)), etc., modified polybetaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycole), etc.

In other embodiments, the composition is not associated with a cationic or polycationic compound.

### Vaccines

Described herein are vaccines including the compositions described herein. In some aspects, described herein are vaccines including a nucleic acid sequence (e.g., mRNA) encoding an infection agent antigenic polypeptide and a nucleic acid sequence (e.g., mRNA) encoding at least one universal T-cell epitope, as well as, vaccines including a nucleic acid sequence (e.g., mRNA) encoding an infection agent antigenic polypeptide and at least one universal T-cell epitope.

The vaccines described herein, can include multiple nucleic acid sequences (e.g., mRNA), each encoding a single infection agent antigenic polypeptide and multiple nucleic acid sequences (e.g., mRNA), each encoding a single universal T-cell epitope, as well as, vaccines including a single nucleic acid sequence (e.g., mRNA) encoding one or more infection agent antigenic polypeptide and a single nucleic acid sequence (e.g., mRNA) encoding one or more universal T-cell epitope.

The vaccines described herein, can include multiple nucleic acid sequences (e.g., mRNA), each encoding a single infection agent antigenic polypeptide and a single universal T-cell epitope, as well as, vaccines including a single nucleic acid sequence (e.g., mRNA) encoding one or more infection agent antigenic polypeptide and one or more universal T-cell epitope.

The vaccines of the present disclosure, in some embodiments, include 2-10 (e.g., 2, 3, 4, 5, 6, 7, 8, 9 or 10), or more, nucleic acid sequences having an open reading frame, each of which encodes a different infection agent antigenic polypeptide (or a single nucleic acid sequence encoding 2-10, or more, different infection agent antigenic polypeptides). The vaccines of the present disclosure, in some embodiments, include 2-10 (e.g., 2, 3, 4, 5, 6, 7, 8, 9 or 10), or more, nucleic acid sequence having an open reading frame, each of which encodes a different universal T-cell epitope (or a single nucleic acid sequence encoding 2-10, or more, different universal T-cell epitopes).

In some embodiments, vaccines can be formulated in a nanoparticle described herein. In some embodiments, vaccines can be formulated in a lipid nanoparticle described herein. In some embodiments, vaccines can be formulated in a lipid-polycation complex described herein, referred to as a cationic lipid nanoparticle. The vaccines of the disclosure can be formulated using one or more liposomes, lipoplexes, or lipid nanoparticles described herein. In some embodiments, pharmaceutical compositions of vaccines can include liposomes described herein. In some embodiments, the vaccines may be formulated in a lipid vesicle, which may have crosslinks between functionalized lipid bilayers. In some embodiments, the vaccines may be formulated in a lipid-polycation complex.

In some embodiments, the nucleic acid sequence encoding the infection agent antigenic polypeptide can elicit an antibody response in a subject. In some embodiments, the nucleic acid sequence encoding the infection agent antigenic polypeptide can elicit a broad T-cell response in a subject. In some embodiments, the nucleic acid sequence encoding the infection agent antigenic polypeptide can elicit both an antibody response and a T-cell response in a subject.

In some embodiments, the nucleic acid sequence encoding the infection agent antigenic polypeptide can elicit a cellular immune response, a humoral immune response, or a combination thereof. In some embodiments, the nucleic acid sequence encoding the infection agent antigenic polypeptide can elicit a cellular immune response, a humoral immune response, or a combination thereof, without risking the possibility of insertional mutagenesis.

In some embodiments, the universal T-cell epitope can provide broader protection against infection agent variants.

In some embodiments, the vaccine can include a nucleic acid sequence (e.g., mRNA) encoding a human Metapneumovirus (hMPV) antigenic polypeptide, human parainfluenza viruses (hPIV) types 1, 2, and 3 (hPIV1, hPIV2 and hPIV3, respectively) antigenic polypeptide, respiratory syncytial virus (RSV) antigenic polypeptide, measles virus (MeV) antigenic polypeptide, varicella-zoster antigenic polypeptide, influenza virus antigenic polypeptide, herpes simplex virus 1 (HSV1) antigenic polypeptide, herpes simplex virus 2 (HSV2) antigenic polypeptide, poxvirus (e.g., smallpox, monkeypox) antigenic polypeptide, cytomegalovirus antigenic polypeptide, Epstein-Barr virus antigenic polypeptide, rotavirus antigenic polypeptide, rhinovirus antigenic polypeptide, adenovirus antigenic polypeptide, papillomavirus antigenic polypeptide, poliovirus antigenic polypeptide, mumps antigenic polypeptide, rabies antigenic polypeptide, rubella antigenic polypeptide, coxsackieviruses antigenic polypeptide, equine encephalitis antigenic polypeptide, Japanese encephalitis antigenic polypeptide, yellow fever antigenic polypeptide, Rift Valley fever antigenic polypeptide, hepatitis A, B, C, D, and E virus antigenic polypeptide, coronaviruses (e.g., MERS-COV, SARS-COV, SARS-COV2, HCoV-OC43, HCOV-229E, HCoV-NL63, HCoV-NL, HCoV-NH, HCoV-HKU1) antigenic polypeptide (see, e.g., Esper F. et al. Emerging Infectious Diseases, 12 (5). 2006; and Pyrc K. et al. Journal of Virology, 81 (7): 3051-57, 2007, the contents of each of which is here incorporated by reference in their entirety), African swine fever (ASF) antigenic polypeptide, foot-and-mouth disease virus (FMDV) antigenic polypeptide, feline herpesvirus-1/feline viral rhinotracheitis antigenic polypeptide, canine distemper antigenic polypeptide, feline coronavirus (FCOV) antigenic polypeptide, or any combination thereof.

In some embodiments, the vaccine can include a nucleic acid sequence (e.g., mRNA) encoding monkeypox antigenic polypeptide, herpes simplex virus 1 (HSV1) antigenic polypeptide, herpes simplex virus 2 (HSV2) antigenic polypeptide, coronavirus (e.g., SARS-CoV2) antigenic polypeptide, or any combination thereof.

In some embodiments, the vaccine can include a nucleic acid sequence (e.g., mRNA) encoding a human Metapneumovirus (hMPV) antigenic polypeptide, human parainfluenza viruses (hPIV) types 1, 2, and 3 (hPIV1, hPIV2 and hPIV3, respectively) antigenic polypeptide, respiratory syncytial virus (RSV) antigenic polypeptide, measles virus (MeV) antigenic polypeptide, varicella-zoster antigenic polypeptide, influenza virus antigenic polypeptide, herpes simplex virus 1 (HSV1) antigenic polypeptide, herpes simplex virus 2 (HSV2) antigenic polypeptide, poxvirus (e.g., smallpox, monkeypox) antigenic polypeptide, cytomegalovirus antigenic polypeptide, Epstein-Barr virus antigenic polypeptide, rotavirus antigenic polypeptide, rhinovirus antigenic polypeptide, adenovirus antigenic polypeptide, papillomavirus antigenic polypeptide, poliovirus antigenic polypeptide, mumps antigenic polypeptide, rabies antigenic polypeptide, rubella antigenic polypeptide, coxsackieviruses antigenic polypeptide, equine encephalitis antigenic polypeptide, Japanese encephalitis antigenic polypeptide, yellow fever antigenic polypeptide, Rift Valley fever antigenic polypeptide, hepatitis A, B, C, D, and E virus antigenic polypeptide, coronaviruses (e.g., MERS-COV, SARS-COV, SARS-COV2, HCoV-OC43, HCOV-229E, HCoV-NL63, HCoV-NL, HCOV-NH, HCoV-HKU1) antigenic polypeptide (see, e.g., Esper F. et al. Emerging Infectious Diseases, 12 (5), 2006; and Pyrc K. et al. Journal of Virology, 81(7):3051-57, 2007, the contents of each of which is here incorporated by reference in their entirety), African swine fever (ASF) antigenic polypeptide, foot-and-mouth disease virus (FMDV) antigenic polypeptide, feline herpesvirus-1/feline viral rhinotracheitis antigenic polypeptide, canine distemper antigenic polypeptide, or any combination thereof.

In some embodiments, the vaccine can include a nucleic acid sequence (e.g., mRNA) encoding a human Metapneumovirus (hMPV) antigenic polypeptide, human parainfluenza viruses (hPIV) types 1, 2, and 3 (hPIV1, hPIV2 and hPIV3, respectively) antigenic polypeptide, respiratory syncytial virus (RSV) antigenic polypeptide, measles virus (MeV) antigenic polypeptide, varicella-zoster antigenic polypeptide, influenza virus antigenic polypeptide, herpes simplex virus 1 (HSV1) antigenic polypeptide, herpes simplex virus 2 (HSV2) antigenic polypeptide, poxvirus (e.g., smallpox, monkeypox) antigenic polypeptide, cytomegalovirus antigenic polypeptide, Epstein-Barr virus antigenic polypeptide, rotavirus antigenic polypeptide, rhinovirus antigenic polypeptide, adenovirus antigenic polypeptide, papillomavirus antigenic polypeptide, poliovirus antigenic polypeptide, mumps antigenic polypeptide, rabies antigenic polypeptide, rubella antigenic polypeptide, coxsackieviruses antigenic polypeptide, equine encephalitis antigenic polypeptide, Japanese encephalitis antigenic polypeptide, yellow fever antigenic polypeptide, Rift Valley fever antigenic polypeptide, hepatitis A, B, C, D, and E virus antigenic polypeptide, African swine fever (ASF) antigenic polypeptide, foot-and-mouth disease virus (FMDV) antigenic polypeptide, feline herpesvirus-1/feline viral rhinotracheitis antigenic polypeptide, canine distemper antigenic polypeptide, feline coronavirus (FCoV) antigenic polypeptide, or any combination thereof.

In some embodiments, the vaccine can include a nucleic acid sequence (e.g., mRNA) encoding a human Metapneumovirus (hMPV) antigenic polypeptide, human parainfluenza viruses (hPIV) types 1, 2, and 3 (hPIV1, hPIV2 and hPIV3, respectively) antigenic polypeptide, respiratory syncytial virus (RSV) antigenic polypeptide, measles virus (MeV) antigenic polypeptide, varicella-zoster antigenic polypeptide, influenza virus antigenic polypeptide, poxvirus (e.g., smallpox. monkeypox) antigenic polypeptide, cytomegalovirus antigenic polypeptide, Epstein-Barr virus antigenic polypeptide, rotavirus antigenic polypeptide, rhinovirus antigenic polypeptide, adenovirus antigenic polypeptide, papillomavirus antigenic polypeptide, poliovirus antigenic polypeptide, mumps antigenic polypeptide, rabies antigenic polypeptide, rubella antigenic polypeptide, coxsackieviruses antigenic polypeptide, equine encephalitis antigenic polypeptide, Japanese encephalitis antigenic polypeptide, yellow fever antigenic polypeptide, Rift Valley fever antigenic polypeptide, hepatitis A, B, C, D, and E virus antigenic polypeptide, coronaviruses (e.g., MERS-COV, SARS-COV, SARS-COV2, HCoV-OC43, HCOV-229E, HCoV-NL63, HCOV-NL, HCOV-NH, HCoV-HKU1) antigenic polypeptide (see, e.g., Esper F. et al. Emerging Infectious Diseases, 12 (5), 2006; and Pyrc K. et al. Journal of Virology, 81 (7): 3051-57, 2007, the contents of each of which is here incorporated by reference in their entirety), African swine fever (ASF) antigenic polypeptide, foot-and-mouth disease virus (FMDV) antigenic polypeptide, feline herpesvirus-1/feline viral rhinotracheitis antigenic polypeptide, canine distemper antigenic polypeptide, feline coronavirus (FCoV) antigenic polypeptide, or any combination thereof.

In some embodiments, the vaccine can include a nucleic acid sequence (e.g., mRNA) encoding a human Metapneumovirus (hMPV) antigenic polypeptide, human parainfluenza viruses (hPIV) types 1, 2, and 3 (hPIV1, hPIV2 and hPIV3, respectively) antigenic polypeptide, respiratory syncytial virus (RSV) antigenic polypeptide, measles virus (MeV) antigenic polypeptide, varicella-zoster antigenic polypeptide, influenza virus antigenic polypeptide, poxvirus (e.g., smallpox, monkeypox) antigenic polypeptide, cytomegalovirus antigenic polypeptide, Epstein-Barr virus antigenic polypeptide, rotavirus antigenic polypeptide, rhinovirus antigenic polypeptide, adenovirus antigenic polypeptide, papillomavirus antigenic polypeptide, poliovirus antigenic polypeptide, mumps antigenic polypeptide, rabies antigenic polypeptide, rubella antigenic polypeptide, coxsackieviruses antigenic polypeptide, equine encephalitis antigenic polypeptide, Japanese encephalitis antigenic polypeptide, yellow fever antigenic polypeptide, Rift Valley fever antigenic polypeptide, hepatitis A, B, C, D, and E virus antigenic polypeptide, coronaviruses (e.g., MERS-COV, SARS-COV, SARS-COV2, HCoV-OC43, HCOV-229E, HCoV-NL63, HCOV-NL, HCOV-NH, HCoV-HKU1) antigenic polypeptide (see, e.g., Esper F. et al. Emerging Infectious Diseases, 12 (5), 2006; and Pyrc K. et al. Journal of Virology, 81 (7): 3051-57, 2007, the contents of each of which is here incorporated by reference in their entirety), African swine fever (ASF) antigenic polypeptide, foot-and-mouth disease virus (FMDV) antigenic polypeptide, feline herpesvirus-1/feline viral rhinotracheitis antigenic polypeptide, canine distemper antigenic polypeptide, or any combination thereof.

In some embodiments, the vaccine can include a nucleic acid sequence (e.g., mRNA) encoding a human Metapneumovirus (hMPV) antigenic polypeptide, human parainfluenza viruses (hPIV) types 1, 2, and 3 (hPIV1, hPIV2 and hPIV3, respectively) antigenic polypeptide, respiratory syncytial virus (RSV) antigenic polypeptide, measles virus (MeV) antigenic polypeptide, varicella-zoster antigenic polypeptide, influenza virus antigenic polypeptide, poxvirus (e.g., smallpox, monkeypox) antigenic polypeptide, cytomegalovirus antigenic polypeptide, Epstein-Barr virus antigenic polypeptide, rotavirus antigenic polypeptide, rhinovirus antigenic polypeptide, adenovirus antigenic polypeptide, papillomavirus antigenic polypeptide, poliovirus antigenic polypeptide, mumps antigenic polypeptide, rabies antigenic polypeptide, rubella antigenic polypeptide, coxsackieviruses antigenic polypeptide, equine encephalitis antigenic polypeptide, Japanese encephalitis antigenic polypeptide, yellow fever antigenic polypeptide, Rift Valley fever antigenic polypeptide, hepatitis A, B, C, D, and E virus antigenic polypeptide, African swine fever (ASF) antigenic polypeptide. foot-and-mouth disease virus (FMDV) antigenic polypeptide, feline herpesvirus-1/feline viral rhinotracheitis antigenic polypeptide, canine distemper antigenic polypeptide, feline coronavirus (FCoV) antigenic polypeptide, or any combination thereof.

In some embodiments, the vaccine can include a nucleic acid sequence (e.g., mRNA) encoding a human Metapneumovirus (hMPV) antigenic polypeptide, human parainfluenza viruses (hPIV) types 1, 2, and 3 (hPIV1, hPIV2 and hPIV3, respectively) antigenic polypeptide, respiratory syncytial virus (RSV) antigenic polypeptide, measles virus (MeV) antigenic polypeptide, varicella-zoster antigenic polypeptide, influenza virus antigenic polypeptide, herpes simplex virus 1 (HSV1) antigenic polypeptide, herpes simplex virus 2 (HSV2) antigenic polypeptide, poxvirus (e.g., smallpox, monkeypox) antigenic polypeptide, cytomegalovirus antigenic polypeptide, Epstein-Barr virus antigenic polypeptide, rotavirus antigenic polypeptide, rhinovirus antigenic polypeptide, adenovirus antigenic polypeptide, papillomavirus antigenic polypeptide, poliovirus antigenic polypeptide, mumps antigenic polypeptide, rabies antigenic polypeptide, rubella antigenic polypeptide, coxsackieviruses antigenic polypeptide, equine encephalitis antigenic polypeptide, Japanese encephalitis antigenic polypeptide, yellow fever antigenic polypeptide, Rift Valley fever antigenic polypeptide, hepatitis A, B, C, D, and E virus antigenic polypeptide, African swine fever (ASF) antigenic polypeptide, foot-and-mouth disease virus (FMDV) antigenic polypeptide, feline herpesvirus-1/feline viral rhinotracheitis antigenic polypeptide, canine distemper antigenic polypeptide, or any combination thereof.

In some embodiments, the vaccine can include a nucleic acid sequence (e.g., mRNA) encoding monkeypox antigenic polypeptide. In some embodiments, the vaccine can include a nucleic acid sequence (e.g., mRNA) encoding herpes simplex virus 1 (HSV1) antigenic polypeptide. In some embodiments, the vaccine can include a nucleic acid sequence (e.g., mRNA) encoding herpes simplex virus 2 (HSV2) antigenic polypeptide. In some embodiments, the vaccine can include a nucleic acid sequence (e.g., mRNA) encoding coronavirus (e.g., SARS-COV2) antigenic polypeptide. In some embodiments, the vaccine can include a nucleic acid sequence (e.g., mRNA) encoding feline coronavirus (FCOV) antigenic polypeptide.

The present disclosure also provides, in some embodiments, combination vaccines where the nucleic acid sequence (e.g., mRNA) encodes more than one antigenic polypeptides selected from human Metapneumovirus (hMPV) antigenic polypeptide, human parainfluenza viruses (hPIV) types 1, 2, and 3 (hPIV1, hPIV2 and hPIV3, respectively) antigenic polypeptide, respiratory syncytial virus (RSV) antigenic polypeptide, measles virus (MeV) antigenic polypeptide, varicella-zoster antigenic polypeptide, influenza virus antigenic polypeptide, herpes simplex virus 1 (HSV1) antigenic polypeptide, herpes simplex virus 2 (HSV2) antigenic polypeptide, poxvirus (e.g., smallpox, monkeypox) antigenic polypeptide, cytomegalovirus antigenic polypeptide, Epstein-Barr virus antigenic polypeptide, rotavirus antigenic polypeptide, rhinovirus antigenic polypeptide, adenovirus antigenic polypeptide, papillomavirus antigenic polypeptide, poliovirus antigenic polypeptide, mumps antigenic polypeptide, rabies antigenic polypeptide, rubella antigenic polypeptide, coxsackieviruses antigenic polypeptide, equine encephalitis antigenic polypeptide, Japanese encephalitis antigenic polypeptide, yellow fever antigenic polypeptide, Rift Valley fever antigenic polypeptide, hepatitis A, B, C, D, and E virus antigenic polypeptide, coronaviruses (e.g., MERS-COV, SARS-COV2, SARS-COV2, HCoV-OC43, HCOV-229E, HCoV-NL63, HCoV-NL, HCOV-NH, HCoV-HKU1) antigenic polypeptide), African swine fever (ASF) antigenic polypeptide, foot-and-mouth disease virus (FMDV) antigenic polypeptide, feline herpesvirus-1/feline viral rhinotracheitis antigenic polypeptide, canine distemper antigenic polypeptide, feline coronavirus (FCoV) antigenic polypeptide, or any combination thereof.

In some embodiments, the combination vaccines where the nucleic acid sequence (e.g., mRNA) encodes more than one antigenic polypeptides selected from human Metapneumovirus (hMPV) antigenic polypeptide, human parainfluenza viruses (hPIV) types 1, 2, and 3 (hPIV1, hPIV2 and hPIV3, respectively) antigenic polypeptide, respiratory syncytial virus (RSV) antigenic polypeptide, measles virus (MeV) antigenic polypeptide, varicella-zoster antigenic polypeptide, influenza virus antigenic polypeptide, herpes simplex virus 1 (HSV1) antigenic polypeptide, herpes simplex virus 2 (HSV2) antigenic polypeptide, poxvirus (e.g., smallpox, monkeypox) antigenic polypeptide, cytomegalovirus antigenic polypeptide, Epstein-Barr virus antigenic polypeptide, rotavirus antigenic polypeptide, rhinovirus antigenic polypeptide, adenovirus antigenic polypeptide, papillomavirus antigenic polypeptide, poliovirus antigenic polypeptide, mumps antigenic polypeptide, rabies antigenic polypeptide, rubella antigenic polypeptide, coxsackieviruses antigenic polypeptide, equine encephalitis antigenic polypeptide, Japanese encephalitis antigenic polypeptide, yellow fever antigenic polypeptide, Rift Valley fever antigenic polypeptide, hepatitis A, B, C, D, and E virus antigenic polypeptide, coronaviruses (e.g., MERS-COV, SARS-COV, SARS-COV2, HCoV-OC43, HCOV-229E, HCoV-NL63, HCOV-NL, HCoV-NH, HCoV-HKU1) antigenic polypeptide, African swine fever (ASF) antigenic polypeptide, foot-and-mouth disease virus (FMDV) antigenic polypeptide, feline herpesvirus-1/feline viral rhinotracheitis antigenic polypeptide, canine distemper antigenic polypeptide, or any combination thereof.

In some embodiments, the combination vaccines where the nucleic acid sequence (e.g., mRNA) encodes more than one antigenic polypeptides selected from human Metapneumovirus (hMPV) antigenic polypeptide, human parainfluenza viruses (hPIV) types 1, 2, and 3 (hPIV1, hPIV2 and hPIV3, respectively) antigenic polypeptide, respiratory syncytial virus (RSV) antigenic polypeptide, measles virus (MeV) antigenic polypeptide, varicella-zoster antigenic polypeptide, influenza virus antigenic polypeptide, herpes simplex virus 1 (HSV1) antigenic polypeptide, herpes simplex virus 2 (HSV2) antigenic polypeptide, poxvirus (e.g., smallpox, monkeypox) antigenic polypeptide, cytomegalovirus antigenic polypeptide, Epstein-Barr virus antigenic polypeptide, rotavirus antigenic polypeptide, rhinovirus antigenic polypeptide, adenovirus antigenic polypeptide, papillomavirus antigenic polypeptide, poliovirus antigenic polypeptide, mumps antigenic polypeptide, rabies antigenic polypeptide, rubella antigenic polypeptide, coxsackieviruses antigenic polypeptide, equine encephalitis antigenic polypeptide, Japanese encephalitis antigenic polypeptide, yellow fever antigenic polypeptide, Rift Valley fever antigenic polypeptide, hepatitis A, B, C, D, and E virus antigenic polypeptide, African swine fever (ASF) antigenic polypeptide, foot-and-mouth disease virus (FMDV) antigenic polypeptide, feline herpesvirus-1/feline viral rhinotracheitis antigenic polypeptide, canine distemper antigenic polypeptide, feline coronavirus (FCoV) antigenic polypeptide, or any combination thereof.

In some embodiments, the combination vaccines where the nucleic acid sequence (e.g., mRNA) encodes more than one antigenic polypeptides selected from human Metapneumovirus (hMPV) antigenic polypeptide, human parainfluenza viruses (hPIV) types 1, 2, and 3 (hPIV1, hPIV2 and hPIV3, respectively) antigenic polypeptide, respiratory syncytial virus (RSV) antigenic polypeptide, measles virus (MeV) antigenic polypeptide, varicella-zoster antigenic polypeptide, influenza virus antigenic polypeptide, poxvirus (e.g., smallpox, monkeypox) antigenic polypeptide, cytomegalovirus antigenic polypeptide, Epstein-Barr virus antigenic polypeptide, rotavirus antigenic polypeptide, rhinovirus antigenic polypeptide, adenovirus antigenic polypeptide, papillomavirus antigenic polypeptide, poliovirus antigenic polypeptide, mumps antigenic polypeptide, rabies antigenic polypeptide, rubella antigenic polypeptide, coxsackieviruses antigenic polypeptide, equine encephalitis antigenic polypeptide, Japanese encephalitis antigenic polypeptide, yellow fever antigenic polypeptide, Rift Valley fever antigenic polypeptide, hepatitis A, B, C, D, and E virus antigenic polypeptide, coronaviruses (e.g., MERS-COV, SARS-COV, SARS-COV2, HCoV-OC43, HCoV-229E, HCoV-NL63, HCOV-NL, HCoV-NH, HCoV-HKU1) antigenic polypeptide, African swine fever (ASF) antigenic polypeptide, foot-and-mouth disease virus (FMDV) antigenic polypeptide, feline herpesvirus-1/feline viral rhinotracheitis antigenic polypeptide, canine distemper antigenic polypeptide, feline coronavirus (FCOV) antigenic polypeptide, or any combination thereof.

In some embodiments, the combination vaccines where the nucleic acid sequence (e.g., mRNA) encodes more than one antigenic polypeptides selected from human Metapneumovirus (hMPV) antigenic polypeptide, human parainfluenza viruses (hPIV) types 1, 2, and 3 (hPIV1, hPIV2 and hPIV3, respectively) antigenic polypeptide, respiratory syncytial virus (RSV) antigenic polypeptide, measles virus (MeV) antigenic polypeptide, varicella-zoster antigenic polypeptide, influenza virus antigenic polypeptide, poxvirus (e.g., smallpox, monkeypox) antigenic polypeptide, cytomegalovirus antigenic polypeptide, Epstein-Barr virus antigenic polypeptide, rotavirus antigenic polypeptide, rhinovirus antigenic polypeptide, adenovirus antigenic polypeptide, papillomavirus antigenic polypeptide, poliovirus antigenic polypeptide, mumps antigenic polypeptide, rabies antigenic polypeptide, rubella antigenic polypeptide, coxsackieviruses antigenic polypeptide, equine encephalitis antigenic polypeptide. Japanese encephalitis antigenic polypeptide, yellow fever antigenic polypeptide, Rift Valley fever antigenic polypeptide, hepatitis A, B, C, D, and E virus antigenic polypeptide, coronaviruses (e.g., MERS-COV, SARS-COV, SARS-COV2, HCoV-OC43, HCOV-229E, HCoV-NL63, HCOV-NL, HCOV-NH, HCoV-HKU1) antigenic polypeptide, African swine fever (ASF) antigenic polypeptide, foot-and-mouth disease virus (FMDV) antigenic polypeptide, feline herpesvirus-1/feline viral rhinotracheitis antigenic polypeptide. canine distemper antigenic polypeptide, or any combination thereof.

In some embodiments, the combination vaccines where the nucleic acid sequence (e.g., mRNA) encodes more than one antigenic polypeptides selected from human Metapneumovirus (hMPV) antigenic polypeptide, human parainfluenza viruses (hPIV) types 1, 2, and 3 (hPIV1, hPIV2 and hPIV3, respectively) antigenic polypeptide, respiratory syncytial virus (RSV) antigenic polypeptide, measles virus (MeV) antigenic polypeptide, varicella-zoster antigenic polypeptide, influenza virus antigenic polypeptide, poxvirus (e.g., smallpox, monkeypox) antigenic polypeptide, cytomegalovirus antigenic polypeptide, Epstein-Barr virus antigenic polypeptide, rotavirus antigenic polypeptide, rhinovirus antigenic polypeptide, adenovirus antigenic polypeptide, papillomavirus antigenic polypeptide, poliovirus antigenic polypeptide, mumps antigenic polypeptide, rabies antigenic polypeptide, rubella antigenic polypeptide, coxsackieviruses antigenic polypeptide, equine encephalitis antigenic polypeptide, Japanese encephalitis antigenic polypeptide, yellow fever antigenic polypeptide, Rift Valley fever antigenic polypeptide, hepatitis A, B, C, D, and E virus antigenic polypeptide, African swine fever (ASF) antigenic polypeptide, foot-and-mouth disease virus (FMDV) antigenic polypeptide, feline herpesvirus-1/feline viral rhinotracheitis antigenic polypeptide, canine distemper antigenic polypeptide, feline coronavirus (FCOV) antigenic polypeptide, or any combination thereof.

In some embodiments, the combination vaccines where the nucleic acid sequence (e.g., mRNA) encodes more than one antigenic polypeptides selected from human Metapneumovirus (hMPV) antigenic polypeptide, human parainfluenza viruses (hPIV) types 1, 2, and 3 (hPIV1, hPIV2 and hPIV3, respectively) antigenic polypeptide, respiratory syncytial virus (RSV) antigenic polypeptide, measles virus (MeV) antigenic polypeptide, varicella-zoster antigenic polypeptide, influenza virus antigenic polypeptide, herpes simplex virus 1 (HSV1) antigenic polypeptide, herpes simplex virus 2 (HSV2) antigenic polypeptide, poxvirus (e.g., smallpox, monkeypox) antigenic polypeptide, cytomegalovirus antigenic polypeptide, Epstein-Barr virus antigenic polypeptide, rotavirus antigenic polypeptide, rhinovirus antigenic polypeptide, adenovirus antigenic polypeptide, papillomavirus antigenic polypeptide, poliovirus antigenic polypeptide, mumps antigenic polypeptide, rabies antigenic polypeptide, rubella antigenic polypeptide, coxsackieviruses antigenic polypeptide, equine encephalitis antigenic polypeptide, Japanese encephalitis antigenic polypeptide, yellow fever antigenic polypeptide, Rift Valley fever antigenic polypeptide, hepatitis A, B, C, D, and E virus antigenic polypeptide, African swine fever (ASF) antigenic polypeptide, foot-and-mouth disease virus (FMDV) antigenic polypeptide, feline herpesvirus-1/feline viral rhinotracheitis antigenic polypeptide, canine distemper antigenic polypeptide, or any combination thereof.

In some embodiments, combination vaccines where the nucleic acid sequence (e.g., mRNA) encodes more than one infection agent antigenic polypeptides selected from hMPV antigenic polypeptides, PIV3 antigenic polypeptides, RSV antigenic polypeptides, MeV antigenic polypeptides and coronavirus antigenic polypeptides.

In some embodiments, the combination vaccines where the nucleic acid sequence (e.g. mRNA) encodes more than one infection agent antigenic polypeptide selected from monkeypox antigenic polypeptide, herpes simplex virus 1 (HSV1) antigenic polypeptide, herpes simplex virus 2 (HSV2) antigenic polypeptide, coronavirus (e.g., SARS-COV-2) antigenic polypeptide, or any combination thereof.

In some embodiments, the vaccines can further include pharmaceutically acceptable carrier.

In some embodiments, vaccine comprises an adjuvant, such as a flagellin adjuvant, as provided herein.

### Multiprotein and Multicomponent Vaccines

The compositions described herein, can include multiple nucleic acid sequences (e.g., mRNA), each encoding a single infection agent antigenic polypeptide and multiple nucleic acid sequences (e.g., mRNA), each encoding a single universal T-cell epitope, as well as compositions including a single nucleic acid sequence (e.g., mRNA) encoding one or more infection agent antigenic polypeptide and a single nucleic acid sequence (e.g., mRNA) encoding one or more universal T-cell epitope(s).

The compositions described herein, can include multiple nucleic acid sequences (e.g., mRNA) each encoding a single infection agent antigenic polypeptide and a single universal T-cell epitope, as well as, compositions including a single nucleic acid sequence (e.g., mRNA) encoding one or more infection agent antigenic polypeptide and one or more universal T-cell epitope(s).

Thus, a composition including a nucleic acid sequence encoding an infection agent antigenic polypeptide and at least one universal T-cell epitope, encompasses compositions that include a nucleic acid sequence encoding a first infection agent antigenic polypeptide, a second infection agent antigenic polypeptide, a first universal T-cell epitope, and a second universal T-cell epitope. In some embodiments, a composition including a nucleic acid sequence encoding an infection agent antigenic polypeptide and a nucleic acid sequence encoding at least one universal T-cell epitope, encompasses compositions that include a first nucleic acid sequence encoding a first infection agent antigenic polypeptide, a second nucleic acid sequence encoding a second infection agent antigenic polypeptide, a third nucleic acid sequence encoding a first universal T-cell epitope, and a fourth nucleic acid sequence encoding a second universal T-cell epitope.

The compositions described herein, in some embodiments, include 2-10 (e.g., 2, 3, 4, 5, 6, 7, 8, 9 or 10), or more, nucleic acid sequence (e.g. mRNA) having an open reading frame, each of which encodes a different infection agent antigenic polypeptide (or a single nucleic acid sequence (e.g. mRNA) encoding 2-10, or more, different infection agent antigenic polypeptides). The compositions described herein, in some embodiments, include 2-10 (e.g., 2, 3, 4, 5, 6, 7, 8, 9 or 10), or more, nucleic acid sequence (e.g. mRNA) having an open reading frame, each of which encodes a different universal T-cell epitope (or a single nucleic acid sequence (e.g. mRNA) encoding 2-10, or more, different universal T-cell epitopes).

In some embodiments, a vaccine comprises a nucleic acid sequence (e.g., mRNA) having an open reading frame encoding a viral capsid protein, a nucleic acid sequence (e.g., mRNA) having an open reading frame encoding a viral premembrane/membrane protein, and a nucleic acid sequence (e.g., mRNA) having an open reading frame encoding a viral envelope protein. In some embodiments, a vaccine comprises a nucleic acid sequence (e.g., mRNA) having an open reading frame encoding a viral fusion (F) protein and a nucleic acid sequence having an open reading frame encoding a viral major surface glycoprotein (G protein). In some embodiments, a vaccine comprises a nucleic acid sequence (e.g., mRNA) having an open reading frame encoding a viral F protein. In some embodiments, a vaccine comprises a nucleic acid sequence (e.g., mRNA) having an open reading frame encoding a viral G protein. In some embodiments, a vaccine comprises a nucleic acid sequence (e.g., mRNA) having an open reading frame encoding a HN protein.

In some embodiments, a multicomponent vaccine comprises a nucleic acid sequence (e.g., mRNA) encoding an infection agent antigenic polypeptide, and at least one universal T-cell epitope and a signal peptide (e.g., any one of SEQ ID NO: 15-19). In some embodiments, a multicomponent vaccine comprises a nucleic acid sequence (e.g., mRNA) encoding an infection agent antigenic polypeptide fused to a signal peptide (e.g., any one of SEQ ID NO: 15-19). In some embodiments, a multicomponent vaccine comprises a nucleic acid sequence (e.g., mRNA) encoding at least one universal T-cell epitope fused to a signal peptide (e.g., any one of SEQ ID NO: 15-19). The signal peptide may be fused at the N-terminus or the C-terminus of an infection agent antigenic polypeptide and/or universal T-cell epitope.

### Broad Spectrum Vaccines

There may be situations where subjects are at risk for infection with more than one strain of human Metapneumovirus (hMPV), human parainfluenza viruses (hPIV) types 1, 2, and 3 (hPIV1, hPIV2 and hPIV3, respectively), respiratory syncytial virus (RSV), measles virus (MeV), varicella-zoster, influenza virus, herpes simplex virus 1 (HSV1), herpes simplex virus 2 (HSV2), poxvirus (e.g., smallpox, monkeypox), cytomegalovirus, Epstein-Barr virus, rotavirus. rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, coronaviruses (e.g., MERS-COV, SARS-COV, SARS-COV2, HCoV-OC43, HCOV-229E, HCoV-NL63, HCOV-NL, HCOV-NH, HCoV-HKU1), African swine fever (ASF), foot-and-mouth disease virus (FMDV), feline herpesvirus-1/feline viral rhinotracheitis, canine distemper, feline coronavirus (FCOV) or any combination thereof.

Vaccines are particularly amenable to combination vaccination approaches due to a number of factors including, but not limited to, speed of manufacture, ability to rapidly tailor vaccines to accommodate perceived geographical threat, and the like. Moreover, because the vaccines utilize the human body to produce the infection agent antigenic polypeptide, the vaccines are amenable to the production of larger, more complex antigenic proteins, allowing for proper folding, surface expression, antigen presentation, etc. in the human subject. To protect against more than one strain a combination vaccine can be administered that includes a nucleic acid sequence (e.g., mRNA) encoding an infection agent antigenic polypeptide (or antigenic portion thereof), a nucleic acid encoding at least one second infection agent antigenic polypeptide (or antigenic portion thereof), and a nucleic acid encoding at least one universal T-cell epitope. Nucleic acid sequence (e.g., mRNA) can be co-formulated, for example, in a single lipid nanoparticle (LNP) or can be formulated in separate LNPs for co-administration.

### Combination Vaccines

Embodiments of the present disclosure also provide combination vaccines. A “combination vaccine” of the present disclosure refers to a vaccine comprising at least one (e.g., at least 2, 3, 4, or 5) nucleic acid sequence (e.g., mRNA) having an open reading frame encoding a combination of any two or more (or all of) infection agent antigenic polypeptides selected from human Metapneumovirus (hMPV), human parainfluenza viruses (hPIV) types 1, 2, and 3 (hPIV1, hPIV2 and hPIV3, respectively), respiratory syncytial virus (RSV), measles virus (MeV), varicella-zoster, influenza virus, herpes simplex virus 1 (HSV1), herpes simplex virus 2 (HSV2), poxvirus (e.g., smallpox, monkeypox), cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, coronaviruses (e.g., MERS-COV, SARS-COV, SARS-COV2, HCoV-OC43, HCoV-229E, HCoV-NL63, HCOV-NL, HCOV-NH, HCOV-HKU1), African swine fever (ASF), foot-and-mouth disease virus (FMDV), feline herpesvirus-1/feline viral rhinotracheitis, canine distemper, feline coronavirus (FCoV), or any combination thereof, and a nucleic acid sequence (e.g., mRNA) having an open reading frame encoding at least one universal T-cell epitope.

In some embodiments, a combination vaccine comprises a nucleic acid sequence (e.g., mRNA) encoding a hMPV antigenic polypeptide, a PIV3 antigenic polypeptide, a RSV antigenic polypeptide, a MeV antigenic polypeptide, monkeypox antigenic polypeptide, herpes simplex virus 1 (HSV1) antigenic polypeptide, herpes simplex virus 2 (HSV2) antigenic polypeptide, a coronavirus antigenic polypeptide (e.g., selected from MERS-COV, SARS-CoV-2, HCoV-OC43, HCOV-229E, HCoV-NL63, HCOV-NL, HCOV-NH and HCoV-HKU1), and at least one universal T-cell epitope. In some embodiments, a combination vaccine comprises a nucleic acid sequence (e.g., mRNA) encoding monkeypox antigenic polypeptide, herpes simplex virus 1 (HSV1) antigenic polypeptide, herpes simplex virus 2 (HSV2) antigenic polypeptide, SARS-COV-2 antigenic polypeptide, or any combination thereof, and at least one universal T-cell epitope.

In some embodiments, a combination vaccine comprises a nucleic acid sequence (e.g., mRNA) encoding a hMPV antigenic polypeptide. a PIV3 antigenic polypeptide, a RSV antigenic polypeptide, a MeV antigenic polypeptide, monkeypox antigenic polypeptide, herpes simplex virus 1 (HSV1) antigenic polypeptide, herpes simplex virus 2 (HSV2) antigenic polypeptide, a coronavirus antigenic polypeptide (e.g., selected from MERS-COV, SARS-CoV-2, HCoV-OC43, HCoV-229E, HCoV-NL63, HCoV-NL, HCOV-NH and HCoV-HKU1), and a nucleic acid sequence (e.g., mRNA) encoding at least one universal T-cell epitope. In some embodiments, a combination vaccine comprises a nucleic acid sequence (e.g., mRNA) encoding monkeypox antigenic polypeptide, herpes simplex virus 1 (HSV1) antigenic polypeptide, herpes simplex virus 2 (HSV2) antigenic polypeptide, SARS-COV-2 antigenic polypeptide, or any combination thereof, and a nucleic acid sequence encoding at least one universal T-cell epitope.

In some embodiments, a combination vaccine comprising at least one (e.g., at least 2, 3, 4, or 5) nucleic acid sequence (e.g., mRNA) having an open reading frame encoding a combination of any two or more (or all of) infection agent antigenic polypeptides selected from human Metapneumovirus (hMPV), human parainfluenza viruses (hPIV) types 1, 2, and 3 (hPIV1, hPIV2 and hPIV3, respectively), respiratory syncytial virus (RSV), measles virus (MeV), varicella-zoster, influenza virus, herpes simplex virus 1 (HSV1), herpes simplex virus 2 (HSV2), poxvirus (e.g., smallpox, monkeypox), cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, coronaviruses (e.g., MERS-COV, SARS-COV2, SARS-COV2, HCoV-OC43, HCoV-229E, HCoV-NL63, HCOV-NL, HCOV-NH, HCOV-HKU1), African swine fever (ASF), foot-and-mouth disease virus (FMDV), feline herpesvirus-1/feline viral rhinotracheitis, canine distemper, feline coronavirus (FCoV), or any combination thereof.

In some embodiments, a combination vaccine comprising at least one (e.g., at least 2, 3, 4, or 5) nucleic acid sequence (e.g., mRNA) having an open reading frame encoding a combination of any two or more (or all of) infection agent antigenic polypeptides selected from human Metapneumovirus (hMPV), human parainfluenza viruses (hPIV) types 1, 2, and 3 (hPIV1, hPIV2 and hPIV3, respectively), respiratory syncytial virus (RSV), measles virus (MeV), varicella-zoster, influenza virus, herpes simplex virus 1 (HSV1), herpes simplex virus 2 (HSV2), poxvirus (e.g., smallpox, monkeypox), cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, coronaviruses (e.g., MERS-COV, SARS-COV2, SARS-COV2, HCoV-OC43, HCOV-229E, HCoV-NL63, HCOV-NL, HCOV-NH, HCOV-HKU1), African swine fever (ASF), foot-and-mouth disease virus (FMDV), feline herpesvirus-1/feline viral rhinotracheitis, canine distemper, or any combination thereof.

In some embodiments, a combination vaccine comprising at least one (e.g., at least 2, 3, 4, or 5) nucleic acid sequence (e.g., mRNA) having an open reading frame encoding a combination of any two or more (or all of) infection agent antigenic polypeptides selected from human Metapneumovirus (hMPV), human parainfluenza viruses (hPIV) types 1, 2, and 3 (hPIV1, hPIV2 and hPIV3, respectively), respiratory syncytial virus (RSV), measles virus (MeV). varicella-zoster, influenza virus, herpes simplex virus 1 (HSV1), herpes simplex virus 2 (HSV2), poxvirus (e.g., smallpox, monkeypox), cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus. African swine fever (ASF), foot-and-mouth disease virus (FMDV), feline herpesvirus-1/feline viral rhinotracheitis, canine distemper, feline coronavirus (FCoV), or any combination thereof.

In some embodiments, a combination vaccine comprising at least one (e.g., at least 2, 3, 4, or 5) nucleic acid sequence (e.g., mRNA) having an open reading frame encoding a combination of any two or more (or all of) infection agent antigenic polypeptides selected from human Metapneumovirus (hMPV), human parainfluenza viruses (hPIV) types 1, 2, and 3 (hPIV1, hPIV2 and hPIV3, respectively), respiratory syncytial virus (RSV), measles virus (MeV), varicella-zoster, influenza virus, poxvirus (e.g., smallpox, monkeypox), cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, coronaviruses (e.g., MERS-COV, SARS-COV2, SARS-COV2, HCoV-OC43, HCOV-229E, HCoV-NL63, HCoV-NL, HCOV-NH, HCOV-HKU1), African swine fever (ASF), foot-and-mouth disease virus (FMDV), feline herpesvirus-1/feline viral rhinotracheitis, canine distemper, feline coronavirus (FCoV), or any combination thereof.

In some embodiments, a combination vaccine comprising at least one (e.g., at least 2, 3, 4, or 5) nucleic acid sequence (e.g., mRNA) having an open reading frame encoding a combination of any two or more (or all of) infection agent antigenic polypeptides selected from human Metapneumovirus (hMPV), human parainfluenza viruses (hPIV) types 1, 2, and 3 (hPIV1, hPIV2 and hPIV3, respectively), respiratory syncytial virus (RSV), measles virus (MeV), varicella-zoster, influenza virus, herpes simplex virus 1 (HSV1), herpes simplex virus 2 (HSV2), poxvirus (e.g., smallpox, monkeypox), cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever. Rift Valley fever, hepatitis A, B, C, D, and E virus, African swine fever (ASF), foot-and-mouth disease virus (FMDV), feline herpesvirus-1/feline viral rhinotracheitis, canine distemper, or any combination thereof.

In some embodiments, a combination vaccine comprising at least one (e.g., at least 2, 3, 4, or 5) nucleic acid sequence (e.g., mRNA) having an open reading frame encoding a combination of any two or more (or all of) infection agent antigenic polypeptides selected from human Metapneumovirus (hMPV), human parainfluenza viruses (hPIV) types 1, 2, and 3 (hPIV1, hPIV2 and hPIV3, respectively), respiratory syncytial virus (RSV), measles virus (MeV), varicella-zoster, influenza virus, poxvirus (e.g., smallpox, monkeypox), cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, coronaviruses (e.g., MERS-COV, SARS-COV2, SARS-COV2, HCoV-OC43, HCOV-229E, HCoV-NL63, HCoV-NL, HCOV-NH, HCoV-HKU1), African swine fever (ASF), foot-and-mouth disease virus (FMDV), feline herpesvirus-1/feline viral rhinotracheitis, canine distemper, or any combination thereof.

In some embodiments, a combination vaccine comprising at least one (e.g., at least 2, 3, 4, or 5) nucleic acid sequence (e.g., mRNA) having an open reading frame encoding a combination of any two or more (or all of) infection agent antigenic polypeptides selected from human Metapneumovirus (hMPV), human parainfluenza viruses (hPIV) types 1, 2, and 3 (hPIV1, hPIV2 and hPIV3, respectively), respiratory syncytial virus (RSV), measles virus (MeV), varicella-zoster, influenza virus, poxvirus (e.g., smallpox, monkeypox), cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever. Rift Valley fever, hepatitis A, B, C, D, and E virus, African swine fever (ASF), foot-and-mouth disease virus (FMDV), feline herpesvirus-1/feline viral rhinotracheitis, canine distemper, feline coronavirus (FCoV), or any combination thereof. In some embodiments, a combination vaccine comprises a nucleic acid sequence (e.g., mRNA) encoding a coronavirus (e.g., SARS-COV2) antigenic polypeptide, an Influenza viral antigenic polypeptide, and at least one universal T-cell epitope.

In some embodiments, a combination vaccine comprises nucleic acid sequence (e.g., mRNA) encoding a coronavirus (e.g., SARS-COV2) antigenic polypeptide, an Influenza viral antigenic polypeptide, and at least one universal T-cell epitope from SARS-COV-2 cross-reactive with other common human coronavirus.

In some embodiments, a combination vaccine comprises a nucleic acid sequence (e.g., mRNA) encoding a coronavirus (e.g., SARS-COV2) antigenic polypeptide, monkeypox antigenic polypeptide, and a nucleic acid sequence encoding at least one universal T-cell epitope.

In some embodiments, a combination vaccine comprises a nucleic acid sequence (e.g., mRNA) encoding a coronavirus (e.g., SARS-COV2) antigenic polypeptide, a monkeypox antigenic polypeptide, and a nucleic acid sequence encoding at least one universal T-cell epitope.

In some embodiments, a combination vaccine comprises a nucleic acid sequence (e.g., mRNA) encoding a coronavirus (e.g., SARS-COV2) antigenic polypeptide, monkeypox antigenic polypeptide, and a nucleic acid sequence encoding at least one universal T-cell epitope.

In some embodiments, a combination vaccine comprises a nucleic acid sequence (e.g., mRNA) encoding a coronavirus (e.g., SARS-COV2) antigenic polypeptide, a monkeypox antigenic polypeptide, and a nucleic acid sequence encoding at least one universal T-cell epitope.

In some embodiments, a combination vaccine comprises a nucleic acid sequence (e.g., mRNA) encoding a hMPV antigenic polypeptide, a PIV3 antigenic polypeptide, and at least one universal T-cell epitope.

In some embodiments, a combination vaccine comprises a nucleic acid sequence (e.g., mRNA) encoding a HSV-1 antigenic polypeptide, a HSV-2 antigenic polypeptide, and at least one universal T-cell epitope, substantially cross-reactive to the homologous epitope between HSV-2 and HSV-1.

In some embodiments, a combination vaccine comprises a nucleic acid sequence (e.g., mRNA) encoding a hMPV antigenic polypeptide, a MeV antigenic polypeptide, and at least one universal T-cell epitope.

In some embodiments, a combination vaccine comprises a nucleic acid sequence (e.g., mRNA) encoding a hMPV antigenic polypeptide, a coronavirus antigenic polypeptide, and at least one universal T-cell epitope.

In some embodiments, a combination vaccine comprises a nucleic acid sequence (e.g., mRNA) encoding a PIV3 antigenic polypeptide, a RSV antigenic polypeptide, and at least one universal T-cell epitope.

In some embodiments, a combination vaccine comprises a nucleic acid sequence (e.g., mRNA) encoding a PIV3 antigenic polypeptide, a MeV antigenic polypeptide, and at least one universal T-cell epitope.

In some embodiments, a combination vaccine comprises a nucleic acid sequence (e.g., mRNA) encoding a PIV3 antigenic polypeptide, a coronavirus antigenic polypeptide (e.g., selected from MERS-COV, SARS-COV, HCoV-OC43, HCoV-229E, HCoV-NL63, HCoV-NL, HCoV-NH and HCoV-HKU1), and at least one universal T-cell epitope.

In some embodiments, a combination vaccine comprises a nucleic acid sequence (e.g., mRNA) encoding a RSV antigenic polypeptide, a MeV antigenic polypeptide, and at least one universal T-cell epitope.

In some embodiments, a combination vaccine comprises a nucleic acid sequence (e.g., mRNA) encoding a RSV antigenic polypeptide, a coronavirus antigenic polypeptide (e.g., selected from MERS-COV, SARS-COV, HCoV-OC43, HCoV-229E, HCoV-NL63, HCoV-NL, HCOV-NH and HCoV-HKU1), and at least one universal T-cell epitope.

In some embodiments, a combination vaccine comprises a nucleic acid sequence (e.g., mRNA) encoding a MeV antigenic polypeptide, a coronavirus antigenic polypeptide (e.g., selected from MERS-COV, SARS-COV, HCoV-OC43, HCoV-229E, HCoV-NL63, HCoV-NL, HCOV-NH and HCoV-HKU1), and at least one universal T-cell epitope.

In some embodiments, a combination vaccine comprises a nucleic acid sequence (e.g., mRNA) encoding a hMPV antigenic polypeptide, a PIV3 antigenic polypeptide, a RSV antigenic polypeptide, a MeV antigenic polypeptide, and at least one universal T-cell epitope.

In some embodiments, a combination vaccine comprises a nucleic acid sequence (e.g., mRNA) encoding a hMPV antigenic polypeptide, a PIV3 antigenic polypeptide, a RSV antigenic polypeptide, a coronavirus antigenic polypeptide (e.g., selected from MERS-COV, SARS-COV, HCoV-OC43, HCOV-229E, HCoV-NL63, HCOV-NL, HCOV-NH and HCoV-HKU1), and at least one universal T-cell epitope.

In some embodiments, a combination vaccine comprises a nucleic acid sequence (e.g., mRNA) encoding a hMPV antigenic polypeptide, a PIV3 antigenic polypeptide, a MeV antigenic polypeptide, a coronavirus antigenic polypeptide (e.g., selected from MERS-COV, SARS-COV, HCoV-OC43, HCOV-229E, HCoV-NL63, HCOV-NL, HCOV-NH and HCoV-HKU1), and at least one universal T-cell epitope.

In some embodiments, a combination vaccine comprises a nucleic acid sequence (e.g., mRNA) encoding a hMPV antigenic polypeptide, a RSV antigenic polypeptide, a MeV antigenic polypeptide, a coronavirus antigenic polypeptide (e.g., selected from MERS-COV, SARS-COV, HCoV-OC43, HCOV-229E, HCoV-NL63, HCoV-NL, HCOV-NH and HCoV-HKU1), and at least one universal T-cell epitope.

In some embodiments, a combination vaccine comprises a nucleic acid sequence (e.g., mRNA) encoding a PIV3 antigenic polypeptide, a RSV antigenic polypeptide, a MeV antigenic polypeptide, a coronavirus antigenic polypeptide (e.g., selected from MERS-COV, SARS-COV, HCoV-OC43, HCoV-229E, HCoV-NL63, HCoV-NL, HCOV-NH and HCoV-HKU1), and at least one universal T-cell epitope.

In some embodiments, a combination vaccine comprises a nucleic acid sequence (e.g., mRNA) encoding a hMPV antigenic polypeptide, a PIV3 antigenic polypeptide, a RSV antigenic polypeptide, and at least one universal T-cell epitope.

In some embodiments, a combination vaccine comprises a nucleic acid sequence (e.g., mRNA) encoding a hMPV antigenic polypeptide, a PIV3 antigenic polypeptide, a MeV antigenic polypeptide, and at least one universal T-cell epitope.

In some embodiments, a combination vaccine comprises a nucleic acid sequence (e.g., mRNA) encoding a hMPV antigenic polypeptide, a PIV3 antigenic polypeptide, a coronavirus antigenic polypeptide (e.g., selected from MERS-COV, SARS-COV, HCoV-OC43, HCOV-229E, HCoV-NL63, HCOV-NL, HCoV-NH and HCoV-HKU1), and at least one universal T-cell epitope.

In some embodiments, a combination vaccine comprises a nucleic acid sequence (e.g., mRNA) encoding a hMPV antigenic polypeptide, a RSV antigenic polypeptide, a MeV antigenic polypeptide, and at least one universal T-cell epitope.

In some embodiments, a combination vaccine comprises a nucleic acid sequence (e.g., mRNA) encoding a hMPV antigenic polypeptide, a RSV antigenic polypeptide, a coronavirus antigenic polypeptide (e.g., selected from MERS-COV, SARS-COV, HCoV-OC43, HCOV-229E, HCoV-NL63, HCoV-NL, HCOV-NH and HCoV-HKU1), and at least one universal T-cell epitope.

In some embodiments, a combination vaccine comprises a nucleic acid sequence (e.g., mRNA) encoding a hMPV antigenic polypeptide, a MeV antigenic polypeptide, a coronavirus antigenic polypeptide (e.g., selected from MERS-COV, SARS-COV, HCoV-OC43, HCoV-229E, HCoV-NL63, HCOV-NL, HCOV-NH and HCoV-HKU1), and at least one universal T-cell epitope.

In some embodiments, a combination vaccine comprises a nucleic acid sequence (e.g., mRNA) encoding a PIV3 antigenic polypeptide, a RSV antigenic polypeptide, a MeV antigenic polypeptide, and at least one universal T-cell epitope.

In some embodiments, a combination vaccine comprises a nucleic acid sequence (e.g., mRNA) encoding a PIV3 antigenic polypeptide, a RSV antigenic polypeptide, a coronavirus antigenic polypeptide (e.g., selected from MERS-COV, SARS-COV, HCoV-OC43, HCOV-229E, HCoV-NL63, HCOV-NL, HCOV-NH and HCoV-HKU1), and at least one universal T-cell epitope.

In some embodiments, a combination vaccine comprises a nucleic acid sequence (e.g., mRNA) encoding a RSV antigenic polypeptide, a MeV antigenic polypeptide, a coronavirus antigenic polypeptide (e.g., selected from MERS-COV, SARS-COV, HCoV-OC43, HCoV-229E, HCoV-NL63, HCoV-NL, HCOV-NH and HCoV-HKU1), and at least one universal T-cell epitope.

In some embodiments, a combination vaccine comprises a nucleic acid sequence (e.g., mRNA) encoding a hMPV antigenic polypeptide, a PIV3 antigenic polypeptide, a RSV antigenic polypeptide, a MeV antigenic polypeptide, a coronavirus antigenic polypeptide (e.g., selected from MERS-COV. SARS-COV, HCoV-OC43, HCOV-229E, HCoV-NL63, HCOV-NL, HCOV-NH and HCoV-HKU1), and at least one universal T-cell epitope.

In some embodiments, a combination vaccine comprises a nucleic acid sequence (e.g., mRNA) encoding a hMPV antigenic polypeptide, a PIV3 antigenic polypeptide, a RSV antigenic polypeptide, a MeV antigenic polypeptide, and a coronavirus antigenic polypeptide (e.g., selected from MERS-COV, SARS-COV, HCoV-OC43, HCOV-229E, HCoV-NL63, HCOV-NL, HCOV-NH and HCoV-HKU1), and a nucleic acid sequence (e.g., mRNA) encoding at least one universal T-cell epitope.

In some embodiments, a combination vaccine comprises a nucleic acid sequence (e.g., mRNA) encoding a hMPV antigenic polypeptide and a PIV3 antigenic polypeptide, and a nucleic acid sequence (e.g., mRNA) encoding at least one universal T-cell epitope.

In some embodiments, a combination vaccine comprises a nucleic acid sequence (e.g., mRNA) encoding a hMPV antigenic polypeptide and a RSV antigenic polypeptide, and a nucleic acid sequence (e.g., mRNA) encoding at least one universal T-cell epitope.

In some embodiments, a combination vaccine comprises a nucleic acid sequence (e.g., mRNA) encoding a hMPV antigenic polypeptide and a MeV antigenic polypeptide, and a nucleic acid sequence (e.g., mRNA) encoding at least one universal T-cell epitope.

In some embodiments, a combination vaccine comprises a nucleic acid sequence (e.g., mRNA) encoding a hMPV antigenic polypeptide and a coronavirus antigenic polypeptide, and a nucleic acid sequence (e.g., mRNA) encoding at least one universal T-cell epitope.

In some embodiments, a combination vaccine comprises a nucleic acid sequence (e.g., mRNA) encoding a PIV3 antigenic polypeptide and a RSV antigenic polypeptide, and a nucleic acid sequence (e.g., mRNA) encoding at least one universal T-cell epitope.

In some embodiments, a combination vaccine comprises a nucleic acid sequence (e.g., mRNA) encoding a PIV3 antigenic polypeptide and a MeV antigenic polypeptide, and a nucleic acid sequence (e.g., mRNA) encoding at least one universal T-cell epitope.

In some embodiments, a combination vaccine comprises a nucleic acid sequence (e.g., mRNA) encoding a PIV3 antigenic polypeptide and a coronavirus antigenic polypeptide (e.g., selected from MERS-COV, SARS-COV, HCoV-OC43, HCoV-229E, HCoV-NL63, HCoV-NL, HCOV-NH and HCoV-HKU1), and at least one universal nucleic acid sequence (e.g., mRNA) encoding at least one universal T-cell epitope.

In some embodiments, a combination vaccine comprises a nucleic acid sequence (e.g., mRNA) encoding a RSV antigenic polypeptide and a MeV antigenic polypeptide, and a nucleic acid sequence (e.g., mRNA) encoding at least one universal T-cell epitope.

In some embodiments, a combination vaccine comprises a nucleic acid sequence (e.g., mRNA) encoding a RSV antigenic polypeptide and a coronavirus antigenic polypeptide (e.g., selected from MERS-COV, SARS-COV, HCoV-OC43, HCOV-229E, HCoV-NL63, HCOV-NL, HCOV-NH and HCoV-HKU1), and a nucleic acid sequence (e.g., mRNA) encoding at least one universal T-cell epitope.

In some embodiments, a combination vaccine comprises a nucleic acid sequence (e.g., mRNA) encoding a MeV antigenic polypeptide and a coronavirus antigenic polypeptide (e.g., selected from MERS-COV, SARS-COV, HCoV-OC43, HCOV-229E, HCoV-NL63, HCOV-NL, HCOV-NH and HCoV-HKU1), and a nucleic acid sequence (e.g., mRNA) encoding at least one universal T-cell epitope.

In some embodiments, a combination vaccine comprises a nucleic acid sequence (e.g., mRNA) encoding a hMPV antigenic polypeptide, a PIV3 antigenic polypeptide, a RSV antigenic polypeptide and a MeV antigenic polypeptide, and a nucleic acid sequence (e.g., mRNA) encoding at least one universal T-cell epitope.

In some embodiments, a combination vaccine comprises a nucleic acid sequence (e.g., mRNA) encoding a hMPV antigenic polypeptide, a PIV3 antigenic polypeptide, a RSV antigenic polypeptide and a coronavirus antigenic polypeptide (e.g., selected from MERS-CoV, SARS-COV, HCoV-OC43, HCOV-229E, HCoV-NL63, HCoV-NL, HCOV-NH and HCoV-HKU1), and a nucleic acid sequence (e.g., mRNA) encoding at least one universal T-cell epitope.

In some embodiments, a combination vaccine comprises a nucleic acid sequence (e.g., mRNA) encoding a hMPV antigenic polypeptide, a PIV3 antigenic polypeptide, a MeV antigenic polypeptide and a coronavirus antigenic polypeptide (e.g., selected from MERS-CoV, SARS-COV, HCoV-OC43, HCOV-229E, HCoV-NL63, HCOV-NL, HCOV-NH and HCoV-HKU1), and a nucleic acid sequence (e.g., mRNA) encoding at least one universal T-cell epitope.

In some embodiments, a combination vaccine comprises a nucleic acid sequence (e.g., mRNA) encoding a hMPV antigenic polypeptide, a RSV antigenic polypeptide, a MeV antigenic polypeptide and a coronavirus antigenic polypeptide (e.g., selected from MERS-CoV, SARS-COV, HCoV-OC43, HCOV-229E, HCoV-NL63, HCOV-NL, HCOV-NH and HCoV-HKU1), and a nucleic acid sequence (e.g., mRNA) encoding at least one universal T-cell epitope.

In some embodiments, a combination vaccine comprises a nucleic acid sequence (e.g., mRNA) encoding a PIV3 antigenic polypeptide, a RSV antigenic polypeptide, a MeV antigenic polypeptide and a coronavirus antigenic polypeptide (e.g., selected from MERS-CoV, SARS-COV, HCoV-OC43, HCOV-229E, HCoV-NL63, HCoV-NL, HCOV-NH and HCoV-HKU1), and a nucleic acid sequence (e.g., mRNA) encoding at least one universal T-cell epitope.

In some embodiments, a combination vaccine comprises a nucleic acid sequence (e.g., mRNA) encoding a hMPV antigenic polypeptide, a PIV3 antigenic polypeptide and a RSV antigenic polypeptide, and a nucleic acid sequence (e.g., mRNA) encoding at least one universal T-cell epitope.

In some embodiments, a combination vaccine comprises a nucleic acid sequence (e.g., mRNA) encoding a hMPV antigenic polypeptide, a PIV3 antigenic polypeptide and a MeV antigenic polypeptide, and a nucleic acid sequence (e.g., mRNA) encoding at least one universal T-cell epitope.

In some embodiments, a combination vaccine comprises a nucleic acid sequence (e.g., mRNA) encoding a hMPV antigenic polypeptide, a PIV3 antigenic polypeptide and a coronavirus antigenic polypeptide (e.g., selected from MERS-COV, SARS-COV, HCoV-OC43, HCoV-229E, HCoV-NL63, HCOV-NL, HCOV-NH and HCoV-HKU1), and at least one universal nucleic acid sequence (e.g., mRNA) encoding at least one universal T-cell epitope.

In some embodiments, a combination vaccine comprises a nucleic acid sequence (e.g., mRNA) encoding a hMPV antigenic polypeptide, a RSV antigenic polypeptide and a

MeV antigenic polypeptide, and a nucleic acid sequence (e.g., mRNA) encoding at least one universal T-cell epitope.

In some embodiments, a combination vaccine comprises a nucleic acid sequence (e.g., mRNA) encoding a hMPV antigenic polypeptide, a RSV antigenic polypeptide and a coronavirus antigenic polypeptide (e.g., selected from MERS-COV, SARS-COV, HCoV-OC43, HCOV-229E, HCoV-NL63, HCOV-NL, HCOV-NH and HCoV-HKU1), and a nucleic acid sequence (e.g., mRNA) encoding at least one universal T-cell epitope.

In some embodiments, a combination vaccine comprises a nucleic acid sequence (e.g., mRNA) encoding a hMPV antigenic polypeptide, a MeV antigenic polypeptide and a coronavirus antigenic polypeptide (e.g., selected from MERS-COV, SARS-COV, HCoV-OC43, HCOV-229E, HCoV-NL63, HCOV-NL, HCOV-NH and HCoV-HKU1), and a nucleic acid sequence (e.g., mRNA) encoding at least one universal T-cell epitope.

In some embodiments, a combination vaccine comprises a nucleic acid sequence (e.g., mRNA) encoding a PIV3 antigenic polypeptide, a RSV antigenic polypeptide and a MeV antigenic polypeptide, and a nucleic acid sequence (e.g., mRNA) encoding at least one universal T-cell epitope.

In some embodiments, a combination vaccine comprises a nucleic acid sequence (e.g., mRNA) encoding a PIV3 antigenic polypeptide, a RSV antigenic polypeptide and a coronavirus antigenic polypeptide (e.g., selected from MERS-COV, SARS-COV, HCoV-OC43, HCOV-229E, HCoV-NL63, HCOV-NL, HCOV-NH and HCoV-HKU1), and a nucleic acid sequence (e.g., mRNA) encoding at least one universal T-cell epitope.

In some embodiments, a combination vaccine comprises a nucleic acid sequence (e.g., mRNA) encoding a RSV antigenic polypeptide, a MeV antigenic polypeptide and a coronavirus antigenic polypeptide (e.g., selected from MERS-COV, SARS-COV, HCoV-OC43, HCOV-229E, HCoV-NL63, HCOV-NL, HCOV-NH and HCoV-HKU1), and a nucleic acid sequence (e.g., mRNA) encoding at least one universal T-cell epitope.

Other combination vaccines are encompassed by the present disclosure.

### Infection Agents

In some embodiments, the mRNA can encode an infection agent antigenic polypeptide. In some embodiments, the mRNA can encode a viral antigenic polypeptide. In some embodiments, the virus can be of the family Paramyxoviridae such as human Metapneumovirus (hMPV), parainfluenza viruses (PIV), respiratory syncytial virus (RSV), measles virus (MeV), HIV, varicella-zoster, influenza virus (e.g., influenza A and B), herpes simplex virus 1 (HSV1), herpes simplex virus 2 (HSV2), poxvirus (e.g., smallpox, monkeypox), cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, coronaviruses, or any combination thereof.

Representative examples of parainfluenza viruses (PIV) can include, but are not limited to, human parainfluenza viruses (hPIV) types 1, 2, and 3 (hPIV1, hPIV2 and hPIV3, respectively).

Coronaviruses are enveloped viruses with a positive-sense single-stranded RNA genome and with a nucleocapsid of helical symmetry. Coronaviruses are species of virus belonging to the subfamily Coronavirinae in the family Coronaviridae, in the order Nidovirales.

Representative examples of betacoronaviruses include, but are not limited to an embecovirus 1 (e.g., Betacoronavirus 1, Human coronavirus OC43, China Rattus coronavirus HKU24, Human coronavirus HKU1, Murine coronavirus), a hibecovirus (e.g., Bat Hp-betacoronavirus Zhejiang2013), a merbecovirus (e.g., Hedgehog coronavirus 1, Middle East respiratory syndrome-related coronavirus (MERS-COV), Pipistrellus bat coronavirus HKU5, Tylonycteris bat coronavirus HKU4), a nobecovirus (e.g., Rousettus bat coronavirus GCCDC1, Rousettus bat coronavirus HKU9), a sarbecovirus (e.g., severe acute respiratory syndrome coronavirus (SARS-COV), severe acute respiratory syndrome coronavirus 2 (SARS-COV-2).

Representative examples of gammacoronaviruses include, but are not limited to, a cegacovirus (e.g., Beluga whale coronavirus SQ1) and an Igacovirus (e.g., Avian coronavirus (IBV)).

Representative examples of deltacoronaviruses include, but are not limited to, an andecovirus (e.g., Wigeon coronavirus HKU20), a buldecovirus (e.g., Bulbul coronavirus HKU11, Porcine coronavirus HKU15 (PorCoV HKU15), Munia coronavirus HKU13, White-eye coronavirus HKU16), a herdecovirus (e.g., Night heron coronavirus HKU19), and a moordecovirus (e.g., Common moorhen coronavirus HKU21).

In some embodiments, the coronavirus is a human coronavirus. Representative examples of human coronaviruses include, but are not limited to, severe acute respiratory syndrome coronavirus (SARS-COV), severe acute respiratory syndrome coronavirus 2 (SARS-CoV-2), and Middle East respiratory syndrome-related coronavirus (MERS-COV), human coronavirus 229E (HCoV-229E), human coronavirus OC43 (HCoV-OC43), human coronavirus HKU1 (HCoV-HKU1), Human coronavirus NL63 (HCoV-NL63).

In some embodiments, the mRNA can have an open reading frame encoding at least one (e.g., at least 2, 3, 4 or 5) human Metapneumovirus (hMPV) antigenic polypeptide, human parainfluenza viruses (hPIV) types 1, 2, and 3 (hPIV1, hPIV2 and hPIV3, respectively) antigenic polypeptide, respiratory syncytial virus (RSV) antigenic polypeptide, measles virus (MeV) antigenic polypeptide, varicella-zoster antigenic polypeptide, influenza virus antigenic polypeptide, herpes simplex virus 1 (HSV1) antigenic polypeptide, herpes simplex virus 2 (HSV2) antigenic polypeptide, poxvirus (e.g., smallpox, monkeypox) antigenic polypeptide, cytomegalovirus antigenic polypeptide. Epstein-Barr virus antigenic polypeptide, rotavirus antigenic polypeptide, rhinovirus antigenic polypeptide, adenovirus antigenic polypeptide, papillomavirus antigenic polypeptide, poliovirus antigenic polypeptide, mumps antigenic polypeptide, rabies antigenic polypeptide, rubella antigenic polypeptide, coxsackieviruses antigenic polypeptide, equine encephalitis antigenic polypeptide, Japanese encephalitis antigenic polypeptide, yellow fever antigenic polypeptide, Rift Valley fever antigenic polypeptide, hepatitis A, B, C, D, and E virus antigenic polypeptide, or coronaviruses (e.g., MERS-COV, SARS-COV, SARS-COV2, HCoV-OC43, HCOV-229E, HCoV-NL63, HCoV-NL, HCOV-NH, HCoV-HKU1) antigenic polypeptide, or any combination of two or more of the antigenic polypeptides.

### Universal T-Cell Epitope (UTE)

In some embodiments, the nucleic acid sequence (e.g., an mRNA) can encode at least one universal T-cell epitope. In some embodiments, the nucleic acid sequence can encode a recombinant or heterologous antigen containing at least one universal T-cell epitope, which will be processed and presented on the surface of the cell in the context of a major histocompatibility complex (MHC) molecule. Suitable T-cell epitope can include, but are not limited to, MHC class I (MHC I) and MHC class II restricted (MHC II) T cell epitopes. The universal T-cell epitopes amino acid sequence can be of from 8 amino acid (aa) to 15 aa for MHC I restrict CD8 T cell and 10 aa to 28 aa for MHC II restricted CD4 T cell epitopes. The universal T-cell epitopes can be any sequences that have been identified, or based on epitope prediction through computational tools.

In one embodiment, the composition can include at least one universal T-cell epitope. In some embodiments, the T-cell epitope can bind to specific HLA-A1, HLA-A2, HLA-B7, HLA-B40, HLA-Cw7 alleles, or any combination thereof.

### MHC 1 and MHC II

There are two subtypes of MHC molecules, MHC Class I and II molecules (MHC I and MHC II). These subtypes correspond to two subsets of T-lymphocytes: 1) CD8+ cytotoxic T-cells, which usually recognize peptides presented by MHC I, and kill infected cells or cells harboring a mutation(s), and 2) CD4+ helper T-cells, which usually recognize peptides presented by MHC Class II molecules, and regulate the responses of other cells of the immune system. A “MHC Class I molecule” as used everywhere herein is defined as a molecule which comprises 1-3 subunits, including a heavy chain, a heavy chain combined with a light chain (B2m). A “MHC Class II molecule” as used everywhere herein is defined as a molecule which comprises 2-3 subunits including an a-chain and a P-chain (oc/β-dimer).

MHC Class I like molecules (including non-classical MHC Class I molecules) can include, but are not limited to, CDId, HLA E, HLA G, HLA F, HLA H, MIC A, MIC B, ULBP-1, ULBP-2, and ULBP-3. MHC Class II like molecules (including non-classical MHC Class II molecules) can include, but are not limited to, HLA DM, HLA D0, I-A beta2, and I-E beta2.

The MHC molecule can suitably be a vertebrate MHC molecule such as a human, a mouse, a rat, a canine, a porcine, a feline, a bovine or an avian MHC molecule. Such MHC molecules from different species have different names. For example, in humans, the term “MHC” is used interchangeably with HLA herein.

In general, the term “MHC molecule” is intended to include alleles. By way of example, in humans e. g. HLA A, HLA B, HLA C, HLA D, HLA E, HLA F, HLA G, HLA H, HLA DR, HLA DQ and HLA DP alleles are of interest, and in the mouse system, H-2 alleles are of interest. Likewise, in the rat system RT1-alleles, in the porcine system SLA-alleles, in the bovine system BoLA, in the avian system e. g. chicken-B alleles, are of interest.

### Infection Agent Antigenic Polypeptides

Herein, use of the term “antigen” encompasses immunogenic fragments of the antigen (an immunogenic fragment that induces (or is capable of inducing) an immune response to hMPV, PIV, RSV, MeV, influenza virus, HSV1, HSV2, poxvirus, or coronaviruses (e.g., SARS-COV2), unless otherwise stated.

Human Metapneumovirus (hMPV)

hMPV shares substantial homology with respiratory syncytial virus (RSV) in its surface glycoproteins. hMPV fusion protein (F) is related to other paramyxovirus fusion proteins and appears to have homologous regions that may have similar functions. The hMPV fusion protein amino acid sequence contains features characteristic of other paramyxovirus F proteins, including a putative cleavage site and potential N-linked glycosylation sites. Paramyxovirus fusion proteins are synthesized as inactive precursors (FO) that are cleaved by host cell proteases into the biologically fusion-active F1 and F2 domains (see, e.g., Cseke G. et al. Journal of Virology 2007; 81 (2):698-707, incorporated herein by reference). hMPV has one putative cleavage site, in contrast to the two sites established for RSV F, and only shares 34% amino acid sequence identity with RSV F. F2 is extracellular and disulfide linked to F1. Fusion proteins are type I glycoproteins existing as trimers, with two 4-3 heptad repeat domains at the N- and C-terminal regions of the protein (HR 1 and HR2), which form coiled-coil alpha-helices. These coiled coils become apposed in an antiparallel fashion when the protein undergoes a conformational change into the fusogenic state. There is a hydrophobic fusion peptide N proximal to the N-terminal heptad repeat, which is thought to insert into the target cell membrane, while the association of the heptad repeats brings the transmembrane domain into close proximity, inducing membrane fusion (see, e.g., Baker, K A et al. Mol. Cell 1999; 3:309-319). This mechanism has been proposed for a number of different viruses, including RSV, influenza virus, and human immunodeficiency virus. Fusion proteins are major antigenic determinants for all known paramyxoviruses and for other viruses that possess similar fusion proteins such as human immunodeficiency virus, influenza virus, and Ebola virus.

In some embodiments, a hMPV vaccine of the present disclosure comprises a nucleic acid sequence (e.g., mRNA) encoding hMPV fusion protein (F), and a nucleic acid sequence (e.g., mRNA) encoding at least one universal T-cell epitope. In some embodiments, a hMPV vaccine of the present disclosure comprises a nucleic acid sequence (e.g., mRNA) encoding a F1 or F2 subunit of a hMPV F protein, and a nucleic acid sequence (e.g., mRNA) encoding at least one universal T-cell epitope. In some embodiments, a hMPV vaccine of the present disclosure comprises a nucleic acid sequence (e.g., mRNA) encoding hMPV glycoprotein (G), and a nucleic acid sequence (e.g., mRNA) encoding at least one universal T-cell epitope. In some embodiments, a hMPV vaccine of the present disclosure comprises a nucleic acid sequence (e.g., mRNA) encoding hMPV matrix protein (M), and a nucleic acid sequence (e.g., mRNA) encoding at least one universal T-cell epitope. In some embodiments, a hMPV vaccine of the present disclosure comprises a nucleic acid sequence (e.g., mRNA) encoding hMPV phosphoprotein (P), and a nucleic acid sequence (e.g., mRNA) encoding at least one universal T-cell epitope. In some embodiments, a hMPV vaccine of the present disclosure comprises a nucleic acid sequence (e.g., mRNA) encoding hMPV nucleoprotein (N), and a nucleic acid sequence (e.g., mRNA) encoding at least one universal T-cell epitope. In some embodiments, a hMPV vaccine of the present disclosure comprises a nucleic acid sequence (e.g., mRNA) encoding hMPV SH protein (SH), and a nucleic acid sequence (e.g., mRNA) (e.g., mRNA) polynucleotide encoding at least one universal T-cell epitope.

In some embodiments, a hMPV vaccine of the present disclosure comprises a nucleic acid sequence (e.g., mRNA) encoding hMPV fusion protein (F), and at least one T-cell epitope. In some embodiments, a hMPV vaccine of the present disclosure comprises a nucleic acid sequence (e.g., mRNA) encoding a Fl or F2 subunit of a hMPV F protein, and at least one T-cell epitope. In some embodiments, a hMPV vaccine of the present disclosure comprises a nucleic acid sequence (e.g., mRNA) encoding hMPV glycoprotein (G), and at least one universal T-cell epitope. In some embodiments, a hMPV vaccine of the present disclosure comprises a nucleic acid sequence (e.g., mRNA) encoding hMPV matrix protein (M), and at least one universal T-cell epitope. In some embodiments, a hMPV vaccine of the present disclosure comprises a nucleic acid sequence (e.g., mRNA) encoding hMPV phosphoprotein (P), and at least one universal T-cell epitope. In some embodiments, a hMPV vaccine of the present disclosure comprises a nucleic acid sequence (e.g., mRNA) encoding hMPV nucleoprotein (N), and at least one universal T-cell epitope. In some embodiments, a hMPV vaccine of the present disclosure comprises a nucleic acid sequence (e.g., mRNA) encoding hMPV SH protein (SH), and at least one universal T-cell epitope.

In some embodiments, a hMPV vaccine of the present disclosure comprises a nucleic acid sequence (e.g., mRNA) encoding F protein, G protein, M protein, P protein, N protein and SH protein, and a nucleic acid sequence (e.g., mRNA) encoding at least one universal T-cell epitope.

In some embodiments, a hMPV vaccine of the present disclosure comprises a nucleic acid sequence (e.g., mRNA) encoding F protein, G protein, M protein, P protein, N protein, SH protein, and at least one universal T-cell epitope.

In some embodiments, a hMPV vaccine of the present disclosure comprises a nucleic acid sequence (e.g., mRNA) encoding F protein and G protein, and a nucleic acid sequence (e.g., mRNA) encoding at least one universal T-cell epitope. In some embodiments, a hMPV vaccine of the present disclosure comprises a nucleic acid sequence (e.g., mRNA) encoding F protein and M protein, and a nucleic acid sequence (e.g., mRNA) encoding at least one universal T-cell epitope. In some embodiments, a hMPV vaccine of the present disclosure comprises a nucleic acid sequence (e.g., mRNA) encoding F protein and P protein, and a nucleic acid sequence (e.g., mRNA) encoding at least one universal T-cell epitope. In some embodiments, a hMPV vaccine of the present disclosure comprises a nucleic acid sequence (e.g., mRNA) encoding F protein and N protein, and a nucleic acid sequence (e.g., mRNA) encoding at least one universal T-cell epitope. In some embodiments, a hMPV vaccine of the present disclosure comprises a nucleic acid sequence (e.g., mRNA) encoding F protein and SH protein, and a nucleic acid sequence (e.g., mRNA) encoding at least one universal T-cell epitope.

In some embodiments, a hMPV vaccine of the present disclosure comprises a nucleic acid sequence (e.g., mRNA) encoding G protein and M protein, and a nucleic acid sequence (e.g., mRNA) encoding at least one universal T-cell epitope. In some embodiments, a hMPV vaccine of the present disclosure comprises a nucleic acid sequence (e.g., mRNA) encoding G protein and P protein, and a nucleic acid sequence (e.g., mRNA) encoding at least one universal T-cell epitope. In some embodiments, a hMPV vaccine of the present disclosure comprises a nucleic acid sequence (e.g., mRNA) encoding G protein and N protein, and a nucleic acid sequence (e.g., mRNA) encoding at least one universal T-cell epitope. In some embodiments, a hMPV vaccine of the present disclosure comprises a nucleic acid sequence (e.g., mRNA) encoding G protein and SH protein, and a nucleic acid sequence (e.g., mRNA) encoding at least one universal T-cell epitope.

In some embodiments, a hMPV vaccine of the present disclosure comprises a nucleic acid sequence (e.g., mRNA) encoding F protein, G protein and M protein, and a nucleic acid sequence (e.g., mRNA) encoding at least one universal T-cell epitope. In some embodiments, a hMPV vaccine of the present disclosure comprises a nucleic acid sequence (e.g., mRNA) encoding F protein, G protein and P protein, and a nucleic acid sequence (e.g., mRNA) encoding at least one universal T-cell epitope. In some embodiments, a hMPV vaccine of the present disclosure comprises a nucleic acid sequence (e.g., mRNA) encoding F protein, G protein and N protein, and a nucleic acid sequence (e.g., mRNA) encoding at least one universal T-cell epitope. In some embodiments, a hMPV vaccine of the present disclosure comprises a nucleic acid sequence (e.g., mRNA) encoding F protein, G protein and SH protein, and a nucleic acid sequence (e.g., mRNA) encoding at least one universal T-cell epitope.

A hMPV vaccine may comprise, for example, at least one RNA (e.g., mRNA) polynucleotide having an open reading frame encoding at least one hMPV antigenic polypeptide identified by any one of SEQ ID NO: 5-8, and a nucleic acid sequence (e.g., mRNA) encoding at least one universal T-cell epitope.

A hMPV vaccine may comprise, for example, at least one RNA (e.g., mRNA) polynucleotide encoded by a nucleic acid (e.g., DNA) identified by any one of SEQ ID NO: 1-4, and a nucleic acid sequence (e.g., mRNA) encoding at least one universal T-cell epitope.

In some embodiments, a hMPV vaccine of the present disclosure comprises a nucleic acid sequence (e.g., mRNA) encoding F protein, G protein, and at least one universal T-cell epitope. In some embodiments, a hMPV vaccine of the present disclosure comprises a nucleic acid sequence (e.g., mRNA) encoding F protein, M protein, and at least one universal T-cell epitope. In some embodiments, a hMPV vaccine of the present disclosure comprises a nucleic acid sequence (e.g., mRNA) encoding F protein, P protein, and at least one universal T-cell epitope. In some embodiments, a hMPV vaccine of the present disclosure comprises a nucleic acid sequence (e.g., mRNA) encoding F protein, N protein, and at least one universal T-cell epitope. In some embodiments, a hMPV vaccine of the present disclosure comprises a nucleic acid sequence (e.g., mRNA) encoding F protein, SH protein, and at least one universal T-cell epitope.

In some embodiments, a hMPV vaccine of the present disclosure comprises a nucleic acid sequence (e.g., mRNA) encoding G protein, M protein, and at least one universal T-cell epitope. In some embodiments, a hMPV vaccine of the present disclosure comprises a nucleic acid sequence (e.g., mRNA) encoding G protein, P protein, and at least one universal T-cell epitope. In some embodiments, a hMPV vaccine of the present disclosure comprises a nucleic acid sequence (e.g., mRNA) encoding G protein, N protein, and at least one universal T-cell epitope. In some embodiments, a hMPV vaccine of the present disclosure comprises a nucleic acid sequence (e.g., mRNA) encoding G protein, SH protein, and at least one universal T-cell epitope.

In some embodiments, a hMPV vaccine of the present disclosure comprises a nucleic acid sequence (e.g., mRNA) encoding F protein, G protein, M protein, and at least one universal T-cell epitope. In some embodiments, a hMPV vaccine of the present disclosure comprises a nucleic acid sequence (e.g., mRNA) encoding F protein, G protein, P protein, and at least one universal T-cell epitope. In some embodiments, a hMPV vaccine of the present disclosure comprises a nucleic acid sequence (e.g., mRNA) encoding F protein, G protein, N protein, and at least one universal T-cell epitope. In some embodiments, a hMPV vaccine of the present disclosure comprises a nucleic acid sequence (e.g., mRNA) encoding F protein, G protein, SH protein, and at least one universal T-cell epitope.

A hMPV vaccine may comprise, for example, at least one RNA (e.g., mRNA) polynucleotide having an open reading frame encoding at least one hMPV antigenic polypeptide identified by any one of SEQ ID NO: 5-8, and at least one universal T-cell epitope.

A hMPV vaccine may comprise, for example, at least one RNA (e.g., mRNA) polynucleotide encoded by a nucleic acid (e.g., DNA) identified by any one of SEQ ID NO: 1-4, and at least one universal T-cell epitope.

The present disclosure is not limited by a particular strain of hMPV. The strain of hMPV used in a vaccine may be any strain of hMPV. Non-limiting examples of strains of hMPV for use as provide herein include the CAN98-75 (CAN75) and the CAN97-83 (CAN83) hMPV strains (Skiadopoulos M H et al. J Virol. 20014; 78 (13) 6927-37, incorporated herein by reference), a hMPV A1, A2, B1 or B2 strain (see, e.g., de Graaf M et al. The Journal of General Virology 2008; 89:975-83; Peret T C T et al. The Journal of Infectious Disease 2002; 185:1660-63. incorporated herein by reference), a hMPV isolate TN/92-4 (e.g., SEQ ID NO: 1 and 5), a hMPV isolate NL/1/99 (e.g., SEQ ID NO: 2 and 6), or a hMPV isolate PER/CFI0497/2010/B (e.g., SEQ ID NO: 3 and 7).

In some embodiments, at least one hMPV antigenic polypeptide is obtained from a hMPV A1, A2, B1 or B2 strain (see, e.g., de Graaf M et al. The Journal of General Virology 2008; 89:975-83; Peret T C T et al. The Journal of Infectious Disease 2002; 185:1660-63, incorporated herein by reference). In some embodiments, at least one antigenic polypeptide is obtained from the CAN98-75 (CAN75) hMPV strain. In some embodiments, at least one antigenic polypeptide is obtained from the CAN97-83 (CAN83) hMPV strain. In some embodiments, at least one antigenic polypeptide is obtained from hMPV isolate TN/92-4 (e.g., SEQ ID NO: 1 and 5). In some embodiments, at least one antigenic polypeptide is obtained from hMPV isolate NL/1/99 (e.g., SEQ ID NO: 2 and 6). In some embodiments, at least one antigenic polypeptide is obtained from hMPV isolate PER/CFI0497/2010/B (e.g., SEQ ID NO: 3 and 7).

In some embodiments, hMPV vaccines comprise RNA (e.g., mRNA) polynucleotides encoding a hMPV antigenic polypeptides having at least 95%, at least 96%, at least 97%, at least 98% or at least 99% identity with hMPV F protein and having F protein activity.

A protein is considered to have F protein activity if, for example, the protein acts to fuse the viral envelope and host cell plasma membrane, mediates viral entry into a host cell via an interaction with arginine-glycine-aspartate RGD-binding integrins, or a combination thereof (see, e.g., Cox R G et al. J Virol. 2012; 88 (22): 12148-60, incorporated herein by reference).

In some embodiments, hMPV vaccines comprise RNA (e.g., mRNA) polynucleotides encoding hMPV antigenic polypeptides having at least 95%, at least 96%, at least 97%, at least 98% or at least 99% identity with hMPV G protein and having G protein activity.

A protein is considered to have G protein activity if, for example, the protein acts to modulate (e.g., inhibit) hMPV-induced cellular (immune) responses (see, e.g., Bao X et al. PLOS Pathog. 2008; 4 (5): e1000077, incorporated herein by reference).

### Human Parainfluenza Virus Type 3 (PIV3)

Parainfluenza viruses belong to the family Paramyxoviridae. These are enveloped viruses with a negative-sense single-stranded RNA genome. Parainfluenza viruses belong to the subfamily Paramyxoviridae, which is subdivided into three genera: Respirovirus (PIV-1, PIV-3, and Sendai virus (SeV)), Rubulavirus (PIV-2, PIV-4 and mumps virus) and Morbillivirus (measles virus, rinderpest virus and canine distemper virus (CDV)). Their genome, a ˜15 500 nucleotide-long negative-sense RNA molecule, encodes two envelope glycoproteins, the hemagglutinin-neuraminidase (HN), the fusion protein (F or FO), which is cleaved into F1 and F2 subunits, a matrix protein (M), a nucleocapsid protein (N) and several nonstructural proteins including the viral replicase (L). All parainfluenza viruses, except for PIV-1, express a non-structural V protein that blocks IFN signaling in the infected cell and acts therefore as a virulence factor (see, e.g., Nishio M et al. J Virol. 2008; 82 (13): 6130-38).

PIV3 hemagglutinin-neuraminidase (HN), a structural protein, is found on the viral envelope, where it is necessary for attachment and cell entry. It recognizes and binds to sialic acid-containing receptors on the host cell's surface. As a neuroaminidase, HN removes sialic acid from virus particles, preventing self-aggregation of the virus, and promoting the efficient spread of the virus. Furthermore, HN promotes the activity of the fusion (F or FO) protein, contributing to the penetration of the host cell's surface.

PIV3 fusion protein (PIV3 F) is located on the viral envelope, where it facilitates the viral fusion and cell entry. The F protein is initially inactive, but proteolytic cleavage leads to its active forms, F1 and F2, which are linked by disulfide bonds. This occurs when the HN protein binds its receptor on the host cell's surface. During early phases of infection, the F glycoprotein mediates penetration of the host cell by fusion of the viral envelope to the plasma membrane. In later stages of the infection, the F protein facilitates the fusion of the infected cells with neighboring uninfected cells, which leads to the formation of a syncytium and spread of the infection.

PIV3 matrix protein (M) is found within the viral envelope and assists with viral assembly. It interacts with the nucleocapsid and envelope glycoproteins, where it facilitates the budding of progeny viruses through its interactions with specific sites on the cytoplasmic tail of the viral glycoproteins and nucleocapsid. It also plays a role in transporting viral components to the budding site.

PIV3 phosphoprotein (P) and PIV3 large polymerase protein (L) are found in the nucleocapsid where they form part of the RNA polymerase complex. The L protein, a viral RNA-dependent RNA polymerase, facilitates genomic transcription, while the host cell's ribosomes translate the viral mRNA into viral proteins.

PIV3 V is a non-structural protein that blocks IFN signaling in the infected cell, therefore acting as a virulence factor.

PIV3 nucleoprotein (N) encapsidates the genome in a ratio of 1 N per 6 ribonucleotides, protecting it from nucleases. The nucleocapsid (NC) has a helical structure. The encapsidated genomic RNA is termed the NC and serves as template for transcription and replication. During replication, encapsidation by PIV3 N is coupled to RNA synthesis and all replicative products are resistant to nucleases. PIV3 N homo-multimerizes to form the nucleocapsid and binds to viral genomic RNA. PIV3 N binds the P protein and thereby positions the polymerase on the template.

In some embodiments, a PIV3 vaccine of the present disclosure comprises a nucleic acid sequence (e.g., mRNA) encoding PIV3 fusion protein (F), and a nucleic acid sequence (e.g., mRNA) encoding at least one universal T-cell epitope. In some embodiments, a PIV3 vaccine of the present disclosure comprises a nucleic acid sequence (e.g., mRNA) encoding a Fl or F2 subunit of a PIV3 F protein, and a nucleic acid sequence (e.g., mRNA) encoding at least one universal T-cell epitope. In some embodiments, a PIV3 vaccine of the present disclosure comprises a nucleic acid sequence (e.g., mRNA) encoding PIV3 hemagglutinin-neuraminidase (HN) (see, e.g., van Wyke Coelingh K L et al. J Virol. 1987; 61 (5): 1473-77, incorporated herein by reference), and a nucleic acid sequence (e.g., mRNA) encoding at least one universal T-cell epitope. In some embodiments, a PIV3 vaccine of the present disclosure comprises a nucleic acid sequence (e.g., mRNA) encoding PIV3 matrix protein (M), and a nucleic acid sequence (e.g., mRNA) encoding at least one universal T-cell epitope. In some embodiments, a PIV3 vaccine of the present disclosure comprises a nucleic acid sequence (e.g., mRNA) encoding PIV3 phosphoprotein (P), and a nucleic acid sequence (e.g., mRNA) encoding at least one universal T-cell epitope. In some embodiments, a PIV3 vaccine of the present disclosure comprises a nucleic acid sequence (e.g., mRNA) encoding PIV3 nucleoprotein (N), and a nucleic acid sequence (e.g., mRNA) encoding at least one universal T-cell epitope.

In some embodiments, a PIV3 vaccine of the present disclosure comprises a nucleic acid sequence (e.g., mRNA) encoding F protein, HN protein, M protein, P protein, and N protein, and a nucleic acid sequence (e.g., mRNA) encoding at least one universal T-cell epitope.

In some embodiments, a PIV3 vaccine of the present disclosure comprises a nucleic acid sequence (e.g., mRNA) encoding F protein and HN protein, and a nucleic acid sequence (e.g., mRNA) encoding at least one universal T-cell epitope. In some embodiments, a PIV3 vaccine of the present disclosure comprises a nucleic acid sequence (e.g., mRNA) encoding F protein and M protein, and a nucleic acid sequence (e.g., mRNA) encoding at least one universal T-cell epitope. In some embodiments, a PIV3 vaccine of the present disclosure comprises a nucleic acid sequence (e.g., mRNA) encoding F protein and P protein, and a nucleic acid sequence (e.g., mRNA) encoding at least one universal T-cell epitope. In some embodiments, a PIV3 vaccine of the present disclosure comprises a nucleic acid sequence (e.g., mRNA) encoding F protein and N protein, and a nucleic acid sequence (e.g., mRNA) encoding at least one universal T-cell epitope.

In some embodiments, a PIV3 vaccine of the present disclosure comprises a nucleic acid sequence (e.g., mRNA) encoding HN protein and M protein, and a nucleic acid sequence (e.g., mRNA) encoding at least one universal T-cell epitope. In some embodiments, a PIV3 vaccine of the present disclosure comprises a nucleic acid sequence (e.g., mRNA) encoding HN protein and P protein, and a nucleic acid sequence (e.g., mRNA) encoding at least one universal T-cell epitope. In some embodiments, a PIV3 vaccine of the present disclosure comprises a nucleic acid sequence (e.g., mRNA) encoding HN protein and N protein, and a nucleic acid sequence (e.g., mRNA) encoding at least one universal T-cell epitope.

In some embodiments, a PIV3 vaccine of the present disclosure comprises a nucleic acid sequence (e.g., mRNA) encoding F protein, HN protein and M protein, and a nucleic acid sequence (e.g., mRNA) encoding at least one universal T-cell epitope. In some embodiments, a PIV3 vaccine of the present disclosure comprises a nucleic acid sequence (e.g., mRNA) encoding F protein, HN protein and P protein, and a nucleic acid sequence (e.g., mRNA) encoding at least one universal T-cell epitope. In some embodiments, a PIV3 vaccine of the present disclosure comprises a nucleic acid sequence (e.g., mRNA) encoding F protein, HN protein and N protein, and a nucleic acid sequence (e.g., mRNA) encoding at least one universal T-cell epitope.

A PIV3 vaccine may comprise, for example, at least one RNA (e.g., mRNA) polynucleotide having an open reading frame encoding at least one PIV3 antigenic polypeptide identified by any one of SEQ ID NO: 12-13, and a nucleic acid sequence (e.g., mRNA) encoding at least one universal T-cell epitope.

A PIV3 vaccine may comprise, for example, at least one RNA (e.g., mRNA) polynucleotide encoded by a nucleic acid (e.g., DNA) identified by any one of SEQ ID NO: 9-12, and a nucleic acid sequence (e.g., mRNA) encoding at least one universal T-cell epitope.

In some embodiments, a PIV3 vaccine of the present disclosure comprises a nucleic acid sequence (e.g., mRNA) encoding PIV3 fusion protein (F), and at least one universal T-cell epitope. In some embodiments, a PIV3 vaccine of the present disclosure comprises a nucleic acid sequence (e.g., mRNA) encoding a F1 or F2 subunit of a PIV3 F protein, and at least one universal T-cell epitope. In some embodiments, a PIV3 vaccine of the present disclosure comprises a nucleic acid sequence (e.g., mRNA) encoding PIV3 hemagglutinin-neuraminidase (HN) (see, e.g., van Wyke Coelingh K L et al. J Virol. 1987; 61 (5): 1473-77, incorporated herein by reference), and at least one universal T-cell epitope. In some embodiments, a PIV3 vaccine of the present disclosure comprises a nucleic acid sequence (e.g., mRNA) encoding PIV3 matrix protein (M), and at least one universal T-cell epitope. In some embodiments, a PIV3 vaccine of the present disclosure comprises a nucleic acid sequence (e.g., mRNA) encoding PIV3 phosphoprotein (P), and at least one universal T-cell epitope. In some embodiments, a PIV3 vaccine of the present disclosure comprises a nucleic acid sequence (e.g., mRNA) encoding PIV3 nucleoprotein (N), and at least one universal T-cell epitope.

In some embodiments, a PIV3 vaccine of the present disclosure comprises a nucleic acid sequence (e.g., mRNA) encoding F protein, HN protein, M protein, P protein, N protein, and at least one universal T-cell epitope.

In some embodiments, a PIV3 vaccine of the present disclosure comprises a nucleic acid sequence (e.g., mRNA) encoding F protein, HN protein, and at least one universal T-cell epitope. In some embodiments, a PIV3 vaccine of the present disclosure comprises a nucleic acid sequence (e.g., mRNA) encoding F protein, M protein, and at least one universal T-cell epitope. In some embodiments, a PIV3 vaccine of the present disclosure comprises a nucleic acid sequence (e.g., mRNA) encoding F protein, P protein, and at least one universal T-cell epitope. In some embodiments, a PIV3 vaccine of the present disclosure comprises a nucleic acid sequence (e.g., mRNA) encoding F protein, N protein, and at least one universal T-cell epitope.

In some embodiments, a PIV3 vaccine of the present disclosure comprises a nucleic acid sequence (e.g., mRNA) encoding HN protein, M protein, and at least one universal T-cell epitope. In some embodiments, a PIV3 vaccine of the present disclosure comprises a nucleic acid sequence (e.g., mRNA) encoding HN protein, P protein, and at least one universal T-cell epitope. In some embodiments, a PIV3 vaccine of the present disclosure comprises a nucleic acid sequence (e.g., mRNA) encoding HN protein, N protein, and at least one universal T-cell epitope.

In some embodiments, a PIV3 vaccine of the present disclosure comprises a nucleic acid sequence (e.g., mRNA) encoding F protein, HN protein, M protein, and at least one universal T-cell epitope. In some embodiments, a PIV3 vaccine of the present disclosure comprises a nucleic acid sequence (e.g., mRNA) encoding F protein, HN protein, P protein, and at least one universal T-cell epitope. In some embodiments, a PIV3 vaccine of the present disclosure comprises a nucleic acid sequence (e.g., mRNA) encoding F protein, HN protein, N protein, and at least one universal T-cell epitope.

A PIV3 vaccine may comprise, for example, at least one RNA (e.g., mRNA) polynucleotide having an open reading frame encoding at least one PIV3 antigenic polypeptide identified by any one of SEQ ID NO: 12-13, and at least one universal T-cell epitope.

A PIV3 vaccine may comprise, for example, at least one RNA (e.g., mRNA) polynucleotide encoded by a nucleic acid (e.g., DNA) identified by any one of SEQ ID NO: 9-12, and at least one universal T-cell epitope.

The present disclosure is not limited by a particular strain of PIV3. The strain of PIV3 used in a vaccine may be any strain of PIV3. A non-limiting example of a strain of PIV3 for use as provide herein includes HPIV3/Homo sapiens/PER/FLA4815/2008.

In some embodiments, PIV3 vaccines comprise RNA (e.g., mRNA) polynucleotides encoding a PIV3 antigenic polypeptides having at least 95%, at least 96%, at least 97%, at least 98% or at least 99% identity with PIV3 F protein and having F protein activity.

In some embodiments, PIV3 vaccines comprise RNA (e.g., mRNA) polynucleotides encoding PIV3 antigenic polypeptides having at least 95%, at least 96%, at least 97%, at least 98% or at least 99% identity with PIV3 hemagglutinin-neuraminidase (HN) and having hemagglutinin-neuraminidase activity.

A protein is considered to have hemagglutinin-neuraminidase activity if, for example, it is capable of both receptor binding and receptor cleaving. Such proteins are major surface glycoproteins that have functional sites for cell attachment and for neuraminidase activity. They are able to cause red blood cells to agglutinate and to cleave the glycosidic linkages of neuraminic acids, so they have the potential to both bind a potential host cell and then release the cell if necessary, for example, to prevent self-aggregation of the virus.

In some embodiments, PIV3 vaccines comprise RNA (e.g., mRNA) polynucleotides encoding PIV3 antigenic polypeptides having at least 95%, at least 96%, at least 97%, at least 98% or at least 99% identity with PIV3 HN, F (e.g., F, Fl or F2), M, N, L or V and having HN, F (e.g., F, Fl or F2), M, N, L or V activity, respectively.

### Respiratory Syncytial Virus (RSV)

RSV is a negative-sense, single-stranded RNA virus of the genus Pneumovirinae. The virus is present in at least two antigenic subgroups, known as Group A and Group B, primarily resulting from differences in the surface G glycoproteins. Two RSV surface glycoproteins-G and F-mediate attachment with and attachment to cells of the respiratory epithelium. F surface glycoproteins mediate coalescence of neighboring cells. This results in the formation of syncytial cells. RSV is the most common cause of bronchiolitis. Most infected adults develop mild cold-like symptoms such as congestion, low-grade fever, and wheezing. Infants and small children may suffer more severe symptoms such as bronchiolitis and pneumonia. The disease may be transmitted among humans via contact with respiratory secretions.

The genome of RSV encodes at least three surface glycoproteins, including F, G, and SH, four nucleocapsid proteins, including L, P, N, and M2, and one matrix protein, M. Glycoprotein F directs viral penetration by fusion between the virion and the host membrane. Glycoprotein G is a type II transmembrane glycoprotein and is the major attachment protein. SH is a short integral membrane protein. Matrix protein M is found in the inner layer of the lipid bilayer and assists virion formation. Nucleocapsid proteins L, P, N, and M2 modulate replication and transcription of the RSV genome. It is thought that glycoprotein G tethers and stabilizes the virus particle at the surface of bronchial epithelial cells, while glycoprotein F interacts with cellular glycosaminoglycans to mediate fusion and delivery of the RSV virion contents into the host cell (Krzyzaniak M A et al. PLoS Pathog 2013; 9 (4)).

In some embodiments, a RSV vaccine of the present disclosure comprises a nucleic acid sequence (e.g., mRNA) encoding F protein, and a nucleic acid sequence (e.g., mRNA) encoding at least one universal T-cell epitope. In some embodiments, a PIV3 vaccine of the present disclosure comprises a nucleic acid sequence (e.g., mRNA) encoding G protein, and a nucleic acid sequence (e.g., mRNA) encoding at least one universal T-cell epitope. In some embodiments, a PIV3 vaccine of the present disclosure comprises a nucleic acid sequence (e.g., mRNA) encoding L protein, and a nucleic acid sequence (e.g., mRNA) encoding at least one universal T-cell epitope. In some embodiments, a PIV3 vaccine of the present disclosure comprises a nucleic acid sequence (e.g., mRNA) encoding P protein. In some embodiments, a PIV3 vaccine of the present disclosure comprises a nucleic acid sequence (e.g., mRNA) encoding N protein, and a nucleic acid sequence (e.g., mRNA) encoding at least one universal T-cell epitope. In some embodiments, a PIV3 vaccine of the present disclosure comprises a nucleic acid sequence (e.g., mRNA) encoding M2 protein, and a nucleic acid sequence (e.g., mRNA) encoding at least one universal T-cell epitope. In some embodiments, a PIV3 vaccine of the present disclosure comprises a nucleic acid sequence (e.g., mRNA) encoding M protein, and a nucleic acid sequence (e.g., mRNA) encoding at least one universal T-cell epitope.

In some embodiments, a RSV vaccine of the present disclosure comprises a nucleic acid sequence (e.g., mRNA) encoding F protein, G protein, L protein, P protein, N protein, M2 protein and M protein, and a nucleic acid sequence (e.g., mRNA) encoding at least one universal T-cell epitope.

In some embodiments, a RSV vaccine of the present disclosure comprises a nucleic acid sequence (e.g., mRNA) encoding F protein and G protein, and a nucleic acid sequence (e.g., mRNA) encoding at least one universal T-cell epitope. In some embodiments, a RSV vaccine of the present disclosure comprises a nucleic acid sequence (e.g., mRNA) encoding F protein and L protein, and a nucleic acid sequence (e.g., mRNA) encoding at least one universal T-cell epitope. In some embodiments, a RSV vaccine of the present disclosure comprises a nucleic acid sequence (e.g., mRNA) encoding F protein and P protein, and a nucleic acid sequence (e.g., mRNA) encoding at least one universal T-cell epitope. In some embodiments, a RSV vaccine of the present disclosure comprises a nucleic acid sequence (e.g., mRNA) encoding F protein and N protein, and a nucleic acid sequence (e.g., mRNA) encoding at least one universal T-cell epitope. In some embodiments, a RSV vaccine of the present disclosure comprises a nucleic acid sequence (e.g., mRNA) encoding F protein and M2 protein, and a nucleic acid sequence (e.g., mRNA) encoding at least one universal T-cell epitope. In some embodiments, a RSV vaccine of the present disclosure comprises a nucleic acid sequence (e.g., mRNA) encoding F protein and M protein, and a nucleic acid sequence (e.g., mRNA) encoding at least one universal T-cell epitope.

In some embodiments, a RSV vaccine of the present disclosure comprises a nucleic acid sequence (e.g., mRNA) encoding G protein and L protein, and a nucleic acid sequence (e.g., mRNA) encoding at least one universal T-cell epitope. In some embodiments, a RSV vaccine of the present disclosure comprises a nucleic acid sequence (e.g., mRNA) encoding G protein and P protein, and a nucleic acid sequence (e.g., mRNA) encoding at least one universal T-cell epitope. In some embodiments, a RSV vaccine of the present disclosure comprises a nucleic acid sequence (e.g., mRNA) encoding G protein and N protein, and a nucleic acid sequence (e.g., mRNA) encoding at least one universal T-cell epitope. In some embodiments, a RSV vaccine of the present disclosure comprises a nucleic acid sequence (e.g., mRNA) encoding G protein and M2 protein, and a nucleic acid sequence (e.g., mRNA) encoding at least one universal T-cell epitope. In some embodiments, a RSV vaccine of the present disclosure comprises a nucleic acid sequence (e.g., mRNA) encoding G protein and M protein, and a nucleic acid sequence (e.g., mRNA) encoding at least one universal T-cell epitope.

In some embodiments, a RSV vaccine of the present disclosure comprises a nucleic acid sequence (e.g., mRNA) encoding F protein, G protein and L protein, and a nucleic acid sequence (e.g., mRNA) encoding at least one universal T-cell epitope. In some embodiments, a RSV vaccine of the present disclosure comprises a nucleic acid sequence (e.g., mRNA) encoding F protein, G protein and P protein, and a nucleic acid sequence (e.g., mRNA) encoding at least one universal T-cell epitope. In some embodiments, a RSV vaccine of the present disclosure comprises a nucleic acid sequence (e.g., mRNA) encoding F protein, G protein and N protein, and a nucleic acid sequence (e.g., mRNA) encoding at least one universal T-cell epitope. In some embodiments, a RSV vaccine of the present disclosure comprises a nucleic acid sequence (e.g., mRNA) encoding F protein, G protein and M2 protein, and a nucleic acid sequence (e.g., mRNA) encoding at least one universal T-cell epitope. In some embodiments, a RSV vaccine of the present disclosure comprises a nucleic acid sequence (e.g., mRNA) encoding F protein, G protein and M protein, and a nucleic acid sequence (e.g., mRNA) encoding at least one universal T-cell epitope.

In some embodiments, a RSV vaccine of the present disclosure comprises a nucleic acid sequence (e.g., mRNA) encoding F protein, and a nucleic acid sequence (e.g., mRNA) encoding at least one universal T-cell epitope. In some embodiments, a PIV3 vaccine of the present disclosure comprises a nucleic acid sequence (e.g., mRNA) encoding G protein, and a nucleic acid sequence (e.g., mRNA) encoding at least one universal T-cell epitope. In some embodiments, a PIV3 vaccine of the present disclosure comprises a nucleic acid sequence (e.g., mRNA) encoding L protein, and a nucleic acid sequence (e.g., mRNA) encoding at least one universal T-cell epitope. In some embodiments, a PIV3 vaccine of the present disclosure comprises a nucleic acid sequence (e.g., mRNA) encoding P protein. In some embodiments, a PIV3 vaccine of the present disclosure comprises a nucleic acid sequence (e.g., mRNA) encoding N protein, and a nucleic acid sequence (e.g., mRNA) encoding at least one universal T-cell epitope. In some embodiments, a PIV3 vaccine of the present disclosure comprises a nucleic acid sequence (e.g., mRNA) encoding M2 protein, and a nucleic acid sequence (e.g., mRNA) encoding at least one universal T-cell epitope. In some embodiments, a PIV3 vaccine of the present disclosure comprises a nucleic acid sequence (e.g., mRNA) encoding M protein, and a nucleic acid sequence (e.g., mRNA) encoding at least one universal T-cell epitope.

In some embodiments, a RSV vaccine of the present disclosure comprises a nucleic acid sequence (e.g., mRNA) encoding F protein, G protein, L protein, P protein, N protein, M2 protein and M protein, and a nucleic acid sequence (e.g., mRNA) encoding at least one universal T-cell epitope.

In some embodiments, a RSV vaccine of the present disclosure comprises a nucleic acid sequence (e.g., mRNA) encoding F protein and G protein, and a nucleic acid sequence (e.g., mRNA) encoding at least one universal T-cell epitope. In some embodiments, a RSV vaccine of the present disclosure comprises a nucleic acid sequence (e.g., mRNA) encoding F protein and L protein, and a nucleic acid sequence (e.g., mRNA) encoding at least one universal T-cell epitope. In some embodiments, a RSV vaccine of the present disclosure comprises a nucleic acid sequence (e.g., mRNA) encoding F protein and P protein, and a nucleic acid sequence (e.g., mRNA) encoding at least one universal T-cell epitope. In some embodiments, a RSV vaccine of the present disclosure comprises a nucleic acid sequence (e.g., mRNA) encoding F protein and N protein, and a nucleic acid sequence (e.g., mRNA) encoding at least one universal T-cell epitope. In some embodiments, a RSV vaccine of the present disclosure comprises a nucleic acid sequence (e.g., mRNA) encoding F protein and M2 protein, and a nucleic acid sequence (e.g., mRNA) encoding at least one universal T-cell epitope. In some embodiments, a RSV vaccine of the present disclosure comprises a nucleic acid sequence (e.g., mRNA) encoding F protein and M protein, and a nucleic acid sequence (e.g., mRNA) encoding at least one universal T-cell epitope.

In some embodiments, a RSV vaccine of the present disclosure comprises a nucleic acid sequence (e.g., mRNA) encoding G protein and L protein, and a nucleic acid sequence (e.g., mRNA) encoding at least one universal T-cell epitope. In some embodiments, a RSV vaccine of the present disclosure comprises a nucleic acid sequence (e.g., mRNA) encoding G protein and P protein, and a nucleic acid sequence (e.g., mRNA) encoding at least one universal T-cell epitope. In some embodiments, a RSV vaccine of the present disclosure comprises a nucleic acid sequence (e.g., mRNA) encoding G protein and N protein, and a nucleic acid sequence (e.g., mRNA) encoding at least one universal T-cell epitope. In some embodiments, a RSV vaccine of the present disclosure comprises a nucleic acid sequence (e.g., mRNA) encoding G protein and M2 protein, and a nucleic acid sequence (e.g., mRNA) encoding at least one universal T-cell epitope. In some embodiments, a RSV vaccine of the present disclosure comprises a nucleic acid sequence (e.g., mRNA) encoding G protein and M protein, and a nucleic acid sequence (e.g., mRNA) encoding at least one universal T-cell epitope.

In some embodiments, a RSV vaccine of the present disclosure comprises a nucleic acid sequence (e.g., mRNA) encoding F protein, G protein and L protein, and a nucleic acid sequence (e.g., mRNA) encoding at least one universal T-cell epitope. In some embodiments, a RSV vaccine of the present disclosure comprises a nucleic acid sequence (e.g., mRNA) encoding F protein, G protein and P protein, and a nucleic acid sequence (e.g., mRNA) encoding at least one universal T-cell epitope. In some embodiments, a RSV vaccine of the present disclosure comprises a nucleic acid sequence (e.g., mRNA) encoding F protein, G protein and N protein, and a nucleic acid sequence (e.g., mRNA) encoding at least one universal T-cell epitope. In some embodiments, a RSV vaccine of the present disclosure comprises a nucleic acid sequence (e.g., mRNA) encoding F protein, G protein and M2 protein, and a nucleic acid sequence (e.g., mRNA) encoding at least one universal T-cell epitope. In some embodiments, a RSV vaccine of the present disclosure comprises a nucleic acid sequence (e.g., mRNA) encoding F protein, G protein and M protein, and a nucleic acid sequence (e.g., mRNA) encoding at least one universal T-cell epitope.

In some embodiments, a RSV vaccine of the present disclosure comprises a nucleic acid sequence (e.g., mRNA) encoding F protein, and at least one universal T-cell epitope. In some embodiments, a PIV3 vaccine of the present disclosure comprises a nucleic acid sequence (e.g., mRNA) encoding G protein, and at least one universal T-cell epitope. In some embodiments, a PIV3 vaccine of the present disclosure comprises a nucleic acid sequence (e.g., mRNA) encoding L protein, and at least one universal T-cell epitope. In some embodiments, a PIV3 vaccine of the present disclosure comprises a nucleic acid sequence (e.g., mRNA) encoding P protein. In some embodiments, a PIV3 vaccine of the present disclosure comprises a nucleic acid sequence (e.g., mRNA) encoding N protein, and at least one universal T-cell epitope. In some embodiments, a PIV3 vaccine of the present disclosure comprises a nucleic acid sequence (e.g., mRNA) encoding M2 protein, and at least one universal T-cell epitope. In some embodiments, a PIV3 vaccine of the present disclosure comprises a nucleic acid sequence (e.g., mRNA) encoding M protein, and at least one universal T-cell epitope.

In some embodiments, a RSV vaccine of the present disclosure comprises a nucleic acid sequence (e.g., mRNA) encoding F protein, G protein, L protein, P protein, N protein, M2 protein, M protein, and at least one universal T-cell epitope.

In some embodiments, a RSV vaccine of the present disclosure comprises a nucleic acid sequence (e.g., mRNA) encoding F protein, G protein, and at least one universal T-cell epitope. In some embodiments, a RSV vaccine of the present disclosure comprises a nucleic acid sequence (e.g., mRNA) encoding F protein, L protein, and at least one universal T-cell epitope. In some embodiments, a RSV vaccine of the present disclosure comprises a nucleic acid sequence (e.g., mRNA) encoding F protein, P protein, and at least one universal T-cell epitope. In some embodiments, a RSV vaccine of the present disclosure comprises a nucleic acid sequence (e.g., mRNA) encoding F protein, N protein, and at least one universal T-cell epitope. In some embodiments, a RSV vaccine of the present disclosure comprises a nucleic acid sequence (e.g., mRNA) encoding F protein, M2 protein, and at least one universal T-cell epitope. In some embodiments, a RSV vaccine of the present disclosure comprises a nucleic acid sequence (e.g., mRNA) encoding F protein, M protein, and at least one universal T-cell epitope.

In some embodiments, a RSV vaccine of the present disclosure comprises a nucleic acid sequence (e.g., mRNA) encoding G protein, L protein, and at least one universal T-cell epitope. In some embodiments, a RSV vaccine of the present disclosure comprises a nucleic acid sequence (e.g., mRNA) encoding G protein, P protein, and at least one universal T-cell epitope. In some embodiments, a RSV vaccine of the present disclosure comprises a nucleic acid sequence (e.g., mRNA) encoding G protein, N protein, and at least one universal T-cell epitope. In some embodiments, a RSV vaccine of the present disclosure comprises a nucleic acid sequence (e.g., mRNA) encoding G protein, M2 protein, and at least one universal T-cell epitope. In some embodiments, a RSV vaccine of the present disclosure comprises a nucleic acid sequence (e.g., mRNA) encoding G protein, M protein, and at least one universal T-cell epitope.

In some embodiments, a RSV vaccine of the present disclosure comprises a nucleic acid sequence (e.g., mRNA) encoding F protein, G protein, L protein, and at least one universal T-cell epitope. In some embodiments, a RSV vaccine of the present disclosure comprises a nucleic acid sequence (e.g., mRNA) encoding F protein, G protein, P protein, and at least one universal T-cell epitope. In some embodiments, a RSV vaccine of the present disclosure comprises a nucleic acid sequence (e.g., mRNA) encoding F protein, G protein. N protein, and at least one universal T-cell epitope. In some embodiments, a RSV vaccine of the present disclosure comprises a nucleic acid sequence (e.g., mRNA) encoding F protein, G protein, M2 protein, and at least one universal T-cell epitope. In some embodiments, a RSV vaccine of the present disclosure comprises a nucleic acid sequence (e.g., mRNA) encoding F protein, G protein, M protein, and at least one universal T-cell epitope.

The present disclosure is not limited by a particular strain of RSV. The strain of RSV used in a vaccine may be any strain of RSV.

In some embodiments, RSV vaccines comprise RNA (e.g., mRNA) polynucleotides encoding a RSV antigenic polypeptides having at least 95%, at least 96%, at least 97%, at least 98% or at least 99% identity with RSV F protein and having F protein activity.

In some embodiments, RSV vaccines comprise RNA (e.g., mRNA) polynucleotides encoding RSV antigenic polypeptides having at least 95%, at least 96%, at least 97%, at least 98% or at least 99% identity with RSV G protein and having G protein activity.

A protein is considered to have G protein activity if, for example, the protein acts to modulate (e.g., inhibit) hMPV-induced cellular (immune) responses (see, e.g., Bao X et al. PLoS Pathog. 2008; 4 (5): e 1000077, incorporated herein by reference).

### Measles Virus (MeV)

Molecular epidemiologic investigations and virologic surveillance contribute notably to the control and prevention of measles. Nearly half of measles-related deaths worldwide occur in India, yet virologic surveillance data are incomplete for many regions of the country. Previous studies have documented the presence of measles virus genotypes D4, D7, and D8 in India, and genotypes D5, D9, D11, H1, and G3 have been detected in neighboring countries. Recently, MeV genotype B3 was detected in India (Kuttiatt V S et al. Emerg Infect Dis. 2014; 20(10):1764-66).

The glycoprotein complex of paramyxoviruses mediates receptor binding and membrane fusion. In particular, the MeV fusion (F) protein executes membrane fusion, after receptor binding by the hemagglutinin (HA) protein (Muhlebach M D et al. Journal of Virology 2008; 82 (22): 11437-45). The MeV P gene codes for three proteins: P, an essential polymerase cofactor, and V and C, which have multiple functions but are not strictly required for viral propagation in cultured cells. V shares the amino-terminal domain with P but has a zinc-binding carboxyl-terminal domain, whereas C is translated from an overlapping reading frame. The MeV C protein is an infectivity factor. During replication, the P protein binds incoming monomeric nucleocapsid (N) proteins with its amino-terminal domain and positions them for assembly into the nascent ribonucleocapsid. The P protein amino-terminal domain is natively unfolded (Deveaux P et al. Journal of Virology 2004; 78 (21): 11632-40).

In some embodiments, a MeV vaccine of the present disclosure comprises a nucleic acid sequence (e.g., mRNA) encoding HA protein and a nucleic acid sequence (e.g., mRNA) encoding at least one universal T-cell epitope. In some embodiments, a MeV vaccine of the present disclosure comprises a nucleic acid sequence (e.g., mRNA) encoding F protein and a nucleic acid sequence (e.g., mRNA) encoding at least one universal T-cell epitope. In some embodiments, a MeV vaccine of the present disclosure comprises a nucleic acid sequence (e.g., mRNA) encoding P protein and a nucleic acid sequence (e.g., mRNA) encoding at least one universal T-cell epitope. In some embodiments, a MeV vaccine of the present disclosure comprises a nucleic acid sequence (e.g., mRNA) encoding V protein and a nucleic acid sequence (e.g., mRNA) encoding at least one universal T-cell epitope. In some embodiments, a MeV vaccine of the present disclosure comprises a nucleic acid sequence (e.g., mRNA) encoding C protein and a nucleic acid sequence (e.g., mRNA) encoding at least one universal T-cell epitope.

In some embodiments, a MeV vaccine of the present disclosure comprises a nucleic acid sequence (e.g., mRNA) encoding HA protein, F protein, P protein, V protein and C protein and a nucleic acid sequence (e.g., mRNA) encoding at least one universal T-cell epitope.

In some embodiments, a MeV vaccine of the present disclosure comprises a nucleic acid sequence (e.g., mRNA) encoding HA protein and F protein and a nucleic acid sequence (e.g., mRNA) encoding at least one universal T-cell epitope. In some embodiments, a MeV vaccine of the present disclosure comprises a nucleic acid sequence (e.g., mRNA) encoding HA protein and P protein and a nucleic acid sequence (e.g., mRNA) encoding at least one universal T-cell epitope. In some embodiments, a MeV vaccine of the present disclosure comprises a nucleic acid sequence (e.g., mRNA) encoding HA protein and V protein and a nucleic acid sequence (e.g., mRNA) encoding at least one universal T-cell epitope. In some embodiments, a MeV vaccine of the present disclosure comprises a nucleic acid sequence (e.g., mRNA) encoding HA protein and C protein and a nucleic acid sequence (e.g., mRNA) encoding at least one universal T-cell epitope.

some embodiments, a MeV vaccine of the present disclosure comprises a nucleic acid sequence (e.g., mRNA) encoding F protein and P protein and a nucleic acid sequence (e.g., mRNA) encoding at least one universal T-cell epitope. In some embodiments, a MeV vaccine of the present disclosure comprises a nucleic acid sequence (e.g., mRNA) encoding F protein and V protein and a nucleic acid sequence (e.g., mRNA) encoding at least one universal T-cell epitope. In some embodiments, a MeV vaccine of the present disclosure comprises a nucleic acid sequence (e.g., mRNA) encoding F protein and C protein and a nucleic acid sequence (e.g., mRNA) encoding at least one universal T-cell epitope.

In some embodiments, a MeV vaccine of the present disclosure comprises a nucleic acid sequence (e.g., mRNA) encoding HA protein, F protein and P protein and a nucleic acid sequence (e.g., mRNA) encoding at least one universal T-cell epitope. In some embodiments, a MeV vaccine of the present disclosure comprises a nucleic acid sequence (e.g., mRNA) encoding HA protein, F protein and V protein and a nucleic acid sequence (e.g., mRNA) encoding at least one universal T-cell epitope. In some embodiments, a MeV vaccine of the present disclosure comprises a nucleic acid sequence (e.g., mRNA) encoding HA protein, F protein and C protein and a nucleic acid sequence (e.g., mRNA) encoding at least one universal T-cell epitope.

In some embodiments, MeV vaccines comprise RNA (e.g., mRNA) encoding a MeV antigenic polypeptide having at least 95%, at least 96%, at least 97%, at least 98% or at least 99% identity with MeV HA protein and having MeV HA protein activity.

In some embodiments, MeV vaccines comprise RNA (e.g., mRNA) encoding a MeV antigenic polypeptide having at least 95%, at least 96%, at least 97%, at least 98% or at least 99% identity with MeV F protein and having MeV F protein activity.

A protein is considered to have HA protein activity if the protein mediates receptor binding and/or membrane fusion. MeV F protein executes membrane fusion, after receptor binding by the MeV HA protein.

A MeV vaccine may comprise, for example, at least one RNA (e.g., mRNA) polynucleotide having an open reading frame encoding at least one MeV antigenic polypeptide identified by any one of SEQ ID NO: 47-50 and a nucleic acid sequence (e.g., mRNA) encoding at least one universal T-cell epitope.

A MeV vaccine may comprise, for example, at least one RNA (e.g., mRNA) polynucleotide identified by any one of SEQ ID NO: 37, 40, 43, 46.

A MeV vaccine may comprise, for example, at least one RNA (e.g., mRNA) polynucleotide encoded by a nucleic acid (e.g., DNA) identified by any one of SEQ ID NO: 35, 36, 38, 39, 41, 42, 44 and 45 and a nucleic acid sequence (e.g., mRNA) encoding at least one universal T-cell epitope.

In some embodiments, a MeV vaccine of the present disclosure comprises a nucleic acid sequence (e.g., mRNA) encoding HA protein and at least one universal T-cell epitope. In some embodiments, a MeV vaccine of the present disclosure comprises a nucleic acid sequence (e.g., mRNA) encoding F protein and at least one universal T-cell epitope. In some embodiments, a MeV vaccine of the present disclosure comprises a nucleic acid sequence (e.g., mRNA) encoding P protein and at least one universal T-cell epitope. In some embodiments, a MeV vaccine of the present disclosure comprises a nucleic acid sequence (e.g., mRNA) encoding V protein and at least one universal T-cell epitope. In some embodiments, a MeV vaccine of the present disclosure comprises a nucleic acid sequence (e.g., mRNA) encoding C protein and at least one universal T-cell epitope.

In some embodiments, a MeV vaccine of the present disclosure comprises a nucleic acid sequence (e.g., mRNA) encoding HA protein, F protein, P protein, V protein, C protein and at least one universal T-cell epitope.

In some embodiments, a MeV vaccine of the present disclosure comprises a nucleic acid sequence (e.g., mRNA) encoding HA protein, F protein and at least one universal T-cell epitope. In some embodiments, a MeV vaccine of the present disclosure comprises a nucleic acid sequence (e.g., mRNA) encoding HA protein, P protein and at least one universal T-cell epitope. In some embodiments, a MeV vaccine of the present disclosure comprises a nucleic acid sequence (e.g., mRNA) encoding HA protein, V protein and at least one universal T-cell epitope. In some embodiments, a MeV vaccine of the present disclosure comprises a nucleic acid sequence (e.g., mRNA) encoding HA protein, C protein and at least one universal T-cell epitope.

some embodiments, a MeV vaccine of the present disclosure comprises a nucleic acid sequence (e.g., mRNA) encoding F protein, P protein, and at least one universal T-cell epitope. In some embodiments, a MeV vaccine of the present disclosure comprises a nucleic acid sequence (e.g., mRNA) encoding F protein, V protein, and at least one universal T-cell epitope. In some embodiments, a MeV vaccine of the present disclosure comprises a nucleic acid sequence (e.g., mRNA) encoding F protein, C protein, and at least one universal T-cell epitope.

In some embodiments, a MeV vaccine of the present disclosure comprises a nucleic acid sequence (e.g., mRNA) encoding HA protein, F protein, P protein, and at least one universal T-cell epitope. In some embodiments, a MeV vaccine of the present disclosure comprises a nucleic acid sequence (e.g., mRNA) encoding HA protein, F protein, V protein, and at least one universal T-cell epitope. In some embodiments, a MeV vaccine of the present disclosure comprises a nucleic acid sequence (e.g., mRNA) encoding HA protein, F protein, C protein, and at least one universal T-cell epitope.

A MeV vaccine may comprise, for example, at least one RNA (e.g., mRNA) polynucleotide having an open reading frame encoding at least one MeV antigenic polypeptide identified by any one of SEQ ID NO: 47-50 and at least one universal T-cell epitope.

A MeV vaccine may comprise, for example, at least one RNA (e.g., mRNA) polynucleotide identified by any one of SEQ ID NO: 37, 40, 43, 46.

A MeV vaccine may comprise, for example, at least one RNA (e.g., mRNA) polynucleotide encoded by a nucleic acid (e.g., DNA) identified by any one of SEQ ID NO: 35, 36, 38, 39, 41, 42, 44 and 45 and a nucleic acid sequence (e.g., mRNA) encoding at least one universal T-cell epitope.

The present disclosure is not limited by a particular strain of MeV. The strain of MeV used in a vaccine may be any strain of MeV. Non-limiting examples of strains of MeV for use as provide herein include B3/B3.1, C2, D4, D6, D7, D8, G3, H1, Moraten, Rubeovax, MVi/New Jersey. USA/45.05, MVi/Texas. USA/4.07, AIK-C, MVi/New York. USA/26.09/3, MVi/California.USA/16.03, MVi/Virginia.USA/15.09, MVi/California.USA/8.04, and MVi/Pennsylvania.USA/20.09.

MeV proteins may be from MeV genotype D4, D5, D7, D8, D9, D11, H1, G3 or B3. In some embodiments, a MeV HA protein or a MeV F protein is from MeV genotype D8. In some embodiments, a MeV HA protein or a MeV F protein is from MeV genotype B3.

### Coronaviruses

MERS-Co V. MERS-COV is a positive-sense, single-stranded RNA virus of the genus Betacoronavirus. The genomes are phylogenetically classified into two clades, clade A and clade B. It has a strong tropism for non-ciliated bronchial epithelial cells, evades the innate immune response and antagonizes interferon (IFN) production in infected cells. Dipeptyl peptidase 4 (DDP4, also known as CD26) has been identified as a functional cellular receptor for MERS-COV. Its enzymatic activity is not required for infection, although its amino acid sequence is highly conserved across species and is expressed in the human bronchial epithelium and kidneys. Most infected individuals develop severe acute respiratory illnesses, including fever, cough, and shortness of breath, and the virus can be fatal. The disease may be transmitted among humans, generally among those in close contact.

The genome of MERS-COV encodes at least four unique accessory proteins, such as 3, 4a, 4b and 5, two replicase proteins (open reading frame 1a and 1b), and four major structural proteins, including spike(S), envelope (E), nucleocapsid (N), and membrane (M) proteins (Almazan F et al. MBio 2013; 4 (5): e00650-13). The accessory proteins play nonessential roles in MERS-COV replication, but they are likely structural proteins or interferon antagonists, modulating in vivo replication efficiency and/or pathogenesis, as in the case of SARS-COV (Almazan F et al. MBio 2013; 4 (5): e00650-13; Totura A L et al. Curr Opin Virol 2012; 2(3):264-75; Scobey T et al. Proc Natl Acad Sci USA 2013; 110(40):16157-62). The other proteins of MERS-COV maintain different functions in virus replication. The E protein, for example, involves in virulence, and deleting the E-coding gene results in replication-competent and propagation-defective viruses or attenuated viruses (Almazan F et al. MBio 2013; 4(5):e00650-13). The S protein is particularly essential in mediating virus binding to cells expressing receptor dipeptidyl peptidase-4 (DPP4) through receptor-binding domain (RBD) in the S1 subunit, whereas the S2 subunit subsequently mediates virus entry via fusion of the virus and target cell membranes (Li F. J Virol 2015; 89(4):1954-64; Raj V S et al. Nature 2013; 495 (7440): 251-4).

In some embodiments, a MERS-COV vaccine of the present disclosure comprises a nucleic acid sequence (e.g., mRNA) encoding S protein, and a nucleic acid sequence (e.g., mRNA) encoding at least one universal T-cell epitope. In some embodiments, a MERS-CoV vaccine of the present disclosure comprises a nucleic acid sequence (e.g., mRNA) encoding the SI subunit of the S protein, and a nucleic acid sequence (e.g., mRNA) encoding at least one universal T-cell epitope. In some embodiments, a MERS-COV vaccine of the present disclosure comprises a nucleic acid sequence (e.g., mRNA) encoding the S2 subunit of the S protein, and a nucleic acid sequence (e.g., mRNA) encoding at least one universal T-cell epitope. In some embodiments, a MERS-COV vaccine of the present disclosure comprises a nucleic acid sequence (e.g., mRNA) encoding E protein, and a nucleic acid sequence (e.g., mRNA) encoding at least one universal T-cell epitope. In some embodiments, a MERS-CoV vaccine of the present disclosure comprises a nucleic acid sequence (e.g., mRNA) encoding N protein, and a nucleic acid sequence (e.g., mRNA) encoding at least one universal T-cell epitope. In some embodiments, a MERS-COV vaccine of the present disclosure comprises a nucleic acid sequence (e.g., mRNA) encoding M protein, and a nucleic acid sequence (e.g., mRNA) encoding at least one universal T-cell epitope.

In some embodiments, a MERS-COV vaccine of the present disclosure comprises a nucleic acid sequence (e.g., mRNA) encoding S protein (S, S1 and/or S2), E protein, N protein and M protein, and a nucleic acid sequence (e.g., mRNA) encoding at least one universal T-cell epitope.

In some embodiments, a MERS-COV vaccine of the present disclosure comprises a nucleic acid sequence (e.g., mRNA) encoding S protein (S, S1 and/or S2) and E protein, and a nucleic acid sequence (e.g., mRNA) encoding at least one universal T-cell epitope. In some embodiments, a MERS-COV vaccine of the present disclosure comprises a nucleic acid sequence (e.g., mRNA) encoding S protein (S, S1 and/or S2) and N protein, and a nucleic acid sequence (e.g., mRNA) encoding at least one universal T-cell epitope. In some embodiments, a MERS-COV vaccine of the present disclosure comprises a nucleic acid sequence (e.g., mRNA) encoding S protein (S, S1 and/or S2) and M protein, and a nucleic acid sequence (e.g., mRNA) encoding at least one universal T-cell epitope.

In some embodiments, a MERS-COV vaccine of the present disclosure comprises a nucleic acid sequence (e.g., mRNA) encoding S protein (S, S1 and/or S2), E protein and M protein, and a nucleic acid sequence (e.g., mRNA) encoding at least one universal T-cell epitope. In some embodiments, a MERS-COV vaccine of the present disclosure comprises a nucleic acid sequence (e.g., mRNA) encoding S protein (S, S1 and/or S2), E protein and N protein, and a nucleic acid sequence (e.g., mRNA) encoding at least one universal T-cell epitope. In some embodiments, a MERS-COV vaccine of the present disclosure comprises a nucleic acid sequence (e.g., mRNA) encoding S protein (S, S1 and/or S2), M protein and N protein, and a nucleic acid sequence (e.g., mRNA) encoding at least one universal T-cell epitope. In some embodiments, a MERS-COV vaccine of the present disclosure comprises a nucleic acid sequence (e.g., mRNA) encoding E protein, M protein and N protein, and a nucleic acid sequence (e.g., mRNA) encoding at least one universal T-cell epitope.

A MERS-COV vaccine may comprise, for example, at least one RNA (e.g., mRNA) polynucleotide having an open reading frame encoding at least one MERS-COV antigenic polypeptide identified by any one of SEQ ID NO: 24-38 or 33, and a nucleic acid sequence (e.g., mRNA) encoding at least one universal T-cell epitope.

A MERS-COV vaccine may comprise, for example, at least one RNA (e.g., mRNA) polynucleotide encoded by a nucleic acid (e.g., DNA) identified by any one of SEQ ID NO: 20-23, and a nucleic acid sequence (e.g., mRNA) encoding at least one universal T-cell epitope.

In some embodiments, a MERS-COV vaccine of the present disclosure comprises a nucleic acid sequence (e.g., mRNA) encoding S protein, and at least one universal T-cell epitope. In some embodiments, a MERS-COV vaccine of the present disclosure comprises a nucleic acid sequence (e.g., mRNA) encoding the SI subunit of the S protein, and at least one universal T-cell epitope. In some embodiments, a MERS-COV vaccine of the present disclosure comprises a nucleic acid sequence (e.g., mRNA) encoding the S2 subunit of the S protein, and at least one universal T-cell epitope. In some embodiments, a MERS-CoV vaccine of the present disclosure comprises a nucleic acid sequence (e.g., mRNA) encoding E protein, and at least one universal T-cell epitope. In some embodiments, a MERS-CoV vaccine of the present disclosure comprises a nucleic acid sequence (e.g., mRNA) encoding N protein, and at least one universal T-cell epitope. In some embodiments, a MERS-CoV vaccine of the present disclosure comprises a nucleic acid sequence (e.g., mRNA) encoding M protein, and at least one universal T-cell epitope.

In some embodiments, a MERS-COV vaccine of the present disclosure comprises a nucleic acid sequence (e.g., mRNA) encoding S protein (S, S1 and/or S2), E protein, N protein, M protein, and at least one universal T-cell epitope.

In some embodiments, a MERS-COV vaccine of the present disclosure comprises a nucleic acid sequence (e.g., mRNA) encoding S protein (S, S1 and/or S2), E protein, and at least one universal T-cell epitope. In some embodiments, a MERS-COV vaccine of the present disclosure comprises a nucleic acid sequence (e.g., mRNA) encoding S protein (S, S1 and/or S2), N protein, and at least one universal T-cell epitope. In some embodiments, a MERS-CoV vaccine of the present disclosure comprises a nucleic acid sequence (e.g., mRNA) encoding S protein (S, S1 and/or S2), M protein, and at least one universal T-cell epitope.

In some embodiments, a MERS-COV vaccine of the present disclosure comprises a nucleic acid sequence (e.g., mRNA) encoding S protein (S, S1 and/or S2), E protein. M protein, and at least one universal T-cell epitope. In some embodiments, a MERS-COV vaccine of the present disclosure comprises a nucleic acid sequence (e.g., mRNA) encoding S protein (S, S1 and/or S2), E protein, N protein, and at least one universal T-cell epitope. In some embodiments, a MERS-COV vaccine of the present disclosure comprises a nucleic acid sequence (e.g., mRNA) encoding S protein (S. S1 and/or S2), M protein, N protein, and at least one universal T-cell epitope. In some embodiments, a MERS-COV vaccine of the present disclosure comprises a nucleic acid sequence (e.g., mRNA) encoding E protein, M protein, N protein, and at least one universal T-cell epitope.

A MERS-COV vaccine may comprise, for example, at least one RNA (e.g., mRNA) polynucleotide having an open reading frame encoding at least one MERS-COV antigenic polypeptide identified by any one of SEQ ID NO: 24-38 or 33 and at least one universal T-cell epitope.

A MERS-COV vaccine may comprise, for example, at least one RNA (e.g., mRNA) polynucleotide encoded by a nucleic acid (e.g., DNA) identified by any one of SEQ ID NO: 20-23, and at least one universal T-cell epitope.

The present disclosure is not limited by a particular strain of MERS-COV. The strain of MERS-COV used in a vaccine may be any strain of MERS-COV. Non-limiting examples of strains of MERS-COV for use as provide herein include Riyadh_14_2013, and 2cEMC/2012, Hasa_1_2013.

SARS-COV. The genome of SARS-COV includes of a single, positive-strand RNA that is approximately 29,700 nucleotides long. The overall genome organization of SARS-COV is similar to that of other coronaviruses. The reference genome includes 13 genes, which encode at least 14 proteins. Two large overlapping reading frames (ORFs) encompass 71% of the genome. The remainder has 12 potential ORFs, including genes for structural proteins S (spike), E (small envelope), M (membrane), and N (nucleocapsid). Other potential ORFs code for unique putative SARS-COV-specific polypeptides that lack obvious sequence similarity to known proteins. A detailed analysis of the SARS-COV genome has been published in J Mol Biol 2003; 331:991-1004.

In some embodiments, a SARS-COV vaccine of the present disclosure comprises a RNA (e.g., mRNA) polynucleotide encoding S protein (S, S1 and/or S2), E protein, N protein and M protein, and a RNA (e.g., mRNA) polynucleotide encoding a T-cell epitope.

In some embodiments, a SARS-COV vaccine of the present disclosure comprises a RNA (e.g., mRNA) polynucleotide encoding S protein (S, S1 and/or S2) and E protein, and a RNA (e.g., mRNA) polynucleotide encoding a T-cell epitope. In some embodiments, a SARS-CoV vaccine of the present disclosure comprises a RNA (e.g., mRNA) polynucleotide encoding S protein (S, S1 and/or S2) and N protein, and a RNA (e.g., mRNA) polynucleotide encoding a T-cell epitope. In some embodiments, a SARS-COV vaccine of the present disclosure comprises a RNA (e.g., mRNA) polynucleotide encoding S protein (S, S1 and/or S2) and M protein, and a RNA (e.g., mRNA) polynucleotide encoding a T-cell epitope.

In some embodiments, a SARS-COV vaccine of the present disclosure comprises a RNA (e.g., mRNA) polynucleotide encoding S protein (S, S1 and/or S2), E protein and M protein, and a RNA (e.g., mRNA) polynucleotide encoding a T-cell epitope. In some embodiments, a SARS-COV vaccine of the present disclosure comprises a RNA (e.g., mRNA) polynucleotide encoding S protein (S, S1 and/or S2), E protein and N protein, and a RNA (e.g., mRNA) polynucleotide encoding a T-cell epitope. In some embodiments, a SARS-CoV vaccine of the present disclosure comprises a RNA (e.g., mRNA) polynucleotide encoding S protein (S, S1 and/or S2), M protein and N protein, and a RNA (e.g., mRNA) polynucleotide encoding a T-cell epitope. In some embodiments, a SARS-COV vaccine of the present disclosure comprises a RNA (e.g., mRNA) polynucleotide encoding E protein, M protein and N protein, and a RNA (e.g., mRNA) polynucleotide encoding a T-cell epitope.

A SARS-COV vaccine may comprise, for example, at least one RNA (e.g., mRNA) polynucleotide having an open reading frame encoding at least one SARS-COV antigenic polypeptide identified by any one of SEQ ID NO: 29, 32 or 34, and a RNA (e.g., mRNA) polynucleotide encoding a T-cell epitope.

In some embodiments, a SARS-COV vaccine of the present disclosure comprises a RNA (e.g., mRNA) polynucleotide encoding S protein (S. S1 and/or S2), E protein, N protein, M protein, and a T-cell epitope.

In some embodiments, a SARS-COV vaccine of the present disclosure comprises a RNA (e.g., mRNA) polynucleotide encoding S protein (S, S1 and/or S2), E protein, and a T-cell epitope. In some embodiments, a SARS-COV vaccine of the present disclosure comprises a RNA (e.g., mRNA) polynucleotide encoding S protein (S, S1 and/or S2), N protein, and a T-cell epitope. In some embodiments, a SARS-COV vaccine of the present disclosure comprises a RNA (e.g., mRNA) polynucleotide encoding S protein (S, S1 and/or S2), M protein, and a T-cell epitope.

In some embodiments, a SARS-COV vaccine of the present disclosure comprises a RNA (e.g., mRNA) polynucleotide encoding S protein (S, S1 and/or S2), E protein, M protein, and a T-cell epitope. In some embodiments, a SARS-COV vaccine of the present disclosure comprises a RNA (e.g., mRNA) polynucleotide encoding S protein (S, S1 and/or S2), E protein, N protein, and a T-cell epitope. In some embodiments, a SARS-COV vaccine of the present disclosure comprises a RNA (e.g., mRNA) polynucleotide encoding S protein (S. S1 and/or S2), M protein, N protein, and a T-cell epitope. In some embodiments, a SARS-CoV vaccine of the present disclosure comprises a RNA (e.g., mRNA) polynucleotide encoding E protein, M protein, N protein, and a T-cell epitope.

A SARS-COV vaccine may comprise, for example, at least one RNA (e.g., mRNA) polynucleotide having an open reading frame encoding at least one SARS-COV antigenic polypeptide identified by any one of SEQ ID NO: 29, 32 or 34, and a T-cell epitope.

The present disclosure is not limited by a particular strain of SARS-COV. The strain of SARS-COV used in a vaccine may be any strain of SARS-COV.

SARS-COV-2. Severe acute respiratory syndrome coronavirus 2 (SARS-COV-2, commonly known as COVID-19) is a novel enveloped betacoronavirus closely related to two bat-derived severe acute respiratory syndrome (SARS)-like coronaviruses. (Lu et al., 2020) Infection with COVID-19 was first described in China in December of 2019, but in the ensuing months became a worldwide pandemic. The genome of SARS-COV-2 includes of a single, positive-strand RNA that is approximately 29,700 nucleotides long. The overall genome organization of SARS-COV-2 is similar to that of other coronaviruses. The reference genome includes 13 genes, which encode at least 14 proteins. Two large overlapping reading frames (ORFs) encompass 71% of the genome. The remainder has 12 potential ORFs, including genes for structural proteins S (spike), E (small envelope), M (membrane), and N (nucleocapsid). Other potential ORFs code for unique putative SARS-COV-specific polypeptides that lack obvious sequence similarity to known proteins. A detailed analysis of the SARS-COV genome has been published in J Mol Biol 2003; 331:991-1004.

In some embodiments, a SARS-COV-2 vaccine of the present disclosure comprises a nucleic acid sequence (e.g., mRNA) encoding S protein (S, S1 and/or S2), and a nucleic acid sequence (e.g., mRNA) encoding at least one universal T-cell epitope. In some embodiments, the universal T-cell epitope can be derived from viral proteins other than S, that are highly conserved among the variants or/and in other coronaviruses.

In some embodiments, a SARS-COV-2 vaccine of the present disclosure can include a nucleic acid sequence (e.g., mRNA) encoding S protein (S, S1 and/or S2) with two proline substitutions for preventing the pre-fusion to post-fusion confirmation transition (SCIENCE, Vol 369, Issue 6510, pp. 1501-1505), and a nucleic acid sequence (e.g., mRNA) encoding at least one universal T-cell epitope. In some embodiments, the T-cell epitope can be highly conserved among the variants or/and in other coronaviruses.

In some embodiments, a SARS-COV-2 vaccine of the present disclosure comprises a nucleic acid sequence (e.g., mRNA) encoding S protein (S, S1 and/or S2) and full-length or truncated N protein and/or M protein, a nucleic acid sequence (e.g., mRNA) encoding at least one universal T-cell epitope. The universal T-cell epitope can be highly conserved among the variants or/and in other coronaviruses (e.g., SEQ ID NO: 323, 324, 335, 336, 337 and 338). In some embodiments, a SARS-COV-2 vaccine of the present disclosure comprises a nucleic acid sequence (e.g., mRNA) encoding S protein (S, S1 and/or S2) (e.g., SEQ ID NO: 321, 322, 325, 326, 327, 328, 323, 324) and a nucleic acid sequence (e.g., mRNA) encoding at least one universal T-cell epitopes (e.g., SEQ ID NO: 329, 330, and 351-450). In some embodiments, a SARS-COV-2 vaccine of the present disclosure comprises a nucleic acid sequence (e.g., mRNA) encoding S protein (S, S1 and/or S2) (e.g., SEQ ID NO: 321, 322, 325, 326, 327, 328, 323, 324) and a nucleic acid sequence (e.g., mRNA) encoding multiple universal T-cell epitopes (e.g., SEQ ID NO: 329, 330, and 351-450). In some embodiments, a SARS-COV-2 vaccine of the present disclosure comprises a nucleic acid sequence (e.g., mRNA) encoding S protein (S, S1 and/or S2) and M protein, and a nucleic acid sequence (e.g., mRNA) encoding at least one universal T-cell epitope.

A SARS-COV-2 vaccine may include, for example, at least one nucleic acid sequence (e.g., mRNA) having an open reading frame encoding at least one SARS-COV-2 antigenic polypeptide identified by any one of SEQ ID NO: 321, 322, 325, 326, 327, or 328 (see Table 1) and a nucleic acid sequence (e.g., mRNA) encoding at least one universal T-cell epitope identified by any one of SEQ ID NO: 329-332, 351-450, in the Table 1, and in the Table 2, all the T cell epitopes in the mRNA are listed.

A SARS-COV-2 vaccine may comprise, for example, at least one nucleic acid sequence (e.g., mRNA) having an open reading frame encoding at least one SARS-COV2 antigenic polypeptide identified by any one of SEQ ID NO: 321, 322, 325 or 326 (Table 1) and at least one universal T-cell epitope identified by any one of SEQ ID NO: 329, 330, 351-450 (Table 1 and Table 2).

In some embodiments, a SARS-COV-2 antigenic polypeptide is encoded by at least one nucleic acid sequence selected from any of SEQ ID NO: 322-324, 326, 328, 330-332, 334, or 336 (Table 1) and homologs having at least 80% identity with a nucleic acid sequence selected from any one of SEQ ID NO: 322-324, 326, 328, 330-332, 334, or 336 (Table 1). In some embodiments, a SARS-COV-2 antigenic polypeptide is encoded by at least one nucleic acid sequence selected from any one of SEQ ID NO: 322-324, 326, 328, 330-332, 334, or 336 (Table 1) and homologs having at least 90% (e.g. 90%, 91%, 92%, 93%, 94%, 95%, 96%, 97%, 98%, 99%, 99.8%, or 99.9%) identity with a nucleic acid sequence selected from any one of SEQ ID NO: 322-324, 326, 328, 330-332, 334, or 336 (Table 1). In some embodiments, a SARS-COV-2 antigenic polypeptide is encoded by at least one fragment of a nucleic acid sequence selected from any one of SEQ ID NO: 322-324, 326, 328, 330-332, 334, or 336 (Table 1). In some embodiments, the at least one nucleic acid has a chemical modification.

In some embodiments, a universal T-cell epitope is encoded by at least one nucleic acid sequence selected from any of SEQ ID NO: 329, 330, 351-450 (Table 1 and Table 2) and homologs having at least 80% identity with a nucleic acid sequence selected from any one of SEQ ID NO: 329, 330, 351-450 (Table 1 and Table 2). In some embodiments a universal T-cell epitope is encoded by at least one nucleic acid sequence selected from any one of SEQ ID NO: 329, 330, 351-450 (Table 1 and Table 2) and homologs having at least 90% (e.g. 90%, 91%, 92%, 93%, 94%, 95%, 96%, 97%, 98%, 99%, 99.8%, or 99.9%) identity with a nucleic acid sequence selected from any one of SEQ ID NO: 329, 330, 351-450 (Table 1 and Table 2). In some embodiments, a universal T-cell epitope is encoded by at least one fragment of a nucleic acid sequence selected from any one of SEQ ID NO: 329, 330, 351-450 (Table 1 and Table 2). In some embodiments, the at least one nucleic acid has a chemical modification.

In some embodiments, the SARS-COV-2 antigenic polypeptide is selected from any of SEQ ID NO: 321, 325, 327, 329, 333, or 335 (Table 1) and homologs having at least 80% identity with a SARS-COV-2 antigenic polypeptide selected from any one of SEQ ID NO: 321, 325, 327, 329, 333, or 335 (Table 1). In some embodiments, SARS-COV-2 antigenic polypeptide selected from any one of SEQ ID NO: 321, 325, 327, 329, 333, or 335 (Table 1) and homologs having at least 90% (e.g. 90%, 91%, 92%, 93%, 94%, 95%, 96%, 97%, 98%, 99%, 99.8%, or 99.9%) identity with a SARS-COV-2 antigenic polypeptide selected from any one of SEQ ID NO: 321, 325, 327, 329, 333, or 335 (Table 1). In some embodiments, a SARS-CoV-2 antigenic polypeptide is selected from any one of SEQ ID NO: 321, 325, 327, 329, 333, or 335 (Table 1).

The present disclosure is not limited by a particular strain of SARS-COV-2. The strain of SARS-COV-2 used in a vaccine may be any variants of SARS-COV-2 and strain of SARS-CoV, which includes HCoV-OC43, HCoV-HKU1, HCoV-NL63 or HCoV-229E

HCoV-OC43. Human coronavirus OC43 is an enveloped, positive-sense, single-stranded RNA virus in the species Betacoronavirus-1 (genus Betacoronavirus, subfamily Coronavirinae, family Coronaviridae, order Nidovirales). Four HCoV-OC43 genotypes (A to D), have been identified with genotype D most likely arising from recombination. The complete genome sequencing of two genotype C and D strains and bootscan analysis shows recombination events between genotypes B and C in the generation of genotype D. Of 29 strains identified, none belong to the more ancient genotype A. Along with HCoV-229E, a species in the Alphacoronavirus genus, HCoV-OC43 are among the known viruses that cause the common cold. Both viruses can cause severe lower respiratory tract infections, including pneumonia in infants, the elderly, and immunocompromised individuals such as those undergoing chemotherapy and those with HIV-AIDS.

HCoV-HKU1. Human coronavirus HKU1 (HCoV-HKU1) is a positive-sense, single-stranded RNA virus with the HE gene, which distinguishes it as a group 2, or Betacoronavirus. It was discovered in January 2005 in two patients in Hong Kong. The genome of HCoV-HKU1 is a 29,926-nucleotide, polyadenylated RNA. The GC content is 32%, the lowest among all known coronaviruses. The genome organization is the same as that of other group II coronaviruses, with the characteristic gene order 1a, 1b, HE, S, E, M, and N. Furthermore, accessory protein genes are present between the S and E genes (ORF4) and at the position of the N gene (ORF8). The TRS is presumably located within the AAUCUAAAC sequence, which precedes each ORF except E. As in sialodacryoadenitis virus and mouse hepatitis virus (MHV), translation of the E protein possibly occurs via an internal ribosomal entry site. The 3′ untranslated region contains a predicted stem-loop structure immediately downstream of the N ORF (nucleotide position 29647 to 29711). Further downstream, a pseudoknot structure is present at nucleotide position 29708 to 29760. Both RNA structures are conserved in group II coronaviruses and are critical for virus replication.

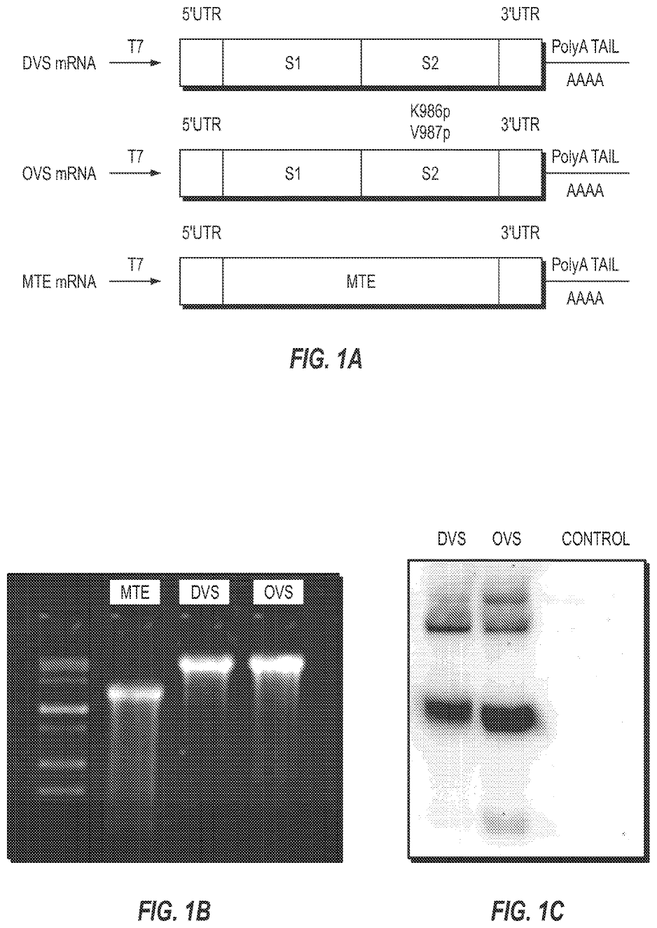

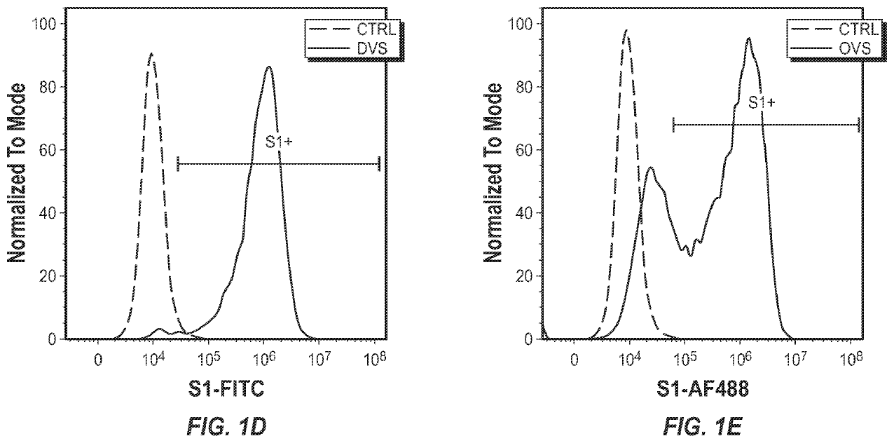

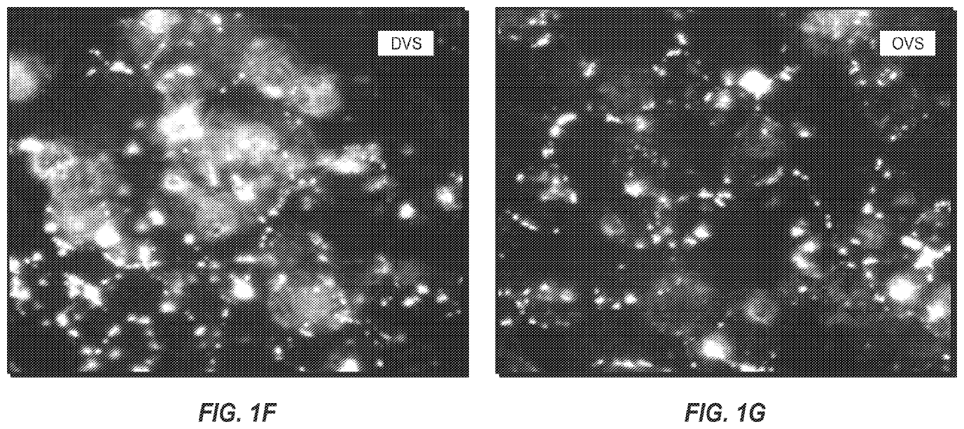

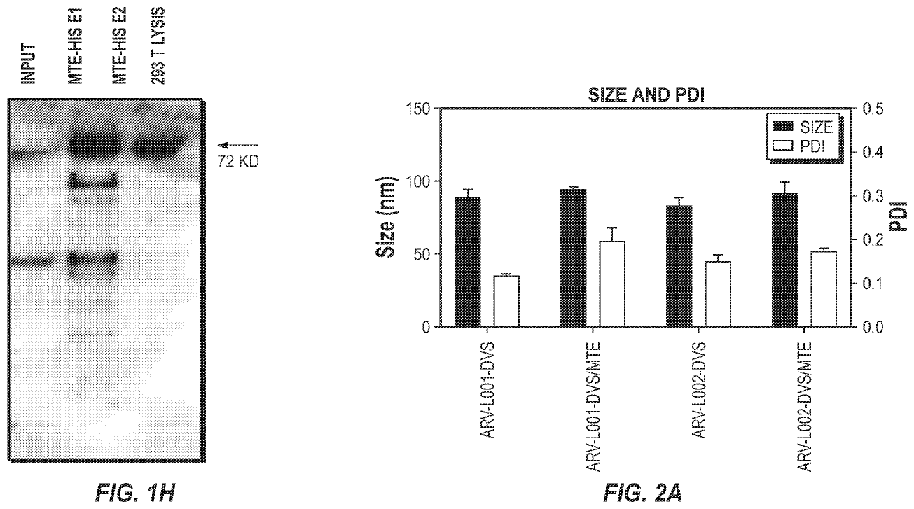

HCoV-NL63. The RNA genome of human coronavirus NL63 (HCoV-NL63) is 27,553 nucleotides, with a poly(A) tail (FIG. 1). With a GC content of 34%, HCoV-NL63 has one of the lowest GC contents of the coronaviruses, for which GC content ranges from 32 to 42%. Untranslated regions of 286 and 287 nucleotides are present at the 5′ and 3′ termini, respectively. Genes predicted to encode the S, E, M, and N proteins are found in the 3′ part of the HCoV-NL63 genome. The HE gene, which is present in some group II coronaviruses, is absent, and there is only a single, monocistronic accessory protein ORF (ORF3) located between the S and E genes. Subgenomic mRNAs are generated for all ORFs (S, ORF3, E, M, and N), and the core sequence of the TRS of HCoV-NL63 is defined as AACUAAA. This sequence is situated upstream of every ORF except for the E ORF, which contains the suboptimal core sequence AACUAUA. Interestingly, a 13-nucleotide sequence with perfect homology to the leader sequence is situated upstream of the suboptimal E TRS. Annealing of this 13-nucleotide sequence to the leader sequence may act as a compensatory mechanism for the disturbed leader-TRS/body-TRS interaction.

HCoV-229E. Human coronavirus 229E (HCoV-229E) is a single-stranded, positive-sense, RNA virus species in the Alphacoronavirus genus of the subfamily Coronavirinae, in the family Coronaviridae, of the order Nidovirales. Along with Human coronavirus OC43, it is responsible for the common cold. HCoV-NL63 and HCoV-229E are two of the four human coronaviruses that circulate worldwide. These two viruses are unique in their relationship towards each other. Phylogenetically, the viruses are more closely related to each other than to any other human coronavirus, yet they only share 65% sequence identity. Moreover, the viruses use different receptors to enter their target cell. HCoV-NL63 is associated with croup in children, whereas all signs suggest that the virus probably causes the common cold in healthy adults. HCoV-229E is a proven common cold virus in healthy adults, so it is probable that both viruses induce comparable symptoms in adults, even though their mode of infection differs (HCoV-NL63 and HCoV-229E are two of the four human coronaviruses that circulate worldwide. These two viruses are unique in their relationship towards each other. Phylogenetically, the viruses are more closely related to each other than to any other human coronavirus, yet they only share 65% sequence identity. Moreover, the viruses use different receptors to enter their target cell. HCoV-NL63 is associated with croup in children, whereas all signs suggest that the virus probably causes the common cold in healthy adults. HCoV-229E is a proven common cold virus in healthy adults, so it is probable that both viruses induce comparable symptoms in adults, even though their mode of infection differs (Dijkman R. et al. J Formos Med Assoc. 2009 April; 108 (4): 270-9, the contents of which is incorporated herein by reference in their entirety).

Feline coronavirus (FCoV). Feline infectious peritonitis (FIP) is a viral disease of cats caused by feline coronavirus. It is a coronavirus of the species Alphacoronavirus 1 which has two different forms: feline enteric coronavirus (FECV) that infects the intestines and feline infectious peritonitis virus (FIPV) that causes the disease feline infectious peritonitis (FIP). Most strains of feline coronavirus are found in the gastrointestinal tract and do not cause significant disease. These are referred to as feline enteric coronavirus (FeCV). Cats infected with FeCV usually do not show any symptoms during the initial viral infection, and in approximately 10 percent of cats infected with FeCV, one or more mutations of the virus can alter its biological behavior, resulting in white blood cells becoming infected with virus and spreading it throughout the cat's body. When this occurs, the virus is referred to as the FIPV, and this disease is usually progressive and almost always fatal without therapy that has recently become available.

In some embodiments, compositions can include a nucleic acid sequence (e.g., mRNA) encoding FCoV antigenic polypeptide and at least one universal T-cell epitope.

In some embodiments, compositions can include a nucleic acid sequence (e.g., SEQ ID NO: 541 (Table 4)) (e.g., mRNA) encoding FCOV Spike protein antigenic polypeptide (e.g., SEQ ID NO: 540 (Table 4)) and at least one universal T-cell epitope. SEQ ID NOs: 542-602 (Table 5)

In some embodiments, a FCOV antigenic polypeptide is encoded by at least one nucleic acid sequence selected from any of SEQ ID NOs: 541, 634, 636,638, 640, 642, and 644 (Table 4) and homologs having at least 80% identity with a nucleic acid sequence selected from any one of SEQ ID NOs: 541, 634, 636,638, 640, 642, and 644 (Table 4). In some embodiments, a FCoV antigenic polypeptide is encoded by at least one nucleic acid sequence selected from any one of SEQ ID NOs: 541, 634, 636,638, 640, 642, and 644 (Table 4) and homologs having at least 90% (e.g. 90%, 91%, 92%, 93%, 94%, 95%, 96%, 97%, 98%, 99%, 99.8%, or 99.9%) identity with a nucleic acid sequence selected from any one of SEQ ID NOs: 541, 634, 636,638, 640, 642, and 644 (Table 4). In some embodiments, a FCOV antigenic polypeptide is encoded by at least one fragment of a nucleic acid sequence selected from any one of SEQ ID NOs: 541, 634, 636,638, 640, 642, and 644 (Table 4). In some embodiments, the at least one nucleic acid has a chemical modification.

In some embodiments, a universal T-cell epitope is encoded by at least one nucleic acid sequence selected from any of SEQ ID NOs: 542-602 (Table 5) and homologs having at least 80% identity with a nucleic acid sequence selected from any one of SEQ ID NOs: 542-602 (Table 5). In some embodiments, a universal T-cell epitope is encoded by at least one nucleic acid sequence selected from any one of SEQ ID NOs: 542-602 (Table 5) and homologs having at least 90% (e.g. 90%, 91%, 92%, 93%, 94%, 95%, 96%, 97%, 98%, 99%, 99.8%, or 99.9%) identity with a nucleic acid sequence selected from any one of SEQ ID NOs: 542-602 (Table 5). In some embodiments, a universal T-cell epitope is encoded by at least one fragment of a nucleic acid sequence selected from any one of SEQ ID NOs: 542-602 (Table 5). In some embodiments, the at least one nucleic acid has a chemical modification.

A FCoV vaccine may include, for example, at least one nucleic acid sequence (e.g., mRNA) having an open reading frame encoding at least one FCoV antigenic polypeptide identified by any one of SEQ ID NOs: 540, 633, 635, 637, 639, 641, and 643 (see Table 4) and a nucleic acid sequence (e.g., mRNA) encoding at least one universal T-cell epitope identified by any one of SEQ ID NOs: 542-602 (Table 5).

A FCoV vaccine may comprise, for example, at least one nucleic acid sequence (e.g., mRNA) having an open reading frame encoding at least one FCOV antigenic polypeptide identified by any one of SEQ ID NOs: 540, 633, 635, 637, 639, 641, and 643 (see Table 4) and at least one universal T-cell epitope identified by any one of SEQ ID NOs: 542-602 (Table 5).

In some embodiments, the FCOV antigenic polypeptide is selected from any of SEQ ID NOs: 540, 633, 635, 637, 639, 641, and 643 (Table 4) and homologs having at least 80% identity with a FCOV antigenic polypeptide selected from any one of SEQ ID NOs: 540, 633, 635, 637, 639, 641, and 643 (Table 4). In some embodiments, FCOV antigenic polypeptide selected from any one of SEQ ID NOs: 540, 633, 635, 637, 639, 641, and 643 (Table 4) and homologs having at least 90% (e.g. 90%, 91%, 92%, 93%, 94%, 95%, 96%, 97%, 98%, 99%, 99.8%, or 99.9%) identity with a FCOV antigenic polypeptide selected from any one of SEQ ID NOs: 540, 633, 635, 637, 639, 641, and 643 (Table 4). In some embodiments, a FCoV antigenic polypeptide is selected from any one of SEQ ID NOs: 540, 633, 635, 637, 639, 641, and 643 (Table 4).

### Poxvirus

Monkeypox. Monkeypox (MPX) is a disease that is caused by infection with the monkeypox virus, which belongs to the Orthopoxvirus genus, which includes the variola (smallpox) virus, Ectromelia virus (mousepox) virus and vaccinia virus. All the members of the poxviridae comprise a large family of complex DNA viruses that replicate in the cytoplasm of vertebrate and invertebrate cells. In humans, smallpox was by far the most important poxvirus infection, however, human also can be infected with other poxviruses such as monkeypox, and recently there has been a marked increase in reported cases where monkeypox is not commonly seen, such as in United States, Canada and Europe. Vaccinia virus was used as live vaccine to immunize against smallpox. Successful worldwide vaccination with Vaccinia virus worldwide eventually led to the eradication of variola virus. However, vaccinia vaccine can produce severe disease and even death in elderly or immunocompromised people, therefore, there is unmet need for development of a universal vaccine against poxvirus infection including but not limited to monkeypox.

All the members in Orthopoxviruses (OPVs), including MPX, form two types of infectious viral particles: the mature virus (MV), which is cytosolic, and the enveloped virus (EV), which is extracellular. It is believed that MVs are required for viral entry into the host, while EVs are responsible for spread within the host. Previous studies have found that antibodies to an MV proteins, i.e., A33R, LIR, B5R, as well as viral secreted protein such as B16R, were highly effective for preventing virus spreading in vivo as well as curing a mousepox infection when administered post-exposure to virus J Virol. 2013 June; 87 (12): 7046-7053., which indicates that including these viral proteins might be a good strategy for a new vaccine against poxvirus infection, i.e., MPX.

In some embodiments, composition can include a nucleic acid sequence (e.g., mRNA) encoding MPX sequences and at least one universal T-cell epitope.

In some embodiments, composition can include a nucleic acid sequence (e.g., mRNA) encoding MPX EV surface membrane protein B6R (similar to Vaccinia virus strain Copenhagen B5R), and at least one universal T-cell epitope.

In some embodiments, composition can include a nucleic acid sequence (e.g., mRNA) encoding MPX MV surface membrane protein MIR (similar to Vaccinia virus strain Copenhagen LIR), and at least one universal T-cell epitope.

In some embodiments, composition can include a nucleic acid sequence (e.g., mRNA) encoding MPX EV surface membrane protein A35R (similar to Vaccinia virus strain Copenhagen A33R), and at least one universal T-cell epitope.

In some embodiments, composition can include a nucleic acid sequence (e.g., mRNA) encoding MPX B16R (similar to Vaccinia virus strain Copenhagen B19R), and at least one universal T-cell epitope.

In some embodiments, composition comprise a nucleic acid sequence (e.g., mRNA) encoding MPX sequences and a nucleic acid sequence (e.g., mRNA) including at least one universal T-cell epitope.

In some embodiments, composition can include a nucleic acid sequence (e.g., mRNA) encoding MPX EV surface membrane protein B6R (similar to Vaccinia virus strain Copenhagen B5R), and a nucleic acid sequence (e.g., mRNA) including at least one universal T-cell epitope.

In some embodiments, composition comprise a nucleic acid sequence (e.g., mRNA) encoding MPX MV surface membrane protein MIR (similar to Vaccinia virus strain Copenhagen LIR), and a nucleic acid sequence (e.g., mRNA) including at least one universal T-cell epitope.

In some embodiments, composition comprise a nucleic acid sequence (e.g., mRNA) encoding MPX EV surface membrane protein A35R (similar to Vaccinia virus strain Copenhagen A33R), and a nucleic acid sequence (e.g., mRNA) including at least one universal T-cell epitope.

In some embodiments, composition comprise a nucleic acid sequence (e.g., mRNA) encoding MPX B16R (similar to Vaccinia virus strain Copenhagen B19R), and a nucleic acid sequence (e.g., mRNA) including at least one universal T-cell epitope.

In some embodiments, a MPX antigenic polypeptide is encoded by at least one nucleic acid sequence selected from any of SEQ ID NO: 348 and 350 (Table 1) and homologs having at least 80% identity with a nucleic acid sequence selected from any one of SEQ ID NO: 348 and 350 (Table 1). In some embodiments, MPX antigenic polypeptide is encoded by at least one nucleic acid sequence selected from any one of SEQ ID NO: 348 and 350 (Table 1) and homologs having at least 90% (e.g. 90%, 91%, 92%, 93%, 94%, 95%, 96%, 97%, 98%, 99%, 99.8%, or 99.9%) identity with a nucleic acid sequence selected from any one of SEQ ID NO: 348 and 350 (Table 1). In some embodiments, a MPX antigenic polypeptide is encoded by at least one fragment of a nucleic acid sequence selected from any one of SEQ ID NO: 348 and 350 (Table 1). In some embodiments, the at least one nucleic acid has a chemical modification.

In some embodiments, the MPX antigenic polypeptide is selected from any of SEQ ID NO: 347 and 349 (Table 1) and homologs having at least 80% identity with a MPX antigenic polypeptide selected from any one of SEQ ID NO: 347 and 349 (Table 1). In some embodiments, MPX antigenic polypeptide selected from any one of SEQ ID NO: 347 and 349 (Table 1) and homologs having at least 90% (e.g. 90%, 91%, 92%, 93%, 94%, 95%, 96%, 97%, 98%, 99%, 99.8%, or 99.9%) identity with a MPX antigenic polypeptide selected from any one of SEQ ID NO: 347 and 349 (Table 1). In some embodiments, a MPX antigenic polypeptide is selected from any one of SEQ ID NO: 347 and 349 (Table 1).

In some embodiments, the universal T-cell epitope can be identical in other poxviruses such as Cowpox, smallpox and vaccinia viruses.

### Influenza

In some embodiments, the virus is a strain of Influenza A or Influenza B or combinations thereof. In some embodiments, the strain of Influenza A or Influenza B is associated with birds, pigs, horses, dogs, humans or non-human primates. In some embodiments, the antigenic polypeptide encodes a hemagglutinin protein or fragment thereof. In some embodiments, the hemagglutinin protein is H7 or H10 or a fragment thereof. In some embodiments, the hemagglutinin protein comprises a portion of the head domain (HA1). In some embodiments, the hemagglutinin protein comprises a portion of the cytoplasmic domain. In some embodiments, the truncated hemagglutinin protein. In some embodiments, the protein is a truncated hemagglutinin protein comprises a portion of the transmembrane domain. In some embodiments, the amino acid sequence of the hemagglutinin protein or fragment thereof comprises at least 90%, 91%, 92%, 93%, 94%, 95%, 96%, 97% 98%, or 99% identify with any of the amino acid sequences of SEQ ID NO. 148 or 151. In some embodiments, the virus is selected from the group consisting of H7N9 and H10N8.

Influenza A virus subtype H7N9 is a form of influenza with a high potential to become pandemic. Over 600 cases of H7N9 influenza have been documented in China to date, with a mortality rate of approximately 1 in 3 people. This may have resulted from sporadic, non-sustained human-to-human transmission. Although several vaccines are in development, immunogenicity has been reportedly low without the addition of adjuvants.

Influenza A virus subtype H10N8 has also shown a potential to become pandemic, but is at lower risk than influenza A virus subtype H7N9. In 2013, only 3 cases of H10N8 influenza infection were reported in China, resulting in 2 deaths. To date, no vaccine is available.

In some embodiments the HA7 hemagglutinin antigen is an H7N9 antigen. In other embodiments the RNA polynucleotide comprises a polynucleotide encoding an amino acid sequence having at least 80% sequence identity to SEQ ID NO 148. In other embodiments, the RNA polynucleotide comprises a polynucleotide encoding the amino acid sequence of SEQ ID NO 148. In other embodiments the RNA polynucleotide has at least 80% sequence identity to SEQ ID NO 152, 153, 154, or 156.

In some embodiments, the HA10 hemagglutinin antigen is an H10N8 antigen. In some embodiments, the RNA polynucleotide comprises a polynucleotide encoding an amino acid sequence having at least 80% sequence identity to SEQ ID NO 151. In some embodiments, the RNA polynucleotide comprises a polynucleotide encoding the amino acid sequence of SEQ ID NO 151. In other embodiments, the RNA polynucleotide has at least 80% sequence identity to SEQ ID NO 149, 150, or 155.

In some embodiments, the universal T-cell epitope can be identical in other influenza viruses.

### Herpes Simplex Viruses

The genome of Herpes Simplex Viruses (HSV-1 and HSV-2) contains about 85 open reading frames, such that HSV can generate at least 85 unique proteins. These genes encode 4 major classes of proteins: (1) those associated with the outermost external lipid bilayer of HSV (the envelope), (2) the internal protein coat (the capsid), (3) an intermediate complex connecting the envelope with the capsid coat (the tegument), and (4) proteins responsible for replication and infection.

Examples of envelope proteins include UL1 (gL), ULIO (gM), UL20, UL22, UL27 (gB), UL43, UL44 (gC), UL45, UL49A, UL53 (gK), US 4 (gG), US 5 (gJ), US 6 (gD), US 7 (gl), US 8 (gE), and US 10. Examples of capsid proteins include UL6, UL18, UL19, UL35, and UL38. Tegument proteins include UL11, UL13, UL21, UL36, UL37, UL41, UL45, UL46, UL47, UL48, UL49, US9, and US 10. Other HSV proteins include UL2, UL3, UL4, UL5, UL7, UL8, UL9, UL12, UL14, UL15, UL16, UL17, UL23, UL24, UL25, UL26, UL26.5, UL28, UL29, UL30, UL31, UL32, UL33, UL34, UL39, UL40, UL42, UL50, UL51, UL52, UL54, UL55, UL56, US 1, US2, US3, US81, US 11, US 12, ICP0, and ICP4.

Since the envelope (most external portion of an HSV particle) is the first to encounter target cells, the present disclosure encompasses antigenic polypeptides associated with the envelope as immunogenic agents. In brief, surface and membrane proteins—glycoprotein D (gD), glycoprotein B (gB), glycoprotein H (gH), glycoprotein L (gL)—as single antigens or in combination with or without adjuvants may be used as HSV vaccine antigens.

In some embodiments, composition comprise RNA (e.g., mRNA) encoding HSV (HSV-1 or HSV-2)glycoprotein D.

In some embodiments, HSV vaccines comprise RNA (e.g., mRNA) encoding HSV (HSV-1 or HSV-2)glycoprotein B.

In some embodiments, HSV vaccines comprise RNA (e.g., mRNA) encoding HSV (HSV-1 or HSV-2)glycoprotein D and glycoprotein B.

In some embodiments, HSV vaccines comprise RNA (e.g., mRNA) encoding HSV (HSV-1 or HSV-2)glycoprotein D and glycoprotein C.

In some embodiments, HSV vaccines comprise RNA (e.g., mRNA) encoding HSV (HSV-1 or HSV-2)glycoprotein D and glycoprotein E (or glycoprotein I).

In some embodiments, HSV vaccines comprise RNA (e.g., mRNA) encoding HSV (HSV-1 or HSV-2)glycoprotein B and glycoprotein C.

In some embodiments, HSV vaccines comprise RNA (e.g., mRNA) encoding HSV (HSV-1 or HSV-2)glycoprotein B and glycoprotein E (or glycoprotein I).

In some embodiments, HSV vaccines comprise RNA (e.g., mRNA) encoding a HSV (HSV-1 or HSV-2) antigenic polypeptide having at least 95%, at least 96%, at least 97%, at least 98% or at least 99% identity with HSV (HSV-1 or HSV-2)glycoprotein D and has HSV (HSV-1 or HSV-2)glycoprotein D activity.

In some embodiments, HSV vaccines comprise RNA (e.g., mRNA) encoding a HSV (HSV-1 or HSV-2) antigenic polypeptide having at least 95%, at least 96%, at least 97%, at least 98% or at least 99% identity with HSV (HSV-1 or HSV-2)glycoprotein C and has HSV (HSV-1 or HSV-2)glycoprotein C activity.

In some embodiments, HSV vaccines comprise RNA (e.g., mRNA) encoding a HSV (HSV-1 or HSV-2) antigenic polypeptide having at least 95%, at least 96%, at least 97%, at least 98% or at least 99% identity with HSV (HSV-1 or HSV-2)glycoprotein B and has HSV (HSV-1 or HSV-2)glycoprotein B activity.

In some embodiments, HSV vaccines comprise RNA (e.g., mRNA) encoding a HSV (HSV-1 or HSV-2) antigenic polypeptide having at least 95%, at least 96%, at least 97%, at least 98% or at least 99% identity with HSV (HSV-1 or HSV-2)glycoprotein E and has HSV (HSV-1 or HSV-2)glycoprotein E activity.

In some embodiments, HSV vaccines comprise RNA (e.g., mRNA) encoding a HSV (HSV-1 or HSV-2) antigenic polypeptide having at least 95%, at least 96%, at least 97%, at least 98%, or at least 99% identity with HSV (HSV-1 or HSV-2)glycoprotein I and has HSV (HSV-1 or HSV-2)glycoprotein I activity.

Glycoprotein “activity” of the present disclosure is described below.

Glycoprotein C (gC) is a glycoprotein involved in viral attachment to host cells; e.g., it acts as an attachment protein that mediates binding of the HSV-2 virus to host adhesion receptors, namely cell surface heparan sulfate and/or chondroitin sulfate. gC plays a role in host immune evasion (aka viral immunoevasion) by inhibiting the host complement cascade activation. In particular, gC binds to and/or interacts with host complement component C3b; this interaction then inhibits the host immune response by dysregulating the complement cascade (e.g., binds host complement C3b to block neutralization of virus). SEQ ID NO: 345 and 346.

Glycoprotein D (gD) is an envelope glycoprotein that binds to cell surface receptors and/or is involved in cell attachment via polio virus receptor-related protein and/or herpesvirus entry mediator, facilitating virus entry. gD binds to the potential host cell entry receptors (tumor necrosis factor receptor superfamily, member 14 (TNFRSF14)/herpesvirus entry mediator (HVEM), polio virus receptor-related protein 1 (PVRL1) and or poliovirus receptor-related protein 2 (PVRL2), and is proposed to trigger fusion with host membrane by recruiting the fusion machinery composed of, for example, gB and gH/gL. gD interacts with host cell receptors TNFRSF14 and/or PVRL1 and/or PVRL2 and (1) interacts (via profusion domain) with gB; an interaction which can occur in the absence of related HSV glycoproteins, e.g., gH and/or gL; and (2) gD interacts (via profusion domain) with gH/gL heterodimer, an interaction which can occur in the absence of gB. As such, gD associates with the gB-gH/gL-gD complex. gD also interacts (via C-terrrrinus) with UL11 tegument protein. Receptor binding with gD is postulated to be required for inducing fusion via gB and gH/gL. When cysteine residues are introduced at position 190 (K190C) and 277 (A277C) at C-terminus of gD, the disulfide bond is formed, it remains the ability to bind to receptor(s) but cannot trigger fusion (JOURNAL OF VIROLOGY, January 2008, p. 700-709). Thus, these two substitutions can be included in the HSV-2 mRNA vaccine to stabilize the prefusion confirmation of gD. SEQ ID NO: 341 or 342.

Glycoprotein B (gB) is a viral glycoprotein involved in the viral cell activity of herpes simplex virus (HSV) and is required for the fusion of the HSV's envelope with the cellular membrane. It is the most highly conserved of all surface glycoproteins and primarily acts as a fusion protein, constituting the core fusion machinery. gB, a class III membrane fusion glycoprotein, is a type-1 transmembrane protein trimer of five structural domains. Domain I includes two internal fusion loops and is thought to insert into the cellular membrane during virus-cell fusion. Domain II appears to interact with gH/gL during the fusion process, domain III contains an elongated alpha helix, and domain IV interacts with cellular receptors. The prefusion form of gB presents an attractive target for HSV vaccine development as demonstrated for the class I fusion protein F of RSV and Spike glycoprotein of SARS-COV2, for which the most potent neutralizing antibodies target this conformation only. Previous study (https://www.science.org/doi/10.1126/sciadv.abc1726) with HSV-1 gB has shown that a substitution at position 516, H516P, had no effect on gB expression and posttranslational modifications, but this mutation blocked the transition of gB from pre- to post-fusion state. The H516P substitution can be included in the nucleic acid sequence of the HSV-2 vaccine to elicit neutralizing antibodies more effectively. SEQ ID NO: 341 and 342.

In epithelial cells, the heterodimer glycoprotein E/glycoprotein I (gE/gl) is required for the cell-to-cell spread of the virus, by sorting nascent virions to cell junctions. Once the virus reaches the cell junctions, virus particles can spread to adjacent cells extremely rapidly through interactions with cellular receptors that accumulate at these junctions. By similarity, it is implicated in basolateral spread in polarized cells. In neuronal cells, gE/gl is essential for the anterograde spread of the infection throughout the host nervous system Together with US 9, the heterodimer gE/gl is involved in the sorting and transport of viral structural components toward axon tips. The heterodimer gE/gl serves as a receptor for the Fc part of host IgG. Dissociation of gE/gl from IgG occurs at acidic pH, thus may be involved in anti-HSV antibodies bipolar bridging, followed by intracellular endocytosis and degradation, thereby interfering with host IgG-mediated immune responses. gE/gl interacts (via C-terrrrinus) with VP22 tegument protein; this interaction is necessary for the recruitment of VP22 to the Golgi and its packaging into virions. SEQ ID NO: 341 and 342.

In some embodiments, an antigenic polypeptide has at least 95%, at least 96%, at least 97%, at least 98% or at least 99% identity to HSV (HSV-1 or HSV-2)glycoprotein B, HSV (HSV-1 or HSV-2)glycoprotein C, HSV (HSV-1 or HSV-2)glycoprotein D, HSV (HSV-1 or HSV-2)glycoprotein E, HSV (HSV-1 or HSV-2)glycoprotein I, HSV (HSV-1 or HSV-2)glycoprotein H, HSV (HSV-1 or HSV-2)glycoprotein L, or HSV (HSV-1 or HSV-2) ICP4 protein.

In some embodiments, an antigenic polypeptideis encoded by at least one nucleic acid sequence selected from any of SEQ ID NOs: 157-179, 210-220, 269-272, 340, 342, 344, 346, 604, 606, 608, 610, 612, 614, 616, 618, 620, 622, 624, 626, 628, 630, and 632 homologs having at least 80% identity with a nucleic acid sequence selected from any one of SEQ ID NOs: 157-179, 210-220, 269-272, 340, 342, 344, 346, 604, 606, 608, 610, 612, 614, 616, 618, 620, 622, 624, 626, 628, 630, and 632. In some embodiments, an antigenic polypeptideis encoded by at least one nucleic acid sequence selected from any one of SEQ ID NOs: 157-179, 210-220, 269-272, 340, 342, 344, 346, 604, 606, 608, 610, 612, 614, 616, 618, 620, 622, 624, 626, 628, 630, and 632 and homologs having at least 90% (e.g. 90%, 91%, 92%, 93%, 94%, 95%, 96%, 97%, 98%, 99%, 99.8%, or 99.9%) identity with a nucleic acid sequence selected from any one of SEQ ID NOs: 157-179, 210-220, 269-272, 340, 342, 344, 346, 604, 606, 608, 610, 612, 614, 616, 618, 620, 622, 624, 626, 628, 630, and 632. In some embodiments, an antigenic polypeptideis encoded by at least one fragment of a nucleic acid sequence selected from any one of SEQ ID NOs: 157-179, 210-220, 269-272, 340, 342, 344, 346, 604, 606, 608, 610, 612, 614, 616, 618, 620, 622, 624, 626, 628, 630, and 632. In some embodiments, the at least one nucleic acid has a chemical modification.

In some embodiments, the composition comprises a nucleic acid sequence (e.g., mRNA) having an open reading frame encoding HSV-2 glycoprotein B (e.g., SEQ ID NOs: 341, 342, 157, 162, 168, 174, 222, 227, or 277).

In some embodiments, the composition comprises a nucleic acid sequence (e.g., mRNA) having an open reading frame encoding HSV-2 glycoprotein C (e.g., SEQ ID NOs: 158, 163, 169, 175, 223, 228, 278-285, 345, or 346).

In some embodiments, the composition comprises a nucleic acid sequence (e.g., mRNA) having an open reading frame encoding HSV-2 glycoprotein D (e.g., SEQ ID NOs: 159, 167, 170, 176, 224, 231, 341, or 342).

In some embodiments, the composition comprises a nucleic acid sequence (e.g., mRNA) having an open reading frame encoding HSV-2 glycoprotein E (e.g., SEQ ID NOs: 160, 164, 171, 177, 225, 229, 345 or 346).

In some embodiments, the composition comprises a nucleic acid sequence (e.g., mRNA) having an open reading frame encoding HSV-2 glycoprotein I (e.g., SEQ ID NOs: 161, 166, 169, 172, 178, 226, or 230).

In some embodiments, the composition comprises a nucleic acid sequence (e.g., mRNA) having an open reading frame encoding HSV-2 protein or fragment UL49 (e.g., SEQ ID NOs: 343 or 344). In some embodiments, a HSV vaccine comprises at least one RNA (e.g., mRNA) polynucleotide having an open reading frame encoding HS V-2 ICP4 protein (e.g., SEQ ID NOs: 165, 179, or 233).

In some embodiments, the composition comprises a nucleic acid sequence (e.g., mRNA) having an open reading frame encoding HSV-2 ICPO protein (e.g., SEQ ID NOs: 173 or 232).

In some embodiments, a HSV vaccine comprises at least one RNA (e.g., mRNA) polynucleotide having an open reading frame encoding HS V-2 ICPO protein or fragment (e.g., SEQ ID NOs: 343 or 344).

In some embodiments, the composition comprises a nucleic acid sequence (e.g., mRNA) having an open reading frame encoding HSV-2 UL19 protein (e.g., SEQ ID NOs: 343).

In some embodiments, the composition comprises a nucleic acid sequence (e.g. mRNA) encoding the whole genome of HSV-2 SEQ ID NO: 343 in the Table 1, which contains at least one universal T-cell epitope conserved between HSV-2 and HSV-1 SEQ ID NOs: 452-539, and 649 (Table 3). In some embodiments, the composition comprises a nucleic acid sequence (e.g. mRNA) that comprises a nucleic acid selected from any one of SEQ ID NOs: 234-268, 273-276, or 286-289. In some embodiments, the composition comprises a nucleic acid sequence (e.g. mRNA) that comprises a nucleic acid selected from any one of SEQ ID NOs: 340, 342, 344, 346, 604, 606, 608, 610, 612, 614, 616, 618, 620, 622, 624, 626, 628, 630, and 632.

In some embodiments, a nucleic acid sequence encoding an infection agent antigenic polypeptide having greater than 90% (e.g. 90%, 91%, 92%, 93%, 94%, 95%, 96%, 97%, 98%, 99%, 99.8%, or 99.9%) identity to an amino acid sequence of any one of SEQ ID NOs: 180-209, 222-233, 277-281, or 339, 341, 343, 345, 603, 605, 607, 609, 611, 613, 614, 617, 619, 621, 623, 625, 627, 629, 631.

A HSV vaccine may include, for example, at least one nucleic acid sequence (e.g., mRNA) having an open reading frame encoding at least one HSV antigenic polypeptide identified by any one of SEQ ID NOs: 157-179, 210-220, 269-272, 340, 342, 344, 346, 604, 606, 608, 610, 612, 614, 616, 618, 620, 622, 624, 626, 628, 630, and 632 and a nucleic acid sequence (e.g., mRNA) encoding at least one universal T-cell epitope identified by any one of SEQ ID NOs: 542-539, and 649 (Table 3), all the T cell epitopes in the mRNA are listed.

A HSV vaccine may comprise, for example, at least one nucleic acid sequence (e.g., mRNA) having an open reading frame encoding at least one HSV antigenic polypeptide identified by any one of SEQ ID NOs: 157-179, 210-220, 269-272, 340, 342, 344, 346, 604, 606, 608, 610, 612, 614, 616, 618, 620, 622, 624, 626, 628, 630, and 632 and at least one universal T-cell epitope identified by any one of SEQ ID NOs: 452-539, and 649 (Table 3).

In some embodiments, a HSV antigenic polypeptide is encoded by at least one nucleic acid sequence selected from any of SEQ ID NOs: 340, 342, 344, 346, 604, 606, 608, 610, 612, 614, 616, 618, 620, 622, 624, 626, 628, 630, and 632 (Table 1) and homologs having at least 80% identity with a nucleic acid sequence selected from any one of SEQ ID NOs: 340, 342, 344, 346, 604, 606, 608, 610, 612, 614, 616, 618, 620, 622, 624, 626, 628, 630, and 632 (Table 1). In some embodiments, a HSV antigenic polypeptide is encoded by at least one nucleic acid sequence selected from any one of SEQ ID Nos: 340, 342, 344, 346, 604, 606, 608, 610, 612, 614, 616, 618, 620, 622, 624, 626, 628, 630, and 632 (Table 1) and homologs having at least 90% (e.g. 90%, 91%, 92%, 93%, 94%, 95%, 96%, 97%, 98%, 99%, 99.8%, or 99.9%) identity with a nucleic acid sequence selected from any one of SEQ ID NO: 340, 342, 344, 346, 604, 606, 608, 610, 612, 614, 616, 618, 620, 622, 624, 626, 628, 630, and 632 (Table 1). In some embodiments, a HSV antigenic polypeptide is encoded by at least one fragment of a nucleic acid sequence selected from any one of SEQ ID NO: 340, 342, 344, 346, 604, 606, 608, 610, 612, 614, 616, 618, 620, 622, 624, 626, 628, 630, and 632 (Table 1). In some embodiments, the at least one nucleic acid has a chemical modification.

In some embodiments, the HSV antigenic polypeptide is selected from any of SEQ ID NO: 339, 341, 343, 345 603, 605, 607, 609, 611, 613, 614, 617, 619, 621, 623, 625, 627, 629, and 631 (Table 1) and homologs having at least 80% identity with a HSV antigenic polypeptide selected from any one of SEQ ID NO: 339, 341, 343, 345 603, 605, 607, 609, 611, 613, 614, 617, 619, 621, 623, 625, 627, 629, and 631 (Table 1). In some embodiments, HSV antigenic polypeptide selected from any one of SEQ ID NO: 339, 341, 343, 345 603, 605, 607, 609, 611, 613, 614, 617, 619, 621, 623, 625, 627, 629, and 631 (Table 1) and homologs having at least 90% (e.g. 90%, 91%, 92%, 93%, 94%, 95%, 96%, 97%, 98%, 99%, 99.8%, or 99.9%) identity with a HSV antigenic polypeptide selected from any one of SEQ ID NOs: 339, 341, 343, 345 603, 605, 607, 609, 611, 613, 614, 617, 619, 621, 623, 625, 627, 629, and 631 (Table 1). In some embodiments, a HSV antigenic polypeptide is selected from any one of SEQ ID NOs: 339, 341, 343, 345 603, 605, 607, 609, 611, 613, 614, 617, 619, 621, 623, 625, 627, 629, and 631 (Table 1).

### Varicella Zoster Virus (VZV)

VZV is an alphaherpesvirus that exists as a spherical multilayered structure approximately 200 nm in diameter. The viral genome is surrounded by a protein capsid structure that is covered by an amorphous layer of tegument proteins. These two structures are surrounded by a lipid envelope that is studded with viral glycoproteins, each about 8 nm in length, that are displayed on the exterior of the-virion, and encloses the 100 nm nucleocapsid which is comprised of 162 hexameric and pentameric capsomeres arranged in an icosahedral form. The tegument, which is comprised of virally-encoded proteins and enzymes, is located in the space between the nucleocapsid and the viral envelope. The viral envelope is acquired from host cell membranes and contains viral-encoded glycoproteins.

The VZV genome is a single, linear, duplex DNA molecule of 124,884 base pairs having at least 70 open reading frames. The genome has 2 predominant isomers, depending on the orientation of the S segment, P (prototype) and IS (inverted S), which are present with equal frequency for a total frequency of 90-95%. The L segment can also be inverted resulting in a total of four linear isomers (IL and ILS).

VZV is closely related to the herpes simplex viruses (HSV), sharing much genome homology. The VZV genome is the smallest of the human herpesviruses and encodes at least 71 unique proteins (ORFO-ORF68) with three more opening reading frames (ORF69-ORF71) that duplicate earlier open reading frames (ORF64-62, respectively). Only a fraction of the encoded proteins form the structure of the virus particle. Among those proteins are nine glycoproteins: ORFS (gK), ORF9A (gN), ORF14 (gC), ORF31 (gB), ORF37 (gH), ORF50 (gM), ORF60 (gL), ORF67 (gI), and ORF68 (gE). The known envelope glycoproteins (gB, gC, gE, gH, gl, gK, gL, gN, and gM) correspond with those in HSV; however, there is no equivalent of HSV gD. VZV also fails to produce the LAT (latency-associated transcripts) that play an important role in establishing HSV latency (herpes simplex virus). The encoded glycoproteins gE, gl, gB, gH, gK, gL, gC, gN, and gM function in different steps of the viral replication cycle. The most abundant glycoprotein found in infected cells. as well as in the mature virion, is glycoprotein E (gE, ORF 68), which is a major component of the virion envelope and is essential for viral replication. Glycoprotein I (gI, ORG 67) forms a complex with gE in infected cells, which facilitates the endocytosis of both glycoproteins and directs them to the trans-Golgi network (TGN) where the final viral envelope is acquired. Glycoprotein I (gI) is required within the TGN for VZV envelopment and for efficient membrane fusion during VZV replication. VZV gE and gl are found complexed together on the infected host cell surface. Glycoprotein B (ORF 31), which is the second most prevalent glycoprotein and thought to play a role in virus entry, binds to neutralizing antibodies. Glycoprotein H is thought to have a fusion function facilitating cell to cell spread of the virus. Antibodies to gE, gB, and gH are prevalent after natural infection and following vaccination and have been shown to neutralize viral activity in vitro.

In some embodiments, an antigenic polypeptide is a VZV glycoprotein. For example, a VZV glycoprotein may be VZV gE, gl, gB, gH, gK, gL, gC, gN, or gM. In some embodiments, the antigenic polypeptide is a VZV gE polypeptide. In some embodiments, the antigenic polypeptide is a VZV gl polypeptide. In some embodiments, the antigenic polypeptide is a VZV gB polypeptide. In some embodiments, the antigenic polypeptide is a VZV gH polypeptide. In some embodiments, the antigenic polypeptide is a VZV gK polypeptide. In some embodiments, the antigenic polypeptide is a VZV gL polypeptide. In some embodiments, the antigenic polypeptide is a VZV gC polypeptide. In some embodiments, the antigenic polypeptide is a VZV gN polypeptide. In some embodiments, the antigenic polypeptide is a VZV gM polypeptide.

In some embodiments, an antigenic polypeptide is a VZV glycoprotein. For example, a VZV glycoprotein may be VZV gE, gl, gB, gH, gK, gL, gC, gN, or gM. In some embodiments, the antigenic polypeptide is a VZV gE polypeptide. In some embodiments, the antigenic polypeptide is a VZV gl polypeptide. In some embodiments, the antigenic polypeptide is a VZV gB polypeptide. In some embodiments, the antigenic polypeptide is a VZV gH polypeptide. In some embodiments, the antigenic polypeptide is a VZV gK polypeptide. In some embodiments, the antigenic polypeptide is a VZV gL polypeptide. In some embodiments, the antigenic polypeptide is a VZV gC polypeptide. In some embodiments, the antigenic polypeptide is a VZV gN polypeptide. In some embodiments, the antigenic polypeptide is a VZV gM polypeptide. In some embodiments, the VZV glycoprotein is encoded by a nucleic acid sequence of SEQ ID NO: 290 or SEQ ID NO: 291.

In some embodiments, the VZV glycoprotein is a variant gE polypeptide. In some embodiments, the variant VZV gE polypeptide is a truncated polypeptide lacking the anchor domain (ER retention domain). In some embodiments, the truncated VZV gE polypeptide comprises (or consists of, or consists essentially of) amino acids 1-561 of VZV gE polypeptide. In some embodiments, the truncated VZV gE polypeptide comprises (or consists of, or consists essentially of) amino acids 1-561 of SEQ ID NO: 298. In some embodiments, the truncated VZV gE polypeptide comprises (or consists of, or consists essentially of) amino acids 1-573 of SEQ ID NO: 300. In some embodiments, the truncated VZV gE polypeptide comprises (or consists of, or consists essentially of) amino acids 1-573 of SEQ ID NO: 298. In some embodiments, the variant VZV gE polypeptide is a truncated polypeptide lacking the carboxy terminal tail domain. In some embodiments, the truncated VZV gE polypeptide comprises (or consists of, or consists essentially of) amino acids 1-573 of VZV gE polypeptide. In some embodiments, the truncated VZV gE polypeptide comprises (or consists of, or consists essentially of) amino acids 1-573 of SEQ ID NO: 304.

In some embodiments, the variant VZV gE polypeptide has at least one mutation in one or more motif(s) associated with ER retention, wherein the mutation(s) in one or more motif(s) results in decreased retention of the VZV gE polypeptide in the ER and/or golgi. In some embodiments, the variant VZV gE polypeptide has at least one mutation in one or more motif(s) associated with targeting gE to the golgi or trans-golgi network (TGN), wherein the mutation(s) in one or more motif(s) results in decreased targeting or localization of the VZV gE polypeptide to the golgi or TGN. In some embodiments, the variant VZV gE polypeptide has at least one mutation in one or more motif(s) associated with the internalization of VZV gE or the endocytosis of gE, wherein the mutation(s) in one or more motif(s) results in decreased endocytosis of the VZV gE polypeptide. In some embodiments, the variant VZV gE polypeptide has at least one mutation in one or more phosphorylated acidic motif(s), such as SSTT (SEQ ID NO: 320). In some embodiments, the variant VZV gE polypeptide is a full-length VZV gE polypeptide having a Y582G mutation. In some embodiments, the variant VZV gE polypeptide is a full-length VZV gE polypeptide having a Y569A mutation. In some embodiments, the variant VZV gE polypeptide is a full-length VZV gE polypeptide having a Y582G mutation and a Y569A mutation. In some embodiments, the variant VZV gE polypeptide is an antigenic fragment comprising amino acids 1-573 of VZV gE and having a Y569A mutation. In some embodiments, the variant VZV gE polypeptide is an antigenic fragment comprising SEQ ID NO: 305.

In some embodiments, the variant VZV gE polypeptide is SEQ ID NO: 299. In some embodiments, the variant VZV gE polypeptide is SEQ ID NO: 302. In some embodiments, the variant VZV gE polypeptide is or comprises the amino acid sequence of SEQ ID NO: 303.

In some embodiments, the variant VZV gE polypeptide is a full-length VZV gE polypeptide having an additional sequence at the C-terminus that aids in secretion of the polypeptide or its localization to the cell membrane. In some embodiments, the variant VZV gE polypeptide is a full-length VZV gE polypeptide having an IgKappa sequence at the C-terminus. In some embodiments, the VZV gE polypeptide has additional sequence at the C-terminus that aids in secretion (e.g., has an IgKappa sequence at the C-terminus) and has at least one mutation in one or more motif(s) associated with ER retention, TGN localization, or endocytosis (e.g., has a Y582G mutation, a Y569A mutation, or both a Y582G mutation and a Y569A mutation) and/or has at least one mutation in one or more phosphorylated acidic motif(s), such as the SSTT (SEQ ID NO: 320) motif. In some embodiments, the variant VZV gE polypeptide is a truncated polypeptide lacking the anchor domain (ER retention domain) and having an additional sequence at the C-terminus that aids in secretion of the polypeptide (e.g., an IgKappa sequence at the C-terminus). In some embodiments, the truncated VZV gE polypeptide comprises amino acids 1-561 and has an IgKappa sequence at the C-terminus. In some embodiments, the variant polypeptide is SEQ ID NO: 301.

In some embodiments, at least one RNA polynucleotide is encoded by at least one nucleic acid sequence selected from SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 306 and homologs having at least 80% (e.g., 85%, 90%, 95%, 98%, 99%) identity with a nucleic acid sequence selected from SEQ ID NO: 290-297 and 306. In some embodiments, at least one RNA polynucleotide is encoded by at least one nucleic acid sequence selected from SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 306 and homologs having at least 90% (e.g., 90%, 91%, 92%, 93%, 94%, 95%, 96%, 97%, 98%, 99%, 99.8% or 99.9%) identity with a nucleic acid sequence selected from SEQ ID NO: 1-8 and 41. In some embodiments, at least one RNA polynucleotide is encoded by at least one fragment of a nucleic acid sequence (e.g., a fragment having an antigenic sequence or at least one epitope) selected from SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 306 and homologs having at least 80% (e.g., 85%, 90%, 95%, 98%, 99%) identity with a nucleic acid sequence selected from SEQ ID NO: 290-297 and 306. In some embodiments, at least one RNA polynucleotide is encoded by at least one epitope of a nucleic acid sequence selected from SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 306.

In some embodiments, at least one RNA polynucleotide is a gE polypeptide encoded by SEQ ID NO: 290. In some embodiments, at least one RNA polynucleotide is a gl polypeptide encoded by SEQ ID NO: 291. In some embodiments, at least one RNA polynucleotide is a truncated gE polypeptide encoded by SEQ ID NO: 292. In some embodiments, at least one RNA polynucleotide is a truncated gE polypeptide encoded by SEQ ID NO: 294. In some embodiments, at least one RNA polynucleotide is a truncated gE polypeptide having Y569A mutation encoded by SEQ ID NO: 295. In some embodiments, at least one RNA polynucleotide is a gE polypeptide having an AEAADA sequence SEQ ID NO: 319 encoded by SEQ ID NO: 296. In some embodiments, at least one RNA polynucleotide is a gE polypeptide having a Y582G mutation and a AEAADA sequence (SEQ ID NO: 319) encoded by SEQ ID NO: 297. In some embodiments, at least one RNA polynucleotide is a gE polypeptide encoded by SEQ ID NO: 306.

In some embodiments, at least one RNA (e.g., mRNA) polynucleotide encodes an antigenic polypeptide having at least 90% identity to the amino acid sequence of any one of SEQ ID NO: 298, 299, 300, 301, 302, 303, 304, 305, 307 and 308-318. In some embodiments, at least one RNA (e.g., mRNA) polynucleotide encodes an antigenic polypeptide having at least 95% identity to the amino acid sequence of any one of SEQ ID NO: 298, 299, 300, 301, 302, 303, 304, 305, 307 and 308-318. In some embodiments, at least one RNA polynucleotide encodes an antigenic polypeptide having at least 96% identity to the amino acid sequence of any one of SEQ ID NO: 298, 299, 300, 301, 302, 303, 304, 305, 307 and 308-318. In some embodiments, at least one RNA (e.g., mRNA) polynucleotide encodes an antigenic polypeptide having at least 97% identity to the amino acid sequence of any one of SEQ ID NO: 298, 299, 300, 301, 302, 303, 304, 305, 307 and 308-318. In some embodiments, at least one RNA (e.g., mRNA) polynucleotide encodes an antigenic polypeptide having at least 98% identity to the amino acid sequence of any one of SEQ ID NO: 298, 299, 300, 301, 302, 303, 304, 305, 307 and 308-318. In some embodiments, at least one RNA (e.g., mRNA) polynucleotide encodes an antigenic polypeptide having at least 99% identity to the amino acid sequence of any one of SEQ ID NO: 298, 299, 300, 301, 302, 303, 304, 305, 307 and 308-318.

In some embodiments, the open reading frame from which the VZV polypeptide is encoded is codon-optimized.

In some embodiments, the at least one RNA (e.g., mRNA) polynucleotide encodes an antigenic protein of SEQ ID NO: 298, wherein the RNA (e.g., mRNA) polynucleotide has less than 80% identity to wild-type mRNA sequence. In some embodiments, the at least one RNA polynucleotide encodes an antigenic protein of SEQ ID NO: 298, wherein the RNA (e.g., mRNA) polynucleotide has greater than 80% identity to wild-type mRNA sequence, but does not include wild-type mRNA sequence. In some embodiments, the at least one RNA (e.g., mRNA) polynucleotide encodes an antigenic protein of SEQ ID NO: 307, wherein the RNA e.g., mRNA) polynucleotide has less than 80% identity to wild-type mRNA sequence. In some embodiments, the at least one RNA (e.g., mRNA) polynucleotide encodes an antigenic protein of SEQ ID NO: 307, wherein the RNA polynucleotide has greater than 80% identity to wild-type mRNA sequence, but does not include wild-type mRNA sequence. In some embodiments, the at least one RNA (e.g., mRNA) polynucleotide encodes an antigenic protein of SEQ ID NO: 299, wherein the RNA (e.g., mRNA) polynucleotide has less than 80% identity to wild-type mRNA sequence. In some embodiments, the at least one RNA (e.g., mRNA) polynucleotide encodes an antigenic protein of SEQ ID NO: 299, wherein the RNA (e.g., mRNA) polynucleotide has greater than 80% identity to wild-type mRNA sequence, but does not include wild-type mRNA sequence. In some embodiments, the at least one RNA (e.g., mRNA) polynucleotide encodes an antigenic protein of SEQ ID NO: 302, wherein the RNA polynucleotide has less than 80% identity to wild-type mRNA sequence. In some embodiments, the at least one RNA (e.g., mRNA) polynucleotide encodes an antigenic protein of SEQ ID NO: 302, wherein the RNA (e.g., mRNA) polynucleotide has greater than 80% identity to wild-type mRNA sequence, but does not include wild-type mRNA sequence. In some embodiments, the at least one RNA (e.g., mRNA) polynucleotide encodes an antigenic protein of SEQ ID NO: 303, wherein the RNA polynucleotide has less than 80% identity to wild-type mRNA sequence. In some embodiments, the at least one RNA polynucleotide encodes an antigenic protein of SEQ ID NO: 303, wherein the RNA (e.g., mRNA) polynucleotide has greater than 80% identity to wild-type mRNA sequence, but does not include wild-type mRNA sequence. In some embodiments, the at least one RNA (e.g., mRNA) polynucleotide is encoded by a sequence selected from any one of SEQ ID NO: 290-297 and SEQ ID NO 306 and includes at least one chemical modification.

In some embodiments, the nucleic acids disclosed herein can include at least one chemically modified nucleotide. In some embodiments, the at least one chemically modified nucleotide comprises a chemically modified nucleobase, a chemically modified ribose, a chemically modified phosphodiester linkage, or a combination thereof.

In one embodiment, the at least one chemically modified nucleotide is a chemically modified nucleobase.

In one embodiment, the chemically modified nucleobase is selected from 5-formylcytidine (5fC), 5-methylcytidine (5meC), 5-methoxycytidine (5moC), 5-hydroxycytidine (5hoC), 5-hydroxymethylcytidine (5hmC), 5-formyluridine (5fU), 5-methyluridine (5-meU), 5-methoxyuridine (5moU), 5-carboxymethylesteruridine (5camU), pseudouridine (Ψ), N1-methylpseudouridine (me¹Ψ), N⁶-methyladenosine (me⁶A), or thienoguanosine (^{th}G).

In some embodiments, the chemically modified nucleobase is 5-methoxyuridine (5moU). In some embodiments, the chemically modified nucleobase is pseudouridine (Ψ). In some embodiments, the chemically modified nucleobase is N1-methylpseudouridine (me¹Ψ).

The structures of these modified nucleobases are shown below:

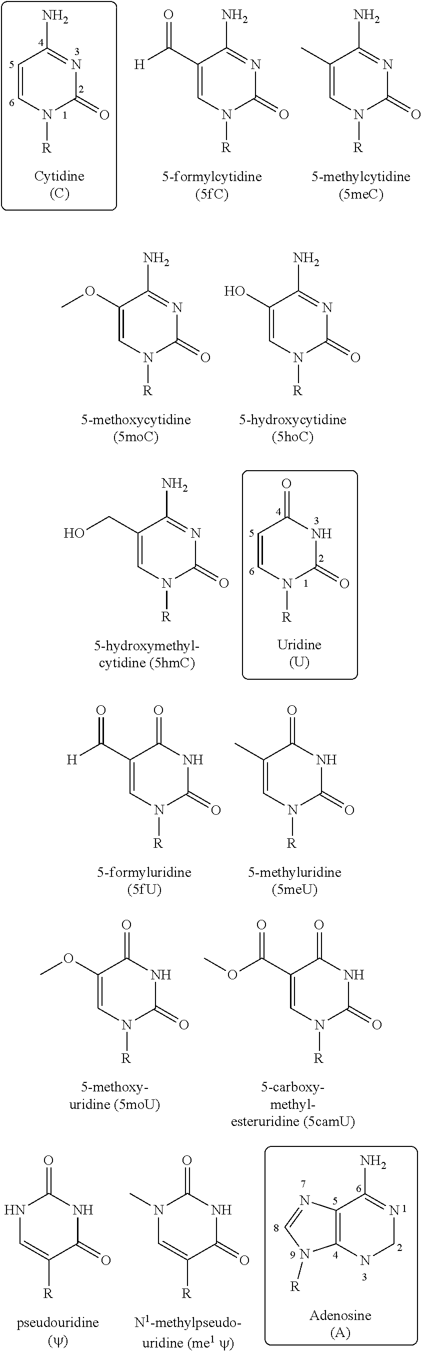

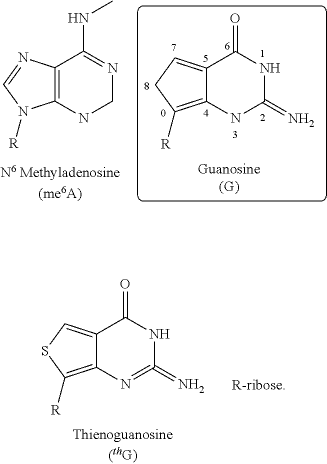

In one embodiment, the at least one chemically modified nucleotide is a chemically modified ribose.

In one embodiment, the chemically modified ribose is selected from 2′-O-methyl(2′-O-Me), 2′-Fluoro (2′-F), 2′-deoxy-2′-fluoro-beta-D-arabino-nucleic acid (2′F-ANA), 4′-S, 4′-SFANA, 2′-azido, UNA, 2′-O-methoxy-ethyl(2′-O-ME), 2′-O-Allyl, 2′-O-Ethylamine, 2′-O-Cyanoethyl, Locked nucleic acid (LAN), Methylene-cLAN, N-MeO-amino BNA, or N-MeO-aminooxy BNA. In one embodiment, the chemically modified ribose is 2′-O-methyl(2′-O-Me). In one embodiment, the chemically modified ribose is 2′-Fluoro (2′-F).

The structures of these modified riboses are shown below:

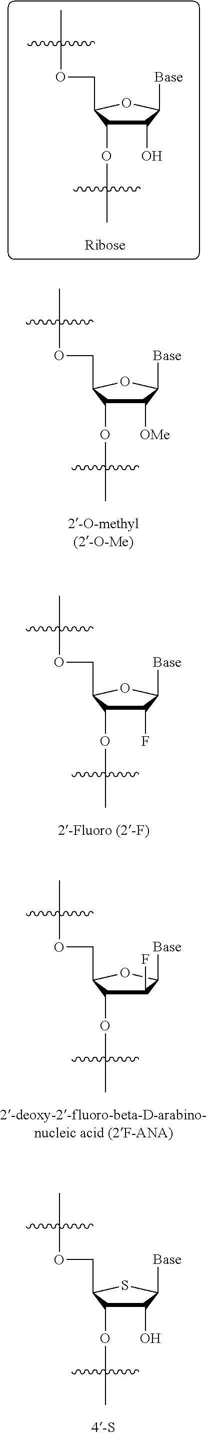

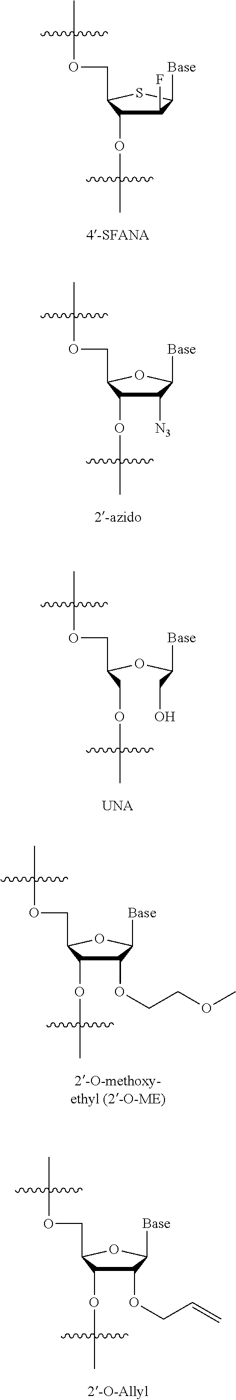

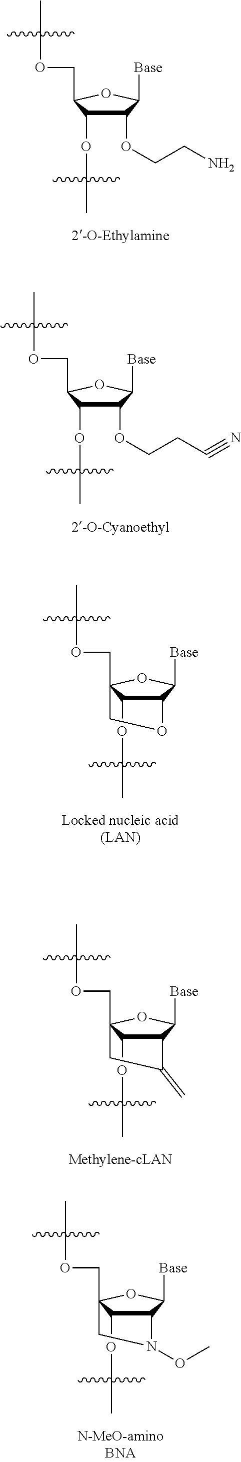

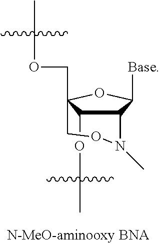

In one embodiment, the at least one chemically modified nucleotide is a chemically modified phosphodiester linkage.

In one embodiment, the chemically modified phosphodiester linkage is selected from phosphorothioate (PS), boranophosphate, phosphodithioate (PS2), 3′,5′-amide, N3′-phosphoramidate (NP), Phosphodiester (PO), or 2′,5′-phosphodiester (2′,5′-PO). In one embodiment, the chemically modified phosphodiester linkage is phosphorothioate.

The structures of these modified phosphodiester linkages are shown below:

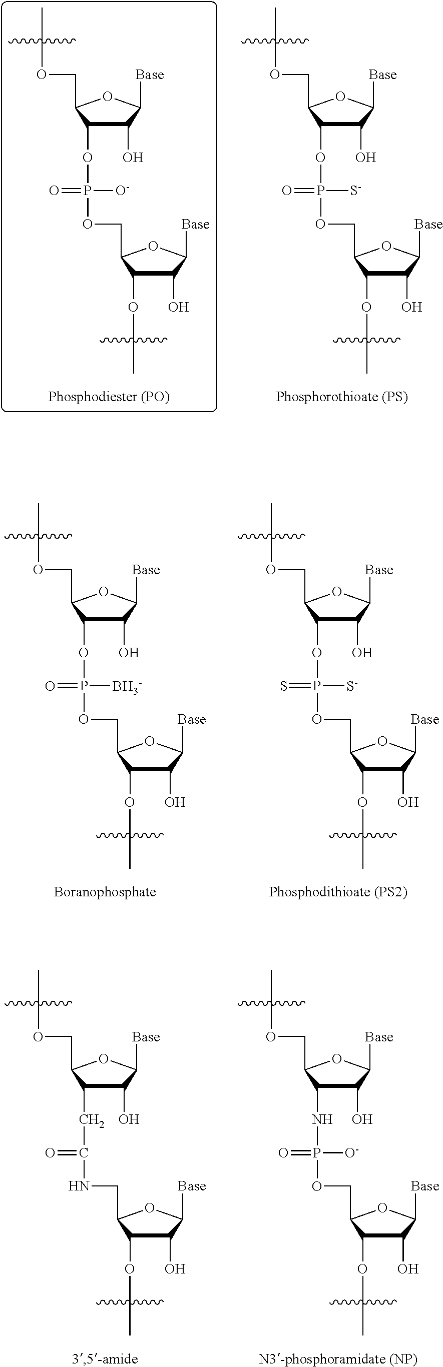

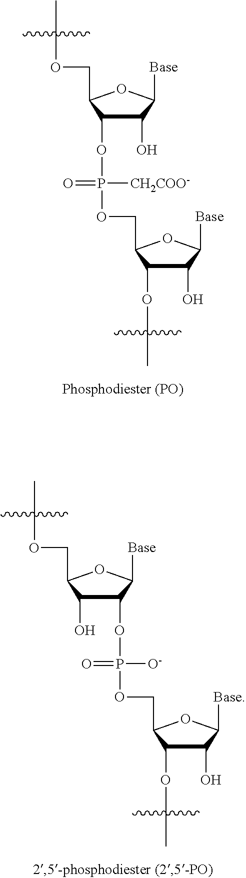

In some embodiments, the mRNA can include a heterologous 5′ untranslated region (5′UTR). In some embodiments, the mRNA can include a heterologous 3′ untranslated region (3′UTR).

### Methods of Administration

The compositions as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate | Myrj 45|, polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus. Phenonip, methylparaben. Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy (Polyethylene glycol)-1000], 1,2-Distearoyl-sn-glycero-3-Phosphoethanolamine-N-[Methoxy (Polyethylene glycol)-2000], and 1,2-Distearoyl-sn-glycero-3-Phosphoethanolamine-N-[Methoxy (Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly(meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80]. sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The compounds can be incorporated microparticles, nanoparticles, or combinations thereof that provide controlled release of the compounds and/or additional active agents. For example, the compounds can be incorporated into polymeric microparticles, which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, may also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the compound can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents may be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

Encapsulation or incorporation of drug into carrier materials to produce drug-containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, drug(s) in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In some embodiments, drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

In certain embodiments, it may be desirable to provide continuous delivery of one or more compounds to a patient in need thereof. For intravenous or intraarterial routes, this can be accomplished using drip systems, such as by intravenous administration. For topical applications, repeated application can be done or a patch can be used to provide continuous administration of the compounds over an extended period of time.

The compounds described herein can be incorporated into injectable/implantable solid or semi-solid implants, such as polymeric implants. In one embodiment, the compounds are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication require polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the compounds can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, wafers, or extruded into a device, such as rods.

The release of the compounds from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

In some embodiments, the compounds or pharmaceutical compositions can be administered locally. In some embodiments, the compounds are incorporated in a delivery system such as gels, nanoparticles, microparticles, or implants such as (e.g., rods, discs, wafers, orthopedic implants) for sustained release. In some embodiments, the compounds can be administered using a local delivery implantable system comprising the compounds incorporated within a gel, nanoparticles, microparticles, or an implant. In some embodiments, the pharmaceutical compositions comprise a delivery system such as gels, nanoparticles, microparticles, or implants such as (e.g., rods, discs, wafers, orthopedic implants) for sustained release of paroxetine or a pharmaceutically acceptable salt or derivative thereof.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions. anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Compositions described herein are typically formulated in dosage unit form for ease of administration and uniformity of dosage. It will be understood, however, that the total daily usage of compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, the compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see, e.g., the range of unit doses described in International Publication No WO2013078199, the contents of which are herein incorporated by reference in their entirety). The desired dosage may be delivered three times a day, two times a day, once a day, every other day, every third day, every week, every two weeks, every three weeks, every four weeks, every 2 months, every three months, every 6 months, etc. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In exemplary embodiments, compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg.

In some embodiments, the compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, the compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, a composition described herein may be administered three or four times.

In some embodiments, the compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.010 mg. 0.025 mg, 0.100 mg or 0.400 mg.

In some embodiments, the composition (e.g., vaccine) for use in a method of vaccinating a subject is administered to the subject as a single dosage of between 10 μg/kg and 400 μg/kg of the nucleic acid vaccine (in an effective amount to vaccinate the subject). In some embodiments, the composition (e.g., vaccine) for use in a method of vaccinating a subject is administered to the subject as a single dosage of between 10 μg and 400 μg of the nucleic acid vaccine (in an effective amount to vaccinate the subject). In some embodiments, a composition (e.g., vaccine) for use in a method of vaccinating a subject is administered to the subject as a single dosage of 25-1000 ug (e.g., a single dosage of mRNA encoding an infection agent and a mRNA encoding T-cell epitope, or an mRNA encoding an infection agent and at least one universal T-cell epitope). In some embodiments, a composition (e.g., vaccine) is administered to the subject as a single dosage of 25, 50, 100, 150, 200, 250, 300, 350, 400, 450, 500, 550, 600, 650, 700, 750, 800, 850, 900, 950 or 1000 ug. For example, a composition (e.g., vaccine) may be administered to a subject as a single dose of 25-100, 25-500, 50-100, 50-500, 50-1000, 100-500, 100-1000, 250-500, 250-1000, or 500-1000 ug. In some embodiments, a composition (e.g., vaccine) for use in a method of vaccinating a subject is administered to the subject as two dosages, the combination of which equals 25-1000 ug of the composition (e.g., vaccine).

A composition (e.g., vaccine pharmaceutical composition) described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### Methods of Use

Provided herein are compositions (e.g., pharmaceutical compositions), methods, kits and reagents for prevention and/or treatment of infections in humans and other mammals. The compositions described herein (e.g., vaccines) can be used as therapeutic or prophylactic agents, alone or in combination with other vaccine(s). They may be used in medicine to prevent and/or treat infection. In exemplary aspects, the compositions of the present disclosure are used to provide prophylactic protection from an infection agent. Prophylactic protection from an infection agent can be achieved following administration of a composition (e.g., vaccine) of the present disclosure. Composition (e.g., vaccine) of the present disclosure may be used to treat or prevent viral “co-infections” containing two or more infections. The composition (e.g., vaccine) can be administered once, twice, three times, four times or more, but it is likely sufficient to administer the composition (e.g., vaccine) once (optionally followed by a single booster). It is possible, although less desirable, to administer the composition (e.g., vaccine) to an infected individual to achieve a therapeutic response. Dosing may need to be adjusted accordingly.

Described herein are methods of treating, inhibiting, reducing, ameliorating, and/or preventing an infection caused by an infection agent in a subject comprising administering to the subject a composition described herein, a nanoparticle described herein, a vector described herein, or a vaccine described herein.

In some embodiments, also described are methods of treating an infection caused by an infection agent in a subject comprising administered to the subject a composition described herein, a nanoparticle described herein, a vector described herein, or a vaccine described herein.

In some embodiments. also described are methods of activating T cells comprising contacting the T cells with a composition described herein, a nanoparticle described herein, a vector described herein, or a vaccine described herein.

In some embodiments, also described are methods of stimulating the proliferation of T cells comprising contacting the T cells with a composition described herein, a nanoparticle described herein, or a vaccine described herein.

In some embodiments, also described are methods of eliciting an immune response in a subject to an infection agent comprising administering to the subject a composition described herein, a nanoparticle described herein, or a vaccine described herein. In some embodiments, the immune response can include a cellular immune response. a humoral immune response, or a combination thereof. In some embodiments, the immune response can include a cellular immune response, a humoral immune response, or a combination thereof, without risking the possibility of insertional mutagenesis.

In some embodiments, also described are methods of enhancing an immune response generated by an mRNA vaccine including introducing an mRNA encoding at least one T-cell epitope into the mRNA vaccine. In some embodiments, introduction of the mRNA encoding at least one T-cell epitope can increase immune response against variants of an infection agent.

In some embodiments, the composition (e.g., vaccine) of the present disclosure are used in the priming of immune effector cells, for example, to activate peripheral blood mononuclear cells (PBMCs) ex vivo, which are then infused (re-infused) into a subject.

In some embodiments, the composition (e.g., vaccine) containing RNA (e.g., mRNA) polynucleotides as described herein can be administered to a subject (e.g., a mammalian subject, such as a human subject), and the RNA (e.g., mRNA) polynucleotides are translated in vivo to produce an antigenic polypeptide and at least one universal T-cell epitope.

The composition (e.g., vaccine) may be induced for translation of a polypeptide (e.g., antigen or immunogen) in a cell, tissue or organism. In some embodiments, such translation occurs in vivo, although such translation may occur ex vivo, in culture or in vitro. In some embodiments, the cell, tissue or organism is contacted with an effective amount of a composition containing one or more RNA (e.g., mRNA) that contains a polynucleotide that has at least one a translatable region encoding an antigenic polypeptide and at least one universal T-cell epitope.

An “effective amount” of a composition (e.g., vaccine) is provided based, at least in part, on the target tissue, target cell type, means of administration, physical characteristics of the polynucleotide (e.g., size, and extent of modified nucleosides) and other components of the composition (e.g., vaccine), and other determinants. In general, an effective amount of the composition (e.g., vaccine) provides an induced or boosted immune response as a function of antigen production in the cell, preferably more efficient than a composition containing a corresponding unmodified polynucleotide encoding the same antigen or a peptide antigen. Increased antigen production may be demonstrated by increased cell transfection (the percentage of cells transfected with the RNA, e.g., mRNA), increased protein translation from the polynucleotide, decreased nucleic acid degradation (as demonstrated, for example, by increased duration of protein translation from a modified polynucleotide), or altered antigen specific immune response of the host cell.

In some embodiments, anti-antigenic polypeptide antibody titer in the subject is increased following vaccination relative to anti-antigenic polypeptide antibody titer in a subject vaccinated with a prophylactically effective dose of a traditional vaccine against the infection agent. An “anti-antigenic polypeptide antibody” is a serum antibody the binds specifically to the antigenic polypeptide.

In some embodiments, a composition (e.g., vaccine) described herein capable of eliciting an immune response is administered intramuscularly via a composition including a compound according to Formula A, I, Ia, or II-V, or any combination thereof described herein.

In some embodiments the anti-antigenic polypeptide antibody titer in the subject is increased 1 log to 10 log following vaccination relative to anti-antigenic polypeptide antibody titer in a subject vaccinated with a prophylactically effective dose of a traditional vaccine an infection agent.

In some embodiments the anti-antigenic polypeptide antibody titer in the subject is increased 1 log, 2 log, 3 log, 5 log or 10 log following vaccination relative to anti-antigenic polypeptide antibody titer in a subject vaccinated with a prophylactically effective dose of a traditional vaccine against infection agent.

A prophylactically effective dose is a therapeutically effective dose that prevents infection with the virus at a clinically acceptable level. In some embodiments, the therapeutically effective dose is a dose listed in a package insert for the composition (e.g., vaccine). A traditional vaccine, as used herein, refers to a vaccine other than the RNA (e.g., mRNA) vaccines described herein. For instance, a traditional vaccine includes but is not limited to RNA (e.g., mRNA) vaccines that do not encode at least one universal T-cell epitope, live/attenuated microorganism vaccines, killed/inactivated microorganism vaccines, subunit vaccines, protein antigen vaccines, DNA vaccines, VLP vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

RNA (e.g. mRNA) vaccines described herein may be administered prophylactically or therapeutically as part of an active immunization scheme to healthy individuals or early in infection during the incubation phase or during active infection after onset of symptoms. In some embodiments, the amount of RNA (e.g., mRNA) vaccine of the present disclosure provided to a cell, a tissue or a subject may be an amount effective for immune prophylaxis.

RNA (e.g. mRNA) vaccines may be administrated with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In some embodiments, the time of administration between the initial administration of the prophylactic composition and the booster may be, but is not limited to, 1 week, 2 weeks, 3 weeks, 1 month, 2 months, 3 months, 6 months or 1 year.

In some embodiments, the immune response in the subject is equivalent to an immune response in a subject vaccinated with a traditional vaccine at 2, 3, 4, 5, 10, 50, 100 times the dosage level relative to the RNA (e.g., mRNA) vaccine described herein.

In some embodiments the immune response in the subject is equivalent to an immune response in a subject vaccinated with a traditional vaccine at 10-100 times, or 100-1000 times, the dosage level relative to the RNA (e.g., mRNA) vaccine described herein.

In some embodiments the immune response is assessed by determining [protein]antibody titer in the subject.

In some embodiments, the immune response in the subject is induced 2 days earlier, or 3 days earlier, relative to an immune response induced in a subject vaccinated with a prophylactically effective dose of a traditional vaccine.

In some embodiments the immune response in the subject is induced 1 week, 2 weeks, 3 weeks, 5 weeks, or 10 weeks earlier relative to an immune response induced in a subject vaccinated with a prophylactically effective dose of a traditional vaccine.

In some embodiments, RNA (e.g. mRNA) vaccines may be administered intramuscularly or intradermally, similarly to the administration of inactivated vaccines known in the art.

Some aspects of the present disclosure provide compositions, wherein the RNA (e.g., mRNA) vaccine is formulated in an effective amount to produce an antigen specific immune response in a subject (e.g., production of antibodies specific to an infection agent antigenic polypeptide). “An effective amount” is a dose of an RNA (e.g., mRNA) vaccine effective to produce an antigen-specific immune response. Also provided herein are methods of inducing an antigen-specific immune response in a subject.

In some embodiments, the antigen-specific immune response is characterized by measuring an anti-infection agent antigenic polypeptide antibody titer produced in a subject administered a composition as provided herein. An antibody titer is a measurement of the amount of antibodies within a subject, for example, antibodies that are specific to a particular antigen (e.g., an anti-infection agent antigenic polypeptide) or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by the RNA (e.g., mRNA) vaccine described herein.

In some embodiments, an anti-antigenic polypeptide antibody titer produced in a subject is increased by at least 1 log relative to a control. For example, anti-antigenic polypeptide antibody titer produced in a subject may be increased by at least 1.5, at least 2, at least 2.5, or at least 3 log relative to a control. In some embodiments, the anti-antigenic polypeptide antibody titer produced in the subject is increased by 1, 1.5, 2, 2.5 or 3 log relative to a control. In some embodiments, the anti-antigenic polypeptide antibody titer produced in the subject is increased by 1-3 log relative to a control. For example, the anti-antigenic polypeptide antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the anti-antigenic polypeptide antibody titer produced in a subject is increased at least 2 times relative to a control. For example, the anti-antigenic polypeptide antibody titer produced in a subject may be increased at least 3 times, at least 4 times, at least 5 times, at least 6 times, at least 7 times, at least 8 times, at least 9 times, or at least 10 times relative to a control. In some embodiments, the anti-antigenic polypeptide antibody titer produced in the subject is increased 2, 3, 4, 5, 6, 7, 8, 9, or 10 times relative to a control. In some embodiments, the anti-antigenic polypeptide antibody titer produced in a subject is increased 2-10 times relative to a control. For example, the anti-antigenic polypeptide antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

A control, in some embodiments, is the anti-antigenic polypeptide antibody titer produced in a subject who has not been administered a RNA (e.g., mRNA) vaccine of the present disclosure. In some embodiments, a control is an anti-antigenic polypeptide antibody titer produced in a subject who has been administered a RNA (e.g., mRNA) vaccine that do not encode at least one universal T-cell epitope. In some embodiments, a control is an anti-antigenic polypeptide antibody titer produced in a subject who has been administered a live attenuated vaccine. An attenuated vaccine is a vaccine produced by reducing the virulence of a viable (live). An attenuated virus is altered in a manner that renders it harmless or less virulent relative to live, unmodified virus. In some embodiments, a control is an anti-antigenic polypeptide antibody titer produced in a subject administered inactivated vaccine. In some embodiments, a control is an anti-antigenic polypeptide antibody titer produced in a subject administered a recombinant or purified protein vaccine. Recombinant protein vaccines typically include protein antigens that either have been produced in a heterologous expression system (e.g., bacteria or yeast) or purified from large amounts of the pathogenic organism. In some embodiments, a control is an anti-antigenic polypeptide antibody titer produced in a subject who has been administered a virus-like particle (VLP) vaccine.

In some embodiments, an effective amount of a RNA (e.g., mRNA) vaccine is a dose that is reduced compared to the standard of care dose of a recombinant protein vaccine. A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a recombinant or purified protein vaccine, or a live attenuated or inactivated vaccine, that a physician/clinician or other medical professional would administer to a subject to treat or prevent an infection agent-related condition, while following the standard of care guideline for treating or preventing an infection agent-related condition.

In some embodiments, the anti-antigenic polypeptide antibody titer produced in a subject administered an effective amount of a RNA (e.g., mRNA) vaccine is equivalent to an anti-antigenic polypeptide antibody titer produced in a control subject administered a standard of care dose of a recombinant or purified protein vaccine or a live attenuated or inactivated vaccine.

In some embodiments, an effective amount of a RNA (e.g., mRNA) vaccine is a dose equivalent to an at least 2-fold reduction in a standard of care dose of a recombinant or purified protein vaccine. For example, an effective amount of a RNA (e.g., mRNA) vaccine may be a dose equivalent to an at least 3-fold, at least 4-fold, at least 5-fold, at least 6-fold, at least 7-fold, at least 8-fold, at least 9-fold, or at least 10-fold reduction in a standard of care dose of a recombinant or purified protein vaccine. In some embodiments, an effective amount of a RNA (e.g., mRNA) vaccine is a dose equivalent to an at least at least 100-fold, at least 500-fold, or at least 1000-fold reduction in a standard of care dose of a recombinant or purified protein vaccine. In some embodiments, an effective amount of a RNA (e.g., mRNA) vaccine is a dose equivalent to a 2-, 3-. 4-, 5-, 6-, 7-, 8-, 9-, 10-, 20-, 50-, 100-, 250-, 500-, or 1000-fold reduction in a standard of care dose of a recombinant or purified protein vaccine. In some embodiments, the anti-antigenic polypeptide antibody titer produced in a subject administered an effective amount of a RNA (e.g., mRNA) vaccine is equivalent to an anti-antigenic polypeptide antibody titer produced in a control subject administered the standard of care dose of a recombinant or protein protein vaccine or a live attenuated or inactivated vaccine. In some embodiments, an effective amount of a RNA (e.g., mRNA) vaccine is a dose equivalent to a 2-fold to 1000-fold (e.g., 2-fold to 100-fold, 10-fold to 1000-fold) reduction in the standard of care dose of a recombinant or purified protein vaccine, wherein the anti-antigenic polypeptide antibody titer produced in the subject is equivalent to an anti-antigenic polypeptide antibody titer produced in a control subject administered the standard of care dose of a recombinant or purified protein vaccine or a live attenuated or inactivated vaccine.

In some embodiments, the effective amount of a RNA (e.g., mRNA) vaccine is a dose equivalent to a 2 to 1000-, 2 to 900-, 2 to 800-, 2 to 700-, 2 to 600-, 2 to 500-, 2 to 400-, 2 to 300-, 2 to 200-, 2 to 100-, 2 to 90-, 2 to 80-, 2 to 70-, 2 to 60-, 2 to 50-, 2 to 40-, 2 to 30-, 2 to 20-, 2 to 10-, 2 to 9-, 2 to 8-, 2 to 7-, 2 to 6-. 2 to 5-, 2 to 4-, 2 to 3-, 3 to 1000-, 3 to 900-, 3 to 800-, 3 to 700-, 3 to 600-, 3 to 500-, 3 to 400-, 3 to 3 to 00-, 3 to 200-, 3 to 100-, 3 to 90-, 3 to 80-, 3 to 70-, 3 to 60-, 3 to 50-, 3 to 40-, 3 to 30-, 3 to 20-, 3 to 10-, 3 to 9-, 3 to 8-, 3 to 7-, 3 to 6-, 3 to 5-, 3 to 4-, 4 to 1000-, 4 to 900-, 4 to 800-, 4 to 700-, 4 to 600-, 4 to 500-, 4 to 400-, 4 to 4 to 00-, 4 to 200-, 4 to 100-, 4 to 90-, 4 to 80-, 4 to 70-, 4 to 60-, 4 to 50-, 4 to 40-, 4 to 30-, 4 to 20-, 4 to 10-, 4 to 9-, 4 to 8-, 4 to 7-, 4 to 6-, 4 to 5-, 4 to 4-, 5 to 1000-, 5 to 900-, 5 to 800-, 5 to 700-, 5 to 600-, 5 to 500-, 5 to 400-, 5 to 300-, 5 to 200-, 5 to 100-, 5 to 90-, 5 to 80-, 5 to 70-, 5 to 60-, 5 to 50-, 5 to 40-, 5 to 30-, 5 to 20-, 5 to 10-, 5 to 9-, 5 to 8-, 5 to 7-, 5 to 6-, 6 to 1000-, 6 to 900-, 6 to 800-, 6 to 700-, 6 to 600-, 6 to 500-, 6 to 400-, 6 to 300-, 6 to 200-, 6 to 100-, 6 to 90-, 6 to 80-, 6 to 70-, 6 to 60-, 6 to 50-, 6 to 40-, 6 to 30-, 6 to 20-, 6 to 10-, 6 to 9-, 6 to 8-, 6 to 7-, 7 to 1000-, 7 to 900-, 7 to 800-, 7 to 700-, 7 to 600-, 7 to 500-, 7 to 400-, 7 to 300-, 7 to 200-, 7 to 100-, 7 to 90-, 7 to 80-, 7 to 70-, 7 to 60-, 7 to 50-, 7 to 40-, 7 to 30-. 7 to 20-, 7 to 10-, 7 to 9-, 7 to 8-, 8 to 1000-, 8 to 900-, 8 to 800-, 8 to 700-, 8 to 600-, 8 to 500-, 8 to 400-, 8 to 300-, 8 to 200-, 8 to 100-, 8 to 90-, 8 to 80-, 8 to 70-, 8 to 60-, 8 to 50-, 8 to 40-, 8 to 30-, 8 to 20-, 8 to 10-, 8 to 9-, 9 to 1000-, 9 to 900-, 9 to 800-, 9 to 700-, 9 to 600-, 9 to 500-, 9 to 400-, 9 to 300-, 9 to 200-, 9 to 100-, 9 to 90-, 9 to 80-, 9 to 70-, 9 to 60-, 9 to 50-, 9 to 40-, 9 to 30-, 9 to 20-, 9 to 10-, 10 to 1000-, 10 to 900-, 10 to 800-, 10 to 700-, 10 to 600-, 10 to 500-, 10 to 400-, 10 to 300-, 10 to 200-. 10 to 100-, 10 to 90-, 10 to 80-, 10 to 70-, 10 to 60-, 10 to 50-, 10 to 40-, 10 to 30-, 10 to 20-, 20 to 1000-, 20 to 900-, 20 to 800-, 20 to 700-, 20 to 600-, 20 to 500-, 20 to 400-, 20 to 300-, 20 to 200-, 20 to 100-, 20 to 90-, 20 to 80-, 20 to 70-, 20 to 60-, 20 to 50-, 20 to 40-, 20 to 30-, 30 to 1000-, 30 to 900-, 30 to 800-, 30 to 700-, 30 to 600-, 30 to 500-, 30 to 400-, 30 to 300-, 30 to 200-, 30 to 100-, 30 to 90-, 30 to 80-, 30 to 70-, 30 to 60-, 30 to 50-, 30 to 40-, 40 to 1000-, 40 to 900-, 40 to 800-, 40 to 700-, 40 to 600-, 40 to 500-, 40 to 400-, 40 to 300-, 40 to 200-, 40 to 100-, 40 to 90-, 40 to 80-, 40 to 70-, 40 to 60-, 40 to 50-, 50 to 1000-, 50 to 900-, 50 to 800-, 50 to 700-, 50 to 600-, 50 to 500-, 50 to 400-, 50 to 300-, 50 to 200-, 50 to 100-, 50 to 90-, 50 to 80-, 50 to 70-, 50 to 60-, 60 to 1000-, 60 to 900-, 60 to 800-, 60 to 700-, 60 to 600-, 60 to 500-, 60 to 400-, 60 to 300-, 60 to 200-, 60 to 100-, 60 to 90-, 60 to 80-, 60 to 70-, 70 to 1000-, 70 to 900-, 70 to 800-, 70 to 700-, 70 to 600-, 70 to 500-, 70 to 400-, 70 to 300-, 70 to 200-. 70 to 100-, 70 to 90-, 70 to 80-, 80 to 1000-, 80 to 900-, 80 to 800-, 80 to 700-, 80 to 600-, 80 to 500-, 80 to 400-, 80 to 300-, 80 to 200-, 80 to 100-, 80 to 90-, 90 to 1000-, 90 to 900-, 90 to 800-, 90 to 700-, 90 to 600-, 90 to 500-, 90 to 400-, 90 to 300-, 90 to 200-, 90 to 100-, 100 to 1000-, 100 to 900-, 100 to 800-, 100 to 700-, 100 to 600-, 100 to 500-, 100 to 400-, 100 to 300-, 100 to 200-, 200 to 1000-, 200 to 900-, 200 to 800-, 200 to 700-, 200 to 600-, 200 to 500-, 200 to 400-, 200 to 300-, 300 to 1000-, 300 to 900-, 300 to 800-, 300 to 700-, 300 to 600-, 300 to 500-, 300 to 400-, 400 to 1000-, 400 to 900-, 400 to 800-, 400 to 700-, 400 to 600-, 400 to 500-, 500 to 1000-, 500 to 900-, 500 to 800-, 500 to 700-, 500 to 600-, 600 to 1000-, 600 to 900-, 600 to 800-, 600 to 700-, 700 to 1000-, 700 to 900-, 700 to 800-, 800 to 1000-, 800 to 900-, or 900 to 1000-fold reduction in the standard of care dose of a recombinant protein vaccine. In some embodiments, the anti-antigenic polypeptide antibody titer produced in the subject is equivalent to an anti-antigenic polypeptide antibody titer produced in a control subject administered the standard of care dose of a recombinant or purified protein vaccine or a live attenuated or inactivated vaccine. In some embodiments, the effective amount is a dose equivalent to (or equivalent to an at least) 2-, 3—, 4-, 5-, 6-, 7-, 8-, 9-, 10-, 20-. 30-, 40-, 50-. 60-, 70-, 80-, 90-, 100-, 110-, 120-, 130-, 140-, 150-, 160-, 170-, 1280-, 190-, 200-, 210-, 220-, 230-, 240-, 250-, 260-, 270-, 280-, 290-, 300-, 310-, 320-, 330-, 340-, 350-, 360-, 370-, 380-, 390-, 400-, 410-, 420-, 430-, 440-, 450-, 4360-, 470-, 480-, 490-, 500-, 510-, 520-, 530-, 540-, 550-, 560-, 5760-. 580-, 590-, 600-, 610-, 620-, 630-, 640-, 650-, 660-, 670-, 680-, 690-, 700-, 710-, 720-, 730-, 740-, 750-, 760-, 770-, 780-, 790-, 800-, 810-, 820-, 830-, 840-, 850-, 860-, 870-, 880-, 890-, 900-, 910-, 920-, 930-, 940-, 950-, 960-, 970-, 980-, 990-, or 1000-fold reduction in the standard of care dose of a recombinant protein vaccine. In some embodiments, an anti-antigenic polypeptide antibody titer produced in the subject is equivalent to an anti-antigenic polypeptide antibody titer produced in a control subject administered the standard of care dose of a recombinant or purified protein vaccine or a live attenuated or inactivated vaccine.

RNA (e.g. mRNA) vaccines may be utilized in various settings depending on the prevalence of the infection or the degree or level of unmet medical need. As a non-limiting example, the RNA (e.g., mRNA) vaccines may be utilized to treat and/or prevent a variety of infections. RNA (e.g., mRNA) vaccines have superior properties in that they produce much larger antibody titers and produce responses early than commercially available anti-viral agents/compositions.

Provided herein are pharmaceutical compositions including RNA (e.g. mRNA) and RNA (e.g. mRNA) vaccine compositions and/or complexes optionally in combination with one or more pharmaceutically acceptable excipients.

The compositions (e.g., vaccines) may be formulated or administered alone or in conjunction with one or more other components. For instance, RNA (e.g., mRNA) vaccines (vaccine compositions) may comprise other components including, but not limited to, adjuvants.

In some embodiments, RNA (e.g. mRNA) compositions do not include an adjuvant (they are adjuvant free).

The compositions (e.g., vaccines) may be formulated or administered in combination with one or more pharmaceutically-acceptable excipients. In some embodiments, compositions comprise at least one additional active substances, such as, for example, a therapeutically-active substance, a prophylactically-active substance, or a combination of both. The compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, the compositions (e.g., vaccines) are administered to humans, human patients or subjects. For the purposes of the present disclosure, the phrase “active ingredient” generally refers to the polynucleotides contained in the compositions described herein, for example, RNA polynucleotides (e.g., mRNA polynucleotides) encoding antigenic polypeptides.

Formulations of the compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

The compositions (e.g., vaccines) can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with RNA (e.g. mRNA) (e.g., for transplantation into a subject), hyaluronidase, nanoparticle mimics and combinations thereof.

### Examples

The following examples are set forth below to illustrate the compounds, compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Described is a platform for generating universal vaccines by eliciting antibodies and T cell-immunity against pathogenic infections.

### Example 1: Universal COVID-19 mRNA Vaccine with Formulation of Lipid Nanoparticles (LNP)

The novel coronavirus, SARS-COV-2 that causes COVID-19 illness, has only recently emerged and has rapidly become a global public health emergency. According to World Health Organization, as of Jun. 1, 2022, there are more than 526 million confirmed cases worldwide with greater than 6 million confirmed deaths and as of 22 May 2022, almost one billion people in lower-income countries remain unvaccinated. All viruses, including SARS-COV-2, the virus that causes COVID-19, change over time and some changes may affect the pathological properties such as virus's spreads and disease severity or the performance of vaccines, therapeutic medicines, diagnostic tools, or other public health and social measures. Currently, there are two variants of concern (VOC). One is delta variant and the other is omicron which includes BA.1, BA.2, BA.3, BA.4, BA.5 and descendent lineages. One of the possible scenarios in the near future is that SARS-COV-2 variants emerge repeatedly with the ability to escape vaccine immunity, so that only adapted novel vaccines for multiple rounds of population reimmunization in pursuit of national control. However, many countries struggle with repeated waves and vaccines that are not sufficiently effective against newly circulating viral variants, Therefore, COVID-19 vaccine development should be conducted continuously in world-wide.

We recently developed a new universal mRNA vaccine, PanCOVAX, which is a mRNA vaccine which contains two mRNAs encoding full-length spike glycoprotein and multiple T cell epitopes (MTEs), respectively, both of which are formulated with T1-LNP to form a vaccine, which will generate humoral and cellular immune responses, including neutralizing antibodies and T cell responses. The mRNA encoding spike will elicit neutralizing antibodies, while the mRNA encoding the MTEs will activate T cells. These T cells can efficiently recognize and eliminate virus infected cells, which is particular important when the antibodies lost their neutralizing ability to new variants. The MTEs in the PANCOVAX vaccine contains hundreds of T cell epitopes derived from conserved region of SARS-COV-2 genome, and these T cell epitopes are unchanged across all variants, also many of them are conserved in other member of coronavirus. Thus, our vaccine will generate humoral immune responses and broad T cell responses and represents a novel safer and universal vaccine against COVID-19, SARS, and other coronaviruses

### Materials and Methods

### Reagents

Plasmid construct PUC-DVS, PUC-MTE and PUC-OVS were synthesized by GenScript. The sequences are listed in the Table 1, SEQ ID NO: 1, 2, 9 or 10.

DVS and MTE mRNAs were made by Honggene Biotech (DVSAC24B1B &MTEAC24BIB). OVS was made by Jane Qin by following ARV_SOP_1. Lipids used for lipid nanoparticles fabrication were ionizable lipid Octanoic acid, 8-[(2-hydroxyethyl) [6-oxo-6-(undecyloxy) hexyl]amino]-, 1-octylnonyl ester was purchased from Broadpharm (BP-25499), helper lipid DSPC (850365C-1g), cholesterol (700100P), and DMG-PEG2000 (880151P-1g) were purchased from Avanti Polar Lipids.

### Antibodies for Western Blot

1.SARS-COV/SARS-COV-2 Spike Protein S2 Monoclonal Antibody for primary antibody was purchased from Thermofisher, (Product #MA5-35946)
2. Goat anti-Mouse IgG (H+L) secondary antibody HRP was purchased from Invitrogen, (Ref #62-6520)
3. Anti-beta-Actin HRP Antibody for protein loading control was purchased from Santa Cruz Biotechnology, (sc-47778 HRP)

Following Antibodies were used in Flow cytometry: CD45-AF750 (eBioscience, Cat #47-0451-82, clone 30-F11), CD3-AF700 (Biolegend, Cat #100216, clone 17A2), CD4-FITC (Biolegend, Cat #100406, clone GK1.5), CD8a-PerCP (Biolegend, Cat #100734, clone 53-6.7), CD44-PE/Cy7 (Biolegend, Cat #103030, clone 1M7), CD62L-BV605 (Biolegend, Cat #104438, clone MEL-14), IFNg-PB (Biolegend, Cat #505818, clone XMG1.2), TNFa-APC (eBioscience, Cat #17-7321-82, clone MP6-XT22).

MTE overlapping peptides were synthesized by GenScript as shown in Table 2.

Formulation of DVS, OVS or/and MTE with ARV L001 LNP

The formulations were prepared by mixing lipids in organic phase with an aqueous phase containing mRNA, using a Nanoassemblr Ignite microfluidic device, mRNA was dissolved in 100 mM citrate buffer (pH 4.0). Lipids were dissolved in ethanol. The molar percentage ratio for the constituent lipids is 50% SM-102 (8-| (2-hydroxyethyl) |6-oxo-6-(undecyloxy)hexyl]amino]-octanoic acid, 1-octylnonyl ester) 10% DSPC (1,2-distearoyl-sn-glycero-3-phosphocholine, 38.5% cholesterol, and 1.5% DMG-PEG (1,2-dimyristoyl-rac-glycero-3-methoxypolyethylene glycol-2000). At a flow ratio of 1:3 organic: aqueous phases, the solutions were combined in the microfluidic device (Precision NanoSystems). The total combined flow rate was 12 mL/min, per microfluidics chip. The mixed material was then dialyzed in 20 mM Tris with 8 wt % sucrose for 16 hrs. The particles were concentrated by centrifugation for 30 minutes at 2000 g using Amicon Protein Concentrator PES (30K MWCO). Particle size and zetapotential were determined by Malvern ZEN3600.

In Vitro Delivery mRNA by LNP an In Vitro Potency

To assess in vitro delivery efficiency, 293T cells are inoculated with LNP formulated indicated mRNA, the expression of Spike proteins were evaluated by Flow cytometry and Western blot.

### Immunization and Serum Collection

Six- to eight-week-old female BALB/c mice bred and maintained at animal facility in Noble Life Science. Mice were immunized with formulated mRNA by IM as scheduled in the below table.

### Study Schedule:

| | JJ/RM | | | | | | | | | |
|---|---|---|---|---|---|---|---|---|---|---|
| | 22-COV-001 validation of LNP formulation with | | | | | | | | | |
| | Luciferase mRNA in BALB/c mice (Noble Life Science) | | | | | | | | | |
| | | | mRNA | No. | | Injection | | | | |
| | Formu- | | amount | of | Dosing | amount | | Serum | Spleen | Total |
| Group | lation | mRNA | (ug/dose) | mouse | route | (ul) | Injection | collection | collection | mRNA(ug) |
| 1 | PBS | N/A | N/A | 5 | IM | 50 | D 0, D 21 | D 14, D 35 | D 35 | N/A |
| 2 | ARV-L001 | DVS | 1 | 5 | IM | 50 | D 0, D 21 | D 14, D 35 | D 35 | 50 |
| 4 | ARV-L001 | DVS/MTE | 1/1 | 5 | IM | 50 | D 0, D 21 | D 14, D 35 | D 35 | 50/50 |
| 5 | ARV-L001 | DVS/MTE | 5/5 | 5 | IM | 50 | D 0, D 21 | D 14, D 35 | D 35 | 50 |

### Evaluation of Antigen-Specific T Cell Response by ELISPOT

To assess antigen-specific T cell response in the vaccinated mice, splenocytes from vaccinated mice were evaluated for antigen-specific IFNγ by Enzyme-linked immunospot (ELISPOT). For ELISPOT assays were performed as per ARV SOP. Briefly, 96-well ELISPOT plate (Millipore, Cat #MSIPS4510) was coated with 10 μg/ml IFNγ antibody (Biolegend, Cat #517902, clone AN18) at 4° C. overnight. Splenocytes were plated at 3×105 cells/well and co-cultured with 0.5 μg/ml spike peptides (JPT, Cat #PM-WCPV-S-1) or 2 μg/ml human MTE overlapping peptides (synthesized by Genscript) or concavalin A (0.125 μg/ml) (Sigma, Cat #C5275-5 MG) or medium alone in a total volume of 200 μl/well T cell media for 48 h at 37° C. in 5% CO₂. The plates were incubated with detection antibodies Biotin-IFNγ (Biolegend, Cat #505714, clone R46A2) and Streptavidin-HRP (Biolegend, Cat #405210) at RT for 1-2 hours respectively. The plates were developed with 50 μl/well AEC development solution (BDbiosciences, Cat #551015) for up to 30 min. Color development was stopped by washing under running tap water. After air dried, colored spots were counted using an AID ELISPOT High-Resolution Reader System and AID ELISPOT Software version 3.5 (Autoimmun Diagnostika GmbH).

### ELISA

The murine antibody response to spike was assessed by indirect ELISA. ELISA plates (Nunc MaxiSorp, Thermofisher, Cat #44-2404-21) were coated with 1 μg/ml recombinant spike protein (Sino Biological Inc, Cat #40591-V08H) overnight and then blocked with 2% BSA in PBS. Serum samples were diluted from 100×, then do 1:5 serial dilution up to 8 wells with 0.2% BSA in PBS. Samples were detected with 1:2000 goat anti-mouse IgG-HRP (Southern Biotech, Cat #1031-05). Reaction was developed with TMB Substrate (Sigma, Cat #T0440-1001) and stopped with TMB Stop Solution (Invitrogen, Cat #SS04). Plates were read (OD450) by using Epoch ELISA reader (BioTek, Winooski, VT).

### Lentivirus Based Pseudovirus Neutralization Assay

The SARS-COV-2 pseudoviruses expressing a luciferase reporter gene were purchased from Jimmy Lab. To determine the neutralization activity of the antisera from vaccinated mice, HEK293T-hACE2 cells were seeded in 384-well tissue culture plates at a density of 7.5×103 cells per well overnight. Two-fold serial dilutions of heat-inactivated serum samples were prepared and mixed 17.5 μl with 7.5 μl of pseudovirus. The mixture was incubated at 37° C. for 1 h before adding to HEK293T-hACE2 cells. After 48 h, cells were lysed in Steady-Glo Luciferase Assay (Promega) according to the manufacturer's instructions. SARS-COV-2 neutralization titres were defined as the sample dilution at which a 50% reduction in relative light units (IC50) was observed relative to the average of the virus control wells.

### Intracellular Cytokine Staining

Single cell suspensions were made from spleens. RBC (red blood cells) were lyzed with 1.5 ACK lysis buffer (Lonza, BP10-548E) for 5 minutes and resuspended in RPMI 1640 complete medium. Splenocytes were filtered by 70 μm cell strainers. 200 ul of IM splenocytes were seed into round-bottom 96-well plate (Corning, Cat #3788) and stimulated with 0.5 μg/ml spike peptides (JPT, Cat #PM-WCPV-S-1) or MTE peptides (synthesized by Genscript) or cell activation cocktail (Biolegend, Cat #423302) or medium alone at 37° C. for 1h. Prepare 50 ul BD golgiplug (BD biosciences, Cat #555029) and 35 ul BD golgistop (BD biosciences, Cat #554724) in 1 ml cell culture medium and add 20 ul/well in 96-well plate. Cells were incubated at 37° C. for 4-5 h and then staining with Zombie Aqua Fixable viability dye in PBS for 20 minutes at RT. Cells were then staining with Fc block, CD45-AF750, CD3-AF700, CD4-FITC. CD8a-PercpCy5.5, CD44-PECy7, CD62L-BV605 in FC buffer (PBS supplemented with 2% heated inactivated FBS) at 4° C. for 30 minutes. Cells were fixed and permeabilized with fixation/permeabilization kit (eBioscience, Cat #00-5521-00) according to the manufacturer's instructions. Intracellular staining was performed with cocktail in 1X Permeabilization Buffer containing IFN-γ-PB and TNFa-APC at 4° C. for 1h. Cells were washed twice with Permeabilization Buffer and resuspend in FC buffer.

### Statistics

Statistical analysis was performed using Prism software. One-way ANOVA was used to compare each group.

### Results

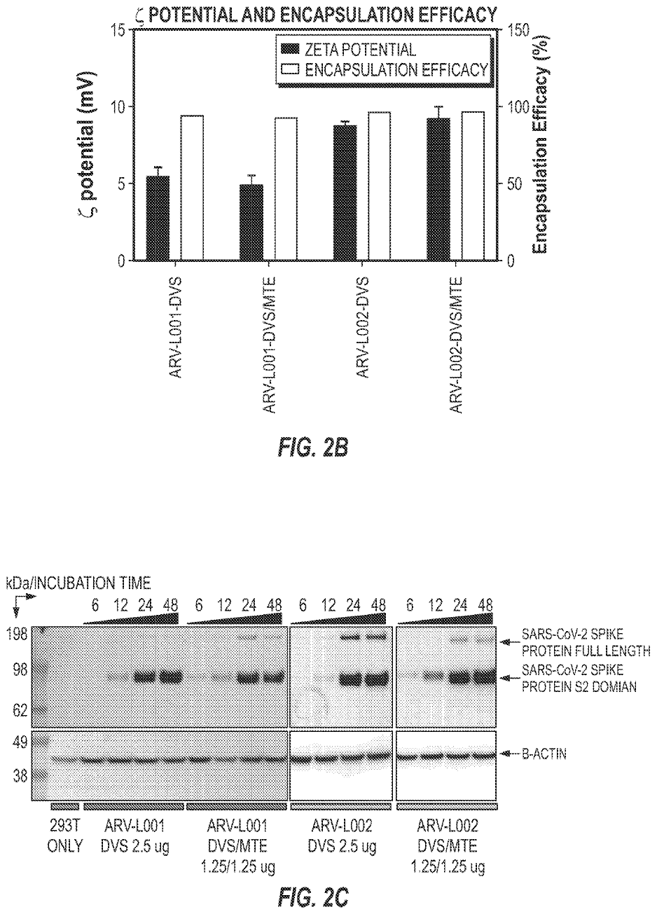

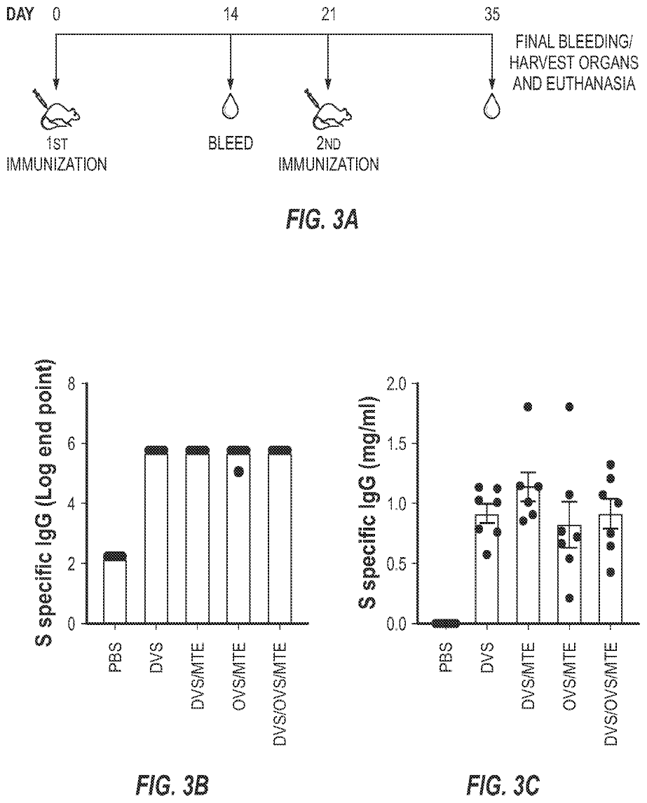

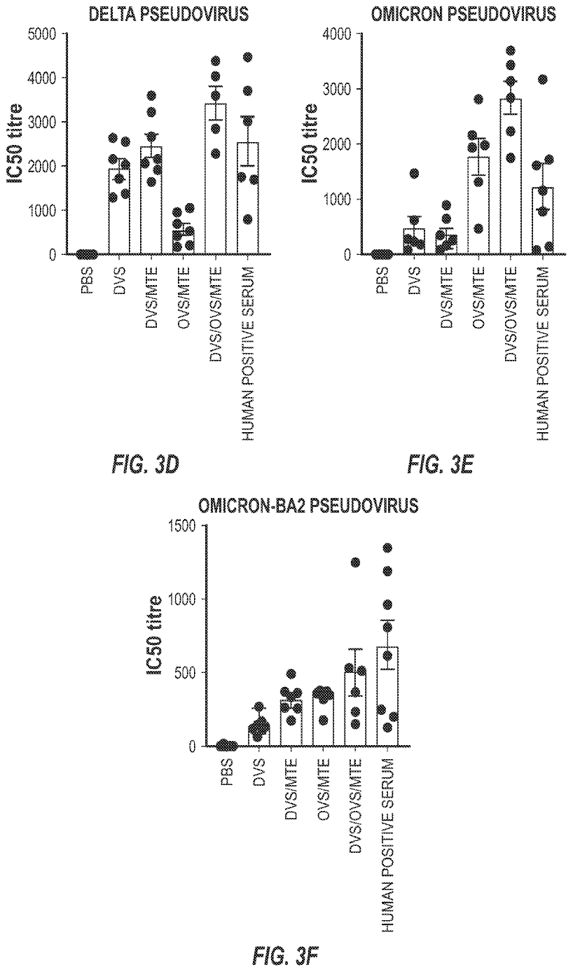

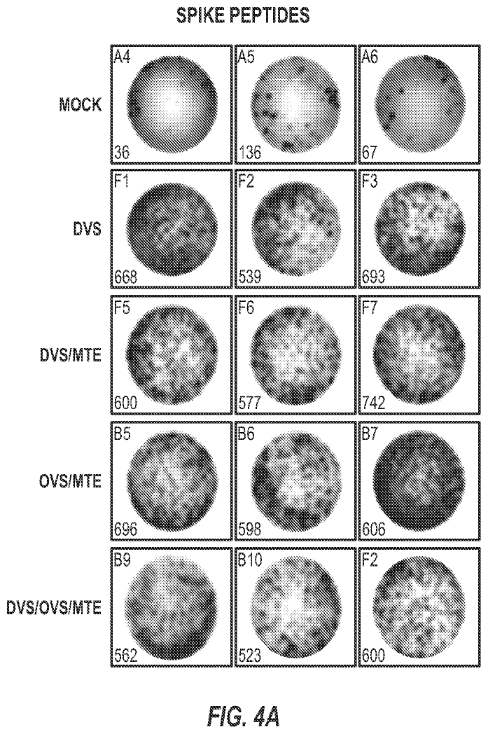

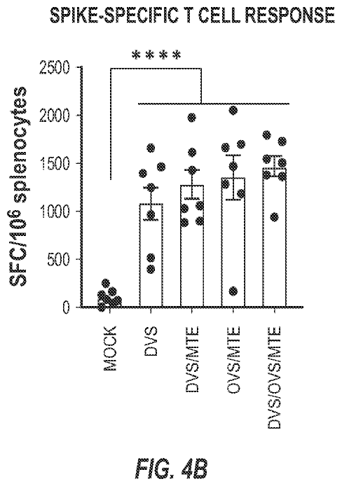

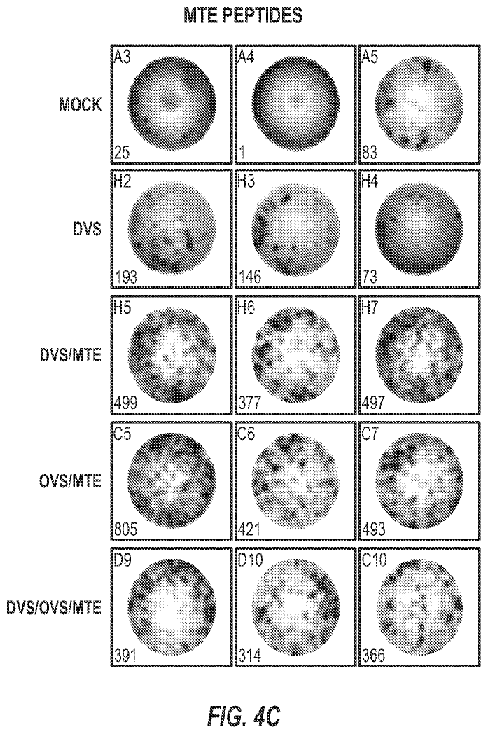

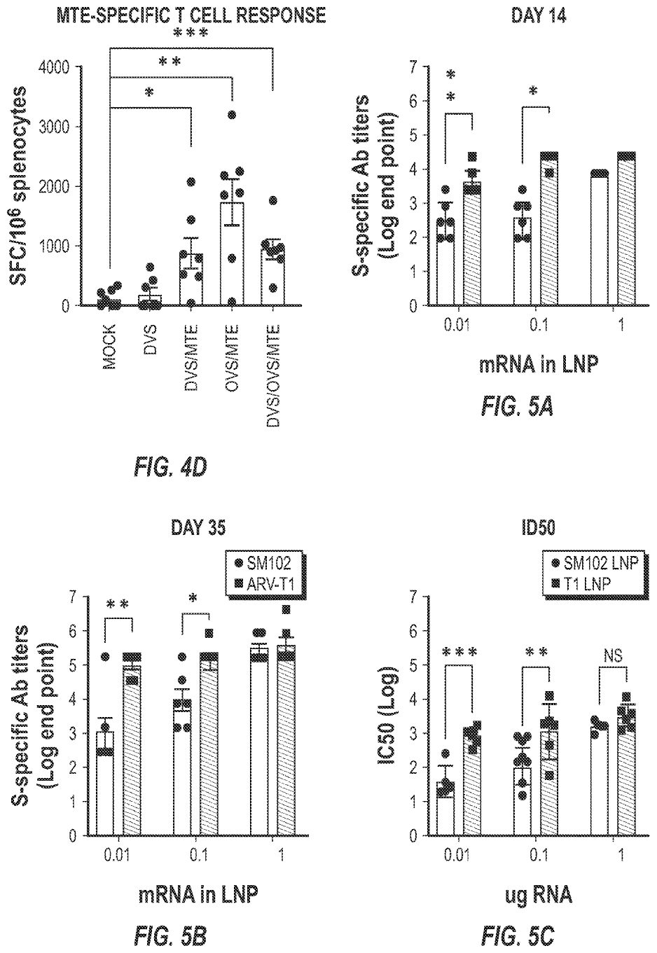

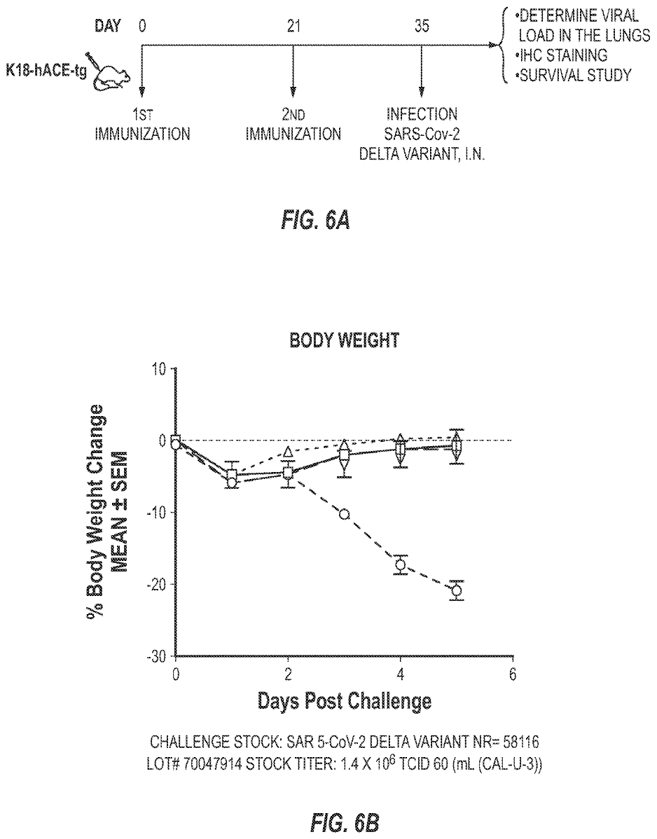

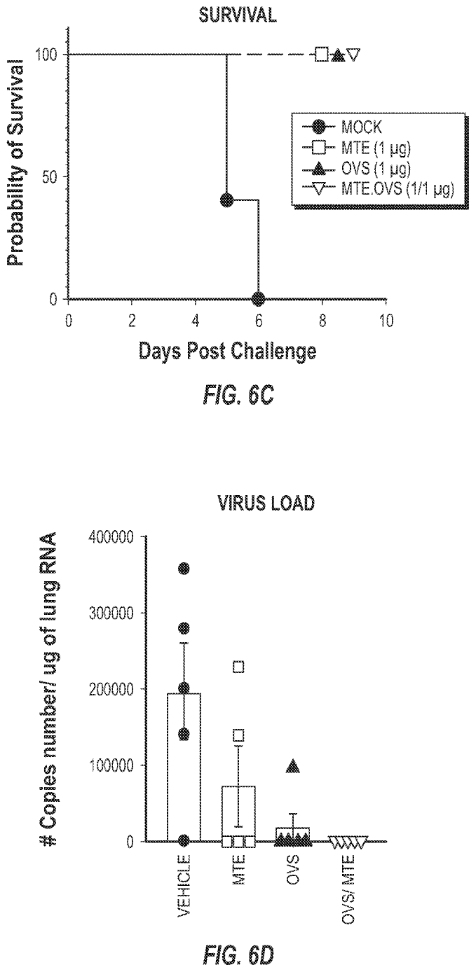

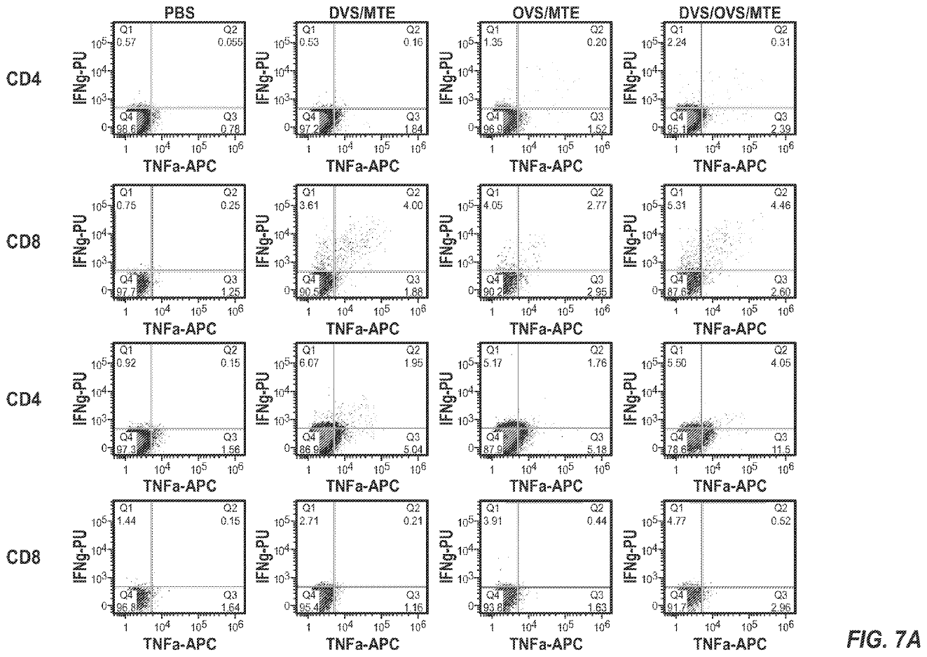

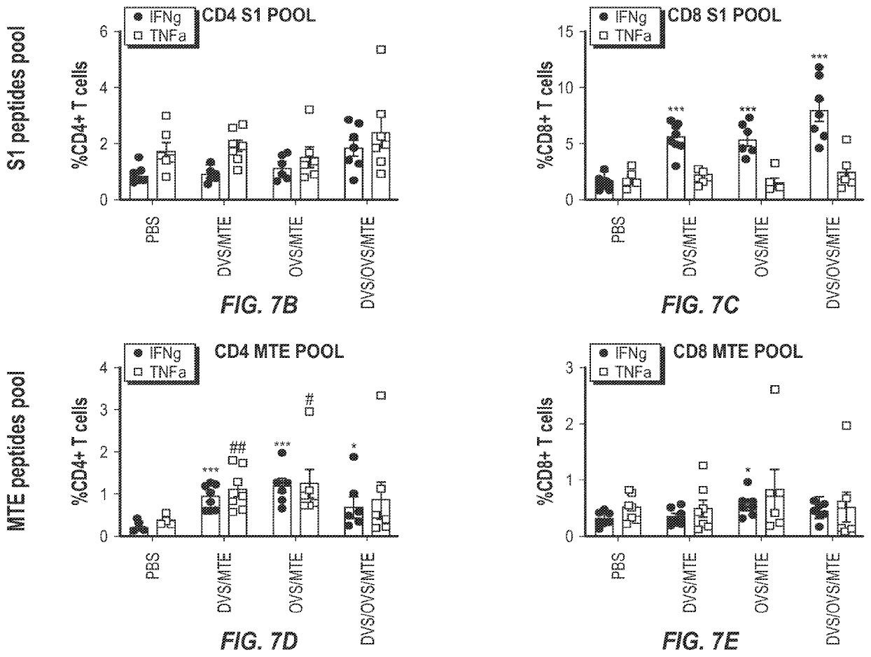

Results for in vitro transcription of mRNA and in vitro validation of the protein expression are shown in FIGS. 1A-1E. The preparation of LNPs and evaluation of delivery efficiency in vitro is shown in FIGS. 2A-2B. The COVID vaccine described herein elicits robust spike-specific antibody responses in vivo results are shown in FIG. 3A-3D. The vaccine described herein elicits robust spike-, and MTE-specific T cell responses results are shown in FIGS. 4A-4D. Results for dosing studies are shown in FIGS. 5A-5C. The vaccine described elicits robust spike-, and MTE-specific T cell responses as shown in FIGS. 6A-6D.

### CONCLUSION

The vaccine described herein formulated with DVS and MTE, elicits robust spike specific binding and neutralizing antibody responses in vivo. The vaccine described herein, DVS/MTE, elicits robust spike- and MTE-specific-T cell response, including CD4 and CD8 T cell responses.

### Example 2: HSV-1/-2 mRNA Vaccine Formulated with Lipid Nanoparticles

The sequences of indicated mRNAs used in HSV-2 vaccine are listed in the Table 1 and the MTE amino acid sequence are listed in the Table 2.

Synthetizations of HSV mRNA vaccine in vitro. HSV gB, gD, gH, gL, UL19, and MTE mRNAs were synthesized with T7 RNA polymerase in vitro transcription and run on 0.8% MOPS agarose gel see FIG. 11.

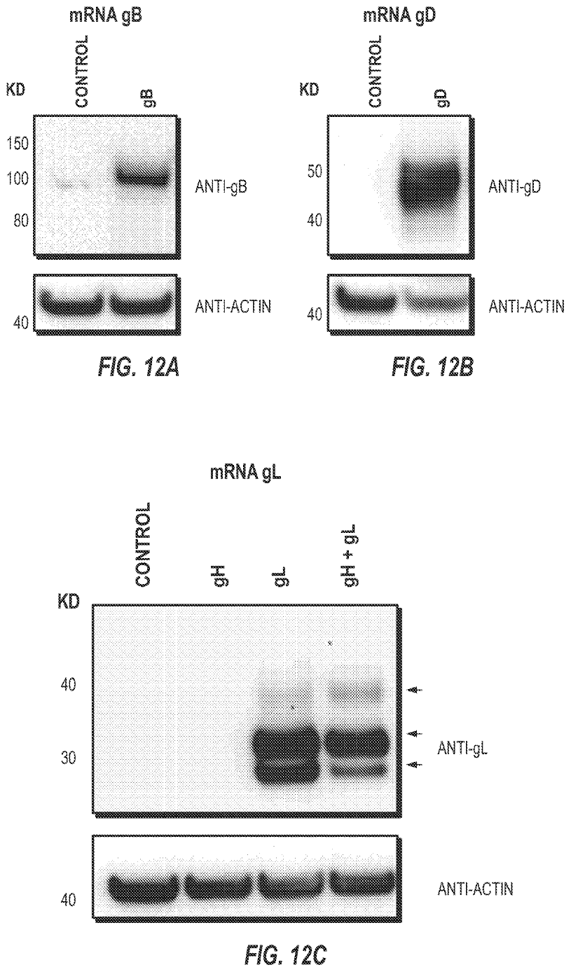

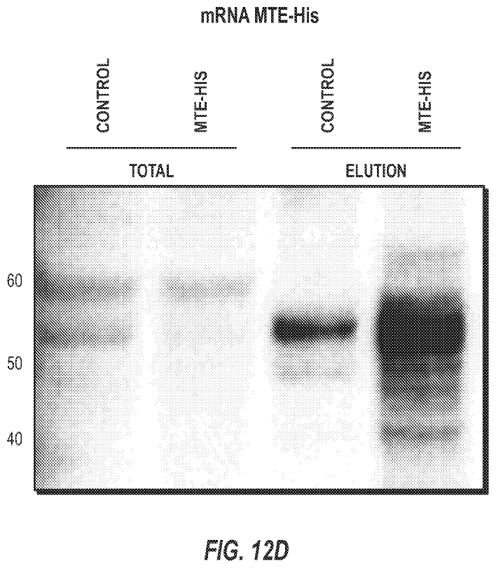

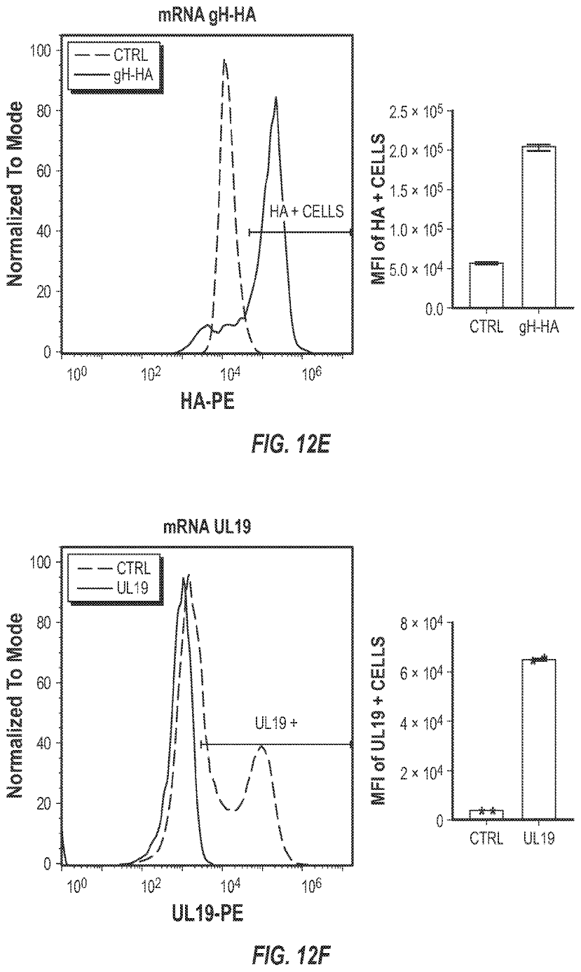

Validation of HSV mRNA vaccine in vitro was performed. HSV mRNA gB (FIG. 12A), gD (FIG. 12B) and gL (FIG. 12C) were transfected into 293T cells with mRNA transfection kit and detected by western blot. HSV mRNA MTE-His was transfected into 293T cells with mRNA transfection kit as shown in FIG. 12D. After 48h, MTE-His protein was concentrated from cell lysis and detected by western blot. HSV mRNA gH-HA (FIG. 12E) and UL-19 (FIG. 12F) were transfected into 293T cells with mRNA transfection kit and detected by flow cytometry.

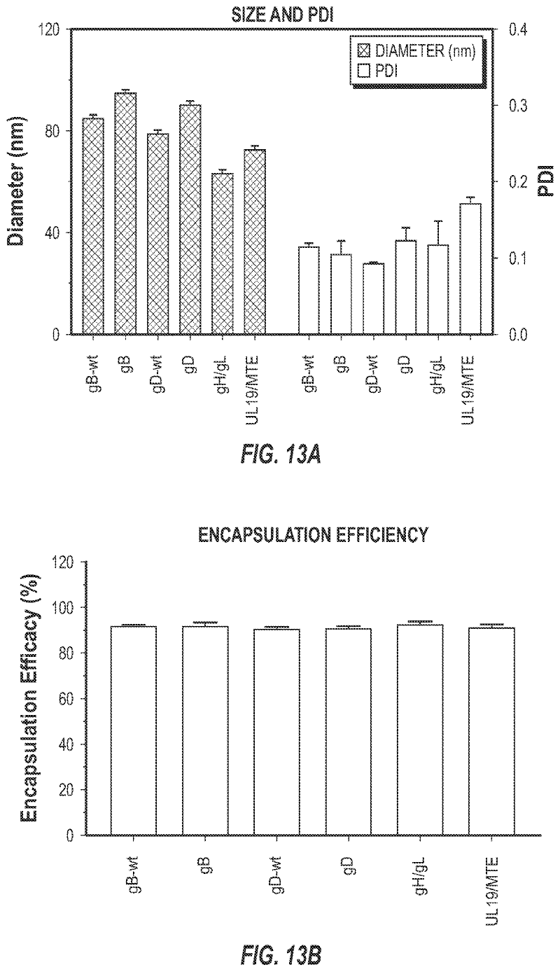

Physiochemical characterization of lipid nanoparticle formulation. Size and polydispersity index (PDI) of LNP formulations with indicated mRNA using ARV-L002 (ARV-T1) are shown in FIG. 13A. Encapsulation efficiency of mRNA vaccine of ARV-L002 LNP formulation with indicated mRNA are shown in FIG. 13B.

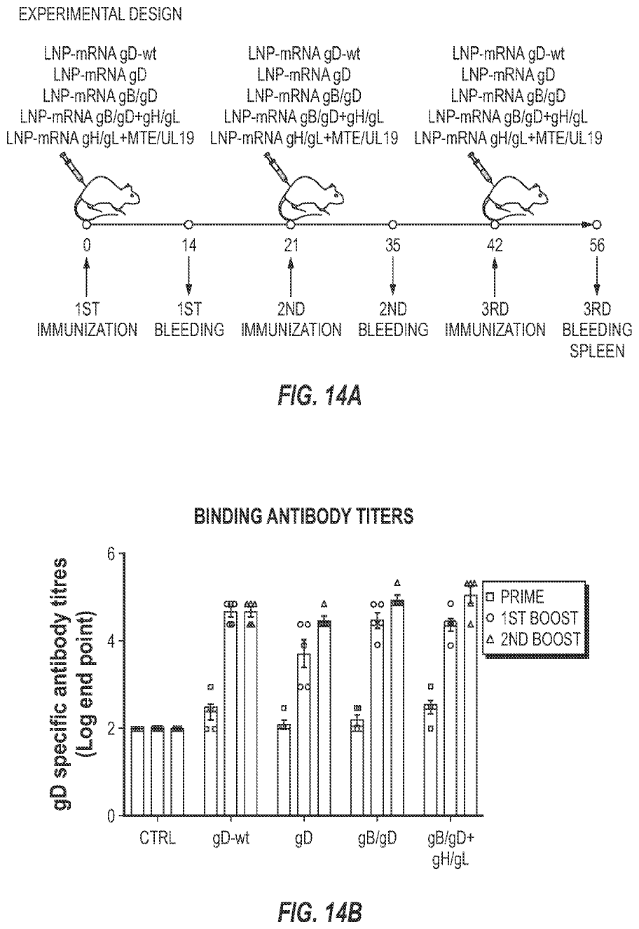

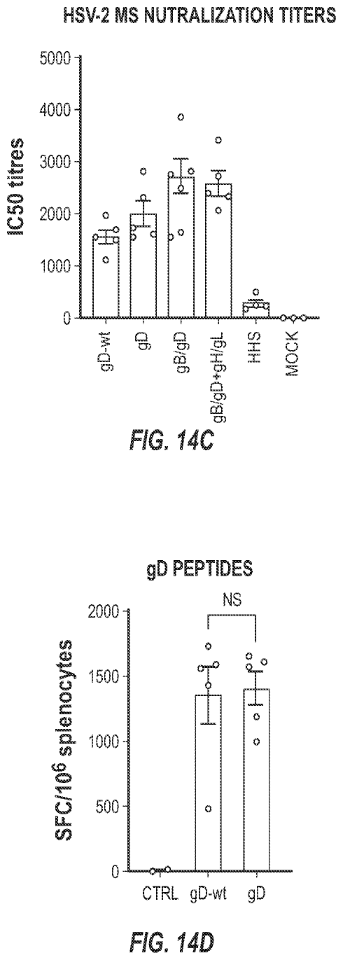

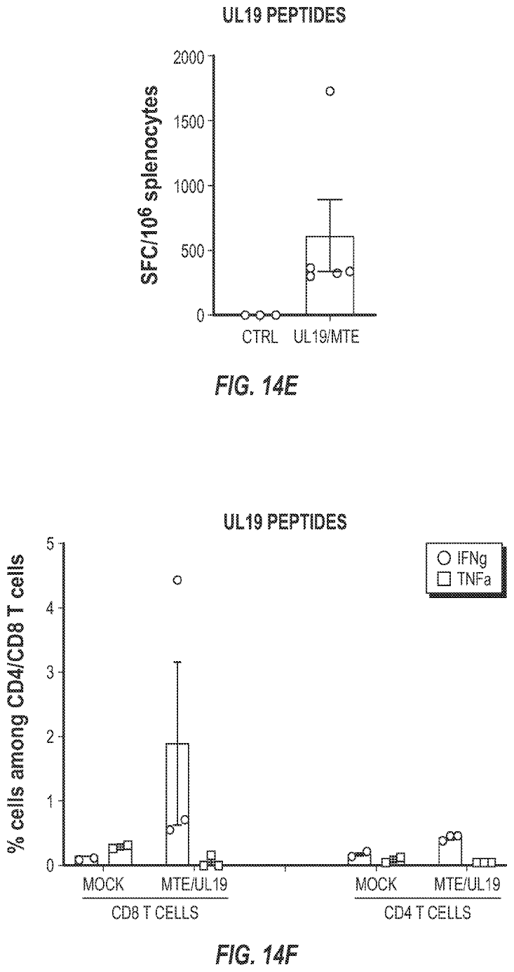

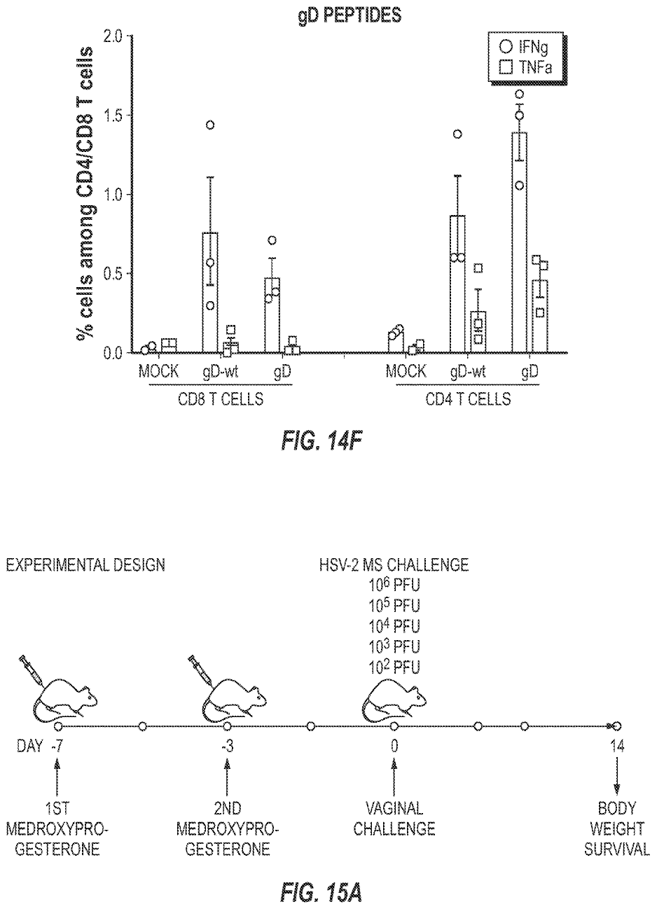

HSV mRNA vaccine elicits robust T cell responses and strong antibody responses in BALB/c mice. BALB/c mice were intramuscularly immunized on DO. D21, and D42 with 5 μg of LNP formulated mRNA vaccines (gD-wt, gD, gB/gD, gB/gD+gH/gL and gH/gL+MTE/UL19) as illustrated in FIG. 14A. Mouse serum was collected on D14, D35, and D56, respectively. Mouse spleens were collected on D56. gD specific IgG were detected by ELISA from serum samples results shown in FIG. 14B. Neutralization assays were performed with HSV-2 MS strain in Vero cells by counting plaque results are shown in FIG. 14C. Splenocytes were isolated from mouse spleen, ELISPOT assay was performed results are shown in FIGS. 14D-14E, flow cytometry intracellular staining with stimulation of a UL-19 peptides pool (FIGS. 14E-14F) or gD peptides pool (FIGS. 14D and 14G).

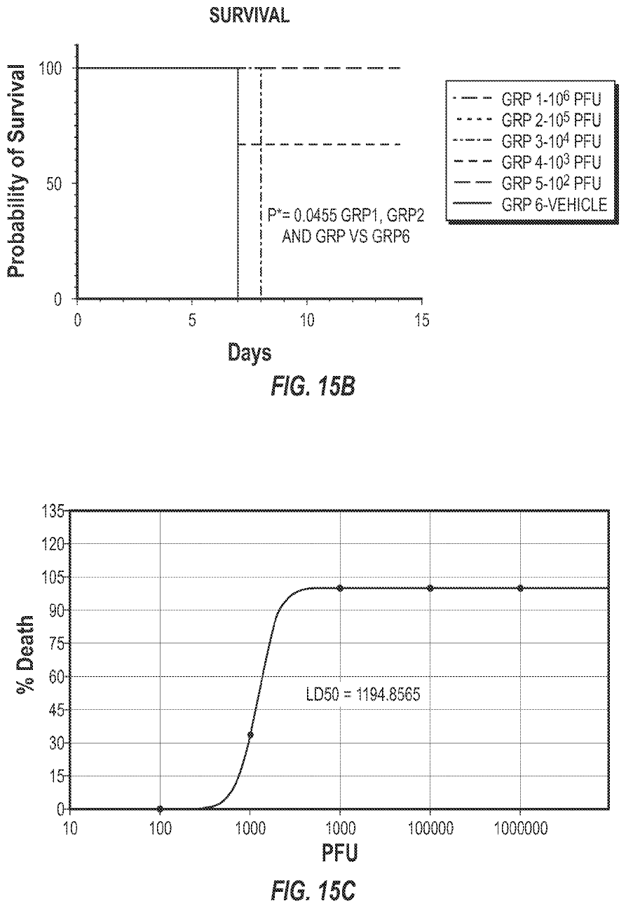

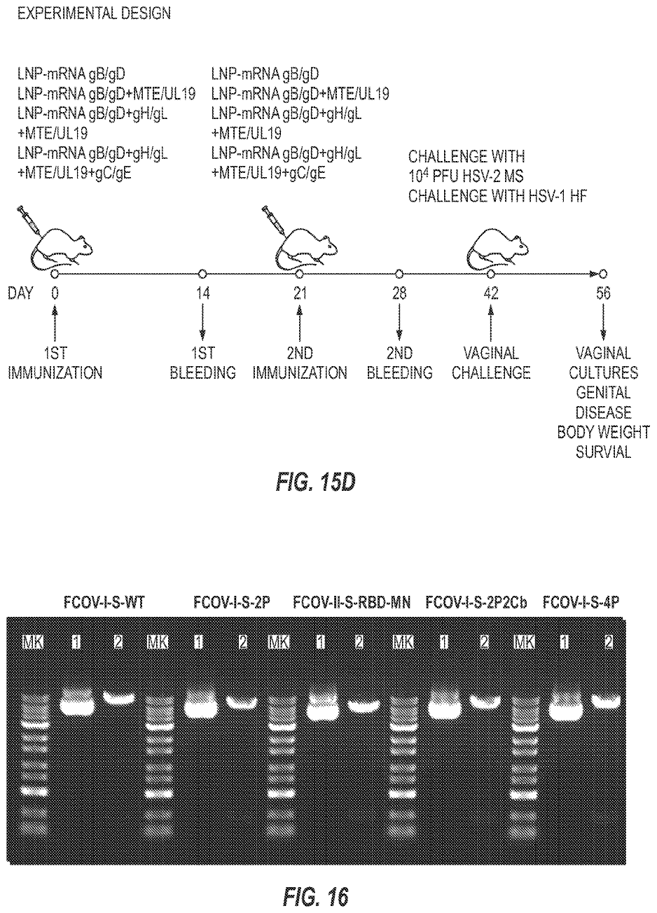

HSV mRNA vaccines protect mice from HSV-2 challenge. 50% lethal dose (LD50) of HSV2 MS in BALB/c. Female BALB/c mice were injected with 2 mg medroxyprogesterone on D-7 and -3 and challenged intravaginally with different PFU of HSV-2 as illustrated in FIG. 15A. Mouse body weight and survival were recorded after the challenge. Mouse survival curve after challenge is shown in FIG. 15B. The value of LD50 of HSV-2 MS strain in BALB/c mice is shown in FIG. 15C. Female BALB/c mice were intramuscularly immunized on DO and D21 with 5 μg of LNP formulated mRNA vaccines (gB/gD, gB/gD+MTE/UL19, gB/gD+gH/gL+MTE/UL19, and gB/gD+gH/gL+gC/gE+MTE/UL19) as illustrated in FIG. 15D. Mouse serum was collected on D14 and D28. Mice were injected with 2 mg medroxyprogesterone on D35 and 39 and challenged intravaginally with 10+PFU HSV2 MS strain or HSV-1 HF on D42. Vaginal cultures were collected on D44 and 46 to determine the copy number of HSV2 or HSV-1. Mice genital disease, body weight and survival were recorded until D56.

### Example 3: Monkeypox (MPX) mRNA Vaccine with LNP

The sequences of four mRNAs used in MPX vaccine are listed in the Table 1 (SEQ ID No. 347-350).

### Example 4: Lipid Nanoparticles for Nucleic Acid Delivery

Lipid nanoparticle (LNP) plays a key role in effectively protecting and delivering nucleic acid to cells for the application of prevention and therapeutics. Despite promising data from ongoing clinical trials, the clinical use of gene medicine requires the discovery and development of more efficient delivery systems. Described herein are nanoparticles for gene and drug delivery applications.

SARS-COV-2 mRNA Lipid Nanoparticles (LNPs).

Lipid nanoparticle (LNP) formulations were prepared herein using either Target 1 lipid (ionizable lipid) (ARV-L002) (ARV-T1) or commercially available SM-102 lipid (ARV-L001) for comparison. LNP formulations were prepared using lipids dissolved in ethanol at molar ratios of 50:10:38.5:1.5 (ionizable lipid: DSPC: cholesterol: PEG-lipid). The lipid mixture was combined with 100 mM sodium citrate buffer (pH 4.0) containing mRNA at a volume ratio of 3:1 (aqueous: ethanol) using a NanoAssemblr Ignite. Formulations were dialyzed against 10 mM Tris (pH 7.4) with 8% sucrose in Slide-A-Lyzer dialysis cassettes for at least 16 h and concentrated using Amicon ultra-centrifugal filters and then passed through a 0.22 μm filter and stored at 4° C. or −20° C. until use. The size, PDI, and charge were measured by dynamic light scattering while mRNA encapsulation efficiency was measured by Ribogreen assay spectrometrically using λₑₓ=480 nm and λₑₘ=535 nm.

FIG. 2A shows the particle diameter and polydispersity index (PDI) of the LNPs. FIG. 2B shows the surface charge (zeta potential) and mRNA encapsulation efficiency of ARV-L001 and ARV-L002 LNPs.

### In Vitro Studies

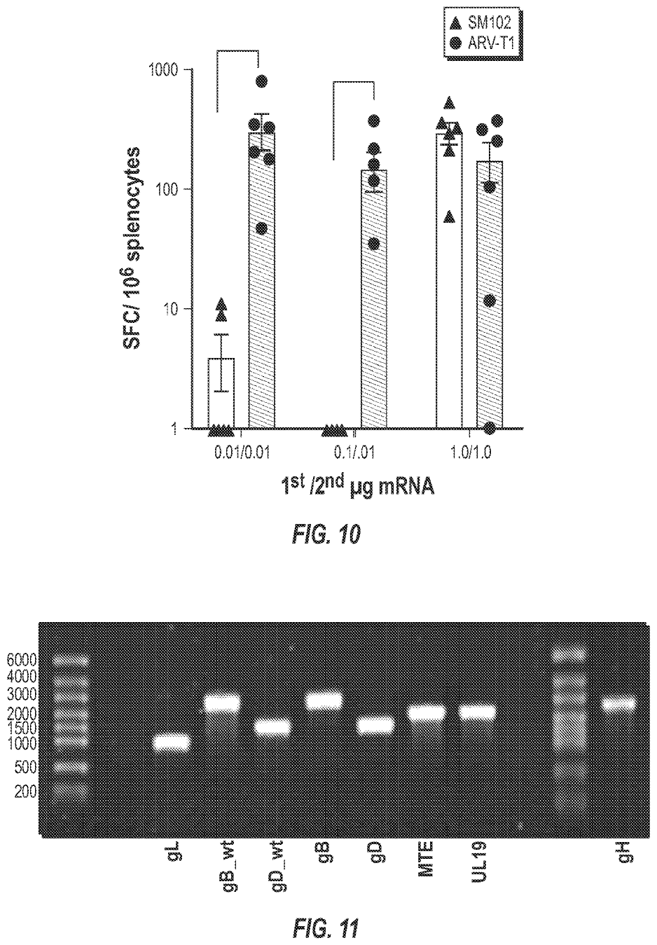

293 T cells were transfected with an mRNA encoding a SARS-COV-2 spike protein. FIG. 10 shows the in vitro expression of the spike protein after transfecting cells with 1.0 μg/mL mRNA over time.

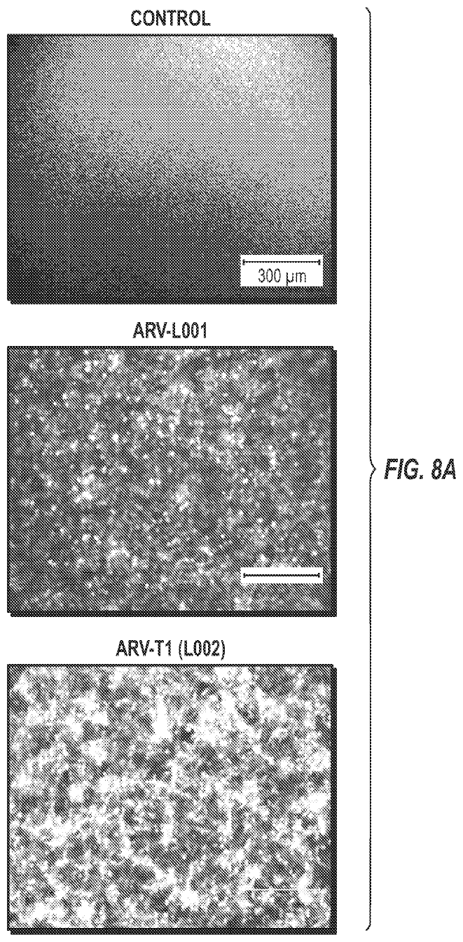

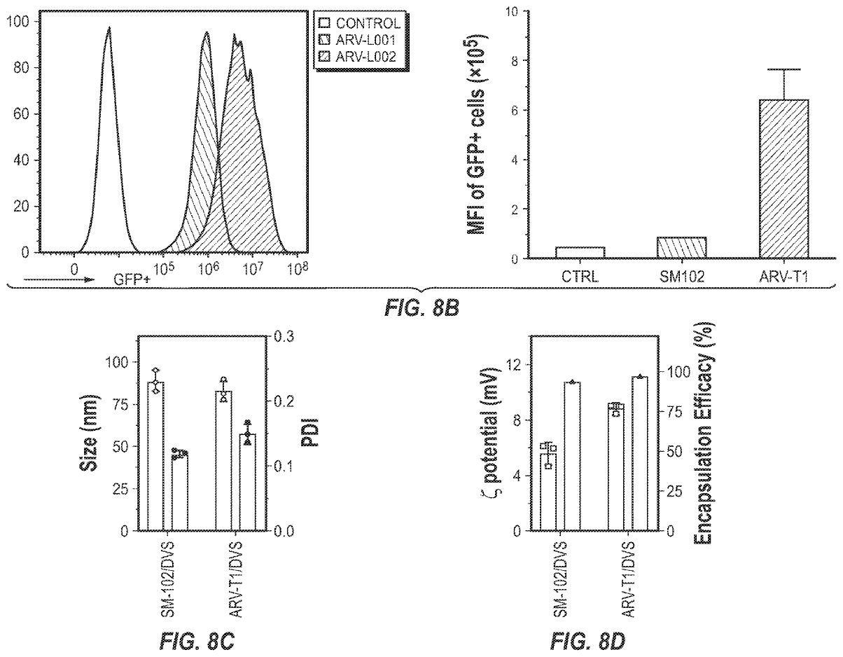

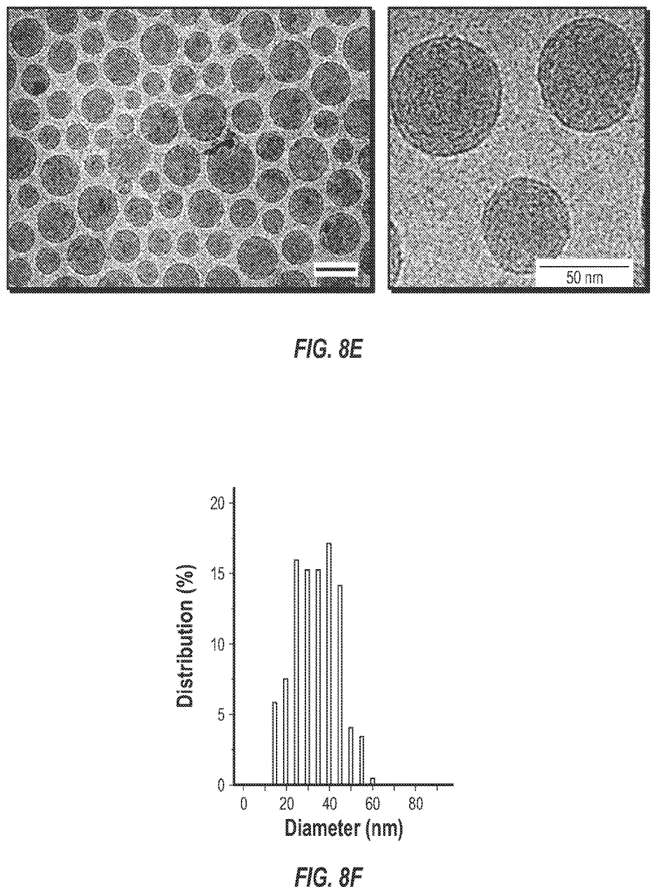

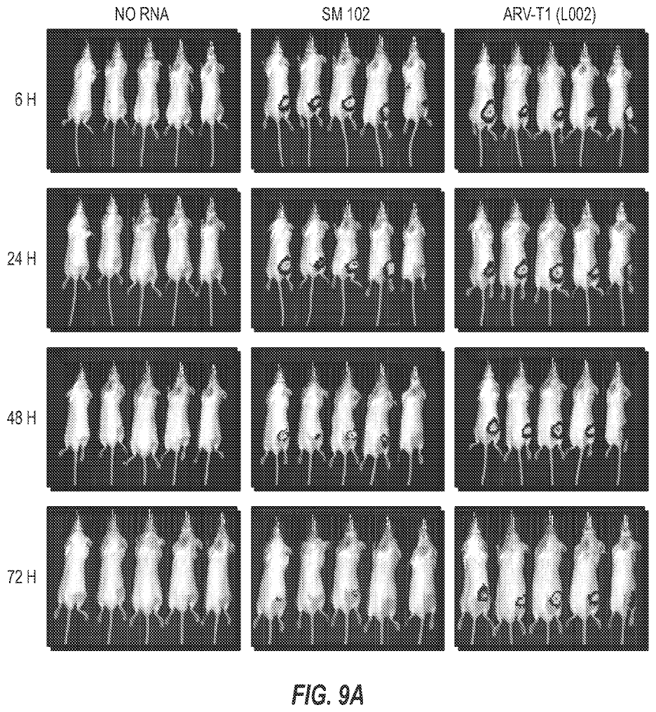

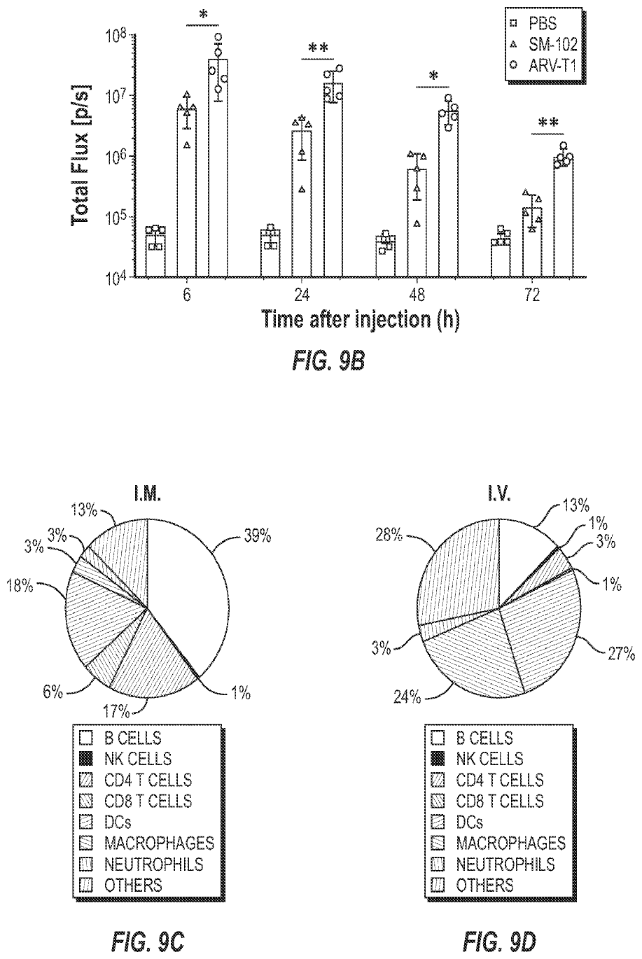

The transfection efficiency was studied by delivering GFP mRNA (1 μg/mL) using LNP deliver into BHK cells. The transfection efficiency was determined after 24 hours by imaging the BHK cells after transfection and analyzing the GFP expression using flow cytometry. The results of the study are shown in FIGS. 8A and 8B.

### In Vivo Studies

LNPs formulated with indicated ionizable lipids and 1 μg of Luciferase-expressing mRNA were injected intramuscularly. After administration, the luciferase expression was determined by whole body bioluminescence imaging using an IVIS Spectrum in vivo imaging system at 6, 24, 48, and 72 hours, respectively. Results of the in vivo transfection efficiency study of luciferase-expressing mRNA is shown in FIG. 9.

1 μg of a vaccine formulated with Target 1 lipid and mRNA encoding full-length spike glycoprotein of SARS-COV-2 (Delta variant) was injected intramuscularly as scheduled. A formulation comprising commercially available lipid SM-102 and mRNA encoding full-length spike glycoprotein of SARS-COV-2 (Delta variant) was prepared and used as comparison. Total Spike-specific total IgG were evaluated on day 14 and 35 after the first immunization results are shown in FIGS. 5A and 5B. Neutralizing antibodies in the serum are evaluated by pseudotyped viruses results are shown in FIG. 5D. Antigen-specific T cell responses were evaluated by Elispot results are shown in FIG. 10. Data was presented as Mean±SD. Statistical comparisons were analyzed using by one-way ANOVA with Tukey's multiple comparison test. *p<0.05, **p<0.01. The results demonstrate efficient elicited immunity in vivo.

### Example 5. FIPV Vaccine Formulated with Lipid Nanoparticles

Synthetization and formulation of FIPV mRNA vaccine. Linearization of DNA plasmids with BspQI before mRNA in vitro transcription results are shown in FIG. 16. 1,2 indicated before and after digestion respectively. FIPV mRNAs ((Fcov-I-S-wt, Fcov-I-S-2P, Fcov-I-S-2P2Cb, Fcov-I-S-4P and Fcov-II-RBD-MN)) were synthesized and formulated into LNP-mRNA and certified by Novoprotein.

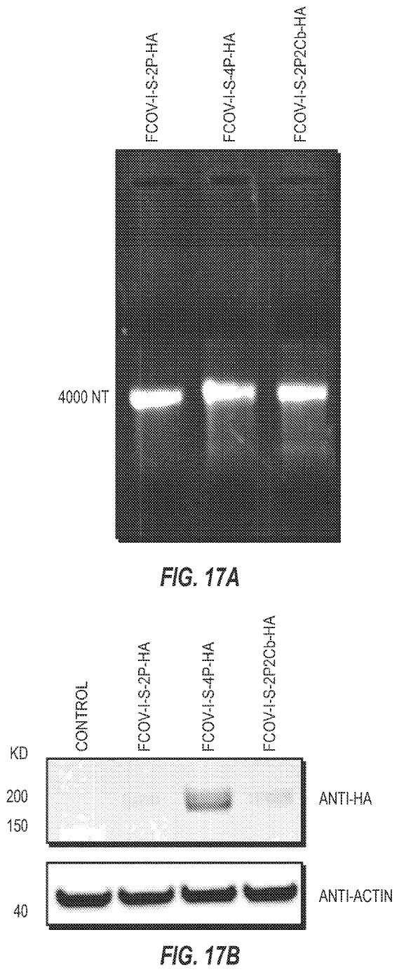

Validation of FIPV mRNA vaccine in vitro. FIPV Spike mRNAs (Fcov-I-S-2P-HA, Fcov-I-S-4P-HA and Fcov-I-S-2P2Cb-HA) were synthesized with T7 RNA polymerase in vitro transcription and run on 0.8% MOPS agarose gel results are shown in FIG. 17A. FIPV Spike mRNAs were transfected into 293T cells with mRNA transfection kit and detected by western blot results shown in FIG. 17B.

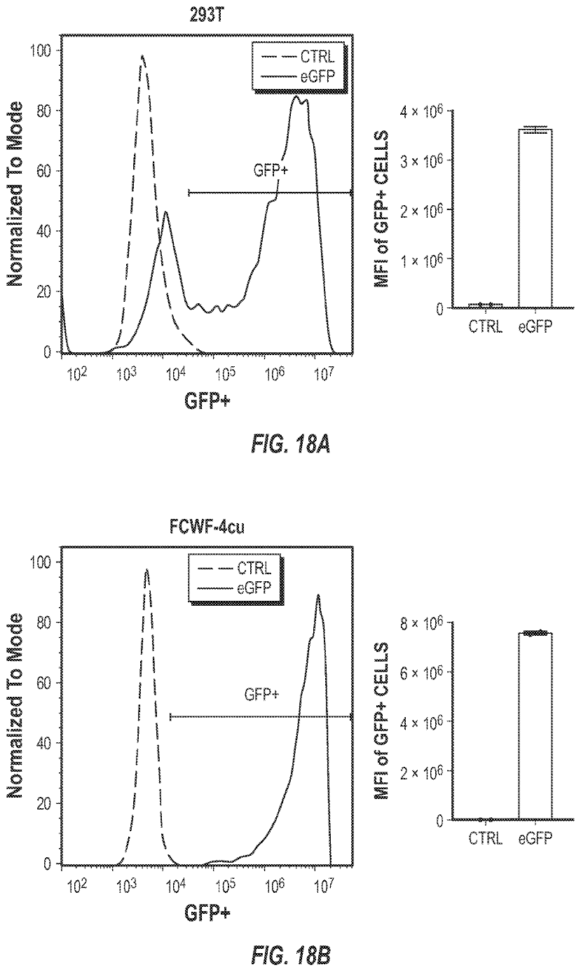

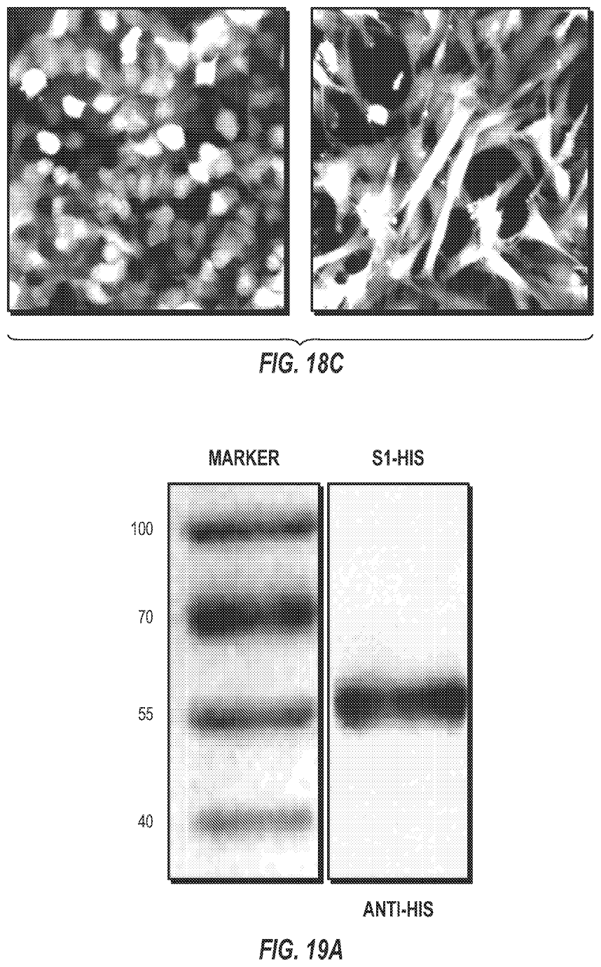

mRNA with human derived UTR can be efficiently expressed in feline cells. eGFP mRNAs with human derived UTR were transfected into feline cell line FCWF-4cu and human cell line 293T cells with mRNA transfection kit. The expression level of eGFP were detected by Flow cytometry (FIG. 18A-18B) and fluorescence microscope (FIG. 18C).

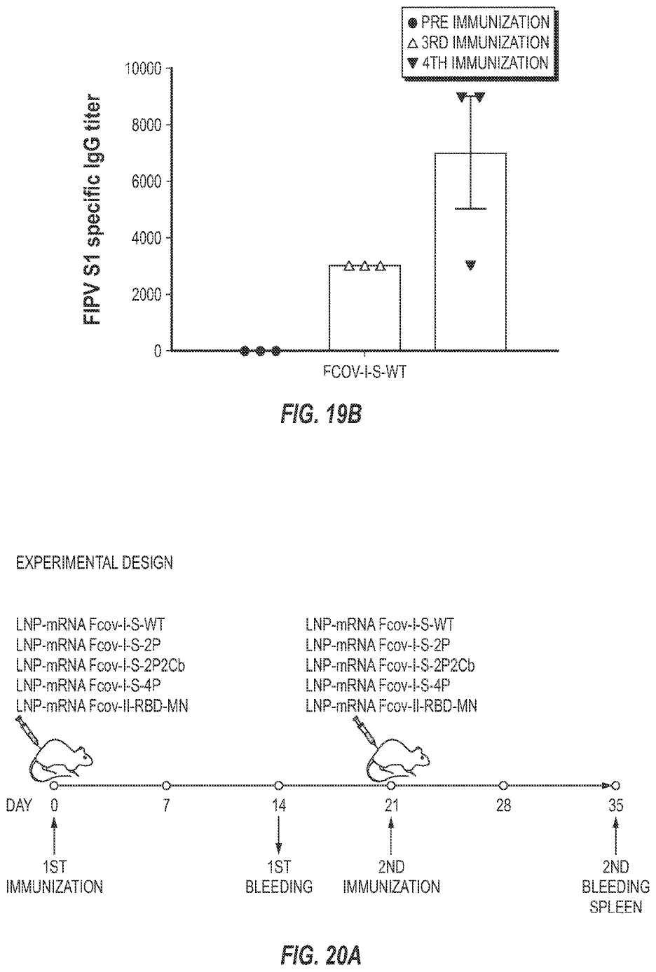

Production of Fcov-1 S1 antigen and antibody. Recombinant Fcov-I S1 subunit His tag protein was produced in vitro and detected with His antibody results shown in FIG. 19A. Rabbits were immunized in 4 doses with recombinant Fcov-I S1-His protein see results in FIG. 19B. Serum was collected 3 weeks after immunizations and performed ELISA assay for s1 antibody titers with recombinant Fcov-I S1-His protein.

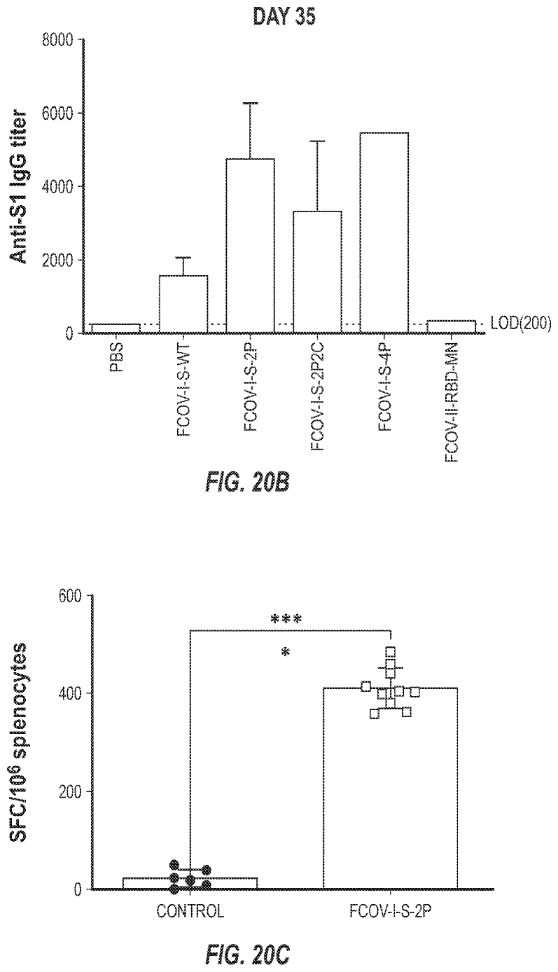

FIPV mRNA vaccine elicits robust T cell responses and strong antibody responses in BALB/c mice. BALB/c mice were intramuscularly immunized on DO and D21 with 2 μg of LNP formulated mRNA vaccines (Fcov-I-S-wt, Fcov-I-S-2P, Fcov-I-S-2P2Cb, Fcov-I-S-4P and Fcov-II-RBD-MN) as illustrated in FIG. 20A. Mouse serum was collected on D14 and D35. Mouse spleens were collected on D35. S1 specific IgG was detected by ELISA from serum samples results shown in FIG. 20B. Splenocytes were isolated from mouse spleen and ELISPOT assay was performed with stimulation of a S1 protein as shown in FIG. 20C or N peptides pool as shown in FIG. 20D.

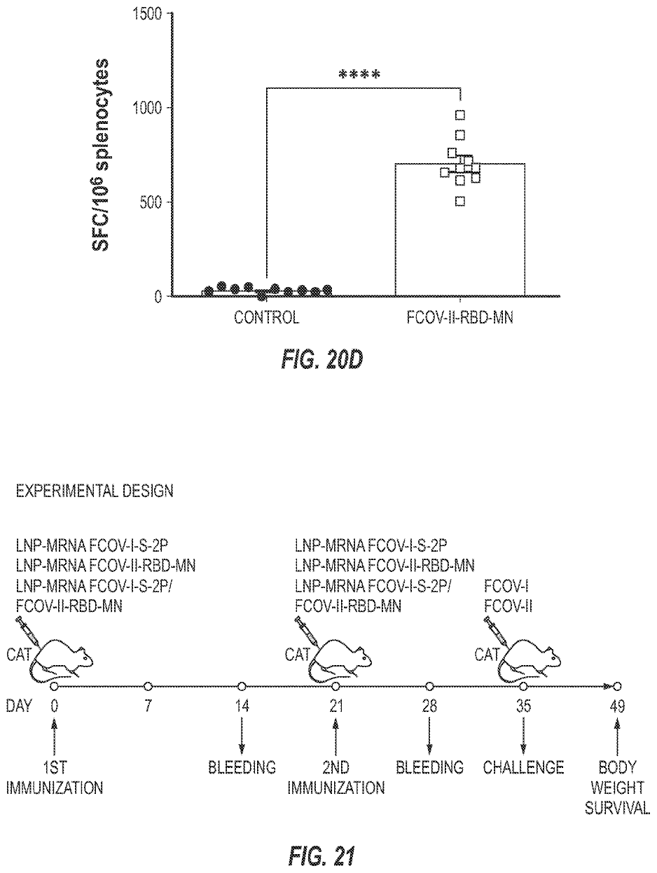

FIPV mRNA vaccine protect cat from Fcov-I and Fcov-II challenge. Cats were intramuscularly immunized on DO and D21 with 10 ug of LNP formulated mRNA vaccines (Fcov-I-S-2P, Fcov-II-RBD-MN and Fcov-I-S-2P/Fcov-II-RBD-MN) as illustrated in FIG. 21. Cat serum was collected on D14 and D28. Cats were challenged with Fcov-I and Fcov-II virus on D35. Cats body weight and survival were recorded until D49.

### Sequences

This application is accompanied by a copy of a sequence listing in electronic form (ST.26 text file) having a title size on disk 5.07 MB, which is incorporated by reference in its entirety.

TABLE 1
Sequences of the constructs
Table 1. DNA and Protein Sequences in Constructs

| Protein | SEQ ID NO: | Sequence |
|---|---|---|
| SARS-COV-2 | 321 | MFVFLVLLPLVSSQCVNLRTRTQLPPAYTNSFTRGVYYPDKVFRSSVLHSTQ |
| B.1.617.2 SPIKE | | DLFLPFFSNVTWFHAIHVSGTNGTKRFDNPVLPFNDGVYFASTEKSNIIRGW |
| (Delta Variant (With | | IFGTTLDSKTQSLLIVNNATNVVIKVCEFQFCNDPFLDVYYHKNNKSWMESG |
| two proline | | VYSSANNCTFEYVSQPFLMDLEGKQGNFKNLREFVFKNIDGYFKIYSKHTPI |
| substitutions); Protein | | NLVRDLPQGFSALEPLVDLPIGINITRFQTLLALHRSYLTPGDSSSGWTAGA |
| Sequence | | AAYYVGYLQPRTFLLKYNENGTITDAVDCALDPLSETKCTLKSFTVEKGIYQ |
| | | TSNFRVQPTESIVRFPNITNLCPFGEVFNATRFASVYAWNRKRISNCVADYS |
| | | VLYNSASFSTFKCYGVSPTKLNDLCFTNVYADSFVIRGDEVRQIAPGQTGKI |
| | | ADYNYKLPDDFTGCVIAWNSNNLDSKVGGNYNYRYRLFRKSNLKPFERDIST |
| | | FTYQAGSKPCNGVEGENCYFPLQSYGFQPTNGVGYQPYRVVVISFETTHAPA |
| | | TVCGPKKSTNLVKNKCVNENENGLIGTGVLIESNKKFLPFQQFGRDIADTTD |
| | | AVRDPQTLEILDITPCSFGGVSVITPGTNTSNQVAVLYQGVNCTEVPVAIHA |
| | | DQLTPTWRVYSTGSNVFQTRAGCLIGAEHVNNSYECDIPIGAGICASYQTQT |
| | | NSRRRARSVASQSIIAYTMSLGAENSVAYSNNSIAIPTNFTISVTTEILPVS |
| | | MTKTSVDCTMYICGDSTECSNLLLQYGSFCTQLNRALTGIAVEQDKNTQEVF |
| | | AQVKQIYKTPPIKDFGGFNFSQILPDPSKPSKRSFIEDLLFNKVTLADAGFI |
| | | KQYGDCLGDIAARDLICAQKFNGLTVLPPLLTDEMIAQYTSALLAGTITSGW |
| | | TFGAGAALQIPFAMQMAYRFNGIGVTQNVLYENQKLIANQFNSAIGKIQDSL |
| | | SSTASALGKLQNVVNQNAQALNTLVKQLSSNFGAISSVLNDILSRLDPPEAE |
| | | VQIDRLITGRLQSLQTYVTQQLIRAAEIRASANLAATKMSECVLGQSKRVDF |
| | | CGKGYHLMSFPQSAPHGVVFLHVTYVPAQEKNFTTAPAICHDGKAHFPREGV |
| | | FVSNGTHWFVTQRNFYEPQIISTDNTFVSGNCDVVIGIVNNTVYDPLQPFLD |
| | | SFKEELDKYFKNHTSPDVDLGDISGINASVVNIQKEIDRLNEVAKNLNESLI |
| | | DLQELGKYEQYIKWPWYIWLGFIAGLIAIVMVTIMLCCMTSCCSCLKGCCSC |
| | | GSCCKFDEDDSEPVLKGVKLHYT |
| SARS-COV-2 | 322 | ATGTTCGTGTTCCTGGTGCTGCTGCCCCTGGTGAGCAGCCAGTGCGTGAACC |
| B.1.617.2 SPIKE | | TGCGGACCCGGACCCAGCTGCCACCAGCCTACACCAACAGCTTCACCCGGGG |
| (Delta Variant (With | | CGTCTACTACCCCGACAAGGTGTTCCGGAGCAGCGTCCTGCACAGCACCCAG |
| two proline | | GACCTGTTCCTGCCCTTCTTCAGCAACGTGACCTGGTTCCACGCCATCCACG |
| substitutions); DNA | | TGAGCGGCACCAACGGCACCAAGCGGTTCGACAACCCCGTGCTGCCCTTCAA |
| Sequence | | CGACGGCGTGTACTTCGCCAGCACCGAGAAGAGCAACATCATCCGGGGCTGG |
| | | ATCTTCGGCACCACCCTGGACAGCAAGACCCAGAGCCTGCTGATCGTGAATA |
| | | ACGCCACCAACGTGGTGATCAAGGTGTGCGAGTTCCAGTTCTGCAACGACCC |
| | | CTTCCTGGACGTGTACTACCACAAGAACAACAAGAGCTGGATGGAGAGCGGC |
| | | GTGTACAGCAGCGCCAACAACTGCACCTTCGAGTACGTGAGCCAGCCCTTCC |
| | | TGATGGACCTGGAGGGCAAGCAGGGCAACTTCAAGAACCTGCGGGAGTTCGT |
| | | GTTCAAGAACATCGACGGCTACTTCAAGATCTACAGCAAGCACACCCCAATC |
| | | AACCTGGTGCGGGATCTGCCCCAGGGCTTCTCAGCCCTGGAGCCCCTGGTGG |
| | | ACCTGCCCATCGGCATCAACASCACCCGGTTCCAGACCCTGCTGGCCCTGCA |
| | | CCGGAGCTACCTGACCCCAGGCGACAGCAGCAGCGGGTGGACAGCAGGCGCG |
| | | GCTGCTTACTACGTGGGCTACCTGCAGCCCCGGACCTTCCTGCTGAAGTACA |
| | | ACGAGAACGGCACCATCACCGACGCCGTGGACTGCGCCCTGGACCCTCTGAG |
| | | CGAGACCAAGTGCACCCTGAAGAGCTTCACCGTGGAGAAGGGCATCTACCAG |
| | | ACCAGCAACTTCCGGGTGCAGCCCACCGAGAGCATCGTGCGGTTCCCCAACA |
| | | TCACCAACCTGTGCCCCTTCGGCGAGGTGTTCAACGCCACCCGGTTCGCCAG |
| | | CGTGTACGCCTGGAACCGGAAGCGGATCAGCAACTGCGTGGCCGACTACAGC |
| | | GTGCTGTACAACAGCGCCAGCTTCAGCACCTTCAAGTGCTACGGCGTGAGCC |
| | | CCACCAAGCTGAACGACCTGTGCTTCACCAACGTGTACGCCGACAGCTTCGT |
| | | GATCCGTGGCGACGAGGTGCGGCAGATCGCACCCGGCCAGACAGGCAAGATC |
| | | GCCGACTACAACTACAAGCTGCCCGACGACTTCACCGGCTGCGTGATCGCCT |
| | | GGAACAGCAACAACCTCGACAGCAAGGTGGGCGGCAACTACAACTACCGGTA |
| | | CCGGCTGTTCCGGAAGAGCAACCTGAAGCCCTTCGAGCGGGACATCAGCACC |
| | | GAGATCTACCAAGCCGGCTCCAAGCCTTGCAACGGCGTGGAGGGCTTCAACT |
| | | GCTACTTCCCTCTGCAGAGCTACGGCTTCCAGCCCACCAACGGCGTGGGCTA |
| | | CCAGCCCTACCGGGTGGTGGTGCTGAGCTTCGAGCTGCTGCACGCCCCAGCC |
| | | ACCGTGTGTGGCCCCAAGAAGAGCACCAACCTGGTGAAGAACAAGTGCGTGA |
| | | ACTTCAACTTCAACGGCCTTACCGGCACCGGCGTGCTGACCGAGAGCAACAA |
| | | GAAATTCCTGCCCTTTCAGCAGTTCGGCCGGGACATCGCCGACACCACCGAC |
| | | GCTGTGCGGGATCCCCAGACCCTGGAGATCCTGGACATCACCCCTTGCAGCT |
| | | TCGGCGGCGTGAGCGTGATCACCCCAGGCACCAACACCAGCAACCAGGTGGC |
| | | CGTGCTGTACCAGGGCGTGAACTGCACCGAGGTGCCCGTGGCCATCCACGCC |
| | | GACCAGCTGACACCCACCTGGCGGGTCTACAGCACCGGCAGCAACGTGTTCC |
| | | AGACCCGGGCCGGTTGCCTGATCGGCGCCGAGCACGTGAACAACAGCTACGA |
| | | GTGCGACATCCCCATCGGCGCCGGCATCTGTGCCAGCTACCAGACCCAGACC |
| | | AATTCACGGCGGAGGGCAAGGAGCGTGGCCAGCCAGAGCATCATCGCCTACA |
| | | CCATGAGCCTGGGCGCCGAGAACAGCGTGGCCTACAGCAACAACAGCATCGC |
| | | CATCCCCACCAACTTCACCATCAGCGTGACCACCGAGATTCTGCCCGTGAGC |
| | | ATGACCAAGACCAGCGTGGACTGCACCATGTACATCTGCGGCGACAGCACCG |
| | | AGTGCAGCAACCTGCTGCTGCAGTACGGCAGCTTCTGCACCCAGCTGAACCG |
| | | GGCCCTGACCGGCATCGCCGTGGAGCAGGACAAGAACACCCAGGAGGTGTTC |
| | | GCCCAGGTGAAGCAGATCTACAAGACCCCTCCCATCAAGGACTTCGGCGGCT |
| | | TCAACTTCAGCCAGATCCTGCCCGACCCCAGCAAGCCCAGCAAGCGGAGCTT |
| | | CATCGAGGACCTGCTGTTCAACAAGGTGACCCTAGCCGACGCCGGCTTCATC |
| | | AAGCAGTACGGCGACTGCCTCGGCGACATAGCCGCCCGGGACCTGATCTGCG |
| | | CCCAGAAGTTCAACGGCCTGACCGTGCTGCCTCCCCTGCTGACCGACGAGAT |
| | | GATCGCCCAGTACACCAGCGCCCTGTTAGCCGGAACCATCACCAGCGGCTGG |
| | | ACTTTCGGCGCTGGAGCCGCTCTGCAGATCCCCTTCGCCATGCAGATGGCCT |
| | | ACCGGTTCAACGGCATCGGCGTGACCCAGAACGTGCTGTACGAGAACCAGAA |
| | | GCTGATCGCCAACCAGTTCAACAGCGCCATCGGCAAGATCCAGGACAGCCTG |
| | | AGCAGCACCGCTAGCGCCCTGGGCAAGCTGCAGAACGTGGTGAACCAGAACG |
| | | CCCAGGCCCTGAACACCCTGGTGAAGCAGCTGAGCAGCAACTTCGGCGCCAT |
| | | CAGCAGCGTGCTGAACGACATCCTGAGCCGGCTGGACCCTCCCGAGGCCGAG |
| | | GTGCAGATCGACCGGCTGATCACTGGCCGGCTGCAGAGCCTGCAGACCTACG |
| | | TGACCCAGCAGCTGATCCGGGCCGCCGAGATTCGGGCCAGCGCCAACCTGGC |
| | | CGCCACCAAGATGAGCGAGTGCGTGCTGGGCCAGAGCAAGCGGGTGGACTTC |
| | | TGCGGCAAGGGCTACCACCTGATGAGCTTTCCCCAGAGCGCACCCCACGGAG |
| | | TGGTGTTCCTGCACGTGACCTACGTGCCCGCCCAGGAGAAGAACTTCACCAC |
| | | CGCCCCAGCCATCTGCCACGACGGCAAGGCCCACTTTCCCCGGGAGGGCGTG |
| | | TTCGTGAGCAACGGCACCCACTGGTTCGTGACCCAGCGGAACTTCTACGAGC |
| | | CCCAGATCATCACCACCGACAACACCTTCGTGAGCGGCAACTGCGACGTGGT |
| | | GATCGGCATCGTGAACAACACCGTGTACGATCCCCTGCAGCCCGAGCTGGAC |
| | | AGCTTCAAGGAGGAGCTGGACAAGTACTTCAAGAATCACACCAGCCCCGACG |
| | | TGGACCTGGGCGACATCAGCGGCATCAACGCCAGCGTGGTGAACATCCAGAA |
| | | GGAGATCGATCGGCTGAACGAGGTGGCCAAGAACCTGAACGAGAGCCTGATC |
| | | GACCTGCAGGAGCTGGGCAAGTACGAGCAGTACATCAAGTGGCCCTGGTACA |
| | | TCTGGCTGGGCTTCATCGCCGGCCTGATCGCCATCGTGATGGTGACCATCAT |
| | | GCTGTGCTGCATGACCAGCTGCTGCAGCTGCCTGAAGGGCTGTTGCAGCTGC |
| | | GGCAGCTGCTGCAAGTTCGACGAGGACGACAGCGAGCCCGTGCTGAAGGGCG |
| | | TGAAGCTGCACTACACC |
| 5′ UTR; DNA | 323 | GGAAATAAGAGAGAAAAGAAGAGTAAGAAGAAATATAAGACCCCGGCGCCGC |
| sequence | | CACC |
| 3′ UTR and PolyA; | 324 | GCTGGAGCCTCGGTGGCCTAGCTTCTTGCCCCTTGGGCCTCCCCCCAGCCCC |
| DNA sequence | | TCCTCCCCTTCCTGCACCCGTACCCCCGTGGTCTTTGAATAAAGTCTGAGTG |
| | | GGCGGCAaaaaaaaaaaaaaaaaaaaaaaaaaaaaagcatatgactaaaaaa |
| | | aaaaaaaaaaaa |
| SARS-COV-2 | 325 | MFVFLVLLPLVSSQCVNLTTRTQLPPAYTNSFTRGVYYPDKVFRSSVLHSTQ |
| B.1.1.529 SPIKE | | DLFLPFFSNVTWFHVISGTNGTKRFDNPVLPFNDGVYFASIEKSNIIRGWIF |
| (Omicron Variant | | GTTLDSKTQSLLIVNNATNVVIKVCEFQFCNDPFLDHKNNKSWMESEFRVYS |
| (With two proline | | SANNCTFEYVSQPFLMDLEGKQGNFKNLREFVFKNIDGYFKIYSKHTPIIVR |
| substitutions); Protein | | EPEDLPQGESALEPLVDLPIGINITRFQTLLALHRSYLTPGDSSSGWTAGAA |
| Sequence | | AYYVGYLQPRTFLLKYNENGTITDAVDCALDPLSETKCTLKSFTVEKGIYQT |
| | | SNFRVQPTESIVRFPNITNLCPFDEVFNATRFASVYAWNRKRISNCVADYSV |
| | | LYNIAPFSTFKCYGVSPTKLNDLCFTNVYADSFVIRGDEVRQIAPGQTGNIA |
| | | DYNYKLPDDFTGCVIAWNSNKLDSKVSGNYNYLYRLERKSNLKPFERDISTE |
| | | IYQAGNKPCNGVAGFNCYFPLRSYSFRPTYGVGYQPYRVVVLSFELLHAPAT |
| | | VCGPKKSTNLVKNKCVNFNFNGLKGTGVLTESNKKFLPFQQFGRDIADTTDA |
| | | VRDPQTLEILDITPCSFGGVSVITPGTNTSNQVAVLYQGVNCTEVPVAIHAD |
| | | QLTPTWRVYSTGSNVFQTRAGCLIGAEYVNNSYECDIPIGAGICASYQTQTK |
| | | SHRRARSVASQSIIAYTMSLGAENSVAYSNNSIAIPTNFTISVTTEILPVSM |
| | | TKTSVDCTMYICGDSTECSNLLLQYGSFCTQLKRALTGIAVEQDKNTQEVFA |
| | | QVKQIYKTPPIKYFGGFNFSQILPDPSKPSKRSFIEDLLFNKVTLADAGFIK |
| | | QYGDCLGDIAARDLICAQKFKGLTVLPPLLIDEMIAQYTSALLAGTITSGWT |
| | | FGAGAALQIPFAMQMAYRFNGIGVTQNVLYENQKLIANQFNSAIGKIQDSLS |
| | | STASALGKLQDVVNHNAQALNTLVKQLSSKFGAISSVLNDELSRLDPPEAEV |
| | | QIDRLITGRLQSLQTYVTQQLIRAAEIRASANLAATKMSECVLGQSKRVDFC |
| | | GKGYHLMSFPQSAPHGVVFLHVTYVPAQEKNFTTAPAICHDGKAHFPREGVF |
| | | VSNGTHWFVTQRNFYEPQIITIDNTFVSGNCDVVIGIVNNTVYDPLQPFLDS |
| | | FKEELDKYFKNHTSPDVDLGDISGINASVVNIQKEIDRLNEVAKNLNESLID |
| | | LQELGKYEQYIKWPWYIWLGFTAGLIAIVMVTIMLCCMTSCCSCLKGCCSCG |
| | | SCCKFDEDDSEPVLKGVKLHYT |
| SARS-COV-2 | 326 | ATGTTCGTGTTCCTGGTGCTGCTGCCCCTGGTGAGCAGCCAGTGCGTGAACC |
| B.1.1.529 SPIKE | | TGACCACCCGGACCCAGCTGCCACCAGCCTACACCAACAGCTTCACCCGGGG |
| (Omicron Variant | | CGTCTACTACCCCGACAAGGTGTTCCGGAGCAGCGTCCTGCACAGCACCCAG |
| (With two proline | | GACCTGTTCCTGCCCTTCTTCAGCAACGTGACCTGGTTCCACGtCATCAGCG |
| substitutions); DNA | | GCACCAACGGCACCAAGCGGTTCGACAACCCCGTGCTGCCCTTCAACGACGG |
| Sequence | | CGTGTACTTCGCCAGCAtCGAGAAGAGCAACATCATCCGGGGCTGGATCTTC |
| | | GGCACCACCCTGGACAGCAAGACCCAGAGCCTGCTGATCGTGAATAACGCCA |
| | | CCAACGTGGTGATCAAGGTGTGCGAGTTCCAGTTCTGCAACGACCCCTTCCT |
| | | GGaCCACAAGAACAACAAGAGCTGGATGGAGAGCGAGTTCCGGGTGTACAGC |
| | | AGCGCCAACAACTGCACCTTCGAGTACGTGAGCCAGCCCTTCCTGATGGACC |
| | | TGGAGGGCAAGCAGGGCAACTTCAAGAACCTGCGGGAGTTCGTGTTCAAGAA |
| | | CATCGACGGCTACTTCAAGATCTACAGCAAGCACACCCCAATCatcGTGCGG |
| | | GAGCCAGAAGATCTGCCCCAGGGCTTCTCAGCCCTGGAGCCCCTGGTGGACC |
| | | TGCCCATCGGCATCAACATCACCCGGTTCCAGACCCTGCTGGCCCTGCACCG |
| | | GAGCTACCTGACCCCAGGCGACAGCAGCAGCGGGTGGACAGCAGGCGCGGCT |
| | | GCTTACTACGTGGGCTACCTGCAGCCCCGGACCTTCCTGCTGAAGTACAACG |
| | | AGAACGGCACCATCACCGACGCCGTGGACTGCGCCCTGGACCCTCTGAGCGA |
| | | GACCAAGTGCACCCTGAAGAGCTTCACCGTGGAGAAGGGCATCTACCAGACC |
| | | AGCAACTTCCGGGTGCAGCCCACCGAGAGCATCGTGCGGTTCCCCAACATCA |
| | | CCAACCTGTGCCCCTTCGaCGAGGTGTTCAACGCCACCCGGTTCGCCAGCGT |
| | | GTACGCCTGGAACCGGAAGCGGATCAGCAACTGCGTGGCCGACTACAGCGTG |
| | | CTGTACAACAtCGCCccCTTCAGCACCTTCAAGTGCTACGGCGTGAGCCCCA |
| | | CCAAGCTGAACGACCTGTGCTTCACCAACGTGTACGCCGACAGCTTCGTGAT |
| | | CCGTGGCGACGAGGTGCGGCAGATCGCACCCGGCCAGACAGGCAAcATCGCC |
| | | GACTACAACTACAAGCTGCCCGACGACTTCACCGGCTGCGTGATCGCCTGGA |
| | | ACAGCAACAAgCTCGACAGCAAGGTGaGCGGCAACTACAACTACCTGTACCG |
| | | GCTGTTCCGGAAGAGCAACCTGAAGCCCTTCGAGCGGGACATCAGCACCGAG |
| | | ATCTACCAAGCCGGCaaCAagCCTTGCAACGGCGTGGGGGGCTTCAACTGCT |
| | | ACTTCCCTCTGCgGAGCTACaGCTTCCgGCCCACCtACGGCGTGGGCTACCA |
| | | GCCCTACCGGGTGGTGGTGCTGAGCTTCGAGCTGCTGCACGCCCCAGCCACC |
| | | GTGTGTGGCCCCAAGAAGAGCACCAACCTGGTGAAGAACAAGTGCGTGAACT |
| | | TCAACTTCAACGGCCTTAagGGCACCGGCGTGCTGACCGAGAGCAACAAGAA |
| | | ATTCCTGCCCTTTCAGCAGTTCGGCCGGGACATCGCCGACACCACCGACGCT |
| | | GTGCGGGATCCCCAGACCCTGGAGATCCTGGACATCACCCCTTGCAGCTTCG |
| | | GCGGCGTGAGCGTGATCACCCCAGGCACCAACACCAGCAACCAGGTGGCCGT |
| | | GCTGTACCAGGgCGTGAACTGCACCGAGGTGCCCGTGGCCATCCACGCCGAC |
| | | CAGCTGACACCCACCTGGCGGGTCTACAGCACCGGCAGCAACGTGTTCCAGA |
| | | CCCGGGCCGGTTGCCTGATCGGCGCCGAGtACGTGAACAACAGCTACGAGTG |
| | | CGACATCCCCATCGGCGCCGGCATCTGTGCCAGCTACCAGACCCAGACCAAg |
| | | TCACaCCGGAGGGCAAGGAGCGTGGCCAGCCAGAGCATCATCGCCTACACCA |
| | | TGAGCCTGGGCGCCGAGAACAGCGTGGCCTACAGCAACAACAGCATCGCCAT |
| | | CCCCACCAACTTCACCATCAGCGTGACCACCGAGATTCTGCCCGTGAGCATG |
| | | ACCAAGACCAGCGTGGACTGCACCATGTACATCTGCGGCGACAGCACCGAGT |
| | | GCAGCAACCTGCTGCTGCAGTACGGCAGCTTCTGCACCCAGCTGAAgCGGGC |
| | | CCTGACCGGCATCGCCGTGGAGCAGGACAAGAACACCCAGGAGGTGTTCGCC |
| | | CAGGTGAAGCAGATCTACAAGACCCCTCCCATCAAGtACTTCGGCGGCTTCA |
| | | ACTTCAGCCAGATCCTGCCCGACCCCAGCAAGCCCAGCAAGCGGAGCTTCAT |
| | | CGAGGACCTGCTGTTCAACAAGGTGACCCTAGCCGACGCCGGCTTCATCAAG |
| | | CAGTACGGCGACTGCCTCGGCGACATAGCCGCCCGGGACCTGATCTGCGCCC |
| | | AGAAGTTCAAgGGCCTGACCGTGCTGCCTCCCCTGCTGACCGACGAGATGAT |
| | | CGCCCAGTACACCAGCGCCCTGTTAGCCGGAACCATCACCAGCGGCTGGACT |
| | | TTCGGCGCTGGAGCCGCTCTGCAGATCCCCTTCGCCATGCAGATGGCCTACC |
| | | GGTTCAACGGCATCGGCGTGACCCAGAACGTGCTGTACGAGAACCAGAAGCT |
| | | GATCGCCAACCAGTTCAACAGCGCCATCGGCAAGATCCAGGACAGCCTGAGC |
| | | AGCACCGCTAGCGCCCTGGGCAAGCTGCAGGACGTGGTGAACCACAACGCCC |
| | | AGGCCCTGAACACCCTGGTGAAGCAGCTGAGCAGCAAgTTCGGCGCCATCAG |
| | | CAGCGTGCTGAACGACATCCTGAGCCGGCTGGACCCTCCCGAGGCCGAGGTG |
| | | CAGATCGACCGGCTGATCACTGGCCGGCTGCAGAGCCTGCAGACCTACGTGA |
| | | CCCAGCAGCTGATCCGGGCCGCCGAGATTCGGGCCAGCGCCAACCTGGCCGC |
| | | CACCAAGATGAGCGAGTGCGTGCTGGGCCAGAGCAAGCGGGTGGACTTCTGC |
| | | GGCAAGGGCTACCACCTGATGAGCTTTCCCCAGAGCGCACCCCACGGAGTGG |
| | | TGTTCCTGCACGTGACCTACGTGCCCGCCCAGGAGAAGAACTTCACCACCGC |
| | | CCCAGCCATCTGCCACGACGGCAAGGCCCACTTTCCCCGGGAGGGCGTGTTC |
| | | GTGAGCAACGGCACCCACTGGTTCGTGACCCAGCGGAACTTCTACGAGCCCC |
| | | AGATCATCACCACCGACAACACCTTCGTGAGCGGCAACTGCGACGTGGTGAT |
| | | CGGCATCGTGAACAACACCGTGTACGATCCCCTGCAGCCCGAGCTGGACAGC |
| | | TTCAAGGAGGAGCTGGACAAGTACTTCAAGAATCACACCAGCCCCGACGTGG |
| | | ACCTGGGCGACATCAGCGGCATCAACGCCAGCGTGGTGAACATCCAGAAGGA |
| | | GATCGATCGGCTGAACGAGGTGGCCAAGAACCTGAACGAGAGCCTGATCGAC |
| | | CTGCAGGAGCTGGGCAAGTACGAGCAGTACATCAAGTGGCCCTGGTACATCT |
| | | GGCTGGGCTTCATCGCCGGCCTGATCGCCATCGTGATGGTGACCATCATGCT |
| | | GTGCTGCATGACCAGCTGCTGCAGCTGCCTGAAGGGCTGTTGCAGCTGCGGC |
| | | AGCTGCTGCAAGTTCGACGAGGACGACAGCGAGCCCGTGCTGAAGGGCGTGA |
| | | AGCTGCACTACACC |
| SARS-COV-2 BA.2 | 327 | MFVFLVLLPLVSSQCVNLITRTQSYTNSFTRGVYYPDKVFRSSVLHSTQDLF |
| SPIKE (Omicron | | LPFFSNVTWFHAIHVSGTNGTKRFDNPVLPFNDGVYFASTEKSNIIRGWIFG |
| BA.2 Variant (With | | TTLDSKTQSLLIVNNATNVVIKVCEFQFCNDPFLDVYYHKNNKSWMESEFRV |
| two proline | | YSSANNCTFEYVSQPFLMDLEGKQGNFKNLREFVFKNTDGYFKTYSKHTPTN |
| substitutions); Protein | | LGRDLPQGFSALEPLVDLPIGINITRFQTLLALHRSYLTPGDSSSGWTAGAA |
| Sequence | | AYYVGYLQPRTFLLKYNENGTITDAVDCALDPLSETKCTLKSFTVEKGIYQT |
| | | SNFRVQPTESIVRFPNITNLCPFDEVFNATRFASVYAWNRKRISNCVADYSV |
| | | LYNFAPFFAFKCYGVSPTKLNDLCFTNVYADSFVIRGNEVSQIAPGQTGNIA |
| | | DYNYKLPDDFTGCVIAWNSNKLDSKVGGNYNYLYRLFRKSNLKPFERDISTE |
| | | IYQAGNKPCNGVAGFNCYFPLRSYGFRPTYGVGHQPYRVVVLSFELLHAPAT |
| | | VCGPKKSTNLVKNKCVNFNFNGLTGTGVLTESNKKFLPFQQFGRDIADTTDA |
| | | VRDPQTLEILDITPCSFGGVSVITPGTNTSNQVAVLYQGVNCTEVPVAIHAD |
| | | QLTPTWRVYSTGSNVFQTRAGCLIGAEYVNNSYECDIPIGAGICASYQTQTK |
| | | SHRRARSVASQSIIAYTMSLGAENSVAYSNNSIAIPTNFTISVTTEILPVSM |
| | | TKTSVDCTMYICGDSTECSNLLLQYGSFCTQLKRALTGIAVEQDKNTQEVFA |
| | | QVKQIYKTPPIKYFGGFNFSQILPDPSKPSKRSFIEDLLFNKVTLADAGFIK |
| | | QYGDCLGDIAARDLICAQKFNGLTVLPPLLIDEMIAQYTSALLAGTITSGWT |
| | | FGAGAALQIPFAMQMAYRFNGIGVTQNVLYENQKLIANQFNSAIGKIQDSLS |
| | | STASALGKLQDVVNHNAQALNTLVKQLSSKFGAISSVLNDILSRLDPPEAEV |
| | | QIDRLITGRLQSLQYTVTQQLIRAAEIRASANLAATKMSECVLGQSKRVDFC |
| | | GKGYHLMSFPQSAPHGVVFLHVTYVPAQEKNFTTAPAICHDGKAHFPREGVF |
| | | VSNGTHWFVTQRNFYEPQIITTDNTFVSGNCDVVIGIVNNTVYDPLQPFLDS |
| | | FKEELDKYFKNHTSPDVDLGDISGINASVVNIQKEIDRLNEVAKNLNESLID |
| | | LQELGKYEQYIKWPWYIWLGFIAGLIAIVMVTIMLCCMTSCCSCLKGCCSCG |
| | | SCCKFDEDDSEPVIKGVKTHYT |
| SARS-COV-2 BA.2 | 328 | ATGTTCGTGTTCCTGGTGCTGCTGCCCCTGGTGAGCAGCCAGTGCGTGAACC |
| SPIKE (Omicron | | TGAtCACCCGGACCCAGagcTACACCAACAGCTTCACCCGGGGCGTCTACTA |
| BA.2 Variant (With | | CCCCGACAAGGTGTTCCGGAGCAGCGTCCTGCACAGCACCCAGGACCTGTTC |
| two proline | | CTGCCCTTCTTCAGCAACGTGACCTGGTTCCACGCCATCCACGTGAGCGGCA |
| | | CCAACGGCACCAAGCGGTTCGACAACCCCGTGCTGCCCTTCAACGACGGCGT |
| | | GTACTTCGCCAGCACCGAaAAGAGCAACATCATCCGGGGCTGGATCTTCGGC |
| | | ACCACCCTGGACAGCAAGACCCAGAGCCTGCTGATCGTGAATAACGCCACCA |
| | | ACGTGGTGATCAAGGTGTGCGAGTTCCAGTTCTGCAACGACCCCTTCCTGGa |
| | | CGTGTACTACCACAAGAACAACAAGAGCTGGATGGAGAGCGAGTTCCGGGTG |
| | | TACAGCAGCGCCAACAACTGCACCTTCGAGTACGTGAGCCAGCCCTTCCTGA |
| | | TGGACCTGGAGGGCAAGCAGGGCAACTTCAAGAACCTGCGGGAGTTCGTGTT |
| | | CAAGAACATCGACGGCTACTTCAAGATCTACAGCAAGCACACCCCAATCAAC |
| | | CTGGgcCGGGATCTGCCCCAGGGCTTCTCAGCCCTGGAGCCCCTGGTGGACC |
| | | TGCCCATCGGCATCAACATCACCCGGTTCCAGACCCTGCTGGCCCTGCACCG |
| | | GAGCTACCTGACCCCAGGCGACAGCAGCAGCGGGTGGACAGCAGGCGCGGCT |
| | | GCTTACTACGTGGGCTACCTGCAGCCCCGGACCTTCCTGCTGAAGTACAACG |
| | | AGAACGGCACCATCACCGACGCCGTGGACTGCGCCCTGGACCCTCTGAGCGA |
| | | GACCAAGTGCACCCTCAAGAGCTTCACCGTGGAGAAGGGCATCTACCAGACC |
| | | AGCAACTTCCGGGTGCAGCCCACCGAGAGCATCGTGCGGTTCCCCAACATCA |
| | | CCAACCTGTGCCCCTTCGaCGAGGTGTTCAACGCCACCCGGTTCGCCAGCGT |
| | | GTACGCCTGGAACCGGAAGCGGATCAGCAACTGCGTGGCCGACTACAGCGTG |
| | | CTGTACAACttCGCCCCATTCttCgCCTTCAAGTGCTACGGCGTGAGCCCCA |
| | | CCAAGCTGAACGACCTGTGCTTCACCAACGTGTACGCCGACAGCTTCGTGAT |
| | | CCGTGGCaACGAGGTGagcCAGATCGCACCCGGCCAGACAGGCAAcATCGCC |
| | | GACTACAACTACAAGCTGCCCGACGACTTCACCGGCTGCGTGATCGCCTGGA |
| | | ACAGCAACAAgCTCGACAGCAAGGTGGGCGGCAACTACAACTACCTGTACCG |
| | | GCTGTTCCGaAAGAGCAACCTGAAGCCCTTCGAGCGGGACATCAGCACCGAG |
| | | ATCTACCAAGCCGGCaaCAagCCTTGCAACGGCGTGGccGGCTTCAACTGCT |
| | | ACTTCCCTCTGCgGAGCTACGGCTTCCgGCCCACCtACGGCGTGGGCCACCA |
| | | GCCCTACCGGGTGGTGGTGCTGAGCTTCGAGCTGCTGCACGCCCCAGCCACC |
| | | GTGTGTGGCCCCAAGAAGAGtACCAACCTGGTGAAGAACAAGTGCGTGAACT |
| | | TCAACTTCAACGGCCTTACCGGCACCGGCGTGCTGACCGAGAGCAACAAGAA |
| | | ATTCCTGCCCTTTCAGCAGTTCGGCCGGGACATCGCCGACACCACCGACGCT |
| | | GTGCGGGATCCCCAGACCCTGGAGATCCTGGACATCACCCCTTGCAGCTTCG |
| | | GCGGCGTGAGCGTGATCACCCCAGGCACCAACACCAGCAACCAGGTGGCCGT |
| | | GCTGTACCAGGgCGTGAACTGCACCGAGGTGCCCGTGGCCATCCACGCCGAC |
| | | CAGCTGACACCCACCTGGCGGGTCTACAGCACCGGCAGCAACGTGTTCCAGA |
| | | CCCGGGCCGGTTGCCTGATCGGCGCCGAGtACGTGAACAACAGCTACGAGTG |
| | | CGACATCCCCATCGGCGCCGGCATCTGTGCCAGCTACCAGACCCAGACCAAg |
| | | TCACaCCGGAGGGCAAGGAGCGTGGCCAGCCAGAGCATCATCGCCTACACCA |
| | | TGAGCCTGGGCGCCGAGAACAGCGTGGCCTACAGCAACAACAGCATCGCCAT |
| | | CCCCACCAACTTCACCATCAGCGTGACCACCGAGATTCTGCCCGTGAGCATG |
| | | ACCAAGACCAGCGTGGACTGCACCATGTACATCTGCGGCGACAGCACCGAGT |
| | | GCAGCAACCTGCTGCTGCAGTACGGCAGCTTCTGCACCCAGCTGAAgCGGGC |
| | | CCTGACCGGCATCGCCGTGGAGCAGGACAAGAACACCCAGGAGGTGTTCGCC |
| | | CAGGTGAAGCAGATCTACAAGACCCCTCCCATCAAGtACTTCGGCGGCTTCA |
| | | ACTTCAGCCAGATCCTGCCCGACCCCAGCAAGCCCAGCAAGCGGAGCTTCAT |
| | | CGAGGACCTGCTGTTCAACAAGGTGACCCTAGCCGACGCCGGCTTCATCAAG |
| | | CAGTACGGCGACTGCCTCGGCGACATAGCCGCCCGGGACCTGATCTGCGCCC |
| | | AGAAGTTCAACGGCCTGACCGTGCTGCCTCCCCTGCTGACCGACGAGATGAT |
| | | CGCCCAGTACACCAGCGCCCTGTTAGCCGGAACCATCACCAGCGGCTGGACT |
| | | TTCGGCGCTGGAGCCGCTCTGCAGATCCCCTTCGCCATGCAGATGGCCTACC |
| | | GGTTCAACGGCATCGGCGTGACCCAGAACGTGCTGTACGAGAACCAGAAGCT |
| | | GATCGCCAACCAGTTCAACAGCGCCATCGGCAAGATCCAGGACAGCCTGAGC |
| | | AGCACCGCTAGCGCCCTGGGCAAGCTGCAGGACGTGGTGAACCACAACGCCC |
| | | AGGCCCTGAACACCCTGGTGAAGCAGCTGAGCAGCAAgTTCGGCGCCATCAG |
| | | CAGCGTGCTGAACGACATCCTGAGCCGGCTGGACCCTCCCGAGGCCGAGGTG |
| | | CAGATCGACCGGCTGATCACTGGCCGGCTGCAGAGCCTGCAGACCTACGTGA |
| | | CCCAGCAGCTGATCCGGGCCGCCGAGATTCGGGCCAGCGCCAACCTGGCCGC |
| | | CACCAAGATGAGCGAGTGCGTGCTGGGCCAGAGCAAGCGGGTGGACTTCTGC |
| | | GGCAAGGGCTACCACCTGATGAGCTTTCCCCAGAGCGCACCCCACGGAGTGG |
| | | TGTTCCTGCACGTGACCTACGTGCCCGCCCAGGAGAAGAACTTCACCACCGC |
| | | CCCAGCCATCTGCCACGACGGCAAGGCCCACTTTCCCCGGGAGGGCGTGTTC |
| | | GTGAGCAACGGCACCCACTGGTTCGTGACCCAGCGGAACTTCTACGAGCCCC |
| | | AGATCATCACCACCGACAACACCTTCGTGAGCGGCAACTGCGACGTGGTGAT |
| | | CGGCATCGTGAACAACACCGTGTACGATCCCCTGCAGCCCGAGCTGGACAGC |
| | | TTCAAGGAGGAGCTGGACAAGTACTTCAAGAATCACACCAGCCCCGACGTGG |
| | | ACCTGGGCGACATCAGCGGCASCAACGCCAGCGTGGTGAACATCCAGAAGGA |
| | | GATCGATCGGCTGAACGAGGTGGCCAAGAACCTGAACGAGAGCCTGATCGAC |
| | | CTGCAGGAGCTGGGCAAGTACGAGCAGTACATCAAGTGGCCCTGGTACATCT |
| | | GGCTGGGCTTCATCGCCGGCCTGATCGCCATCGTGATGGTGACCATCATGCT |
| | | GTGCTGCATGACCAGCTGCTGCAGCTGCCTGAAGGGCTGTTGCAGCTGCGGC |
| | | AGCTGCTGCAAGTTCGACGAGGACGACAGCGAGCCCGTGCTGAAGGGCGTGA |
| | | AGCTGCACTACACC |
| ARV-COV-MTE | 329 | MFLALITLATCELYHYQECVRGITEELKKLLEQWNLVIGFLFLTWICLLQFA |
| (SARS-COV-2 Multi- | | YANRNRFLYIIKLIFLWLLWPVTLACFVLAAVYRINWITGGMACLVGLMWLS |
| T-cell-Epitopes); | | YFIASFRLFARTRSMWSFNPETNILLNVPLHGTILTRPLLESELVIGAVILR |
| Protein Sequence | | GHLRIAGHHLGRCDIKDLPKEITVATSRTLSYYKLGASQRVAGDSGFAAYSR |
| | | YRIGNYKLNTDHSSSSDNIALLVPNNTASWFTALTQHGKEDLKFPRGQGVPI |
| | | NTNSSPDDQIGYYRRATRRIRGGDGKMKDLSPRWYFYYLGTGPEAGLPYGGR |
| | | RGPEQTQGNFGDQELIRQGTDYKHWPQIAQFAPSASAFFGMSRIGMEVTPSG |
| | | TWLTYTGAIKLDDKDPNFKDQVILLNKHIDAYKTFPPTEPKKDKKRSKNPLL |
| | | YDANYFLCWHTNCYDYCIPYNSVTSSIVITYALVYFLQSINFVRISGVKDCV |
| | | VLHSYFTSDYYQLYSTQLSTDIGVEHVTFFIYNKIVDEPEEHVQLEQPYVFI |
| | | KRSDARTAPHGSEKSYELQTPFEIKLVCTNYMPYFFTLLLQLMTYRRLISMM |
| | | GFKMNYQVNREQIDGYVMHANYIFWRNT |
| ARV-COV-MTE | 330 | ATGTTCCTGGCCCTCATCACCCTCGCCACGTGCGAGCTGTACCACTACCAGG |
| (SARS-COV-2 Multi- | | AGTGCGTGCGGGGCACCACCGAGGAGCTGAAGAAGCTCCTCGAGCAGTGGAA |
| T-cell-Epitopes); | | CCTGGTGATCGGCTTCCTGTTCCTGACCTGGATCTGCCTGCTGCAGTTCGCC |
| DNA Sequence | | TATGCCAACCGGAACCGGTTCCTGTACATCATCAAGCTGATCTTCCTGTGGC |
| | | TCCTGTGGCCGGTCACCCTGGCCTGCTTCGTGCTCGCCGCCGTGTACCGGAT |
| | | CAACTGGATCACCGGCGGGATGGCCTGCCTGGTGGGGCTGATGTGGCTGAGC |
| | | TACTTCATCGCCAGCTTCCGGCTGTTCGCCCGGACGCGGAGCATGTGGAGCT |
| | | TTAACCCCGAGACCAACATCCTGCTGAACGTCCCCCTGCACGGCACGATCCT |
| | | GACCCGGCCCCTGCTGGAGAGCGAGCTGGTCATCGGCGCGGTGATCCTGCGG |
| | | GGCCACCTGCGGATCGCCGGCCACCACCTGGGCCGGTGCGACATCAAGGACC |
| | | TGCCCAAGGAGATCACCGTCGCCACCAGCCGGACGCTGAGCTACTACAAGCT |
| | | GGGCGCCAGCCAGCGGGTCGCCGGCGACAGCGGGTTCGCCGCCTACAGCCGG |
| | | TACCGGATCGGCAACTACAAGCTGAACACCGACCACAGCAGCAGCAGCGACA |
| | | ACATCGCCCTGCTGGTGCCCAACAACACCGCGTCCTGGTTCACCGCCCTGAC |
| | | CCAGCACGGGAAAGAGGACCTGAAGTTCCCGAGGGGGCAGGGCGTGCCGATC |
| | | AATACTAATAGTAGCCCAGACGACCAGATAGGCTACTACCGGCGGGCCACCA |
| | | GGCGAATTCGCGGGGGAGACGGGAAGATGAAGGACCTGAGTCCCCGGTGGTA |
| | | CTTCTACTACCTGGGCACCGGGCCAGAGGCCGGCCTGCCATACGGCGGCCGC |
| | | CGGGGCCCAGAGCAGACGCAGGGCAACTTCGGCGACCAAGAGCTGATTCGCC |
| | | AGGGCACGGACTACAAGCACTGGCCTCAGATAGCCCAATTCGCCCCCAGCGC |
| | | GAGCGCGTTTTTCGGGATGAGCAGAATAGGGATGGAGGTGACCCCCAGCGGA |
| | | ACGTGGCTCACCTACACCGGCGCGATCAAGTTGGATGACAAAGACCCAAACT |
| | | TCAAAGACCAGGTCATACTGCTTAACAAACATATCGATGCATACAAAACATT |
| | | CCCGCCGACGGAGCCCAAAAAGGACAAGAAGAGAAGCAAAAACCCGCTGCTG |
| | | TACGACGCCAACTACTTCCTGTGCTGGCACACGAACTGCTACGACTACTGCA |
| | | TCCCCTACAACAGCGTTACCAGCAGCATAGTAATCACCTATGCCTTGGTGTA |
| | | CTTCCTGCAAAGCATCAACTTCGTGAGAATCAGCGGCGTGAAGGACTGCGTA |
| | | GTGCTCCACAGCTACTTCACASCCGACTACTACCAGCTGTACAGCACCCAGC |
| | | TGAGCACCGACACCGGCGTGGAGCACGTAACCTTCTTCATATACAACAAAAT |
| | | CGTCGACGAACCCGAGGAGCACGTACAGCTGGAACAGCCATACGTGTTCATA |
| | | AAACGCTCAGACGCAAGGACCGCCCCCCACGGGAGCGAGAAGAGtTACGAGC |
| | | TGCAAACCCCATTCGAGATCAAGCTGGTCTGCACCAACTACATGCCGTACTT |
| | | CTTCACCCTCCTGCTGCAGCTCATGACGTACCGACGACTGATCAGCATGATG |
| | | GGGTTCAAGATGAACTACCAAGTGAACAGAGAGCAGATCGACGGCTACGTCA |
| | | TGCACGCCAACTACATCTTCTGGAGGAACACA |
| 5′ UTR DNA | 331 | AGAATAAACTAGTATTCTTCTGGTCCCCACAGACTCAGAGAGAACCCGCCAC |
| sequence of ARV- | | C |
| COV-MTE; DNA | | |
| sequence | | |
| 3′ UTR and PolyA; | 332 | CTCGAGCTGGTACTGCATGCACGCAATGCTAGCTGCCCCTTTCCCGTCCTGG |
| DNA sequence of | | GTACCCCGAGTCTCCCCCGACCTCGGGTCCCAGGTATGCTCCCACCTCCACC |
| ARV-COV-MTE; | | TGCCCCACTCACCACCTCTGCTAGTTCCAGACACCTCCCAAGCACGCAGCAA |
| DNA sequence | | TGCAGCTCAAAACGCTTAGCCTAGCCACACCCCCACGGGAAACAGCAGTGAT |
| | | TAACCTTTAGCAATAAACGAAAGTTTAACTAAGCTATACTAACCCCAGGGTT |
| | | GGTCAATTTCGTGCCAGCCACACCCTGGAGCTAGCAaaaaaaaaaaaaaaaa |
| | | aaaaaaaaaaaaagcatatgactaaaaaaaaaaaaaaaaaaaaaaaaaaaaa |
| | | aaaaaaaaaaaaaaaaaaaaaaaaaaaaaaaaaaaaaaaaa |
| SARS-COV-2 | 333 | MSDNGPQNQRNALRITFGGPSDSTGSNQNGGARSKQRRPQGLPNNTASWFTA |
| nucleocapsid | | LTQHGKEDLKFPRGQGVPINTNSSPDDQIGYYRRATRRIRGGDGKMKDLSPR |
| phosphoprotein | | WYFYYLGTGPEAGLPYGANKDGIIWVATEGALNTPKDHIGTRNPANNAAIVL |
| (Omicron B.1.1.529 | | QLPQGTTLPKGFYAEGSRGGSQASSRSSSRSRNSSRNSTPGSSKRTSPARMA |
| Variant; Protein | | GNGGDAALALLLLDRLNQLESKMSGKGQQQQGQTVTKKSAAEASKKPRQKRT |
| sequence | | ATKAYNVTQAFGRRGPEQTQGNFGDQELIRQGTDYKHWPQIAQFAPSASAFF |
| | | GMSRIGMEVTPSGTWLTYTGAIKLDDKDPNFKDQVILLNKHIDAYKTFPPTE |
| | | PKKDKKKKADETQALPQRQKKQQTVTLLPAADLDDFSKQLQQSMSRADSTQA |
| SARS-COV-2 | 334 | ATGAGCGACAACGGCCCCCAGAACCAGAGAAACGCCCTGAGAATCACCTTCG |
| nucleocapsid | | GAGGCCCTAGCGACAGCACCGGCTCTAATCAAAATGGAGGCGCCAGAAGCAA |
| phosphoprotein | | GCAGCGGCGGCCTCAGGGCCTGCCTAACAACACCGCCTCTTGGTTCACCGCT |
| (Omicron B.1.1.529 | | CTTACACAACACGGCAAGGAAGATCTGAAGTTCCCCAGAGGACAGGGTGTTC |
| Variant; DNA | | CAATCAACACCAACAGCTCCCCAGACGATCAGATCGGATATTACAGAAGGGC |
| sequence | | CACTAGACGCATCAGAGGCGGCGACGGCAAGATGAAGGACCTGAGCCCTAGA |
| | | TGGTACTTCTACTACCTGGGCACCGGCCCTGAGGCCGGACTGCCGTACGGCG |
| | | CCAACAAGGACGGCATCATCTGGGTGGCCACAGAGGGCGCCCTGAACACACC |
| | | TAAGGACCACATCGGCACCAGAAATCCTGCCAACAACGCCGCCATCGTGCTG |
| | | CAGCTGCCACAGGGCACCACCCTGCCCAAGGGCTTCTACGCCGAGGGCAGCC |
| | | GGGGCGGCAGCCAGGCCAGCAGCAGATCTTCTTCTCGTTCCCGGAACAGCAG |
| | | CCGGAATTCTACACCTGGCTCCTCCAAGCGGACCAGCCCCGCTAGAATGGCC |
| | | GGCAATGGCGGCGATGCCGCTCTGGCCCTGCTGCTGCTCGATAGACTGAACC |
| | | AGCTGGAAAGCAAGATGAGCGGAAAAGGCCAGCAGCAGCAGGGACAGACAGT |
| | | GACCAAGAAAAGCGCTGCAGAAGCCAGCAAAAAACCTAGACAAAAGAGAACA |
| | | GCCACCAAGGCCTACAACGTGACACAGGCTTTTGGCAGACGGGGCCCCGAGC |
| | | AGACCCAGGGCAACTTCGGCGACCAGGAGCTGATCCGGCAGGGTACAGACTA |
| | | CAAACATTGGCCCCAGATCGCCCAGTTCGCCCCTAGCGCCAGCGCCTTCTTC |
| | | GGAATGTCTAGAATCGGCATGGAAGTGACCCCTTCTGGCACATGGCTGACCT |
| | | ACACCGGCGCTATTAAGCTGGATGACAAGGACCCCAACTTCAAGGACCAAGT |
| | | GATCCTGCTGAACAAGCACATCGACGCCTACAAGACCTTTCCACCTACCGAG |
| | | CCTAAGAAGGATAAGAAAAAGAAAGCTGACGAGACACAGGCCCTGCCTCAAA |
| | | GACAGAAGAAGCAGCAAACCGTGACCCTGCTGCCTGCTGCCGATCTGGACGA |
| | | CTTCAGCAAGCAGCTGCAGCAGAGCATGAGCCGGGCCGACAGCACACAGGCC |
| Exemplary ARV- | 335 | MFVFLVLLPLVSSQCVNLITRTQSYTNSFTRGVYYPDKVFRSSVLHSTQDLF |
| COV-BA2SN+; | | LPFFSNVTWFHAIHVSGTNGTKRFDNPVLPFNDGVYFASTEKSNIIRGWIFG |
| Protein sequence | | TTLDSKTQSLLIVNNATNVVIKVCEFQFCNDPFLDVYYHKNNKSWMESEFRV |
| | | YSSANNCTFEYVSQPFLMDLEFKQGNFKNLREFVFKNIDGYFKIYSKHTPIN |
| | | LGRDLPQGFSALEPLVDLPIGINITRFQTLLALHRSYLTPGDSSSGWTAGAA |
| | | AYYVGYLQPRTFLLKYNENGTITDAVDCALDPLSETKCTLKSFTVEKGIYQT |
| | | SNFRVQPTESIVRFPNITNLCPFDEVFNATRFASVYAWNRKRISNCVADYSV |
| | | LYNFAPFFAFKCYGVSPTKLNDLCFTNVYADSFVIRGNEVSQIAPGQTGNIA |
| | | DYNYKLPDDFTGCVIAWNSNKLDSKVGGNYNYLYRLFRKSNLKPFERDISTE |
| | | IYQAGNKPCNGVAGFNCYFPLRSYGFRPTYGVGHQPYRVVVLSFELLHAPAT |
| | | VCGPKKSTNLVKNKCVNFNFNGLTGTGVLTESNKKFLPFQQFGRDIADTTDA |
| | | VRDPQTLEILDITPCSFGGVSVITPGTNTSNQVAVLYQGVNCTEVPVAIHAD |
| | | QLTPTWRVYSTGSNVFQTRAGCLIGAEYVNNSYECDIPIGAGICASYQTQTK |
| | | SHRRARSVASQSIIAYTMSLGAENSVAYSNNSIAIPTNFTISVTTEILPVSM |
| | | TKTSVDCTMYICGDSTECSNLLLQYGSFCTQLKRALTGIAVEQDKNTQEVFA |
| | | QVKQIYKTPPIKYFGGFNFSQILPDPSKPSKRSFIEDLLFNKVTLADAGFIK |
| | | QYGDCLGDIAARDLICAQKFNGLTVLPPLLTDEMIAQYTSALLAGTITSGWT |
| | | FGAGAALQIPFAMQMAYRFNGIGVTQNVLYENQKLIANQFNSAIGKIQDSLS |
| | | STASALGKLQDVVNHNAQALNTLVKQLSSKFGAISSVLNDILSRLDPPEAEV |
| | | QIDRLITGRLQSLQTYVTQQLIRAAEIRASANLAATKMSECVLGQSKRVDFC |
| | | GKGYHLMSFPQSAPHGVVFLHVTYVPAQEKNFTTAPAICHDGKAHFPREGVE |
| | | VSNGTHWFVTQRNFYEPQIITTDNTFVSGNCDVVIGIVNNTVYDPLQPELDS |
| | | FKEELDKYFKNHTSPDVDLGDISGINASVVNIQKEIDRLNEVAKNLNESLID |
| | | LQELGKYEQYIKWPWYIWLGFIAGLIAIVMVTIMLCCMTSCCSCLKGCCSCG |
| | | SCCKFDEDDSEPVLKGVKLHYTATNFSLLKQAGDVEENPGPMSDNGPQNQRN |
| | | ALRITFGGPSDSTGSNQNGGARSKQRRPQGLPNNTASWFTALTQHGKEDLKF |
| | | PRGQGVPINTNSSPDDQIGYYRRATRRIRGGDGKMKDLSPRWYFYYLGTGPE |
| | | AGLPYGANKDGIIWVATEGALNTPKDHIGTRNPANNAAIVLQLPQGTTLPKG |
| | | FYAEGSRGGSQASSRSSSRSRNSSRNSTPGSSKRTSPARMAGNGGDAALALL |
| | | LLDRLNQLESKMSGKGQQQQGQTVTKKSAAEASKKPRQKRTATKAYNVTQAF |
| | | GRRGPEQTQGNFGDQELIRQGTDYKHWPQIAQFAPSASAFFGMSRIGMEVTP |
| | | SGTWLTYTGAIKLDDKDPNFKDQVILLNKHIDAYKTFPPTEPKKDKKKKADE |
| | | TQALPQRQKKQQTVTLLPAADLDDESKQLQQSMSRADSTQAALVYFLQSINF |
| | | VRIRTRSMWSFNPETNILLNVPL |
| Exemplary ARV- | 336 | ATGTTCGTGTTCCTGGTGCTGCTGCCCCTGGTGAGCAGCCAGTGCGTGAACC |
| COV-BA2SN+; DNA | | TGAtCACCCGGACCCAGagcTACACCAACAGCTTCACCCGGGGCGTCTACTA |
| sequence | | CCCCGACAAGGTGTTCCGGAGCAGCGTCCTGCACAGCACCCAGGACCTGTTC |
| | | CTGCCCTTCTTCAGCAACGTGACCTGGTTCCACGCCATCCACGTGAGCGGCA |
| | | CCAACGGCACCAAGCGGTTCGACAACCCCGTGCTGCCCTTCAACGACGGCGT |
| | | GTACTTCGCCAGCACCGAaAAGAGCAACATCATCCGGGGCTGGATCTTCGGC |
| | | ACCACCCTGGACAGCAAGACCCAGAGCCTGCTGATCGTGAATAACGCCACCA |
| | | ACGTGGTGATCAAGGTGTGCGAGTTCCAGTTCTGCAACGACCCCTTCCTGGa |
| | | CGTGTACTACCACAAGAACAACAAGAGCTGGATGGAGAGCGAGTTCCGGGTG |
| | | TACAGCAGCGCCAACAACTGCACCTTCGAGTACGTGAGCCAGCCCTTCCTGA |
| | | TGGACCTGGAGGGCAAGCAGGGCAACTTCAAGAACCTGCGGGAGTTCGTGTT |
| | | CAAGAACATCGACGGCTACTTCAAGATCTACAGCAAGCACACCCCAATCAAC |
| | | CTGGgcCGGGATCTGCCCCAGGGCTTCTCAGCCCTGGAGCCCCTGGTGGACC |
| | | TGCCCATCGGCATCAACATCACCCGGTTCCAGACCCTGCTGGCCCTGCACCG |
| | | GAGCTACCTGACCCCAGGCGACAGCAGCAGCGGGTGGACAGCAGGCGCGGCT |
| | | GCTTACTACGTGGGCTACCTGCAGCCCCGGACCTTCCTGCTGAAGTACAACG |
| | | AGAACGGCACCATCACCGACGCCGTGGACTGCGCCCTGGACCCTCTGAGCGA |
| | | GACCAAGTGCACCCTCAAGAGCTTCACCGTGGAGAAGGGCATCTACCAGACC |
| | | AGCAACTTCCGGGTGCAGCCCACCGAGAGCATCGTGCGGTTCCCCAACATCA |
| | | CCAACCTGTGCCCCTTCGaCGAGGTGTTCAACGCCACCCGGTTCGCCAGCGT |
| | | GTACGCCTGGAACCGGAAGCGGATCAGCAACTGCGTGGCCGACTACAGCGTG |
| | | CTGTACAACttCGCCCCATTCttCgCCTTCAAGTGCTACGGCGTGAGCCCCA |
| | | CCAAGCTGAACGACCTGTGCTTCACCAACGTGTACGCCGACAGCTTCGTGAT |
| | | CCGTGGCaACGAGGTGagcCAGATCGCACCCGGCCAGACAGGCAAcATCGCC |
| | | GACTACAACTACAAGCTGCCCGACGACTTCACCGGCTGCGTGATCGCCTGGA |
| | | ACAGCAACAAgCTCGACAGCAAGGTGGGCGGCAACTACAACTACCTGTACCG |
| | | GCTGTTCCGaAAGAGCAACCTGAAGCCCTTCGAGCGGGACATCAGCACCGAG |
| | | ATCTACCAAGCCGGCaaCAagCCTTGCAACGGCGTGGccGGCTTCAACTGCT |
| | | ACTTCCCTCTGCgGAGCTACGGCTTCCgGCCCACCtACGGCGTGGGCcACCA |
| | | GCCCTACCGGGTGGTGGTGCTGAGCTTCGAGCTGCTGCACGCCCCAGCCACC |
| | | GTGTGTGGCCCCAAGAAGAGtACCAACCTGGTGAAGAACAAGTGCGTGAACT |
| | | TCAACTTCAACGGCCTTACCGGCACCGGCGTGCTGACCGAGAGCAACAAGAA |
| | | ATTCCTGCCCTTTCAGCAGTTCGGCCGGGACATCGCCGACACCACCGACGCT |
| | | GTGCGGGATCCCCAGACCCTGGAGATCCTGGACATCACCCCTTGCAGCTTCG |
| | | GCGGCGTGAGCGTGATCACCCCAGGCACCAACACCAGCAACCAGGTGGCCGT |
| | | GCTGTACCAGGgCGTGAACTGCACCGAGGTGCCCGTGGCCATCCACGCCGAC |
| | | CAGCTGACACCCACCTGGCGGGTCTACAGCACCGGCAGCAACGTGTTCCAGA |
| | | CCCGGGCCGGTTGCCTGATCGGCGCCGAGtACGTGAACAACAGCTACGAGTG |
| | | CGACATCCCCATCGGCGCCGGCATCTGTGCCAGCTACCAGACCCAGACCAAg |
| | | TCACaCCGGAGGGCAAGGAGCGTGGCCAGCCAGAGCATCATCGCCTACACCA |
| | | TGAGCCTGGGCGCCGAGAACAGCGTGGCCTACAGCAACAACAGCATCGCCAT |
| | | CCCCACCAACTTCACCATCAGCGTGACCACCGAGATTCTGCCCGTGAGCATG |
| | | ACCAAGACCAGCGTGGACTGCACCATGTACATCTGCGGCGACAGCACCGAGT |
| | | GCAGCAACCTGCTGCTGCAGTACGGCAGCTTCTGCACCCAGCTGAAgCGGGC |
| | | CCTGACCGGCATCGCCGTGGAGCAGGACAAGAACACCCAGGAGGTGTTCGCC |
| | | CAGGTGAAGCAGATCTACAAGACCCCTCCCATCAAGtACTTCGGCGGCTTCA |
| | | ACTTCAGCCAGATCCTGCCCGACCCCAGCAAGCCCAGCAAGCGGAGCTTCAT |
| | | CGAGGACCTGCTGTTCAACAAGGTGACCCTAGCCGACGCCGGCTTCATCAAG |
| | | CAGTACGGCGACTGCCTCGGCGACATAGCCGCCCGGGACCTGATCTGCGCCC |
| | | AGAAGTTCAACGGCCTGACCGTGCTGCCTCCCCTGCTGACCGACGAGATGAT |
| | | CGCCCAGTACACCAGCGCCCTGTTAGCCGGAACCATCACCAGCGGCTGGACT |
| | | TTCGGCGCTGGAGCCGCTCTGCAGATCCCCTTCGCCATGCAGATGGCCTACC |
| | | GGTTCAACGGCATCGGCGTGACCCAGAACGTGCTGTACGAGAACCAGAAGCT |
| | | GATCGCCAACCAGTTCAACAGCGCCATCGGCAAGATCCAGGACAGCCTGAGC |
| | | AGCACCGCTAGCGCCCTGGGCAAGCTGCAGGACGTGGTGAACCACAACGCCC |
| | | AGGCCCTGAACACCCTGGTGAAGCAGCTGAGCAGCAAqTTCGGCGCCATCAG |
| | | CAGCGTGCTGAACGACATCCTGAGCCGGCTGGACCCTCCCGAGGCCGAGGTG |
| | | CAGATCGACCGGCTGATCACTGGCCGGCTGCAGAGCCTGCAGACCTACGTGA |
| | | CCCAGCAGCTGATCCGGGCCGCCGAGATTCGGGCCAGCGCCAACCTGGCCGC |
| | | CACCAAGATGAGCGAGTGCGTGCTGGGCCAGAGCAAGCGGGTGGACTTCTGC |
| | | GGCAAGGGCTACCACCTGATGAGCTTTCCCCAGAGCGCACCCCACGGAGTGG |
| | | TGTTCCTGCACGTGACCTACGTGCCCGCCCAGGAGAAGAACTTCACCACCGC |
| | | CCCAGCCATCTGCCACGACGGCAAGGCCCACTTTCCCCGGGAGGGCGTGTTC |
| | | GTGAGCAACGGCACCCACTGGTTCGTGACCCAGCGGAACTTCTACGAGCCCC |
| | | AGATCATCACCACCGACAACACCTTCGTGAGCGGCAACTGCGACGTGGTGAT |
| | | CGGCATCGTGAACAACACCGTGTACGATCCCCTGCAGCCCGAGCTGGACAGC |
| | | TTCAAGGAGGAGCTGGACAAGTACTTCAAGAATCACACCAGCCCCGACGTGG |
| | | ACCTGGGCGACATCAGCGGCATCAACGCCAGCGTGGTGAACATCCAGAAGGA |
| | | GATCGATCGGCTGAACGAGGTGGCCAAGAACCTGAACGAGAGCCTGATCGAC |
| | | CTGCAGGAGCTGGGCAAGTACGAGCAGTACATCAAGTGGCCCTGGTACATCT |
| | | GGCTGGGCTTCATCGCCGGCCTGATCGCCATCGTGATGGTGACCATCATGCT |
| | | GTGCTGCATGACCAGCTGCTGCAGCTGCCTGAAGGGCTGTTGCAGCTGCGGC |
| | | AGCTGCTGCAAGTTCGACGAGGACGACAGCGAGCCCGTGCTGAAGGGCGTGA |
| | | AGCTGCACTACACCGCCACAAACTTTAGCCTGCTGAAGCAGGCCGGGGATGT |
| | | CGAGGAAAACCCCGGCCCTATGAGCGACAACGGCCCCCAGAACCAGAGAAAC |
| | | GCCCTGAGAATCACCTTCGGAGGCCCTAGCGACAGCACCGGCTCTAATCAAA |
| | | ATGGAGGCGCCAGAAGCAAGCAGCGGCGGCCTCAGGGCCTGCCTAACAACAC |
| | | CGCCTCTTGGTTCACCGCTCTTACACAACACGGCAAGGAAGATCTGAAGTTC |
| | | CCCAGAGGACAGGGTGTTCCAATCAACACCAACAGCTCCCCAGACGATCAGA |
| | | TCGGATATTACAGAAGGGCCACTAGACGCATCAGAGGCGGCGACGGCAAGAT |
| | | GAAGGACCTGAGCCCTAGATGGTACTTCTACTACCTGGGCACCGGCCCTGAG |
| | | GCCGGACTGCCGTACGGCGCCAACAAGGACGGCATCATCTGGGTGGCCACAG |
| | | AGGGCGCCCTGAACACACCTAAGGACCACATCGGCACCAGAAATCCTGCCAA |
| | | CAACGCCGCCATCGTGCTGCAGCTGCCACAGGGCACCACCCTGCCCAAGGGC |
| | | TTCTACGCCGAGGGCAGCCGGGGCGGCAGCCAGGCCAGCAGCAGATCTTCTT |
| | | CTCGTTCCCGGAACAGCAGCCGGAATTCTACACCTGGCTCCTCCAAGCGGAC |
| | | CAGCCCCGCTAGAATGGCCGGCAATGGCGGCGATGCCGCTCTGGCCCTGCTG |
| | | CTGCTCGATAGACTGAACCAGCTGGAAAGCAAGATGAGCGGAAAAGGCCAGC |
| | | AGCAGCAGGGACAGACAGTGACCAAGAAAAGCGCTGCAGAAGCCAGCAAAAA |
| | | ACCTAGACAAAAGAGAACAGCCACCAAGGCCTACAACGTGACACAGGCTTTT |
| | | GGCAGACGGGGCCCCGAGCAGACCCAGGGCAACTTCGGCGACCAGGAGCTGA |
| | | TCCGGCAGGGTACAGACTACAAACATTGGCCCCAGATCGCCCAGTTCGCCCC |
| | | TAGCGCCAGCGCCTTCTTCGGAATGTCTAGAATCGGCATGGAAGTGACCCCT |
| | | TCTGGCACATGGCTGACCTACACCGGCGCTATTAAGCTGGATGACAAGGACC |
| | | CCAACTTCAAGGACCAAGTGASCCTGCTGAACAAGCACATCGACGCCTACAA |
| | | GACCTTTCCACCTACCGAGCCTAAGAAGGATAAGAAAAAGAAAGCTGACGAG |
| | | ACACAGGCCCTGCCTCAAAGACAGAAGAAGCAGCAAACCGTGACCCTGCTGC |
| | | CTGCTGCCGATCTGGACGACTTCAGCAAGCAGCTGCAGCAGAGCATGAGCCG |
| | | GGCCGACAGCACACAGGCCGCCTTGGTGTACTTCCTGCAAAGCATCAACTTC |
| | | GTGAGAATCCGGACGCGGAGCATGTGGAGCTTTAACCCCGAGACCAACATCC |
| | | TGCTGAACGTCCCCCTG |
| Exemplary P2A | 337 | ATNFSLLKQAGDVEENPGP |
| Protein Sequence | | |
| Exemplary P2ADNA | 338 | GCCACAAACTTTAGCCTGCTGAAGCAGGCCGGGGATGTCGAGGAAAACCCCG |
| Sequence | | GCCCT |
| ARV-HSV-mRNA-1 | 339 | MGPGLWVVMGVLVGVAGGHDTYWTEQIDPWFIHGLGLARTYWRDTNTGRLWL |
| (HSV-2 glycoprotein | | PNTPDASDPQRGRLAPPGELNLTTASVPMLRWYAERFCFVLVTTAEFPRDPG |
| H and glycoprotein | | QLLYIPKTYLLGRPRNASLPELPEAGPTSRPPAEVTQLKGLSHNPGASALLR |
| L); Protein sequence | | SRAWVTFAAAPDREGLTFPRGDDGATERHPDGRRNAPPPGPPAGAPRHPTTN |
| | | LSAIHLHNASVTWLAARGLLRTPGRYVYLSPSASTWPVGVWTTGGLAFGCDA |
| | | ALVRARYGKGFMGLVISMRDSPPAEIIVVPADKTLARVGNPTDENAPAVLPG |
| | | PPAGPRYRVFVLGAPTPADNGSALDALRRVAGYPEESTNYAQYMSRAYAEFL |
| | | GEDPGSGTDARPSLFWRLAGLLASSGFAFVNAAHAHDAIRLSQLLGFLAHSR |
| | | VLAGLAARGAAGCAADSVFLNVSVLDPAARLRLEARLGHLVAAILEREQSLA |
| | | AHALGYQLAFVLDSPAAYGAVAPSAARLIDALYAEFLGGRALTAPMVRRALF |
| | | YATAVLRAPFLAGAPSAEQRERARRGLLITTALCTSDVAAATHADLRAALAR |
| | | TDHQKNLFWLPDHFSPCAASLRFDLAEGGFILDALAMATRSDIPADVMAQQT |
| | | RGVASALTRWAHYNALIRAFVPEATHQCSGPSHNAEPRILVPITHNASYVVT |
| | | HTPLPRGIGYKLTGVDVRRPLFITYLTATCEGHAREIEPKRLVRTENRRDLG |
| | | LVGAVFLRYTPAGEVMSVLLVDTDATQQQLACGPVAGTPNVFSSDVPSVALL |
| | | LFPNGTVIHLLAFDTLPIATIAPGFLAASALGVVMITAALAGILRVVRTCVP |
| | | FLWRREATNFSLLKQAGDVEENPGPMGFVCLFGLVVMGAWGAWGGSQATEYV |
| | | LRSVIAKEVGDILRVPCMRTPADDVSWRYEAPSVIDYARIDGIFLRYHCPGL |
| | | DTFLWDRHAQRAYLVNPFLFAAGFLEDLSHSVEPADTQETTTRRALYKEIRD |
| | | ALGSRKQAVSHAPVRAGCVNFDYSRTRRCVGRRDLRPANTTSTWEPPVSSDD |
| | | EASSQSKPLATQPPVLALSNAPPRRVSPTRGRRRHTRLRRN |
| ARV-HSV-mRNA-1 | 340 | atgggccccggtctgtgggtggtgatgggcgtactggtaggcgttgccgggg |
| (HSV-2 glycoprotein | | gccatgacacgtactggacagagcaaatcgacccgtggttcttgcacggtct |
| H and glycoprotein | | ggggttggcccgcacgtactggcgcgacacaaacaccgggaggctgtggttg |
| L); DNA sequence | | cccaacacccccgacgccagcgacccccagcgcggacgcttggcgcccccgg |
| | | gcgaactcaacctgacaacggcaagcgtgcccatgcttcggtggtacgccga |
| | | gcgcttttgtttcgtgttggtcaccacggccgagtttccacgggaccccggg |
| | | cagctgctttacatcccaaagacctatctgctcggccggccacggaacgcga |
| | | gcctgcccgagcttcccgaggcggggcccacgtccaggccccccgccgaggt |
| | | gacccagctcaagggactgagccacaaccccggcgcctccgcgctgttgagg |
| | | tcccgggcctgggtaacattcgcggccgcgccagaccgcgaggggcttacgt |
| | | tcccgcggggagacgacggggcgaccgagaggcacccggacggccgacgcaa |
| | | cgcaccgcccccggggccgcccgcgggagcgccgaggcatccgacgacgaac |
| | | ctgagcattgcgcatctgcacaacgcgtccgtgacatggctggccgccaggg |
| | | gcctgctacggacaccgggtcggtacgtgtacctctccccgagcgcctcgac |
| | | gtggcccgtgggagtctggacgacgggcgggctggcgttcgggtgcgatgcc |
| | | gcgcttgtgcgcgcgcgatacgggaagggcttcatggggctcgtgatttcca |
| | | tgcgggacagcccaccggccgagatcattgtggtgccagcggacaagaccct |
| | | cgcacgggtcggaaatccgacagacgaaaacgcccccgcggtgctcccaggg |
| | | ccaccagccggccccaggtatcgcgtcttcgtcctgggggccccgacgccag |
| | | ccgacaatggcagcgcgctggacgcactacggcgggtggccggctaccccga |
| | | ggagagcacgaactacgcacagtatatgagccgggcctatgcggagtttcta |
| | | ggggaggacccgggaagcggcacggacgcgaggccgagcctgttctggcgcc |
| | | tcgcagggctgctcgccagcagcgggtttgcgttcgtcaacgcggcacacgc |
| | | ccacgacgcgattcgcctcagcgacctgctgggctttctagcccacagccgc |
| | | gtgctggccggcctggccgcccggggagcagcgggctgtgcggccgacagcg |
| | | tgttcctgaacgtgagcgtgttggacccggcggccaggctgcggcttgaggc |
| | | gcgcctcggacatctggtggcagcgatcctcgagcgagagcagagcctagcg |
| | | gcgcacgcgctgggctatcagctggcgttcgtgttggacagccccgcggcct |
| | | atggcgcggtggccccgagcgcggcccgcctgatcgacgcactgtacgccga |
| | | gtttctcggcggccgcgcgctaaccgccccgatggtccgccgagcgctgttt |
| | | tacgcaacggccgtcctccgggcaccgttcctggcaggcgcgcccagcgccg |
| | | agcagcgggaacgcgcccgccggggactcctcataaccacggcactgtgtac |
| | | gagcgacgtagcagcggcgacccacgccgatctccgggccgcgctcgccagg |
| | | accgaccaccagaaaaacctcttctggctcccggaccacttttccccatgcg |
| | | cagcatccctgcgcttcgatctcgccgaaggagggttcatcctggacgcact |
| | | ggccatggccacccgatccgacatcccggcggacgtcatggcacaacagacc |
| | | cgcggcgtggcctccgcacttacgcgctgggcgcactacaacgccctgatcc |
| | | gcgccttcgtcccggaggccacccatcagtgtagcggcccgagccacaacgc |
| | | ggagccccggatcctcgtacccatcacccacaacgccagctatgtcgtcaca |
| | | cacacccccttgccacgcgggatcggatacaagctcacgggcgttgacgtcc |
| | | gccgcccactgtttatcacctatctcaccgccacctgcgaagggcacgcgcg |
| | | ggagattgagccgaaacggctggtgcgcaccgaaaaccggcgcgaccttggc |
| | | ctcgtgggggccgtgtttctgcgctacaccccggccggggaggtcatgagcg |
| | | tgctgctagtggacacggatgccacccaacagcagctggcccaggggccggt |
| | | ggcgggcaccccgaacgtgttttccagcgatgtaccaagcgtggccctgtta |
| | | ttgttccccaatggaacagtgattcatctgctggcctttgacacgctgccca |
| | | tcgccaccatcgcccccgggttcctggccgcgtccgcgcttggagtcgttat |
| | | gattaccgcggcactggcaggcatcctcagggtggtccgaacgtgcgtccca |
| | | tttttgtggagacgcgaaGCCACGAACTTCTCACTATTAAAGCAAGCAGGAG |
| | | ACGTGGAAGAAAACCCCGGTCCAatggggttcgtctgtctgtttgggcttgt |
| | | cgtaatgggagcctggggggcgtggggaggatcacaggcaaccgaatatgtt |
| | | cttaggagtgttattgccaaagaggtgggggacatactaagagtaccatgca |
| | | tgcggacccccgcggatgatgtttcatggcgctacgaggccccgagcgttat |
| | | tgactatgccaggatagacggaatatttcttcgctatcactgcccggggctt |
| | | gacacgttcttgtgggataggcacgcccagagggcatatctggttaatccct |
| | | ttctctttgcggcgggatttttggaggacttgagtcactcagtgtttccggc |
| | | cgacacacaggaaacaacaacgcgcagggccctttataaagagatacgcgat |
| | | gcgttgggcagtcgaaaacaggccgtcagccacgcacccgtcagggccgggt |
| | | gtgtaaacttcgactactcaaggacaaggcgctgcgtcgggcgacgcgattt |
| | | acggccagccaacaccacgtcaacgtgggaaccaccagtgagcagcgacgat |
| | | gaagcaagctcgcagagcaagcccctcgccacccagccgcccgtcctcgccc |
| | | tttcgaacgcacccccacgggggtctccccgacgcgaggacggcgccggca |
| | | tacacgcctccgacgcaat |
| ARV-HSV-mRNA-2 | 341 | MGRLTSGVGTAALLVVAVGLRVVCAKYALADPSLKMADPNRFRGKNLPVLDQ |
| (HSV-2 glycoprotein | | LTDPPGVKRVYHIQPSLEDPFQPPSIPITVYYAVLERACRSVLLHAPSEAPQ |
| D and glycoprotein | | IVRGASDEARKHTYNLTIAWYRMGDNCAIPITVMEYTECPYNKSLGVCPIRT |
| B); Protein sequence | | QPRWSYYDSFSAVSEDNLGFLMHAPAFETAGTYLRLVKINDWTEITQFILEH |
| | | RARASCCYALPLRIPPAACLISKAYQQGVTVDSIGMLPRFIPENQRIVALYS |
| | | LKIAGWHGPKPPYTSILLPPELSDITNATQPELVPEDPEDSCLLEDPAGTVS |
| | | SQIPPNWHIPSIQDVAPHHAPAAPSNPGLIIGALAGSTLAVLVIGGIAFWVR |
| | | RRAQMAPKRLRLPHIRDDDAPPSHQPLFYATNFSLLKQAGDVEENPGPMRGG |
| | | GLICALVVGALVAAVASAAPAAPRASGGVAATVAANGGPASRPPPVPSPATT |
| | | RARKRKTKKPPERPEATPPPDANATVAAGHATLRAHLREIKVENADAQFYVC |
| | | PPPTGATVVQFEQPRRCPTRPEGQNYTEGIAVVFKENIAPYKFKATMYYKDV |
| | | TVSQVWFGHRYSQFMGTFEDRAPVPFFEVTDKTNAKGVCRSTAKYVRNNMFT |
| | | TAFHRDDHETDMELKPAKVATRTSRGWHTTDLKYNPSRVEAFHRYGTTVNCI |
| | | VEEVDARSVYPYDEFVLATGDFVYMSPFYGYREGSHTEHTSYAADRFKQVDG |
| | | FYARDLTTKARATSPTTRNLLTTPKFTVAWDWVPKRPAVCTMTKWQEVDEML |
| | | RAEYGGSFRFSSDAISTTFTTNLTQYSLSRVDLGDCIGRDAREAIDRMFARK |
| | | YNATHIKVGQPQYYLATGGFLIAYQPLLSNTLAELYVREYMREQDRKPRNAT |
| | | PAPLREAPSANASVERIKTTSSIEFARLQFTYNHIQRPVNDMLGRIAVAWCE |
| | | LQNHELTLWNEARKLNPNAIASATVGRRVSARMLGDVMAVSTCVPVAPDNVI |
| | | VQNSMRVSSRPGTCYSRPLVSFRYEDQGPLIEGQLGENNELRLTRDALEPCT |
| | | VGHRRYFIFGGGYVYFEEYAYSHQLSRADVITVSTFIDLNITMLEDHEFVPL |
| | | EVYTRHEIKDSGLLDYTEVQRRNQLHDLRFAAIDTVIRADANAAMFAGLCAF |
| | | FEGMGDLGRAVGKVVMGVVGGVVSAVSGVSSFMSNPFGALAVGLLVLAGLVA |
| | | AFFAFRYVLQLQRNPMKALYPLTTKELKTSDPGGVGGEGEEGAEGGGFDEAK |
| | | LAEAREMIRYMALVSAMERTEHKARKKGTSALLSSKVTNMVLRKRNKARYSP |
| | | LHNEDEAGDEDEL |
| ARV-HSV-mRNA-2 | 342 | atggggcgattgacctccggcgtcgggacggcggccctgctagtagtcgcgg |
| (HSV-2 glycoprotein | | tgggactccgcgtcgtctgcgccaaatacgccttagcagaccccagccttaa |
| D and glycoprotein | | gatggccgatcccaatcgatttcgcgggaagaaccttccggtattggaccag |
| B); DNA sequence | | ctgaccgacccccccggggtgaagcgagtataccacatacagccgagcctgg |
| | | aggacccgttccagccccccagcatcccgatcacagtgtactacgcagtgct |
| | | ggaacgagcctgccgcagcgtgctcctacatgccccaagcgaggccccccag |
| | | atcgtgcgcggggcatccgacgaggcccgaaagcacacgtacaacctgacca |
| | | tcgcctggtatcgcatgggagacaattgcgcaatccccatcacggtaatgga |
| | | atacaccgagtgcccctacaacaagagcttgggggtctgccccatccgaacg |
| | | cagccccgctggagctactatgacagctttagcgccgtcagcgaggataacc |
| | | tgggattcctgatgcacgcccccgccttcgagaccgcgggaacgtacctgcg |
| | | gctagtgaagataaacgactggacggagatcacacaatttatcctggagcac |
| | | cgggcccgcgcctcctgctgctacgcactccccctgcgcatccccccggcag |
| | | cgtgcctcaccagcaaggcctaccaacagggcgtgacggtcgacagcatcgg |
| | | gatgctaccccgctttatccccgaaaaccagcgcaccgtcgccctatacagc |
| | | ttaaaaatcgccggctggcacggccccaagcccccgtacaccagcaccctgc |
| | | tgccgccggagctttccgacaccaccaacgccacgcaacccgaactcgtacc |
| | | ggaagaccccgaggacagctgcctcttagaggatcccgccgggacggtgtca |
| | | tcacagatccccccaaactggcacatcccgtcgatccaggacgtcgcgccgc |
| | | accacgcccccgccgcccccagcaacccgggcctgatcatcggcgcgctggc |
| | | cggcagtaccctggcggtgctggtcatcggcggaatagcgttttgggtacgc |
| | | cgccgcgcacagatggcccccaagcgcctacgactcccccacatccgggatg |
| | | acgacgcgccccccagccaccagccattgttttacGCCACGAACTTCTCACT |
| | | GTTAAAGCAAGCAGGAGACGTGGAAGAAAACCCCGGACCAatgcgcgggggg |
| | | ggcttgatatgcgcgctggtcgtgggggcgctggtggccgcggtggcgtcgg |
| | | cggccccggcggccccccgcgccagcggcggcgtggccgcgaccgtcgcggc |
| | | gaacgggggacccgcctcccggccgccccccgtcccgagccccgcgaccacc |
| | | agggcccggaagcggaaaaccaaaaagccgcccgagcggcccgaggcgaccc |
| | | cgccccccgacgccaacgcgaccgtcgccgccggccacgccacgctgcgcgc |
| | | gcacctgcgggaaatcaaggtcgagaacgccgatgcccagttttacgtgtgc |
| | | ccacccccgacaggcgccacggtggtgcagtttgagcagccgcgccgctgcc |
| | | cgacgcgcccggaggggcagaactacacggagggcatcgcggtggtcttcaa |
| | | ggagaacatcgccccgtacaaattcaaggccaccatgtactacaaagacgtg |
| | | accgtgtcgcaggtgtggttcggccaccgctactcccagtttatggggatat |
| | | tcgaggaccgcgcccccgtacccttcgaggaggtgatcgacaagataaacgc |
| | | caagggggtctgccgctccacggccaagtacgtgcggaacaacatggagacc |
| | | accgcgtttcaccgggacgaccacgagaccgacatggagctcaagccggcga |
| | | aggtcgccacgcgcacgagccgggggtggcacaccaccgacctcaagtacaa |
| | | ccccagccgggtggaggcgttccatcggtacggcacgacggtcaactgcatc |
| | | gtcgaggaggtggacgcgcggtcggtgtacccgtacgatgagtttgtgctgg |
| | | cgacgggcgactttgtgtacatgtccccgttttacggctaccgggaggggag |
| | | ccacaccgagcacaccagctacgccgccgaccgcttcaagcaggtcgacggc |
| | | ttctacgcgcgcgacctcaccacgaaggcccgggccacgagcccgacgaccc |
| | | gcaacttgctgacgacccccaagtttaccgtggcctgggactgggtgccgaa |
| | | gcgaccggcggtctgcaccatgaccaagtggcaggaggtggacgagatgctc |
| | | cgcgccgagtacggcggctccttccgcttctcctccgacgccatcagcacca |
| | | ccttcaccaccaacctgacccagtacagcctcagccgcgtcgacctgggcga |
| | | ctgcatcggccgggatgcccgcgaggccatcgaccgcatgtttgcgcgcaag |
| | | tacaacgccacgcacatcaaggtgggccagccgcagtactacctggccacgg |
| | | ggggcttcctcatcgcgtaccagcccctcctcagcaacacgctcgccgagct |
| | | gtacgtgcgggagtacatgcgggagcaggaccgcaagccccggaatgccacg |
| | | cccgcgccactgcgggaggcgcccagcgccaacgcgagcgtggagcgcatca |
| | | agaccaccagcagcatcgagttcgcccggctgcagtttacgtataaccatat |
| | | acagcgccccgtgaacgacatgctggggcgcatcgccgtcgcgtggtgcgag |
| | | ctgcagaaccacgagctgacactctggaacgaggcccgcaagctcaacccca |
| | | acgccatcgccagcgccaccgtcggccggcgggtgagcgcgcgcatgctcgg |
| | | agacgtcatggccgtctccacgtgcgtgcccgtcgccccggacaacgtgatc |
| | | gtgcagaacagcatgcgcgtcagcagccggccggggacgtgctacagccgcc |
| | | ccctggtcagctttcggtacgaagaccagggcccgctgatcgaggggcagct |
| | | gggcgagaacaacgagctgcgcctcacccgcgacgcgctcgagccgtgcacc |
| | | gtgggccaccggcgctacttcatcttcggcgggggctacgtatacttcgagg |
| | | agtacgcgtactcacaccagctgagtcgcgccgacgtcaccaccgtcagcac |
| | | cttcatcgacctgaacatcaccatgctggaggaccacgagtttgtgcccctg |
| | | gaggtctacacgcgccacgagatcaaggacagcggcctgctggactacacgg |
| | | aggtccagcgccgcaaccagctgcacgacctgcgctttgccgccatcgacac |
| | | ggtcatccgcgccgacgccaacgccgccatgttcgcggggctgtgcgcgttc |
| | | ttcgaggggatgggggacttggggcgcgcggtcggcaaggtcgtcatgggag |
| | | tagtggggggcgtggtgagcgccgtcagcggcgtgagctcctttatgtccaa |
| | | ccccttcggggcgcttgccgtggggctactggtcctggccggcctggtcgcg |
| | | gccttcttcgccttccgctacgtcctgcaactgcaacgcaatcccatgaagg |
| | | ccctgtatccgctcaccaccaaggaactcaagacatccgaccccgggggcgt |
| | | gggcggggagggggaggaaggcgcggaggggggcgggtttgacgaggccaag |
| | | ttggccgaggcccgagaaatgatccgatatatggcattggtgagcgccatgg |
| | | agcgcacggaacacaaggccagaaagaagggcacgagcgccctgctcagctc |
| | | caaggtcaccaacatggtactgcgcaagcgcaacaaagccaggtactcaccg |
| | | ctccacaacgaggacgaggccggagacgaagacgagctc |
| ARV-HSV-gL (HSV- | 603 | MGFVCLFGLVVMGAWGAWGGSQATEYVLRSVIAKEVGDILRVPCMRTPADDV |
| 2 glycoprotcin L), | | SWRYEAPSVIDYARIDGIFLRYHCPGLDTELWDRHAQRAYLVNPFLFAAGFL |
| Protein sequence. | | EDLSHSVFPADTQETTTRRALYKEIRDALGSRKQAVSHAPVRAGCVNFDYSR |
| | | TRRCVGRRDLRPANTTSTWEPPVSSDDEASSQSKPLATQPPVLALSNAPPRR |
| | | VSPTRGRRRIITRLRRN* |
| ARV-HSV-gL (HSV- | 604 | GAATTCGGCTTTAATACGACTCACTATAAGGACATTAGCTTCTGACACAACT |
| 2 glycoprotein L), | | GTGTTCACTAGCAACCTCAAACAGCCACCatggggttcgtctgtctgtttgg |
| DNA sequence. | | gcttgtcgtaatgggagcctggggggcgtggggaggatcacaggcaaccgaa |
| | | tatgttcttaggagtgttattgccaaagaggtgggggacatactaagagtac |
| | | catgcatgcggacccccgcggatgatgtttcatggcgctacgaggccccgag |
| | | cgttattgactatgccaggatagacggaatatttcttcgctatcactgcccg |
| | | gggcttgacacgttcttgtgggataggcacgcccagagggcatatctggtta |
| | | atccctttctctttgcggcgggatttttggaggacttgagtcactcagtgtt |
| | | tccggccgacacacaggaaacaacaacgcgcagggccctttataaagagata |
| | | cgcgatgcgttgggcagtcgaaaacaggccgtcagccacgcacccgtcaggg |
| | | ccgggtgtgtaaacttcgactactcaaggacaaggcgctgcgtcgggcgacg |
| | | cgatttacggccagccaacaccacgtcaacgtgggaaccaccagtgagcagc |
| | | gacgatgaagcaagctcgcagagcaagcccctcgccacccagccgcccgtcc |
| | | tcgccctttcgaacgcacccccacggcgggtctccccgacgcgaggacggcg |
| | | ccggcatacacgcctccgacgcaattgataaCGTACGGCTGGAGCCTCGGTG |
| | | GCCTAGCTTCTTGCCCCTTGGGCCTCCCCCCAGCCCCTCCTCCCCTTCCTGC |
| | | ACCCGTACCCCCGTGGTCTTTGAATAAAGTCTGAGTGGGCGGCAaaaaaaaa |
| | | aaaaaaaaaaaaaaaaaaaaagcatatgactaaaaaaaaaaaaaaaaaaaaa |
| | | aaaaaaaaaaaaaaaaaaaaaaaaaaaaaaaaaaaaaaaaaaaaaaaaaAAA |
| | | AGAAGAGCAAGCTT |
| ARV-HSV-gB-WT | 605 | MRGGGLICALVVGALVAAVASAAPAAPRASGGVAATVAANGGPASRPPPVPS |
| (HSV-2 glycoprotein | | PATTRARKRKTKKPPERPEATPPPDANATVAAGHATLRAHLREIKVENADAQ |
| B wildtype), Protein | | FYVCPPPTGATVVQFEQPRRCPTRPEGQNYTEGIAVVFKENIAPYKFKATMY |
| sequence | | YKDVTVSQVWFGHRYSQFMGIFEDRAPVPFEEVIDKINAKGVCRSTAKYVRN |
| | | NMETTAFHRDDHETDMELKPAKVATRTSRGWHTTDLKYNPSRVEAFHRYGTT |
| | | VNCIVEEVDARSVYPYDEFVLATGDFVYMSPFYGYREGSHTEHTSYAADRFK |
| | | QVDGFYARDLTTKARATSPTTRNLLTTPKFTVAWDWVPKRPAVCTMTKWQEV |
| | | DEMLRAEYGGSFRFSSDAISTTFTTNLTQYSLSRVDLGDCIGRDAREAIDRM |
| | | FARKYNATHIKVGQPQYYLATGGFLIAYQPLLSNTLAELYVREYMREQDRKP |
| | | RNATPAPLREAPSANASVERIKTTSSIEFARLQFTYNHIQRHVNDMLGRIAV |
| | | AWCELQNHELTLWNEARKLNPNAIASATVGRRVSARMLGDVMAVSTCVPVAP |
| | | DNVIVQNSMRVSSRPGTCYSRPLVSFRYEDQGPLIEGQLGENNELRLTRDAL |
| | | EPCTVGHRRYFIFGGGYVYFEEYAYSHQLSRADVTTVSTFIDLNITMLEDHE |
| | | FVPLEVYTRHEIKDSGLLDYTEVQRRNQLHDLRFADIDTVIRADANAAMFAG |
| | | LCAFFEGMGDLGRAVGKVVMGVVGGVVSAVSGVSSFMSNPFGALAVGLLVLA |
| | | GLVAAFFAFRYVLQLQRNPMKALYPLTTKELKTSDPGGVGGEGEEGAEGGGF |
| | | DEAKLAEAREMIRYMALVSAMERTEHKARKKGTSALLSSKVTNMVLRKRNKA |
| | | RYSPLHNEDEAGDEDE* |
| ARV-HSV-gB-WT | 606 | GAATTCGGCTTTAATACGACTCACTATAAGGACATTAGCTTCTGACACAACT |
| (HSV-2 glycoprotein | | GTGTTCACTAGCAACCTCAAACAGCCACCatgcgcggggggggcttgatatg |
| B wildtype), DNA | | cgcgctggtcgtgggggcgctggtggccgcggtggcgtcggcggccccggcg |
| sequence | | gccccccgcgccagcggcggcgtggccgcgaccgtcgcggcgaacgggggac |
| | | ccgcctcccggccgccccccgtcccgagccccgcgaccaccagggcccggaa |
| | | gcggaaaaccaaaaagccgcccgagcggcccgaggcgaccccgccccccgac |
| | | gccaacgcgaccgtcgccgccggccacgccacgctgcgcgcgcacctgcggg |
| | | aaatcaaggtcgagaacgccgatgcccagttttacgtgtgcccacccccgac |
| | | aggcgccacggtggtgcagtttgagcagccgcgccgctgcccgacgcgcccg |
| | | gaggggcagaactacacggagggcatcgcggtggtcttcaaggagaacatcg |
| | | ccccgtacaaattcaaggccaccatgtactacaaagacgtgaccgtgtcgca |
| | | ggtgtggttcggccaccgctactcccagtttatggggatattcgaggaccgc |
| | | gcccccgtacccttcgaggaggtgatcgacaagataaacgccaagggggtct |
| | | gccgctccacggccaagtacgtgcggaacaacatggagaccaccgcgtttca |
| | | ccgggacgaccacgagaccgacatggagctcaagccggcgaaggtcgccacg |
| | | cgcacgagccgggggtggcacaccaccgacctcaagtacaaccccagccggg |
| | | tggaggcgttccatcggtacggcacgacggtcaactgcatcgtcgaggaggt |
| | | ggacgcgcggtcggtgtacccgTATgatgagtttgtgctggcgacgggcgac |
| | | tttgtgtacatgtccccgttttacggctaccgggaggggagccacaccgagc |
| | | acaccagctacgccgccgaccgcttcaagcaggtcgacggcttctacgcgcg |
| | | cgacctcaccacgaaggcccgggccacgagcccgacgacccgcaacttgctg |
| | | acgacccccaagtttaccgtggcctgggactgggtgccgaagcgaccggcgg |
| | | tctgcaccatgaccaagtggcaggaggtggacgagatgctccgcgccgagta |
| | | cggcggctccttccgcttctcctccgacgccatcagcaccaccttcaccacc |
| | | aacctgacccagtacagcctcagccgcgtcgacctgggcgactgcatcggcc |
| | | gggatgcccgcgaggccatcgaccgcatgtttgcgcgcaagtacaacgccac |
| | | gcacatcaaggtgggccagccgcagtactacctggccacggggggcttcctc |
| | | atcgcgtaccagcccctcctcagcaacacgctcgccgagctgtacgtgcggg |
| | | agtacatgcgggagcaggaccgcaagccccggaatgccacgcccgcgccact |
| | | gcgggaggcgcccagcgccaacgcgagcgtggagcgcatcaagaccaccagc |
| | | agcatcgagttcgcccggctgcagtttacgtataaccatatacagcgcCACg |
| | | tgaacgacatgctggggcgcatcgccgtcgcgtggtgcgagctgcagaacca |
| | | cgagctgacactctggaacgaggcccgcaagctcaaccccaacgccatcgcc |
| | | agcgccaccgtcggccggcgggtgagcgcgcgcatgctcggagacgtcatgg |
| | | ccgtctccacgtgcgtgcccgtcgccccggacaacgtgatcgtgcagaacag |
| | | catgcgcgtcagcagccggccggggacgtgctacagccgccccctggtcagc |
| | | tttcggtacgaagaccagggcccgctgatcgaggggcagctgggcgagaaca |
| | | acgagctgcgcctcacccgcgacgcgctcgagccgtgcaccgtgggccaccg |
| | | gcgctacttcatcttcggcgggggctacgtatacttcgaggagtacgcgtac |
| | | tcacaccagctgagtcgcgccgacgtcaccaccgtcagcaccttcatcgacc |
| | | tgaacatcaccatgctggaggaccacgagtttgtgcccctggaggtctacac |
| | | gcgccacgagatcaaggacagcggcctgctggactacacggaggtccagcgc |
| | | cgcaaccagctgcacgacctgcgctttgccgacatcgacacggtcatccgcg |
| | | ccgacgccaacgccgccatgttcgcggggctgtgcgcgttcttcgaggggat |
| | | gggggacttggggcgcgcggtcggcaaggtcgtcatgggagtagtggggggc |
| | | gtggtgagcgccgtcagcggcgtgagctcctttatgtccaaccccttcgggg |
| | | cgcttgccgtggggctactggtcctggccggcctggtcgcggccttcttcgc |
| | | cttccgctacgtcctgcaactgcaacgcaatcccatgaaggccctgtatccg |
| | | ctcaccaccaaggaactcaagacatccgaccccgggggcgtgggcggggagg |
| | | gggaggaaggcgcggaggggggcgggtttgacgaggccaagttggccgaggc |
| | | ccgagaaatgatccgatatatggcattggtgagcGCGatggagcgcacggaa |
| | | cacaaggccagaaagaagggcacgagcgccctgctcagctccaaggtcacca |
| | | acatggtactgcgcaagcgcaacaaagccaggtactcaccgctccacaacga |
| | | ggacgaggccggagacgaagacgagctctgaCGTACGGCTGGAGCCTCGGTG |
| | | GCCTAGCTTCTTGCCCCTTGGGCCTCCCCCCAGCCCCTCCTCCCCTTCCTGC |
| | | ACCCGTACCCCCGTGGTCTTTGAATAAAGTCTGAGTGGGCGGCAaaaaaaaa |
| | | aaaaaaaaaaaaaaaaaaaaagcatatgactaaaaaaaaaaaaaaaaaaaaa |
| | | aaaaaaaaaaaaaaaaaaaaaaaaaaaaaaaaaaaaaaaaaaaaaaaaaAAA |
| | | AGAAGAGCAAGCTT |
| ARV-HSV-gB (HSV- | 607 | MRGGGLICALVVGALVAAVASAAPAAPRASGGVAATVAANGGPASRPPPVPS |
| 2 glycoprotein B), | | PATTRARKRKTKKPPERPEATPPPDANATVAAGHATLRAHLREIKVENADAQ |
| Protein sequence | | FYVCPPPTGATVVQFEQPRRCPTRPEGQNYTEGIAVVFKENIAPYKFKATMY |
| | | YKDVTVSQVWFGHRYSQFMGIFEDRAPVPFEEVIDKINAKGVCRSTAKYVRN |
| | | NMETTAFHRDDHETDMELKPAKVATRTSRGWHTTDLKYNPSRVEAFHRYGTT |
| | | VNCIVEEVDARSVYPYDEFVLATGDFVYMSPFYGYREGSHTEHTSYAADRFK |
| | | QVDGFYARDLITKARATSPTTRNLLTTPKFTVAWDWVPKRPAVCTMTKWQEV |
| | | DEMLRAEYGGSFRFSSDAISTTFTTNLTQYSLSRVDLGDCIGRDAREAIDRM |
| | | FARKYNATHTKVGQPQYYLATGGFTTAYQPTTSNTLAFTYVREYMREQDRKP |
| | | RNATPAPLREAPSANASVERIKTTSSIEFARLQFTYNHIQRPVNDMLGRIAV |
| | | AWCELQNHELTLWNEARKLNPNAIASATVGRRVSARMLGDVMAVSTCVPVAP |
| | | DNVIVQNSMRVSSRPGTCYSRPLVSFRYEDQGPLIEGQLGENNELRLTRDAL |
| | | EPCTVGHRRYFIFGGGYVYFEEYAYSHQLSRADVTTVSTFIDLNITMLEDHE |
| | | FVPLEVYTRHEIKDSGLLDYTEVQRRNQLHDIRFAAIDTVIRADANAAMFAG |
| | | LCAFFEGMGDLGRAVGKVVMGVVGGVVSAVSGVSSFMSNPFGALAVGLLVLA |
| | | GLVAAFFAFRYVLQLQRNPMKALYPLTTKELKTSDPGGVGGEGEEGAEGGGF |
| | | DEAKLAEAREMIRYMAVSAMERTEHIKARKKGTSALLSSKVTNMVLRKRNKA |
| | | RYSPLHNEDEAGDEDEL* |
| ARV-HSV-gB (HSV- | 608 | GAATTCGGCTTTAATACGACTCACTATAAGGACATTAGCTECTGACACAACT |
| 2 glycoprotein B), | | GTGTTCACTAGCAACCTCAAACAGCCACCatgcgcggggggggcttgatatg |
| DNA sequence | | cgcgctggtcgtgggggcgctggtggccgcggtggcgtcggcggccccggcg |
| | | gccccccgcgccagcggcggcgtggccgcgaccgtcgcggcgaacgggggac |
| | | ccgcctcccggccgccccccgtcccgagccccgcgaccaccagggcccggaa |
| | | gcggaaaaccaaaaagccgcccgagcggcccgaggcgaccccgccccccgac |
| | | gccaacgcgaccgtcgccgccggccacgccacgctgcgcgcgcacctgcggg |
| | | aaatcaaggtcgagaacgccgatgcccagttttacgtgtgcccacccccgac |
| | | aggcgccacggtggtgcagtttgagcagccgcgccgctgcccgacgcgcccg |
| | | gaggggcagaactacacggagggcatcgcggtggtcttcaaggagaacatcg |
| | | ccccgtacaaattcaaggccaccatgtactacaaagacgtgaccgtgtcgca |
| | | ggtgtggttcggccaccgctactcccagtttatggggatattcgaggaccgc |
| | | gcccccgtacccttcgaggaggtgatcgacaagataaacgccaagggggtct |
| | | gccgctccacggccaagtacgtgcggaacaacatggagaccaccgcgtttca |
| | | ccgggacgaccacgagaccgacatggagctcaagccggcgaaggtcgccacg |
| | | cgcacgagccgggggtggcacaccaccgacctcaagtacaaccccagccggg |
| | | tggaggcgttccatcggtacggcacgacggtcaactgcatcgtcgaggaggt |
| | | ggacgcgcggtcggtgtacccgTATgatgagtttgtgctggcgacgggcgac |
| | | tttgtgtacatgtccccgttttacggctaccgggaggggagccacaccgagc |
| | | acaccagctacgccgccgaccgcttcaagcaggtcgacggcttctacgcgcg |
| | | cgacctcaccacgaaggcccgggccacgagcccgacgacccgcaacttgctg |
| | | acgacccccaagtttaccgtggcctgggactgggtgccgaagcgaccggcgg |
| | | tctgcaccatgaccaagtggcaggaggtggacgagatgctccgcgccgagta |
| | | cggcggctccttccgcttctcctccgacgccatcagcaccaccttcaccacc |
| | | aacctgacccagtacagcctcagccgcgtcgacctgggcgactgcatcggcc |
| | | gggatgcccgcgaggccatcgaccgcatgtttgcgcgcaagtacaacgccac |
| | | gcacatcaaggtgggccagccgcagtactacctggccacggggggcttcctc |
| | | atcgcgtaccagcccctcctcagcaacacgctcgccgagctgtacgtgcggg |
| | | agtacatgcgggagcaggaccgcaagccccggaatgccacgcccgcgccact |
| | | gcgggaggcgcccagcgccaacgcgagcgtggagcgcatcaagaccaccagc |
| | | agcatcgagttcgcccggctgcagtttacgtataaccatatacagcgccccg |
| | | tgaacgacatgctggggcgcatcgccgtcgcgtggtgcgagctgcagaacca |
| | | cgagctgacactctggaacgaggcccgcaagctcaaccccaacgccatcgcc |
| | | agcgccaccgtcggccggcgggtgagcgcgcgcatgctcggagacgtcatgg |
| | | ccgtctccacgtgcgtgcccgtcgccccggacaacgtgatcgtgcagaacag |
| | | catgcgcgtcagcagccggccggggacgtgctacagccgccccctggtcagc |
| | | tttcggtacgaagaccagggcccgctgatcgaggggcagctgggcgagaaca |
| | | acgagctgcgcctcacccgcgacgcgctcgagccgtgcaccgtgggccaccg |
| | | gcgctacttcatcttcggcgggggctacgtatacttcgaggagtacgcgtac |
| | | tcacaccagctgagtcgcgccgacgtcaccaccgtcagcaccttcatcgacc |
| | | tgaacatcaccatgctggaggaccacgagtttgtgcccctggaggtctacac |
| | | gcgccacgagatcaaggacagcggcctgctggactacacggaggtccagcgc |
| | | cgcaaccagctgcacgacctgcgctttgccgccatcgacacggtcatccgcg |
| | | ccgacgccaacgccgccatgttcgcggggctgtgcgcgttcttcgaggggat |
| | | gggggacttggggcgcgcggtcggcaaggtcgtcatgggagtagtggggggc |
| | | gtggtgagcgccgtcagcggcgtgagctcctttatgtccaaccccttcgggg |
| | | cgcttgccgtggggctactggtcctggccggcctggtcgcggccttcttcgc |
| | | cttccgctacgtcctgcaactgcaacgcaatcccatgaaggccctgtatccg |
| | | ctcaccaccaaggaactcaagacatccgaccccgggggcgtgggcggggagg |
| | | gggaggaaggcgcggaggggggcgggtttgacgaggccaagttggccgaggc |
| | | ccgagaaatgatccgatatatggcattggtgagcGCGatggagcgcacggaa |
| | | cacaaggccagaaagaagggcacgagcgccctgctcagctccaaggtcacca |
| | | acatggtactgcgcaagcgcaacaaagccaggtactcaccgctccacaacga |
| | | ggacgaggccggagacgaagacgagctctgaCGTACGGCTGGAGCCTCGGTG |
| | | GCCTAGCTTCTTGCCCCTTGGGCCTCCCCCCAGCCCCTCCTCCCCTTCCTGC |
| | | ACCCGTACCCCCGTGGTCTTTGAATAAAGTCTGAGTGGGCGGCAaaaaaaaa |
| | | aaaaaaaaaaaaaaaaaaaaagcatatgactaaaaaaaaaaaaaaaaaaaaa |
| | | aaaaaaaaaaaaaaaaaaaaaaaaaaaaaaaaaaaaaaaaaaaaaaaaaAAA |
| | | AGAAGAGCAAGCTT |
| ARV-HSV-gB-RS | 609 | MRGGGLICALVVGALVAAVASAAPAAPAAPRASGGVAATVAANGGPASRPPP |
| (HSV-2 glycoprotein | | VPSPATTKARKRKTKKPPKRPEATPPPDANATVAAGHATLRAHLREIKVENA |
| B), Protein sequence | | DAQFYVCPPPTGATVVQFEQPRRCPIRPEGQNYTEGIAVVFKENIAPYKFKA |
| | | TMYYKDVTVSQVWFGHRYSQFMGIFEDRAPVPFEEIIDKINAKGVCRSTAKY |
| | | VRNNMETTAFHRDDHETDMELKPAKFATRTSRGWHTTDLKYNPSRVEAFHRY |
| | | GTTVNCIVEEVDARSVYPYDEFVLATGDFVYMSPFYGYREGSHTEHTSYAAD |
| | | RFKQVDGFYPRQLTTKARATSPTTRNLLTTPKFTVAWDWVPKRPAVCTMTKW |
| | | QEVDEMLRAEYGGSFRFSSDAISTTFTTNLTEYSLSRVDLGDCIGRDAREAI |
| | | DRIFARKYNATHIKVGQPQYYLATGGFLIAYQPLLSNTLAELYVREYMREQD |
| | | RKPRNATPAPLREAPSANASVERIKTTSSIEFARLQFTYNHIQRHVNDMLGR |
| | | IAVAWCELQNHELTLWNEARKLNPNAIASATVGRRVSARMLGDVMAVSTCVP |
| | | VAPDNVIIQNSMRVSSRPGTCYSRPLVSFRYEDQGPLIEGQLGENNEMRLTR |
| | | DALEPCTVGHRRYFIFGGGYVYFEEYAYSHQLSRADVTTVSTFIDLNITMLH |
| | | DHEFVPLEVYTRHEIKDSGLLPYTEVQRRLQLLDLRFADIDTVINYDPNAAM |
| | | FAGLYAFFEGMGDVGRAVGKVVMGVVGGVVSAVSGVSSFMSNPFGALAVGLL |
| | | VLAGLVAAFFAFRYVLRLQRNPMKALYPLTLKELKTSDPGGVGGEGEEGAEG |
| | | GGFDEAKLAEAREMIRYMALVSAMERTEHKARKKGTSALLSSKVINMVLRKR |
| | | NKARYSPLHNEDEAGDEDEL* |
| ARV-HSV-gB-RS | 610 | GAATTCTAATACGACTCACTATAAGGACATTAGCTTCTGACACAACTGTGTT |
| (HSV-2 glycoprotein | | CACTAGCAACCTCAAACAGCCACCatgcgcggggggggcttgatatgcgcgc |
| B), DNA sequence | | tggtcgtgggggcgctggtggccgcggtggcgtcggcggccccggcggcccc |
| | | cgcggccccccgcgccagcggcggcgtggccgcgaccgtcgcggcgaacggg |
| | | ggacccgcctcccggccgccccccgtcccgagccccgcgaccaccaaggccc |
| | | ggaagcggaaaaccaaaaagccgcccaagcggcccgaggcgaccccgccccc |
| | | cgacgccaacgcgaccgtcgccgccggccacgccacgctgcgcgcgcacctg |
| | | cgggaaatcaaggtcgagaacgccgatgcccagttttacgtgtgcccacccc |
| | | cgacaggcgccacggtggtgcagtttgagcagccgcgccgctgcccgacgcg |
| | | cccggaggggcagaactacacggagggcatcgcggtggtcttcaaggagaac |
| | | atcgccccgtacaaattcaaggccaccatgtactacaaagacgtgaccgtgt |
| | | cgcaggtgtggttcggccaccgctactcccagtttatggggatattcgagga |
| | | ccgcgcccccgtacccttcgaggagatcatcgacaagataaacgccaagggg |
| | | gtctgccgctccacggccaagtacgtgcggaacaacatggagaccaccgcgt |
| | | ttcaccgggacgaccacgagaccgacatggagctcaagccggcgaagttcgc |
| | | cacgcgcacgagccgggggtggcacaccaccgacctcaagtacaaccccagc |
| | | cgggtggaggcgttccatcggtacggcacgacggtcaactgcatcgtcgagg |
| | | aggtggacgcgcggtcggtgtacccgTATgatgagtttgtgctggcgacggg |
| | | cgactttgtgtacatgtccccgttttacggctaccgggaggggagccacacc |
| | | gagcacaccagctacgccgccgaccgcttcaagcaggtcgacggcttctacc |
| | | cgcgcgacctcaccacgaaggcccgggccacgagcccgacgacccgcaactt |
| | | gctgacgacccccaagtttaccgtggcctgggactgggtgccgaagcgaccg |
| | | gcggtctgcaccatgaccaagtggcaggaggtggacgagatgctccgcgccg |
| | | agtacggcggctccttccgcttctcctccgacgccatcagcaccaccttcac |
| | | caccaacctgaccgagtacagcctcagccgcgtcgacctgggcgactgcatc |
| | | ggccgggatgcccgcgaggccatcgaccgcatctttgcgcgcaagtacaacg |
| | | ccacgcacatcaaggtgggccagccgcagtactacctggccacggggggctt |
| | | cctcatcgcgtaccagcccctcctcagcaacacgctcgccgagctgtacgtg |
| | | cgggagtacatgcgggagcaggaccgcaagccccggaatgccacgcccgcgc |
| | | cactgcgggaggcgcccagcgccaacgcgagcgtggagcgcatcaagaccac |
| | | cagcagcatcgagttcgcccggctgcagtttacgtataaccatatacagcgc |
| | | CACgtgaacgacatgctggggcgcatcgccgtcgcgtggtgcgagctgcaga |
| | | accacgagctgacactctggaacgaggcccgcaagctcaaccccaacgccat |
| | | cgccagcgccaccgtcggccggcgggtgagcgcgcgcatgctcggagacgtc |
| | | atggccgtctccacgtgcgtgcccgtcgccccggacaacgtgatcatccaga |
| | | acagcatgcgcgtcagcagccggccggggacgtgctacagccgccccctggt |
| | | cagctttcggtacgaagaccagggcccgctgatcgaggggcagctgggcgag |
| | | aacaacgagctgcgcctcacccgcgacgcgctcgagccgtgcaccgtgggcc |
| | | accggcgctacttcatcttcggcgggggctacgtatacttcgaggagtacgc |
| | | gtactcacaccagctgagtcgcgccgacgtcaccaccgtcagcaccttcatc |
| | | gacctgaacatcaccatgctgcacgaccacgagtttgtgcccctggaggtct |
| | | acacgcgccacgagatcaaggacagcggcctgctgccctacacggaggtcca |
| | | gcgccgcctgcagctgctggacctgcgctttgccgacatcgacacggtcatc |
| | | aactacgaccccaacgccgccatgttcgcggggctgtacgcgttcttcgagg |
| | | ggatgggggacgtggggcgcgcggtcggcaaggtcgtcatgggagtagtggg |
| | | gggcgtggtgagcgccgtcagcggcgtgagctcctttatgtccaaccccttc |
| | | ggggcgcttgccgtggggctactggtcctggccggcctggtcgcggccttct |
| | | tcgccttccgctacgtcctgcgcctgcaacgcaatcccatgaaggccctgta |
| | | gagggggaggaaggcgcggaggggggcgggtttgacgaggccaagttggccg |
| | | aggcccgagaaatgatccgatatatggcattggtgagcGCGatggagcgcac |
| | | ggaacacaaggccagaaagaagggcacgagcgccctgctcagctccaaggtc |
| | | accaacatggtactgcgcaagcgcaacaaagccaggtactcaccgctccaca |
| | | acgaggacgaggccggagacgaagacgagctctgaCGTACGGCTGGAGCCTC |
| | | GGTGGCCTAGCTTCTTGCCCCETGGGCCTCCCCCCAGCCCCTCCTCCCCTTC |
| | | CTGCACCCGTACCCCCGTGGTCTTTGAATAAAGTCTGAGTGGGCGGCAaaaa |
| | | aaaaaaaaaaaaaaaaaaaaaaaaagcatatgactaaaaaaaaaaaaaaaaa |
| | | aaaaaaaaaaaaaaaaaaaaaaaaaaaaaaaaaaaaaaaaaaaaaaaaaaaa |
| | | aAAAAGAAGAGCAAGCTT |
| ARV-HSV-gB-RSN | 611 | MRGGGLICALVVGALVAAVASAAPAAPAAPRASGGVAATVAANGGPASRPPP |
| (HSV-2 glycoprotein | | VPSPATTKARKRKTKKPPKRPEATPPPDANATVAAGHATLRAHLRELVDENA |
| B), Protein sequence | | DAQFYVCPPPTGATVVQFEQPRRCPTRPEGQNYTEGIAVVFKENIAPYKFKA |
| | | TMYYKDVTVSQVWFGHRYSQEMGIFEDRAPVPFEEIIDKINAKGVCRSTAKY |
| | | VRNNMETTAFHRDDHETDMELKPAKFATRTSRGWHTTDLKYNPSRVEAFHRY |
| | | GTTVNCIVEEVDARSVYPYDEFVLATGDFVYMSPFYGYREGSHTEHTSYAAD |
| | | RFKQVDGFYPRQLTTKARATSPTTRNLLTTPKFTVAWDWVPKRPAVCTMTKW |
| | | QEVDEMLRAEYGGSFRFSSDAISTTFTTNLTEYSLSRVDLGDCIGRDAREIA |
| | | DRIFARKYNATHIKVGQPQYYLATGGFLIAYQPLLSNTLAELYVREYMREQD |
| | | RKPRNATPAPLREAPSANASVERIKTTSSIEFARLQFTYNHIQRHVNDMLGR |
| | | IAVAWCELQNHELTLWNEARKLNPNAIASATVGRRVSARMLGDVMAVSTCVP |
| | | VAPDNVIIQNSMRVSSRPGTCYSRPLVSFRYEDQGPLIEGQLGENNELRLTR |
| | | DALEPCTVGHRRYFIYGKGYVYFEEYAYSHQLSRADVTTVSTFIDLNITMLH |
| | | DHEFVPLEVYTRHEIKDSGLLPYTEVQRRLQLLDLRFADIDTVINYDPNAAM |
| | | FAGLYAFFEGMGDVGRAVGKVVMGVVGGVVSAVSGVSSFMSNPFGALAVGLL |
| | | VLAGLVAAFFAFRYVLRLQRNPMKALYPLTLKELKTSDPGGVGGEGEEGAEG |
| | | GGFDEAKLAEAREMIRYMALVSAMERTEHKARKKGTSALLSSKVTNMVLRKR |
| | | NKARYSPLHNEDEAGDEDEL* |
| ARV-HSV-gB-RSN | 612 | GAATTCTAATACGACTCACTATAAGGACATTAGCTTCTGACACAACTGTGTT |
| (HSV-2 glycoprotein | | CACTAGCAACCTCAAACAGCCACCatgcgcggggggggcttgatatgcgcgc |
| B), DNA sequence | | tggtcgtgggggcgctggtggccgcggtggcgtcggcggccccggcggcccc |
| | | cgcggccccccgcgccagcggcggcgtggccgcgaccgtcgcggcgaacggg |
| | | ggacccgcctcccggccgccccccgtcccgagccccgcgaccaccaaggccc |
| | | ggaagcggaaaaccaaaaagccgcccaagcggcccgaggcgaccccgccccc |
| | | cgacgccaacgcgaccgtcgccgccggccacgccacgctgcgcgcgcacctg |
| | | cgggaaCTGGTCGACgagaacgccgatgcccagttttacgtgtgcccacccc |
| | | cgacaggcgccacggtggtgcagtttgagcagccgcgccgctgcccgacgcg |
| | | cccggaggggcagaactacacggagggcatcgcggtggtcttcaaggagaac |
| | | atcgccccgtacaaattcaaggccaccatgtactacaaagacgtgaccgtgt |
| | | cgcaggtgtggttcggccaccgctactcccagtttatggggatattcgagga |
| | | ccgcgcccccgtacccttcgaggagatcatcgacaagataaacgccaagggg |
| | | gtctgccgctccacggccaagtacgtgcggaacaacatggagaccaccgcgt |
| | | ttcaccgggacgaccacgagaccgacatggagctcaagccggcgaagttcgc |
| | | cacgcgcacgagccgggggtggcacaccaccgacctcaagtacaaccccagc |
| | | cgggtggaggcgttccatcggtacggcacgacggtcaactgcatcgtcgagg |
| | | aggtggacgcgcggtcggtgtacccgTATgatgagtttgtgctggcgacggg |
| | | cgactttgtgtacatgtccccgttttacggctaccgggaggggagccacacc |
| | | gagcacaccagctacgccgccgaccgcttcaagcaggtcgacggcttctacc |
| | | cgcgcgacctcaccacgaaggcccgggccacgagcccgacgacccgcaactt |
| | | gctgacgacccccaagtttaccgtggcctgggactgggtgccgaagcgaccg |
| | | gcggtctgcaccatgaccaagtggcaggaggtggacgagatgctccgcgccg |
| | | agtacggcggctccttccgcttctcctccgacgccatcagcaccaccttcac |
| | | caccaacctgaccgagtacagcctcagccgcgtcgacctgggcgactgcatc |
| | | ggccgggatgcccgcgaggccatcgaccgcatctttgcgcgcaagtacaacg |
| | | ccacgcacatcaaggtgggccagccgcagtactacctggccacggggggctt |
| | | cctcatcgcgtaccagcccctcctcagcaacacgctcgccgagctgtacgtg |
| | | cgggagtacatgcgggagcaggaccgcaagccccggaatgccacgcccgcgc |
| | | cactgcgggaggcgcccagcgccaacgcgagcgtggagcgcatcaagaccac |
| | | cagcagcatcgagttcgcccggctgcagtttacgtataaccatatacagcgc |
| | | CACgtgaacgacatgctggggcgcatcgccgtcgcgtggtgcgagctgcaga |
| | | accacgagctgacactctggaacgaggcccgcaagctcaaccccaacgccat |
| | | cgccagcgccaccgtcggccggcgggtgagcgcgcgcatgctcggagacgtc |
| | | atggccgtctccacgtgcgtgcccgtcgccccggacaacgtgatcatccaga |
| | | acagcatgcgcgtcagcagccggccggggacgtgctacagccgccccctggt |
| | | cagctttcggtacgaagaccagggcccgctgatcgaggggcagctgggcgag |
| | | aacaacgagctgcgcctcacccgcgacgcgctcgagccgtgcaccgtgggcc |
| | | accggcgctacttcatcTACggcAAGggctacgtatacttcgaggagtacgc |
| | | gtactcacaccagctgagtcgcgccgacgtcaccaccgtcagcaccttcatc |
| | | gacctgaacatcaccatgctgcacgaccacgagtttgtgcccctggaggtct |
| | | acacgcgccacgagatcaaggacagcggcctgctgccctacacggaggtcca |
| | | gcgccgcctgcagctgctggacctgcgctttgccgacatcgacacggtcatc |
| | | aactacgaccccaacgccgccatgttcgcggggctgtacgcgttcttcgagg |
| | | ggatgggggacgtggggcgcgcggtcggcaaggtcgtcatgggagtagtggg |
| | | gggcgtggtgagcgccgtcagcggcgtgagctcctttatgtccaaccccttc |
| | | ggggcgcttgccgtggggctactggtcctggccggcctggtcgcggccttct |
| | | tcgccttccgctacgtcctgcgcctgcaacgcaatcccatgaaggccctgta |
| | | tccgctcaccctcaaggaactcaagacatccgaccccgggggcgtgggcggg |
| | | gagggggaggaaggcgcggaggggggcgggtttgacgaggccaagttggccg |
| | | aggcccgagaaatgatccgatatatggcattggtgagcGCGatggagcgcac |
| | | ggaacacaaggccagaaagaagggcacgagcgccctgctcagctccaaggtc |
| | | accaacatggtactgcgcaagcgcaacaaagccaggtactcaccgctccaca |
| | | acgaggacgaggccggagacgaagacgagctctgaCGTACGGCTGGAGCCTC |
| | | GGTGGCCTAGCTTCTTGCCCCTTGGGCCTCCCCCCAGCCCCTCCTCCCCTTC |
| | | CTGCACCCGTACCCCCGTGGTCTTTGAATAAAGTCTGAGTGGGCGGCAaaaa |
| | | aaaaaaaaaaaaaaaaaaaaaaaaagcatatgactaaaaaaaaaaaaaaaaa |
| | | aaaaaaaaaaaaaaaaaaaaaaaaaaaaaaaaaaaaaaaaaaaaaaaaaaaa |
| | | aAAAAGAAGAGCAAGCTT |
| ARV-HSV-sgB | 613 | MRGGGLICALVVGALVAAVASAAPAAPRASGGVAATVAANGGPASRPPPVPS |
| (HSV-2 glycoprotein | | PATTRARKRKTKKPPERPEATPPPDANATVAAGHATLRAHLREIKVENADAQ |
| B, soluble form with | | FYVCPPPTGATVVQFEQPRRCPTRPEGQNYTEGIAVVFKENIAPYKFKATMY |
| extracellular domain), | | YKDVTVSQVWFGHRYSQFMGIFEDRAPVPFEEVIDKINAKGVCRSTAKYVRN |
| Protein sequence | | NMETTAFHRDDHETDMELKPAKVATRTSRGWHTTDLKYNPSRVEAFHRYGTT |
| | | VNCIVEEVDARSVYPYDEFVLATGDFVYMSPFYGYREGSHTEHTSYAADRFK |
| | | QVDGFYARDLITKARATSPTTRNLLTTPKFTVAWDWVPKRPAVCTMTKWQEV |
| | | DEMLRAEYGGSFRFSSDAISTTFTTNLTQYSLSRVDLGDCIGRDAREAIDRM |
| | | FARKYNATHIKVGQPQYYLATGGFLIAYQPLLSNTLAELYVREYMREQDRKP |
| | | RNATPAPLREAPSANASVERIKTTSSIEFARLQFTYNHIQRPVNDMLGRIAV |
| | | AWCELQNHELTLWNEARKLNPNAIASATVGRRVSARMLGDVMAVSTCVPVAP |
| | | DNVIVQNSMRVSSRPGTCYSRPLVSFRYEDQGPLIEGQLGENNELRLTRDAL |
| | | EPCTVGHRRYFIFGGGYVYFEEYAYSHQLSRADVTTVSTEIDLNITMLEDHE |
| | | FVPLEVYTRHEIKDSGLLDYTEVQRRNQLHDLRFAAIDTVI* |
| ARV-HSV-sgB | 614 | GAATTCTAATACGACTCACTATAAGGACATTAGCTTCTGACACAACTGTGTT |
| (HSV-2 glycoprotein | | CACTAGCAACCTCAAACAGCCACCatgcgcggggggggcttgatatgcgcgc |
| B, soluble form with | | tggtcgtgggggcgctggtggccgcggtggcgtcggccgccccggcggcccc |
| extracellular domain), | | ccgcgccagcggcggcgtggccgcgaccgtcgcggcgaacgggggacccgcc |
| DNA sequence | | tcccggccgccccccgtcccgagccccgcgaccaccagggcccggaagcgga |
| | | aaaccaaaaagccgcccgagcggcccgaggcgaccccgccccccgacgccaa |
| | | cgcgaccgtcgccgccggccacgccacgctgcgcgcgcacctgcgggaaatc |
| | | aaggtcgagaacgccgatgcccagttttacgtgtgcccacccccgacaggcg |
| | | ccacggtggtgcagtttgagcagccgcgccgctgcccgacgcgcccggaggg |
| | | gcagaactacacggagggcatcgcggtggtcttcaaggagaacatcgccccg |
| | | tacaaattcaaggccaccatgtactacaaagacgtgaccgtgtcgcaggtgt |
| | | ggttcggccaccgctactcccagtttatggggatattcgaggaccgcgcccc |
| | | cgtacccttcgaggaggtgatcgacaagataaacgccaagggggtctgccgc |
| | | tccacggccaagtacgtgcggaacaacatggagaccaccgcgtttcaccggg |
| | | acgaccacgagaccgacatggagctcaagccggcgaaggtcgccacgcgcac |
| | | gagccgggggtggcacaccaccgacctcaagtacaaccccagccgggtggag |
| | | gcgttccatcggtacggcacgacggtcaactgcatcgtcgaggaggtggacg |
| | | cgcggtcggtgtacccgTATgatgagtttgtgctggcgacgggcgactttgt |
| | | gtacatgtccccgttttacggctaccgggaggggagccacaccgagcacacc |
| | | agctacgccgccgaccgcttcaagcaggtcgacggcttctacgcgcgcgacc |
| | | tcaccacgaaggcccgggccacgagcccgacgacccgcaacttgctgacgac |
| | | ccccaagtttaccgtggcctgggactgggtgccgaagcgaccggcggtctgc |
| | | accatgaccaagtggcaggaggtggacgagatgctccgcgccgagtacggcg |
| | | gctccttccgcttctcctccgacgccatcagcaccaccttcaccaccaacct |
| | | gacccagtacagcctcagccgcgtcgacctgggcgactgcatcggccgggat |
| | | gcccgcgaggccatcgaccgcatgtttgcgcgcaagtacaacgccacgcaca |
| | | tcaaggtgggccagccgcagtactacctggccacggggggcttcctcatcgc |
| | | gtaccagcccctcctcagcaacacgctcgccgagctgtacgtgcgggagtac |
| | | atgcgggagcaggaccgcaagccccggaatgccacgcccgcgccactgcggg |
| | | aggcgcccagcgccaacgcgagcgtggagcgcatcaagaccaccagcagcat |
| | | cgagttcgcccggctgcagtttacgtataaccatatacagcgccccgtgaac |
| | | gacatgctggggcgcatcgccgtcgcgtggtgcgagctgcagaaccacgagc |
| | | tgacactctggaacgaggcccgcaagctcaaccccaacgccatcgccagcgc |
| | | caccgtcggccggcgggtgagcgcgcgcatgctcggagacgtcatggccgtc |
| | | tccacgtgcgtgcccgtcgccccggacaacgtgatcgtgcagaacagcatgc |
| | | gcgtcagcagccggccggggacgtgctacagccgccccctggtcagctttcg |
| | | gtacgaagaccagggcccgctgatcgaggggcagctgggcgagaacaacgag |
| | | ctgcgcctcacccgcgacgcgctcgagccgtgcaccgtgggccaccggcgct |
| | | acttcatcttcggcgggggctacgtatacttcgaggagtacgcgtactcaca |
| | | ccagctgagtcgcgccgacgtcaccaccgtcagcaccttcatcgacctgaac |
| | | atcaccatgctggaggaccacgagtttgtgcccctggaggtctacacgcgcc |
| | | acgagatcaaggacagcggcctgctggactacacggaggtccagcgccgcaa |
| | | ccagctgcacgacctgcgctttgccgccatcgacacggtcatctgaCGTACG |
| | | GCTGGAGCCTCGGTGGCCTAGCTTCTTGCCCCTTGGGCCTCCCCCCAGCCCC |
| | | TCCTCCCCTTCCTGCACCCGTACCCCCGTGGTCTTTGAATAAAGTCTGAGTG |
| | | GGCGGCAaaaaaaaaaaaaaaaaaaaaaaaaaaaaagcatatgactaaaaaa |
| | | aaaaaaaaaaaaaaaaaaaaaaaaaaaaaaaaaaaaaaaaaaaaaaaaaaaa |
| | | aaaaaaaaaaaaAAAAGAAGAGCAAGCTT |
| ARV-HSV-gD-WT | 615 | MGRLTSGVGTAALLVVAVGLRVVCAKYALADPSLKMADPNRFRGKNLPVLDQ |
| (HSV-2 glycoprotein | | LTDPPGVKRVYHIQPSLEDPFQPPSIPITVYYAVLERACRSVLLHAPSEAPQ |
| D wildtype), Protein | | IVRGASDEARKHTYNLTIAWYRMGDNCAIPITVMEYTECPYNKSLGVCPIRT |
| sequence | | QPRWSYYDSFSAVSEDNLGFLMHAPAFETAGTYLRLVKINDWTEITQFILEH |
| | | RARASCKYALPLRIPPAACLTSKAYQQGVTVDSIGMLPRFIPENQRTVALYS |
| | | LKIAGWHGPKPPYTSTILPPELSDTTNATQPELVPEDPEDSALLEDPAGTVS |
| | | SQIPPNWHIPSIQDVAPHHAPAAPSNPGLIIGALAGSTLAVLVIGGIAFWVR |
| | | RRAQMAPKRLRLPHIRDDDAPPSHQPLFY* |
| ARV-HSV-gD-WT | 616 | GAATTCGGCTTTAATACGACTCACTATAAGGACATTAGCTTCTGACACAACT |
| (HSV-2 glycoprotein | | GTGTTCACTAGCAACCTCAAACAGCCACCatggggcgattgacctccggcgt |
| D wildtype), DNA | | cgggacggcggccctgctagtagtcgcggtgggactccgcgtcgtctgcgcc |
| sequence | | aaatacgccttagcagaccccagccttaagatggccgatcccaatcgatttc |
| | | gcgggaagaaccttccggtattggaccagctgaccgacccccccggggtgaa |
| | | gcgagtataccacatacagccgagcctggaggacccgttccagccccccagc |
| | | atcccgatcacagtgtactacgcagtgctggaacgagcctgccgcagcgtgc |
| | | tcctacatgccccaagcgaggccccccagatcgtgcgcggggcatccgacga |
| | | ggcccgaaagcacacgtacaacctgaccatcgcctggtatcgcatgggagac |
| | | aattgcgcaatccccatcacggtaatggaatacaccgagtgcccctacaaca |
| | | agagcttgggggtctgccccatccgaacgcagccccgctggagctactatga |
| | | cagctttagcgccgtcagcgaggataacctgggattcctgatgcacgccccc |
| | | gccttcgagaccgcgggaacgtacctgcggctagtgaagataaacgactgga |
| | | cggagatcacacaatttatcctggagcaccgggcccgcgcctcctgcAAGta |
| | | cgcactccccctgcgcatccccccggcagcgtgcctcaccagcaaggcctac |
| | | caacagggcgtgacggtcgacagcatcgggatgctaccccgctttatccccg |
| | | aaaaccagcgcaccgtcgccctatacagcttaaaaatcgccggctggcacgg |
| | | ccccaagcccccgtacaccagcaccctgctgccgccggagctttccgacacc |
| | | accaacgccacgcaacccgaactcgtaccggaagaccccgaggacagcGCGc |
| | | tcttagaggatcccgccgggacggtgtcatcacagatccccccaaactggca |
| | | catcccgtcgatccaggacgtcgcgccgcaccacgcccccgccgcccccagc |
| | | aacccgggcctgatcatcggcgcgctggccggcagtaccctggcggtgctgg |
| | | tcatcggcggaatagcgttttgggtacgccgccgcgcacagatggcccccaa |
| | | gcgcctacgactcccccacatccgggatgacgacgcgccccccagccaccag |
| | | ccattgttttactgaCGTACGGCTGGAGCCTCGGTGGCCTAGCTTCTTGCCC |
| | | CTTGGGCCTCCCCCCAGCCCCECCTCCCCTTCCTGCACCCGTACCCCCGTGG |
| | | TCTTTGAATAAAGTCTGAGTGGGCGGCAaaaaaaaaaaaaaaaaaaaaaaaa |
| | | aaaaagcatatgactaaaaaaaaaaaaaaaaaaaaaaaaaaaaaaaaaaaaa |
| | | aaaaaaaaaaaaaaaaaaaaaaaaaaaaaaaaaAAAAGAAGAGCAAGCTT |
| ARV-HSV-gD(HSV- | 617 | MGRLTSGVGTAALLVVAVGLRVVCAKYALADPSLKMADPNRFRGKNLPVLDQ |
| 2 glycoprotein D with | | LTDPPGVKRVYHIQPSLEDPFQPPSIPITVYYAVLERACRSVLLHAPSEAPQ |
| two AA | | IVRGASDEARKHTYNLTIAWYRMGDNCAIPITVMEYTECPYNKSLGVCPIRT |
| substitutions), Protein | | QPRWSYYDSFSAVSEDNLGFLMHAPAFETAGTYLRLVKINDWTEITQFILEH |
| sequence | | RARASCCYALPLRIPPAACLISKAYQQGVTVDSIGMLPRFIPENQRTVALYS |
| | | LKIAGWHGPKPPYTSTALPPELSDTTNATQPELVPEDPEDSCLLEDPAGTVS |
| | | SQIPPNWHIPSIQDVAPHHAPAAPSNPGLIIGALAGSTLAVLVIGGIAFWVR |
| | | RRAQMAPKRLRLPHIRDDDAPPSHQPLFY* |
| ARV-HSV-gD(HSV- | 618 | GAATTCGGCTTTAATACGACTCACTATAAGGACATTAGCTTCTGACACAACT |
| 2 glycoprotein D with | | GTGTTCACTAGCAACCTCAAACAGCCACCatggggcgattgacctccggcgt |
| two AA | | cgggacggcggccctgctagtagtcgcggtgggactccgcgtcgtctgcgcc |
| substitutions), DNA | | aaatacgccttagcagaccccagccttaagatggccgatcccaatcgatttc |
| sequence | | gcgggaagaaccttccggtattggaccagctgaccgacccccccggggtgaa |
| | | gcgagtataccacatacagccgagcctggaggacccgttccagccccccagc |
| | | atcccgatcacagtgtactacgcagtgctggaacgagcctgccgcagcgtgc |
| | | tcctacatgccccaagcgaggccccccagatcgtgcgcggggcatccgacga |
| | | ggcccgaaagcacacgtacaacctgaccatcgcctggtatcgcatgggagac |
| | | aattgcgcaatccccatcacggtaatggaatacaccgagtgcccctacaaca |
| | | agagcttgggggtctgccccatccgaacgcagccccgctggagctactatga |
| | | cagctttagcgccgtcagcgaggataacctgggattcctgatgcacgccccc |
| | | gccttcgagaccgcgggaacgtacctgcggctagtgaagataaacgactgga |
| | | cggagatcacacaatttatcctggagcaccgggcccgcgcctcctgctgcta |
| | | cgcactccccctgcgcatccccccggcagcgtgcctcaccagcaaggcctac |
| | | caacagggcgtgacggtcgacagcatcgggatgctaccccgctttatccccg |
| | | aaaaccagcgcaccgtcgccctatacagcttaaaaatcgccggctggcacgg |
| | | ccccaagcccccgtacaccagcaccctgctgccgccggagctttccgacacc |
| | | accaacgccacgcaacccgaactcgtaccggaagaccccgaggacagctgcc |
| | | tcttagaggatcccgccgggacggtgtcatcacagatccccccaaactggca |
| | | catcccgtcgatccaggacgtcgcgccgcaccacgcccccgccgcccccagc |
| | | aacccgggcctgatcatcggcgcgctggccggcagtaccctggcggtgctgg |
| | | tcatcggcggaatagcgttttgggtacgccgccgcgcacagatggcccccaa |
| | | gcgcctacgactcccccacatccgggatgacgacgcgccccccagccaccag |
| | | ccattgttttactgaCGTACGGCTGGAGCCTCGGTGGCCTAGCTTCTTGCCC |
| | | CTTGGGCCTCCCCCCAGCCCCTCCTCCCCTTCCTGCACCCGTACCCCCGTGG |
| | | TCTTTGAATAAAGTCTGAGTGGGCGGCAaaaaaaaaaaaaaaaaaaaaaaaa |
| | | aaaaagcatatgactaaaaaaaaaaaaaaaaaaaaaaaaaaaaaaaaaaaaa |
| | | aaaaaaaaaaaaaaaaaaaaaaaaaaaaaaaaaAAAAGAAGAGCAAGCTT |
| ARV-HSV-sgD | 619 | MTRLTVLALLAGLLASSRAKYALADPSLKMADPNRFRGKNLPVLDQLTDPPG |
| (HSV-2 glycoprotein | | VKRVYHIQPSLEDPFQPPSIPITVYYAVLERACRSVLLHAPSEAPQIVRGAS |
| D, soluble format with | | DEARKHTYNLTIAWYRMGDNCAIPITVMEYTECPYNKSLGVCPIRTQPRWSY |
| extracellular domain), | | YDSFSAVSEDNLGFLMHAPAFETAGTYLRLVKINDWTEITQFILEHRARASC |
| Protein sequence | | CYALPLRIPPAACLISKAYQQGVTVDSIGMLPRFIPENQRTVALYSLKIAGW |
| | | HGPKPPYTSTLLPPELSDTTNATQPELVPEDPEDSCLLEDPAGTVSSQIPPN |
| | | WHIPSIQDVAPHH* |
| ARV-HSV-sgD | 620 | GAATTCGGCTTTAATACGACTCACTATAAGGACATTAGCTTCTGACACAACT |
| (HSV-2 glycoprotein | | GTGTTCACTAGCAACCTCAAACAGCCACCatgacccgcctgaccgtgctggc |
| D, soluble format with | | cctgctggccggcctgctggcctcctcccgcgccaaatacgccttagcagac |
| extracellular domain), | | cccagccttaagatggccgatcccaatcgatttcgcgggaagaaccttccgg |
| DNA sequence | | tattggaccagctgaccgacccccccggggtgaagcgagtataccacataca |
| | | gccgagcctggaggacccgttccagccccccagcatcccgatcacagtgtac |
| | | tacgcagtgctggaacgagcctgccgcagcgtgctcctacatgccccaagcg |
| | | aggccccccagatcgtgcgcggggcatccgacgaggcccgaaagcacacgta |
| | | caacctgaccatcgcctggtatcgcatgggagacaattgcgcaatccccatc |
| | | acggtaatggaatacaccgagtgcccctacaacaagagcttgggggtctgcc |
| | | ccatccgaacgcagccccgctggagctactatgacagctttagcgccgtcag |
| | | cgaggataacctgggattcctgatgcacgcccccgccttcgagaccgcggga |
| | | acgtacctgcggctagtgaagataaacgactggacggagatcacacaattta |
| | | tcctggagcaccgggcccgcgcctcctgctgctacgcactccccctgcgcat |
| | | ccccccggcagcgtgcctcaccagcaaggcctaccaacagggcgtgacggtc |
| | | gacagcatcgggatgctaccccgctttatccccgaaaaccagcgcaccgtcg |
| | | ccctatacagcttaaaaatcgccggctggcacggccccaagcccccgtacac |
| | | cagcaccctgctgccgccggagctttccgacaccaccaacgccacgcaaccc |
| | | gaactcgtaccggaagaccccgaggacagctgcctcttagaggatcccgccg |
| | | ggacggtgtcatcacagatccccccaaactggcacatcccgtcgatccagga |
| | | cgtcgcgccgcaccactgaCGTACGGCTGGAGCCTCGGTGGCCTAGCTTCTT |
| | | GCCCCTTGGGCCTCCCCCCAGCCCCTCCTCCCCTTCCTGCACCCGTACCCCC |
| | | GTGGTCTTTGAATAAAGTCTGAGTGGGCGGCAaaaaaaaaaaaaaaaaaaaa |
| | | aaaaaaaaagcatatgactaaaaaaaaaaaaaaaaaaaaaaaaaaaaaaaaa |
| | | aaaaaaaaaaaaaaaaaaaaaaaaaaaaaaaaaaaaaAAAAGAAGAGCAAGC |
| | | TT |
| ARV-HSV-gC (HSV- | 621 | MRMQLLLLIALSLALVINSASPGRTITVGPRGNASNAAPSASPRNASAPRTT |
| 2 glycoprotein C), | | PTPPQPRKATKSKASTAKPAPPPKTGPPKISSEPVRCNRHDPLARYGSRVQI |
| Protein sequence | | RCRFPNSTRTEFRLQIWRYATATDAEIGTAPSELLVMVNVSAPPGGQLVYDS |
| | | APNRTDPHVIWAEGAGPGASPRLYSVVGPLGRQRLIIEELTLETQGMYYWVW |
| | | GRTDRPSAYGTWVRVRVFRPPSLTIHPHAVLEGQPFKATCTAATYYPGNRAE |
| | | FVWFEDGRRVFDPAQIHTQTQENPDGFSTVSTVTSAAVGGQGPPRIFTCQLT |
| | | WHRDSVSFSRRNASGTASVLPRPTITMEFTGDHAVCTAGCVPEGVTFAWFLG |
| | | DDSSPAEKVAVASQTSCGRPGTATIRSTLPVSYEQTEYICRLAGYPDGIPVL |
| | | EHHGSHQPPPRQPTERQVIRAVEGAGIGVAVLVAVVLAGTAVVYLTHASSVR |
| | | YRRLR* |
| ARV-HSV-gC (HSV- | 622 | GAATTCGGCTTTAATACGACTCACTATAAGGACATTAGCTTCTGACACAACT |
| 2 glycoprotein C), | | GTGTTCACTAGCAACCTCAAACAGCCACCATGCGCATGCAGCTGCTGCTGCT |
| DNA sequence | | GATCGCCCTGTCCCTGGCCCTGGTGACCAACTCCGCCTCCCCCGGCCGCACC |
| | | ATCACCGTGGGCCCCCGCGGCAACGCCTCCAACGCCGCCCCCTCCGCCTCCC |
| | | CCCGCAACGCCTCCGCCCCCCGCACCACCCCCACCCCCCCCCAGCCCCGCAA |
| | | GGCCACCAAGTCCAAGGCCTCCACCGCCAAGCCCGCCCCCCCCCCCAAGACC |
| | | GGCCCCCCCAAGACCTCCTCCGAGCCCGTGCGCTGCAACCGCCACGACCCCC |
| | | TGGCCCGCTACGGCTCCCGCGTGCAGATCCGCTGCCGCTTCCCCAACTCCAC |
| | | CCGCACCGAGTTCCGCCTGCAGATCTGGCGCTACGCCACCGCCACCGACGCC |
| | | GAGATCGGCACCGCCCCCTCCCTGGAGGAGGTGATGGTGAACGTGTCCGCCC |
| | | CCCCCGGCGGCCAGCTGGTGTACGACTCCGCCCCCAACCGCACCGACCCCCA |
| | | CGTGATCTGGGCCGAGGGCGCCGGCCCCGGCGCCTCCCCCCGCCTGTACTCC |
| | | GTGGTGGGCCCCCTGGGCCGCCAGCGCCTGATCATCGAGGAGCTGACCCTGG |
| | | AGACCCAGGGCATGTACTACTGGGTGTGGGGCCGCACCGACCGCCCCTCCGC |
| | | CTACGGCACCTGGGTGCGCGTGCGCGTGTTCCGCCCCCCCTCCCTGACCATC |
| | | CACCCCCACGCCGTGCTGGAGGGCCAGCCCTTCAAGGCCACCTGCACCGCCG |
| | | CCACCTACTACCCCGGCAACCGCGCCGAGTTCGTGTGGTTCGAGGACGGCCG |
| | | CCGCGTGTTCGACCCCGCCCAGATCCACACCCAGACCCAGGAGAACCCCGAC |
| | | GGCTTCTCCACCGTGTCCACCGTGACCTCCGCCGCCGTGGGCGGCCAGGGCC |
| | | CCCCCCGCACCTTCACCTGCCAGCTGACCTGGCACCGCGACTCCGTGTCCTT |
| | | CTCCCGCCGCAACGCCTCCGGCACCGCCTCCGTGCTGCCCCGCCCCACCATC |
| | | ACGATGGAGTTCACCGGCGACCACGCCGTGTGCACCGCCGGCTGCGTGCCCG |
| | | AGGGCGTGACCTTCGCCTGGTTCCTGGGCGACGACTCCTCCCCCGCCGAGAA |
| | | GGTGGCCGTGGCCTCCCAGACCTCCTGCGGCCGCCCCGGCACCGCCACCATC |
| | | CGCTCCACCCTGCCCGTGTCCTACGAGCAGACCGAGTACATCTGCCGCCTGG |
| | | CCGGCTACCCCGACGGCATCCCCGTGCTGGAGCACCACggcagtcaccagcc |
| | | cccgccgcgggaccccaccgagcggcaggtgatccgggcggtggagggggcg |
| | | gggatcggagtggctgtccttgtcgcggtggttctggccgggaccgcggtag |
| | | tgtacctcacccacgcctcctcggtgcgctatcgtcggctgcggtaaCGTAC |
| | | GGCTGGAGCCTCGGTGGCCTAGCTTCTTGCCCCTTGGGCCTCCCCCCAGCCC |
| | | CTCCTCCCCTTCCTGCACCCGTACCCCCGTGGTCTTTGAATAAAGTCTGAGT |
| | | GGGCGGCAaaaaaaaaaaaaaaaaaaaaaaaaaaaaagcatatgactaaaaa |
| | | aaaaaaaaaaaaaaaaaaaaaaaaaaaaaaaaaaaaaaaaaaaaaaaaaaaa |
| | | aaaaaaaaaaaaaAAAAGAAGAGCAAGCTT |
| ARV-HSV-gE (HSV- | 623 | MRMQLLLLIALSLALVINSRISWKRVTSGEDVVLLPAPAGPEERTRAHKLLW |
| 2 glycoprotein E), | | AAEPLDACGPLRPSWVALWPPRRVLETVVDAACMRAPEPLAIAYSPPFPAGD |
| Protein sequence | | EGLYSELAWRDRVAVVNESLVEYGALETDSGLYTLSVVGLSDEARQVASVVL |
| | | VVEPAPVPTPTPDDYDEEDDAGVSERTPVSVPPPTPPRRPPVAPPTHPRVIP |
| | | EVSHVRGVTVHMETPEAILFAPGETFGTNVSIHAIAHDDGPYAMDVVWMRFD |
| | | VPSSCAEMRIYEACLYHPQLPECLSPADAPCAVSSWAYRLAVRSYAGCSRTT |
| | | PPPRCFAEARMEPVPGLAWLASTVNLEFQHASPQHAGLYLCVVYVDDHIHAW |
| | | GHMTISTAAQYRNAVVEQHLPQRQPEPVEPTRPHVRAPHPAPSARGPERLGA |
| | | VLGAALLLAALGLSAWACMTCWRRRSWRAVKSRA* |
| ARV-HSV-gE (HSV- | 624 | GAATTCTAATACGACTCACTATAAGGACATTAGCTTCTGACACAACTGTGTT |
| 2 glycoprotein E), | | CACTAGCAACCTCAAACAGCCACCATGCGCATGCAGCTGCTGCTGCTGATCG |
| DNA sequence | | CCCTGTCCCTGGCCCTGGTGACCAACTCCCGCACCTCCTGGAAGCGCGTGAC |
| | | CTCCGGCGAGGACGTGGTGCTGCTGCCCGCCCCCGCCGGCCCCGAGGAGCGC |
| | | ACCCGCGCCCACAAGCTGCTGTGGGCCGCCGAGCCCCTGGACGCCTGCGGCC |
| | | CCCTGCGCCCCTCCTGGGTGGCCCTGTGGCCCCCCCGCCGCGTGCTGGAGAC |
| | | CGTGGTGGACGCCGCCTGCATGCGCGCCCCCGAGCCCCTGGCCATCGCCTAC |
| | | TCCCCCCCCTTCCCCGCCGGCGACGAGGGCCTGTACTCCGAGCTGGCCTGGC |
| | | GCGACCGCGTGGCCGTGGTGAACGAGTCCCTGGTGATCTACGGCGCCCTGGA |
| | | GACCGACTCCGGCCTGTACACCCTGTCCGTGGTGGGCCTGTCCGACGAGGCC |
| | | CGCCAGGTGGCCTCCGTGGTGCTGGTGGTGGAGCCCGCCCCCGTGCCCACCC |
| | | CCACCCCCGACGACTACGACGAGGAGGACGACGCCGGCGTGTCCGAGCGCAC |
| | | CCCCGTGTCCGTGCCCCCCCCCACCCCCCCCCGCCGCCCCCCCGTGGCCCCC |
| | | CCCACCCACCCCCGCGTGATCCCCGAGGTGTCCCACGTGCGCGGCGTGACCG |
| | | TGCACATGGAGACCCCCGAGGCCATCCTGTTCGCCCCCGGCGAGACCTTCGG |
| | | CACCAACGTGTCCATCCACGCCATCGCCCACGACGACGGCCCCTACGCAATG |
| | | GACGTGGTGTGGATGCGCTTCGACGTGCCCTCCTCCTGCGCCGAGATGCGCA |
| | | TCTACGAGGCCTGCCTGTACCACCCCCAGCTGCCCGAGTGCCTGTCCCCCGC |
| | | CGACGCCCCCTGCGCCGTGTCCTCCTGGGCCTACCGCCTGGCCGTGCGCTCC |
| | | TACGCCGGCTGCTCCCGCACCACCCCCCCCCCCCGCTGCTTCGCCGAGGCCC |
| | | GCATGGAGCCCGTGCCCGGCCTGGCCTGGCTGGCCTCCACCGTGAACCTGGA |
| | | GTTCCAGCACGCCTCCCCCCAGCACGCCGGCCTGTACCTGTGCGTGGTGTAC |
| | | GTGGACGACCACATCCACGCCTGGGGCCACATGACCATCTCCACCGCCGCCC |
| | | AGTACCGCAACGCCGTGGTGGAGCAGCACCTGCCCCAGCGCCAGCCCGAGCC |
| | | CGTGGAGCCCACCCGCCCCCACGTGCGCGCCccccatcccgcgccctccgcg |
| | | cgcggcccgctgcgcctcggggcggtgctgggggcggccctgttgctggccg |
| | | ccctcgggctgtccgcgtgggcgtgcatgacctgctggcgcaggcgctcctg |
| | | gcgggcggttaaaagccgggccTAACGTACGGCTGGAGCCTCGGTGGCCTAG |
| | | CTTCTTGCCCCTTGGGCCTCCCCCCAGCCCCTCCTCCCCTTCCTGCACCCGT |
| | | ACCCCCGTGGTCTTTGAATAAAGTCTGAGTGGGCGGCAaaaaaaaaaaaaaa |
| | | aaaaaaaaaaaaaaagcatatgactaaaaaaaaaaaaaaaaaaaaaaaaaaa |
| | | aaaaaaaaaaaaaaaaaaaaaaaaaaaaaaaaaaaaaaaaaaaAAAAGAAGA |
| | | GCAAGCTT |
| ARV-HSV-MTE | 625 | MGFEAGLMDAATPPARPPAERQGSPTPADATRYLPIAGASSVVALAPYVNKT |
| (Multi-T-cell- | | VTGDCLPVLDMETGHIGAYVVLVDQTGNVADLLRAAAPAWSRRTLLPEHARN |
| Epitopes derived from | | CVRPPDYPTPPASEWNSLWMTPVGNMLFDQGTLVGALDFGAGPMRARPRGEV |
| HSV-1 and HSV-2); | | RFLHYDEAGYALYRDSSSDDDESRDTARPRRSASVAGSHGPGPARAPPPPGG |
| Protein sequence | | PVGAGGRSHAPPARTPKMTRGAPKASATPAIDPARGRRPAQASGPHETITAL |
| | | VGAARHASPFERVRCLLLRSEGRKSRRPLTTFGSGAAAPAWSRRTLLPEHHN |
| | | LFLWEDQTLLRATKNACPLLIFDRTRKFVLACPYADRLDNRLQLGMLIPGAV |
| | | KRFGGHYMESVFQMYTRIAILGILVHLRIRTREASFEEETHFTQYLIYDASP |
| | | LKGLSAMKTSNALCVRGARPFSHFPALMLEYFCRCAREESKRVDTSMSLADF |
| | | HGEEFEKLYETQYLIYDASPLKGLSLTRMAEYDRVHIYYNHRISNQALLRMA |
| | | CEVRQGNWTGAPDVSALGAQGMGFLVDAIVRVAINGLLGLADTVVACVACRR |
| | | LGPADRRFVALSGSLEVWASDSLNNEYLRLAAQREAAGVYDAVRIWGP* |
| ARV-HSV-MTE | 626 | GAATTCGGCTTTAATACGACTCACTATAAGGACATTAGCTTCTGACACAACT |
| (Multi-T-cell- | | GTGTTCACTAGCAACCTCAAACAGCCACCATGggcTTCGAGGCCGGCCTGAT |
| Epitopes derived from | | GGACGCCGCGACCCCGCCGGCCCGACCGCCCGCGGAACGGCAAGGCAGCCCG |
| HSV-1 and HSV-2); | | ACCCCCGCAGACGCCACCCGGTACCTGCCGATCGCCGGCGCCAGCAGCGTCG |
| DNA sequence | | TGGCGCTGGCCCCCTACGTGAACAAGACCGTGACCGGCGACTGCCTCCCGGT |
| | | CCTGGACATGGAAACCGGCCACATCGGCGCATACGTGGTGCTGGTGGACCAG |
| | | ACAGGCAACGTGGCCGACCTGCTGCGGGCCGCAGCCCCCGCGTGGAGCAGGC |
| | | GGACCCTACTGCCCGAGCACGCCCGCAACTGCGTGCGACCCCCGGACTACCC |
| | | GACCCCGCCAGCGAGCGAGTGGAACAGCCTGTGGATGACCCCGGTGGGCAAC |
| | | ATGCTGTTCGACCAGGGCACACTGGTGGGCGCCCTCGACTTCGGCGCCGGCC |
| | | CGATGAGAGCCCGACCAAGAGGCGAGGTGCGCTTCCTGCACTACGACGAGGC |
| | | CGGGTACGCCCTGTACAGGGACAGCAGCAGCGACGACGACGAGAGCCGGGAC |
| | | ACCGCCCGCCCCAGACGGAGCGCCAGCGTGGCCGGGAGCCACGGCCCGGGCC |
| | | CCGCCAGAGCGCCGCCGCCGCCAGGGGGGCCGGTGGGGGCCGGGGGACGAAG |
| | | CCACGCCCCGCCGGCGAGAACACCGAAGATGACGCGCGGCGCCCCGAAGGCC |
| | | AGCGCGACCCCCGCCACCGACCCGGCCCGGGGACGCCGCCCGGCCCAGGCCA |
| | | GCGGCCCGCACGAAACAATCACAGCCCTGGTGGGCGCCGCGCGGCACGCCAG |
| | | CCCCTTCGAGCGGGICAGGTGCCTGCTGCTCAGAAGCGAGGGCCGGAAAAGC |
| | | CGCAGACCGCTAACGACCTTCGGCAGCGGCGCCGCGGCCCCGGCCTGGAGCC |
| | | GGCGAACCCTGCTGCCCGAGCACCACAACCIGTTCCTGTGGGAGGACCAGAC |
| | | CCTGCTGCGGGCCACCAAGAACGCCTGCCCCCTCCTGATCTTCGACAGAACC |
| | | CGCAAGTTCGTGCTGGCCTGCCCCTACGCCGACAGGCTGGACAACCGCCTGC |
| | | AACTGGGAATGCTGATCCCGGGAGCCGTGAAGCGGTTCGGCGGCCACTACAT |
| | | GGAAAGCGTGTTCCAGATGTACACCCGGATCGCGATCCTGGGCATCCTGGTG |
| | | CACCTGCGGATCAGAACCCGGGAGGCGAGCTTCGAAGAAGAAACCCACTTCA |
| | | CCCAGTACCTGATCTACGACGCCAGCCCGCTGAAGGGCCTGAGCGCCATGAA |
| | | AACAAGCAACGCGCTGTGCGTAAGGGGCGCCCGGCCATTCAGCCACTTCCCG |
| | | GCGCTGATGCTGGAGTACTTCTGCAGATGCGCCAGAGAAGAGAGCAAACGCG |
| | | TGGACACAAGCATGAGCCTGGCAGACTICCACGGCGAGGAGTTCGAGAAGCT |
| | | GTACGAGACACAGTACCTGATCTACGACGCCAGCCCACTGAAGGGCCTGAGC |
| | | CTGACCCGGATGGCAGAGTACGACCGGGTGCACATCTACTACAACCACCGGA |
| | | TCAGCAACCAGGCGCTGCTGAGGATGGCCTGCGAGGTGCGCCAGGGCAACTG |
| | | GACCGGGGCGCCGGACGTGAGCGCGCTGGGCGCGCAGGGCATGGGCTTCCTG |
| | | GTGGACGCGATCGTCCGGGTGGCGATAAACGGCCTCCTGGGGCTGGCGGACA |
| | | CCGTGGTGGCCTGCGTGGCGTGCAGACGCCTGGGCCCCGCGGACAGACGGTT |
| | | CGTGGCCCTGAGCGGCAGCCTGGAGGTGTGGGCCAGCGACAGCCTGAACAAC |
| | | GAGTACCTGCGCCTGGCGGCCCAGCGGGAGGCGGCCGGCGTGTACGACGCGG |
| | | TCCGGACCTGGGGCCCGTGACGTACGGCTGGAGCCTCGGTGGCCTAGCTTCT |
| | | TGCCCCTTGGGCCTCCCCCCAGCCCCTCCTCCCCTTCCTGCACCCGTACCCC |
| | | CGTGGTCTTTGAATAAAGTCTGAGTGGGCGGCAaaaaaaaaaaaaaaaaaaa |
| | | aaaaaaaaaaaaaaaaaaaaaaaaaaaaaaaaaaaaaaAAAAGAAGAGCAAG |
| | | aaaaaaaaaaaaaaaaaaaaaaaaaaaaaaaaaaaaaaAAAAGAAGAGCAAG |
| | | CTT |
| ARV-HSV-UL19UD | 627 | MGRLSMENAVGTVCHPSLMNIDAAVGGVNHDPVEAANPYGAYVAAPAGPGAD |
| (trucated HSV-2 | | MQQRFLNAWRQRLAHGRVRWVAECQMTAEQEMQPDNANLALELHPAFDFFAG |
| UL19 UD sequence), | | VADVELPGGEVPPAGPGAIQATWRVVNGNLPLALCPVAFRDARGLELGVGRH |
| Protein sequence | | AMAPATIAAVRGAFEDRSYPAVFYLLQAAIHGSEHVFCALARLVTQCITSYW |
| | | NNTRCAAFVNDYSLVSYIVTYLGGDLPEECMAVYRDLVAHVEALAQLVDDFT |
| | | LPGPELGGQAQAELNHLMRDPALLPPLVWDCDGLMRHAALDRHRDCRIDAGG |
| | | HEPVYAAACNVATADFNRNDGRLLHNTQARAADAADDRPHRPADWTVHHKIY |
| | | YYVLVPAFSRGRCCTAGVRFDRVYATLQNMVVPEIAPGEECPSDPVTDPAHP |
| | | LHPANLVANTVNAMFHNGRVVVDGPAMLTLQVLAHNMAERTTALLCSAAPDA |
| | | GANTASTANMRIFDGALHAGVLLMAPQHLDHTIQNGEYFYVLPVHALFAGAD |
| | | HVANAPNFPPALRDLARHVPLVPPALGANYESSIRQPVVQHARESAAGENAL |
| | | TYALMAGYFKMSPVALYIIQLKTGLIIPGFGFTVVR* |
| ARV-HSV-UL19UD | 628 | GAATTCGGCTTTAATACGACTCACTATAAGGACATTAGCTTCTGACACAACT |
| (truncated HSV-2 | | GTGTTCACTAGCAACCTCAAACAGCCACCATGggcAGGCTGAGCATGGAAAA |
| UL19 UD sequence), | | CGCGGTGGGCACAGTCTGCCACCCCAGCCTGATGAACATCGACGCCGCCGTG |
| DNA sequence | | GGCGGCGTGAACCACGACCCCGTGGAAGCCGCCAACCCGTATGGCGCCTACG |
| | | TGGCCGCCCCAGCCGGCCCCGGCGCCGACATGCAGCAGCGGTTCCTGAACGC |
| | | CTGGCGGCAACGGCTGGCCCACGGACGGGTGAGATGGGTGGCCGAGTGCCAG |
| | | ATGACCGCGGAGCAGTTCATGCAGCCGGACAACGCCAACCTGGCCCTGGAGC |
| | | TGCACCCCGCCTTCGACTTCTTCGCCGGAGTGGCCGACGTGGAGCTGCCGGG |
| | | CGGAGAGGTGCCGCCGGCCGGACCGGGCGCCATCCAGGCAACGTGGCGGGTA |
| | | GTGAACGGCAACCTGCCCCTGGCCCTCTGCCCCGTGGCCTTCCGGGACGCCA |
| | | GAGGACTCGAGCTGGGCGTAGGCAGACACGCGATGGCCCCGGCGACCATCGC |
| | | GGCGGTGCGCGGCGCATTCGAGGACCGGAGCTACCCCGCCGTGTTCTACCTG |
| | | CTGCAGGCCGCCATCCACGGAAGCGAACACGTGTTCTGCGCCCTCGCCCGGC |
| | | TGGTCACCCAATGCATCACGAGCTACTGGAACAACACCAGATGCGCCGCGTT |
| | | CGTGAACGACTACAGCCTCGTGAGCTACATCGTGACCTACCTGGGCGGCGAC |
| | | CTGCCGGAGGAGTGCATGGCGGTCTACCGAGACCTGGTCGCCCACGTGGAGG |
| | | CCCTGGCCCAGCTGGTGGACGACTTCACCCTGCCCGGCCCGGAGCTGGGCGG |
| | | GCAGGCCCAGGCCGAGCTGAACCACCTGATGCGGGACCCGGCACTGCTCCCG |
| | | CCGCTGGTGTGGGACTGCGACGGCCTGATGCGCCACGCCGCCCTGGACAGAC |
| | | ACCGGGACTGCAGGATCGACGCCGGAGGCCACGAACCGGTGTACGCCGCCGC |
| | | CTGCAACGTGGCCACCGCCGACTTCAACCGCAACGACGGCCGGCTGCTGCAC |
| | | AACACACAGGCCAGGGCGGCAGACGCCGCCGACGACCGGCCCCACAGACCCG |
| | | CCGACTGGACGGTCCACCACAAGATCTACTACTACGTGCTGGTGCCGGCGTT |
| | | CAGCAGGGGCCGGTGCTGCACCGCGGGCGTGCGGTTCGACCGGGTGTACGCC |
| | | ACACTGCAAAACATGGTGGTGCCCGAAATCGCCCCGGGCGAGGAATGCCCGA |
| | | GCGACCCCGTGACGGACCCGGCGCACCCGCTGCACCCGGCGAACCTGGTGGC |
| | | GAACACCGTGAACGCCATGTTCCACAACGGCAGAGTGGTGGTGGACGGCCCG |
| | | GCGATGCTGACCCTGCAGGTGCTGGCGCACAACATGGCCGAGCGGACCACAG |
| | | CGCTGCTGTGCAGCGCCGCGCCCGACGCGGGGGCCAACACCGCCAGCACCGC |
| | | CAACATGCGGATCTTCGACGGCGCCCTGCACGCCGGCGTCCTGCTGATGGCG |
| | | CCCCAGCACCTGGACCACACGATCCAGAACGGAGAGTACTTCTACGTGCTGC |
| | | CGGTGCACGCCCTGTTCGCCGGGGCGGACCACGTCGCGAACGCCCCCAACTT |
| | | CCCGCCCGCGCTGCGGGACCTGGCCCGCCACGTGCCGCTGGTCCCCCCGGCC |
| | | CTGGGCGCGAACTACTTCAGCAGCATCAGACAACCGGTGGTCCAGCACGCGC |
| | | GCGAGAGCGCCGCCGGAGAGAACGCCCTCACCTACGCCCTCATGGCCGGCTA |
| | | CTTCAAGATGAGCCCGGTGGCGCTGTACCACCAGCTGAAAACCGGCCTGCAC |
| | | CCGGGCTTCGGCTTCACCGTGGTGCGGTGATAACGTACGGCTGGAGCCTCGG |
| | | TGGCCTAGCTTCTTGCCCCTTGGGCCTCCCCCCAGCCCCTCCTCCCCTTCCT |
| | | GCACCCGTACCCCCGTGGTCTTTGAATAAAGTCTGAGTGGGCGGCAaaaaaa |
| | | aaaaaaaaaaaaaaaaaaaaaaagcatatgactaaaaaaaaaaaaaaaaaaa |
| | | aaaaaaaaaaaaaaaaaaaaaaaaaaaaaaaaaaaaaaaaaaaaaaaaaaaA |
| | | AAAGAAGAGCAAGCTT |
| ARV-HSV-gH (HSV- | 629 | MGPGLWVVMGVLVGVAGGHDTYWTEQIDPWELHGLGLARTYWRDTNTGRLWL |
| 2 glycoprotein H); | | PNTPDASDPQRGRLAPPGELNLTTASVPMLRWYAERFCFVLVTTAEFPRDPG |
| Protein sequence | | QLLYIPKTYLLGRPRNASLPELPEAGPTSRPPAEVTQLKGLSHNPGASALLR |
| | | SRAWVTFAAAPDREGLTFPRGDDGATERHPDGRRNAPPPGPPAGAPRHPTTN |
| | | LSIAHLHNASVTWLAARGLLRTPGRYVYLSPSASTWPVGVWTTGGLAFGCDA |
| | | ATVRARYGKGFMGTVTSMRDSPPAETTVVPADKTLARVGNPTDENAPAVLPG |
| | | PPAGPRYRVFVLGAPTPADNGSALDALRRVAGYPEESTNYAQYMSRAYAEFL |
| | | GEDPGSGTDARPSLFWRLAGLLASSGFAFVNAAHAHDAIRLSDLLGFLAHSR |
| | | VLAGLAARGAAGCAADSVFLNVSVLDPAARLRLEARLGHLVAAILEREQSLA |
| | | AHALGYQLAFVLDSPAAYGAVAPSAARLIDALYAEFLGGRALTAPMVRRALF |
| | | YATAVLRAPFLAGAPSAEQRERARRGLLITTALCTSDVAAATHADLRAALAR |
| | | TDHQKNLFWLPDHFSPCAASLRFDLAEGGFILDALAMATRSDIPADVMAQQT |
| | | RGVASALTRWAHYNALIRAFVPEATHQCSGPSHNAEPRILVPITHNASYVVT |
| | | HTPLPRGIGYKLTGVDVRRPLFITYLTATCEGHAREIEPKRLVRTENRRDLG |
| | | LVGAVFLRYTPAGEVMSVLLVDTDATQQQLACGPVAGTPNVFSSDVPSVALL |
| | | LFPNGTVIHLLAFDTLPIATIAPGFLAASALGVVMITAALAGILRVVRTCVP |
| | | FLWRRE |
| ARV-HSV-gH (HSV- | 630 | GAATTCGGCTTTAATACGACTCACTATAAGGACATTAGCTTCTGACACAACT |
| 2 glycoprotein H); | | GTGTTCACTAGCAACCTCAAACAGCCACCatgggccccggtctgtgggtggt |
| DNA sequence | | gatgggcgtactggtaggcgttgccgggggccatgacacgtactggacagag |
| | | caaatcgacccgtggttcttgcacggtctggggttggcccgcacgtactggc |
| | | gcgacacaaacaccgggaggctgtggttgcccaacacccccgacgccagcga |
| | | cccccagcgcggacgcttggcgcccccgggcgaactcaacctgacaacggca |
| | | agcgtgcccatgcttcggtggtacgccgagcgcttttgtttcgtgttggtca |
| | | ccacggccgagtttccacgggaccccgggcagctgctttacatcccaaagac |
| | | ctatctgctcggccggccacggaacgcgagcctgcccgagcttcccgaggcg |
| | | gggcccacgtccaggccccccgccgaggtgacccagctcaagggactgagcc |
| | | acaaccccggcgcctccgcgctgttgaggtcccgggcctgggtaacattcgc |
| | | ggccgcgccagaccgcgaggggcttacgttcccgcggggagacgacggggcg |
| | | accgagaggcacccggacggccgacgcaacgcaccgcccccggggccgcccg |
| | | cgggagcgccgaggcatccgacgacgaacctgagcattgcgcatctgcacaa |
| | | cgcgtccgtgacatggctggccgccaggggcctgctacggacaccgggtcgg |
| | | tacgtgtacctctccccgagcgcctcgacgtggcccgtgggagtctggacga |
| | | cgggcgggctggcgttcgggtgcgatgccgcgcttgtgcgcgcgcgatacgg |
| | | gaagggcttcatggggctcgtgatttccatgcgggacagcccaccggccgag |
| | | atcattgtggtgccagcggacaagaccctcgcacgggtcggaaatccgacag |
| | | acgaaaacgcccccgcggtgctcccagggccaccagccggccccaggtatcg |
| | | cgtcttcgtcctgggggccccgacgccagccgacaatggcagcgcgctggac |
| | | gcactacggcgggtggccggctaccccgaggagagcacgaactacgcacagt |
| | | atatgagccgggcctatgcggagtttctaggggaggacccgggaagcggcac |
| | | ggacgcgaggccgagcctgttctggcgcctcgcagggctgctcgccagcagc |
| | | gggtttgcgttcgtcaacgcggcacacgcccacgacgcgattcgcctcagcg |
| | | acctgctgggctttctagcccacagccgcgtgctggccggcctggccgcccg |
| | | gggagcagcgggctgtgcggccgacagcgtgttcctgaacgtgagcgtgttg |
| | | gacccggcggccaggctgcggcttgaggcgcgcctcggacatctggtggcag |
| | | cgatcctcgagcgagagcagagcctagcggcgcacgcgctgggctatcagct |
| | | ggcgttcgtgttggacagccccgcggcctatggcgcggtggccccgagcgcg |
| | | gcccgcctgatcgacgcactgtacgccgagtttctcggcggccgcgcgctaa |
| | | ccgccccgatggtccgccgagcgctgttttacgcaacggccgtcctccgggc |
| | | accgttcctggcaggcgcgcccagcgccgagcagcgggaacgcgcccgccgg |
| | | ggactcctcataaccacggcactgtgtacgagcgacgtagcagcggcgaccc |
| | | acgccgatctccgggccgcgctcgccaggaccgaccaccagaaaaacctctt |
| | | ctggctcccggaccacttttccccatgcgcagcatccctgcgcttcgatctc |
| | | gccgaaggagggttcatcctggacgcactgGCGatggccacccgatccgaca |
| | | tcccggcggacgtcatggcacaacagacccgcggcgtggcctccgcacttac |
| | | gcgctgggcgcactacaacgccctgatccgcgccttcgtcccggaggccacc |
| | | catcagtgtagcggcccgagccacaacgcggagccccggatcctcgtaccca |
| | | tcacccacaacgccagctatgtcgtcacacacacccccttgccacgcgggat |
| | | cggatacaagctcacgggcgttgacgtccgccgcccactgtttatcacctat |
| | | ctcaccgccacctgcgaagggcacgcgcgggagattgagccgaaacggctgg |
| | | tgcgcaccgaaaaccggcgcgaccttggcctcgtgggggccgtgtttctgcg |
| | | ctacaccccggccggggaggtcatgagcgtgctgctagtggacacggatgcc |
| | | acccaacagcagctggcccaggggccggtggcgggcaccccgaacgtgtttt |
| | | ccagcgatgtaccaagcgtggccctgttattgttccccaatggaacagtgat |
| | | tcatctgctggcctttgacacgctgcccatcgccaccatcgcccccgggttc |
| | | ctggccgcgtccgcgcttggagtcgttatgattaccgcggcactggcaggca |
| | | tcctcagggtggtccgaacgtgcgtcccatttttgtggagacgcgaatgaCG |
| | | TACGGCTGGAGCCTCGGTGGCCTAGCTTCTTGCCCCTTGGGCCTCCCCCCAG |
| | | CCCCTCCTCCCCTTCCTGCACCCGTACCCCCGTGGTCTTTGAATAAAGTCTG |
| | | AGTGGGCGGCAaaaaaaaaaaaaaaaaaaaaaaaaaaaaagcatatgactaa |
| | | aaaaaaaaaaaaaaaaaaaaaaaaaaaaaaaaaaaaaaaaaaaaaaaaaaaa |
| | | aaaaaaaaaaaaaaaaAAAAGAAGAGCAAGCTT |
| ARV-HSV-gH-HA | 631 | MGPGLWVVMGVLVGVAGGHDTYWTEQIDPWFLHGLGLARTYWRDTNTGRLWL |
| (HSV-2 glycoprotein | | PNTPDASDPQRGRLAPPGELNLTTASVPMLRWYAERFCFVLVITAEFPRDPG |
| H-HA tag), Protein | | QLLYIPKTYLLGRPRNASLPELPEAGPTSRPPAEVTQLKGLSHNPGASALLR |
| sequence. | | SRAWVTFAAAPDREGLTFPRGDDGATERHPDGRRNAPPPGPPAGAPRHPTTN |
| | | LSIAHLHNASVTWLAARGLLRTPGRYVYLSPSASTWPVGVWTTGGLAFGCDA |
| | | ALVRARYGKGFMGLVISMRDSPPAEIIVVPADKTLARVGNPIDENAPAVLPG |
| | | PPAGPRYRVFVLGAPTPADNGSALDALRRVAGYPEESTNYAQYMSRAYAEFL |
| | | GEDPGSGTDARPSLFWRLAGLLASSGFAFVNAAHAHDAIRLSDLLGFLAHSR |
| | | VLAGLAARGAAGCAADSVFLNVSVLDPAARLRLEARLGHLVAAILEREQSLA |
| | | AHALGYQLAFVLDSPAAYGAVAPSAARLIDALYAEFLGGRALTAPMVRRALF |
| | | YATAVLRAPFLAGAPSAEQRERARRGLLITTALCTSDVAAATHADLRAALAR |
| | | TDHQKNTFWTPQHFSPCAASTRFDLAEGGFIIDATAMATRSDTPADVMAQQT |
| | | RGVASALTRWAHYNALIRAFVPEATHQCSGPSHNAEPRILVPITHNASYVVT |
| | | HTPLPRGIGYKLTGVDVRRPLFITYLTATCEGHAREIEPKRLVRTENRRDLG |
| | | LVGAVFLRYTPAGEVMSVLLVDTDATQQQLAQGPVAGTPNVFSSDVPSVALL |
| | | LFPNGTVIHLLAFDTLPIATIAPGFLAASALGVVMITAALAGILRVVRTCVP |
| | | FLWRREDIYPYDVPDYA* |
| ARV-HSV-gH-HA | 632 | GAATTCGGCTTTAATACGACTCACTATAAGGACATTAGCTTCTGACACAACT |
| (HSV-2 glycoprotein | | GTGTTCACTAGCAACCTCAAACAGCCACCatgggccccggtctgtgggtggt |
| H-HA tag), DNA | | gatgggcgtactggtaggcgttgccgggggccatgacacgtactggacagag |
| sequence. | | caaatcgacccgtggttcttgcacggtctggggttggcccgcacgtactggc |
| | | gcgacacaaacaccgggaggctgtggttgcccaacacccccgacgccagcga |
| | | cccccagcgcggacgcttggcgcccccgggcgaactcaacctgacaacggca |
| | | agcgtgcccatgcttcggtggtacgccgagcgcttttgtttcgtgttggtca |
| | | ccacggccgagtttccacgggaccccgggcagctgctttacatcccaaagac |
| | | ctatctgctcggccggccacggaacgcgagcctgcccgagcttcccgaggcg |
| | | gggcccacgtccaggccccccgccgaggtgacccagctcaagggactgagcc |
| | | acaaccccggcgcctccgcgctgttgaggtcccgggcctgggtaacattcgc |
| | | ggccgcgccagaccgcgaggggcttacgttcccgcggggagacgacggggcg |
| | | accgagaggcacccggacggccgacgcaacgcaccgcccccggggccgcccg |
| | | cgggagcgccgaggcatccgacgacgaacctgagcattgcgcatctgcacaa |
| | | cgcgtccgtgacatggctggccgccaggggcctgctacggacaccgggtcgg |
| | | tacgtgtacctctccccgagcgcctcgacgtggcccgtgggagtctggacga |
| | | cgggcgggctggcgttcgggtgcgatgccgcgcttgtgcgcgcgcgatacgg |
| | | gaagggcttcatggggctcgtgatttccatgcgggacagcccaccggccgag |
| | | atcattgtggtgccagcggacaagaccctcgcacgggtcggaaatccgacag |
| | | acgaaaacgcccccgcggtgctcccagggccaccagccggccccaggtatcg |
| | | cgtcttcgtcctgggggccccgacgccagccgacaatggcagcgcgctggac |
| | | gcactacggcgggtggccggctaccccgaggagagcacgaactacgcacagt |
| | | atatgagccgggcctatgcggagtttctaggggaggacccgggaagcggcac |
| | | ggacgcgaggccgagcctgttctggcgcctcgcagggctgctcgccagcagc |
| | | gggtttgcgttcgtcaacgcggcacacgcccacgacgcgattcgcctcagcg |
| | | acctgctgggctttctagcccacagccgcgtgctggccggcctggccgcccg |
| | | gggagcagcgggctgtgcggccgacagcgtgttcctgaacgtgagcgtgttg |
| | | gacccggcggccaggctgcggcttgaggcgcgcctcggacatctggtggcag |
| | | cgatcctcgagcgagagcagagcctagcggcgcacgcgctgggctatcagct |
| | | ggcgttcgtgttggacagccccgcggcctatggcgcggtggccccgagcgcg |
| | | gcccgcctgatcgacgcactgtacgccgagtttctcggcggccgcgcgctaa |
| | | ccgccccgatggtccgccgagcgctgttttacgcaacggccgtcctccgggc |
| | | accgttcctggcaggcgcgcccagcgccgagcagcgggaacgcgcccgccgg |
| | | ggactcctcataaccacggcactgtgtacgagcgacgtagcagcggcgaccc |
| | | acgccgatctccgggccgcgctcgccaggaccgaccaccagaaaaacctctt |
| | | ctggctcccggaccacttttccccatgcgcagcatccctgcgcttcgatctc |
| | | gccgaaggagggttcatcctggacgcactgGCGatggccacccgatccgaca |
| | | tcccggcggacgtcatggcacaacagacccgcggcgtggcctccgcacttac |
| | | gcgctgggcgcactacaacgccctgatccgcgccttcgtcccggaggccacc |
| | | catcagtgtagcggcccgagccacaacgcggagccccggatcctcgtaccca |
| | | tcacccacaacgccagctatgtcgtcacacacacccccttgccacgcgggat |
| | | cggatacaagctcacgggcgttgacgtccgccgcccactgtttatcacctat |
| | | ctcaccgccacctgcgaagggcacgcgcgggagattgagccgaaacggctgg |
| | | tgcgcaccgaaaaccggcgcgaccttggcctcgtgggggccgtgtttctgcg |
| | | ctacaccccggccggggaggtcatgagcgtgctgctagtggacacggatgcc |
| | | acccaacagcagctggcccaggggccggtggcgggcaccccgaacgtgtttt |
| | | ccagcgatgtaccaagcgtggccctgttattgttccccaatggaacagtgat |
| | | tcatctgctggcctttgacacgctgcccatcgccaccatcgcccccgggttc |
| | | ctggccgcgtccgcgcttggagtcgttatgattaccgcggcactggcaggca |
| | | tcctcagggtggtccgaacgtgcgtcccatttttgtggagacgcgaaGATAT |
| | | CTACCCATACGACGTACCAGATTACGCTtgaCGTACGGCTGGAGCCTCGGTG |
| | | GCCTAGCTTCTTGCCCCTTGGGCCTCCCCCCAGCCCCTCCTCCCCTTCCTGC |
| | | ACCCGTACCCCCGTGGTCTTTGAATAAAGTCTGAGTGGGCGGCAaaaaaaaa |
| | | aaaaaaaaaaaaaaaaaaaaagcatatgactaaaaaaaaaaaaaaaaaaaaa |
| | | aaaaaaaaaaaaaaaaaaaaaaaaaaaaaaaaaaaaaaaaaaaaaaaaaAAA |
| | | AGAAGAGCAAGCTT |
| ARV-HSV-mRNA-3 | 343 | MGFEAGLMDAATPPARPPAERQGSPTPADATRYLPIAGASSVVALAPYVNKT |
| (HSV-2 UL19UD and | | VTGDCLPVLDMETGHIGAYVVLVDQTGNVADLLRAAAPAWSRRILLPEHARN |
| Multi-T-cell- | | CVRPPDYPTPPASEWNSLWMTPVGNMLFDQGTLVGALDFGAGPMRARPRGEV |
| Epitopes); Protein | | RFLHYDEAGYALYRDSSSDDDESRDTARPRRSASVAGSHGPGPARAPPPPGG |
| sequence | | PVGAGGRSHAPPARTPKMTRGAPKASATPATDPARGRRPAQASGPHETITAL |
| | | VGAARHASPFERVRCLLLRSEGRKSRRPTTTFGSGAAAPAWSRRTLLPEHHN |
| | | LFLWEDQTLLRATKNACPLLIFDRTRKFVLACPYADRLDNRLQLGMLIPGAV |
| | | KRFGGHYMESVFQMYTRIAILGILVHLRIRTREASFEEETHFTQYLIYDASP |
| | | LKGLSAMKTSNALCVRGARPFSHFPALMLEYFCRCAREESKRVDTSMSLADF |
| | | HGEEFEKLYETQYLIYDASPLKGLSLTRMAEYDRVHIYYNHRISNQALLRMA |
| | | CEVRQGNWTGAPDVSALGAQGMGFLVDAIVRVAINGLLGLADTVVACVACRR |
| | | LGPADRRFVALSGSLEVWASDSLNNEYLRLAAQREAAGVYDAVRTWGPATNF |
| | | SLLKQAGDVEENPGPRLSMFNAVGTVCHPSLMNIDAAVGGVNHDPVEAANPY |
| | | GAYVAAPAGPGADMQQRFLNAWRQRLAHGRVRWVAECQMTAEQFMQPDNANL |
| | | ALELHPAFDFFAGVADVELPGGEVPPAGPGAIQATWRVVNGNLPLALCPVAF |
| | | RDARGLELGVGRHAMAPATIAAVRGAFEDRSYPAVFYLLQAAIHGSEHVFCA |
| | | LARLVTQCITSYWNNTRCAAFVNDYSLVSYIVTYLGGDLPEECMAVYRDLVA |
| | | HVEALAQLVDDFTLPGPELGGQAQAELNHLMRDPALLPPLVWDCDGLMRHAA |
| | | LDRHRDCRIDAGGHEPVYAAACNVATADFNRNDGRLLHNTQARAADAADDRP |
| | | HRPADWTVHHKIYYYVTVPAFSRGRCCTAGVRFDRVYATLQNMVVPEIAPGE |
| | | ECPSDPVTDPAHPLHPANLVANTVNAMFHNGRVVVDGPAMLTLQVLAHNMAE |
| | | RTTALLCSAAPDAGANTASTANMRIFDGALHAGVLLMAPQHLDHTIQNGEYF |
| | | YVLPVHALFAGADHVANAPNFPPALRDLARHVPLVPPALGANYFSSIRQPVV |
| | | QHARESAAGENALTYALMAGYFKMSPVALYHQLKTGLHPGFGFTVVR |
| ARV-HSV-mRNA-3 | 344 | ATGggcTTCGAGGCCGGCCTGATGGACGCCGCGACCCCGCCGGCCCGACCGC |
| (HSV-2 UL19UD and | | CCGCGGAACGGCAAGGCAGCCCGACCCCCGCAGACGCCACCCGGTACCTGCC |
| Multi-T-cell- | | GATCGCCGGCGCCAGCAGCGTCGTGGCGCTGGCCCCCTACGTGAACAAGACC |
| Epitopes); DNA | | GTGACCGGCGACTGCCTCCCGGTCCTGGACATGGAAACCGGCCACATCGGCG |
| sequence | | CATACGTGGTGCTGGTGGACCAGACAGGCAACGTGGCCGACCTGCTGCGGGC |
| | | CGCAGCCCCCGCGTGGAGCAGGCGGACCCTACTGCCCGAGCACGCCCGCAAC |
| | | TGCGTGCGACCCCCGGACTACCCGACCCCGCCAGCGAGCGAGTGGAACAGCC |
| | | TGTGGATGACCCCGGTGGGCAACATGCTGTTCGACCAGGGCACACTGGTGGG |
| | | CGCCCTCGACTTCGGCGCCGGCCCGATGAGAGCCCGACCAAGAGGCGAGGTG |
| | | CGCTTCCTGCACTACGACGAGGCCGGGTACGCCCTGTACAGGGACAGCAGCA |
| | | GCGACGACGACGAGAGCCGGGACACCGCCCGCCCCAGACGGAGCGCCAGCGT |
| | | GGCCGGGAGCCACGGCCCGGGCCCCGCCAGAGCGCCGCCGCCGCCAGGGGGG |
| | | CCGGTGGGGGCCGGGGGACGAAGCCACGCCCCGCCGGCGAGAACACCGAAGA |
| | | TGACGCGCGGCGCCCCGAAGGCCAGCGCGACCCCCGCCACCGACCCGGCCCG |
| | | GGGACGCCGCCCGGCCCAGGCCAGCGGCCCGCACGAAACAATCACAGCCCTG |
| | | GTGGGCGCCGCGCGGCACGCCAGCCCCTTCGAGCGGGTCAGGTGCCTGCTGC |
| | | TCAGAAGCGAGGGCCGGAAAAGCCGCAGACCGCTAACGACCTTCGGCAGCGG |
| | | CGCCGCGGCCCCGGCCTGGAGCCGGCGAACCCTGCTGCCCGAGCACCACAAC |
| | | CTGTTCCTGTGGGAGGACCAGACCCTGCTGCGGGCCACCAAGAACGCCTGCC |
| | | CCCTCCTGATCTTCGACAGAACCCGCAAGTTCGTGCTGGCCTGCCCCTACGC |
| | | CGACAGGCTGGACAACCGCCTGCAACTGGGAATGCTGATCCCGGGAGCCGTG |
| | | AAGCGGTTCGGCGGCCACTACATGGAAAGCGTGTTCCAGATGTACACCCGGA |
| | | TCGCGATCCTGGGCATCCTGGTGCACCTGCGGATCAGAACCCGGGAGGCGAG |
| | | CTTCGAAGAAGAAACCCACTTCACCCAGTACCTGATCTACGACGCCAGCCCG |
| | | CTGAAGGGCCTGAGCGCCATGAAAACAAGCAACGCGCTGTGCGTAAGGGGCG |
| | | CCCGGCCATTCAGCCACTTCCCGGCGCTGATGCTGGAGTACTTCTGCAGATG |
| | | CGCCAGAGAAGAGAGCAAACGCGTGGACACAAGCATGAGCCTGGCAGACTTC |
| | | CACGGCGAGGAGTTCGAGAAGCTGTACGAGACACAGTACCTGATCTACGACG |
| | | CCAGCCCACTGAAGGGCCTGAGCCTGACCCGGATGGCAGAGTACGACCGGGT |
| | | GCACATCTACTACAACCACCGGATCAGCAACCAGGCGCTGCTGAGGATGGCC |
| | | TGCGAGGTGCGCCAGGGCAACTGGACCGGGGCGCCGGACGTGAGCGCGCTGG |
| | | GCGCGCAGGGCATGGGCTTCCTGGTGGACGCGATCGTCCGGGTGGCGATAAA |
| | | CGGCCTCCTGGGGCTGGCGGACACCGTGGTGGCCTGCGTGGCGTGCAGACGC |
| | | CTGGGCCCCGCGGACAGACGGTTCGTGGCCCTGAGCGGCAGCCTGGAGGTGT |
| | | GGGCCAGCGACAGCCTGAACAACGAGTACCTGCGCCTGGCGGCCCAGCGGGA |
| | | GGCGGCCGGCGTGTACGACGCGGTCCGGACCTGGGGCCCGGCCACCAACTTC |
| | | AGCCTGCTGAAGCAGGCGGGGGACGTGGAAGAGAACCCCGGCCCGAGGCTGA |
| | | GCATGGAAAACGCGGTGGGCACAGTCTGCCACCCCAGCCTGATGAACATCGA |
| | | CGCCGCCGTGGGCGGCGTGAACCACGACCCCGTGGAAGCCGCCAACCCGTAC |
| | | GGCGCCTACGTGGCCGCCCCAGCCGGCCCCGGCGCCGACATGCAGCAGCGGT |
| | | TCCTGAACGCCTGGCGGCAACGGCTGGCCCACGGACGGGTGAGATGGGTGGC |
| | | CGAGTGCCAGATGACCGCGGAGCAGTTCATGCAGCCGGACAACGCCAACCTG |
| | | GCCCTGGAGCTGCACCCCGCCTTCGACTTCTTCGCCGGAGTGGCCGACGTGG |
| | | AGCTGCCGGGCGGAGAGGTGCCGCCGGCCGGACCGGGCGCCATCCAGGCAAC |
| | | GTGGCGGGTAGTGAACGGCAACCTGCCCCTGGCCCTCTGCCCCGTGGCCTTC |
| | | CGGGACGCCAGAGGACTCGAGCTGGGCGTAGGCAGACACGCCATGGCCCCGG |
| | | CGACCATCGCGGCGGTGCGCGGCGCATTCGAGGACCGGAGCTACCCCGCCGT |
| | | GTTCTACCTGCTGCAGGCCGCCATCCACGGAAGCGAACACGTGTTCTGCGCC |
| | | CTCGCCCGGCTGGTCACCCAATGCATCACGAGCTACTGGAACAACACCAGAT |
| | | GCGCCGCGTTCGTGAACGACTACAGCCTCGTGAGCTACATCGTGACCTACCT |
| | | GGGCGGCGACCTGCCGGAGGAGTGCATGGCGGTCTACCGAGACCTGGTCGCC |
| | | CACGTGGAGGCCCTGGCCCAGCTGGTGGACGACTTCACCCTGCCCGGCCCGG |
| | | AGCTGGGCGGGCAGGCCCAGGCCGAGCTGAACCACCTGATGCGGGACCCGGC |
| | | ACTGCTCCCGCCGCTGGTGTGGGACTGCGACGGCCTGATGCGCCACGCCGCC |
| | | CTGGACAGACACCGGGACTGCAGGATCGACGCCGGAGGCCACGAACCGGTGT |
| | | ACGCCGCCGCCTGCAACGTGGCCACCGCCGACTTCAACCGCAACGACGGCCG |
| | | GCTGCTGCACAACACACAGGCCAGGGCGGCAGACGCCGCCGACGACCGGCCC |
| | | CACAGACCCGCCGACTGGACGGTCCACCACAAGATCTACTACTACGTGCTGG |
| | | TGCCGGCGTTCAGCAGGGGCCGGTGCTGCACCGCGGGCGTGCGGTTCGACCG |
| | | GGTGTACGCCACACTGCAAAACATGGTGGTGCCCGAAATCGCCCCGGGCGAG |
| | | GAATGCCCGAGCGACCCCGTGACGGACCCGGCGCACCCGCTGCACCCGGCGA |
| | | ACCTGGTGGCGAACACCGTGAACGCCATGTTCCACAACGGCAGAGTGGTGGT |
| | | GGACGGCCCGGCGATGCTGACCCTGCAGGTGCTGGCGCACAACATGGCCGAG |
| | | CGGACCACAGCGCTGCTGTGCAGCGCCGCGCCCGACGCGGGGGCCAACACCG |
| | | CCAGCACCGCCAACATGCGGATCTTCGACGGCGCCCTGCACGCCGGCGTCCT |
| | | GCTGATGGCGCCCCAGCACCTGGACCACACGATCCAGAACGGAGAGTACTTC |
| | | TACGTGCTGCCGGTGCACGCCCTGTTCGCCGGGGCGGACCACGTCGCGAACG |
| | | CCCCCAACTTCCCGCCCGCGCTGCGGGACCTGGCCCGCCACGTGCCGCTGGT |
| | | CCCCCCGGCCCTGGGCGCGAACTACTTCAGCAGCATCAGACAACCGGTGGTC |
| | | CAGCACGCGCGCGAGAGCGCCGCCGGAGAGAACGCCCTCACCTACGCCCTCA |
| | | TGGCCGGCTACTTCAAGATGAGCCCGGTGGCGCTGTACCACCAGCTGAAAAC |
| | | CGGCCTGCACCCGGGCTTCGGCTTCACCGTGGTGCGG |
| ARV-HSV-mRNA-4 | 345 | MRMQLLLLIALSLALVTNSRTSWKRVTSGEDVVLLPAPAGPEERTRAHKLLW |
| (HSV-2 glycoprotein | | AAEPLDACGPLRPSWVALWPPRRVLETVVDAACMRAPEPLAIAYSPPFPAGD |
| E and glycoprotein | | EGLYSELAWRDRVAVVNESLVEYGALETDSGLYTLSVVGLSDEARQVASVVL |
| C); Protein sequence | | VVEPAPVPTPTPDDYDEEDDAGVSERTPVSVPPPTPPRRPPVAPPTHPRVIP |
| | | EVSHVRGVTVHMETPEAILFAPGETFGTNVSIHAIAHDDGPYAMDVVWMRFD |
| | | VPSSCAEMRIYEACLYHPQLPECLSPADAPCAVSSWAYRLAVRSYAGCSRTT |
| | | PPPRCFAEARMEPVPGLAWLASTVNLEFQHASPQHAGLYLCVVYVDDHIHAW |
| | | GHMTISTAAQYRNAVVEQHLPQRQPEPVEPTRPHVRA*ATNFSLLKQAGDVE |
| | | ENPGPMRMQLLLLIALSLALVINSASPGRTITVGPRGNASNAAPSASPRNAS |
| | | APRTTPTPPQPRKATKSKASTAKPAPPPKTGPPKTSSEPVRCNRHDPLARYG |
| | | SRVQIRCRFPNSTRTEFRLQIWRYATATDAEIGTAPSLEEVMVNVSAPPGGQ |
| | | LVYDSAPNRTDPHVIWAEGAGPGASPRLYSVVGPLGRQRLIIEELTLETQGM |
| | | YYWVWGRTDRPSAYGTWVRVRVFRPPSLTIHPHAVLEGQPFKATCTAATYYP |
| | | GNRAEFVWFEDGRRVFDPAQIHTQTQENPDGFSTVSTVISAAVGGQGPPRTF |
| | | TCQLTWHRDSVSFSRRNASGTASVLPRPTITMEFTGDHAVCTAGCVPEGVTF |
| | | AWFLGDDSSPAEKVAVASQTSCGRPGTATIRSTLPVSYEQTEYICRLAGYPD |
| | | GIPVLEHH |
| ARV-HSV-mRNA-4 | 346 | ATGCGCATGCAGCTGCTGCTGCTGATCGCCCTGTCCCTGGCCCTGGTGACCA |
| (HSV-2 glycoprotein | | ACTCCCGCACCTCCTGGAAGCGCGTGACCTCCGGCGAGGACGTGGTGCTGCT |
| E and glycoprotein | | GCCCGCCCCCGCCGGCCCCGAGGAGCGCACCCGCGCCCACAAGCTGCTGTGG |
| C); DNA sequence | | GCCGCCGAGCCCCTGGACGCCTGCGGCCCCCTGCGCCCCTCCTGGGTGGCCC |
| | | TGTGGCCCCCCCGCCGCGTGCTGGAGACCGTGGTGGACGCCGCCTGCATGCG |
| | | CGCCCCCGAGCCCCTGGCCATCGCCTACTCCCCCCCCTTCCCCGCCGGCGAC |
| | | GAGGGCCTGTACTCCGAGCTGGCCTGGCGCGACCGCGTGGCCGTGGTGAACG |
| | | AGTCCCTGGTGATCTACGGCGCCCTGGAGACCGACTCCGGCCTGTACACCCT |
| | | GTCCGTGGTGGGCCTGTCCGACGAGGCCCGCCAGGTGGCCTCCGTGGTGCTG |
| | | GTGGTGGAGCCCGCCCCCGTGCCCACCCCCACCCCCGACGACTACGACGAGG |
| | | AGGACGACGCCGGCGTGTCCGAGCGCACCCCCGTGTCCGTGCCCCCCCCCAC |
| | | CCCCCCCCGCCGCCCCCCCGTGGCCCCCCCCACCCACCCCCGCGTGATCCCC |
| | | GAGGTGTCCCACGTGCGCGGCGTGACCGTGCACATGGAGACCCCCGAGGCCA |
| | | TCCTGTTCGCCCCCGGCGAGACCTTCGGCACCAACGTGTCCATCCACGCCAT |
| | | CGCCCACGACGACGGCCCCTACGCCATGGACGTGGTGTGGATGCGCTTCGAC |
| | | GTGCCCTCCTCCTGCGCCGAGATGCGCATCTACGAGGCCTGCCTGTACCACC |
| | | CCCAGCTGCCCGAGTGCCTGTCCCCCGCCGACGCCCCCTGCGCCGTGTCCTC |
| | | CTGGGCCTACCGCCTGGCCGTGCGCTCCTACGCCGGCTGCTCCCGCACCACC |
| | | CCCCCCCCCCGCTGCTTCGCCGAGGCCCGCATGGAGCCCGTGCCCGGCCTGG |
| | | CCTGGCTGGCCTCCACCGTGAACCTGGAGTTCCAGCACGCCTCCCCCCAGCA |
| | | CGCCGGCCTGTACCTGTGCGTGGTGTACGTGGACGACCACATCCACGCCTGG |
| | | GGCCACATGACCATCTCCACCGCCGCCCAGTACCGCAACGCCGTGGTGGAGC |
| | | AGCACCTGCCCCAGCGCCAGCCCGAGCCCGTGGAGCCCACCCGCCCCCACGT |
| | | GCGCGCCTAAGCCACGAACTTCTCACTATTAAAGCAAGCAGGAGACGTGGAA |
| | | GAAAACCCCGGTCCAATGCGCATGCAGCTGCTGCTGCTGATCGCCCTGTCCC |
| | | TGGCCCTGGTGACCAACTCCGCCTCCCCCGGCCGCACCATCACCGTGGGCCC |
| | | CCGCGGCAACGCCTCCAACGCCGCCCCCTCCGCCTCCCCCCGCAACGCCTCC |
| | | GCCCCCCGCACCACCCCCACCCCCCCCCAGCCCCGCAAGGCCACCAAGTCCA |
| | | AGGCCTCCACCGCCAAGCCCGCCCCCCCCCCCAAGACCGGCCCCCCCAAGAC |
| | | CTCCTCCGAGCCCGTGCGCTGCAACCGCCACGACCCCCTGGCCCGCTACGGC |
| | | TCCCGCGTGCAGATCCGCTGCCGCTTCCCCAACTCCACCCGCACCGAGTTCC |
| | | GCCTGCAGATCTGGCGCTACGCCACCGCCACCGACGCCGAGATCGGCACCGC |
| | | CCCCTCCCTGGAGGAGGTGATGGTGAACGTGTCCGCCCCCCCCGGCGGCCAG |
| | | CTGGTGTACGACTCCGCCCCCAACCGCACCGACCCCCACGTGATCTGGGCCG |
| | | AGGGCGCCGGCCCCGGCGCCTCCCCCCGCCTGTACTCCGTGGTGGGCCCCCT |
| | | GGGCCGCCAGCGCCTGATCATCGAGGAGCTGACCCTGGAGACCCAGGGCATG |
| | | TACTACTGGGTGTGGGGCCGCACCGACCGCCCCTCCGCCTACGGCACCTGGG |
| | | TGCGCGTGCGCGTGTTCCGCCCCCCCTCCCTGACCATCCACCCCCACGCCGT |
| | | GCTGGAGGGCCAGCCCTTCAAGGCCACCTGCACCGCCGCCACCTACTACCCC |
| | | GGCAACCGCGCCGAGTTCGTGTGGTTCGAGGACGGCCGCCGCGTGTTCGACC |
| | | CCGCCCAGATCCACACCCAGACCCAGGAGAACCCCGACGGCTTCTCCACCGT |
| | | GTCCACCGTGACCTCCGCCGCCGTGGGCGGCCAGGGCCCCCCCCGCACCTTC |
| | | ACCTGCCAGCTGACCTGGCACCGCGACTCCGTGTCCTTCTCCCGCCGCAACG |
| | | CCTCCGGCACCGCCTCCGTGCTGCCCCGCCCCACCATCACCATGGAGTTCAC |
| | | CGGCGACCACGCCGTGTGCACCGCCGGCTGCGTGCCCGAGGGCGTGACCTTC |
| | | GCCTGGTTCCTGGGCGACGACTCCTCCCCCGCCGAGAAGGTGGCCGTGGCCT |
| | | CCCAGACCTCCTGCGGCCGCCCCGGCACCGCCACCATCCGCTCCACCCTGCC |
| | | CGTGTCCTACGAGCAGACCGAGTACATCTGCCGCCTGGCCGGCTACCCCGAC |
| | | GGCATCCCCGTGCTGGAGCACCAC |
| ARV-MPX-mRNA-1 | 347 | MGAAASIQTTVNTLSERISSKLEQEANASAQTKCDIEIGNFYIRQNHGCNIT |
| (MPX viral proteins | | VKNMCSADADAQLDAVLSAATETYSGLTPEQKAYVPAMFTAALNIQTSVNTV |
| of M1R, A35R and | | VRDFENYVKQTCNSSAVVDNKLKIQNVIIDECYGAPGSPTNLEFINTGSSKG |
| B6R); Protein | | NCAIKALMQLTTKATTQIAPRQVAGTGVQFYMIVIGVIILAALFMYYAKRML |
| sequence | | FTSTNDKIKLILANKENVHIWTTYMDTFFRTSPMIIATTDIQNATNFSLKQA |
| | | GDVEENPGPMMTPENDEEQTSVFSATVYGDKIQGKNKRKRVIGLCIRISMVI |
| | | SLLSMITMSAFLIVRQNQCMSANEAAITDSAVAVAAASSTHRKVASSTTQYD |
| | | HKESCNGLYYQGSCYILHADYKSFEDAKANCAAESSTLFNKSDVLTTWLIDY |
| | | VEDTWGSDGNPITKTTSDYQDSDVSQEVRKYFCTATNFSLLKQAGDVEENPG |
| | | PMKTISVVTLLCVLPAVVYSTCTVPTMNNAKLISTETSFNDKQKVTFTCDSG |
| | | YHSLDPNAVCETDKWKYENPCKKMCTVSDYVSELYDKPLYEVNSTMTLSCNG |
| | | ETKYFRCEEKNGNTSWNDTVTCPNAECQPLQLEHGSCQPVKEKYSFGEYMTI |
| | | NCDVGYEVIGVSYISCTANSWNVIPSCQQKCDIPSLSNGLISGSTFSIGGVI |
| | | HLSCKSGFTLTGSPSSTCIDGKWNPILPTCVRSNEEFDPVDDGPDDETDLSK |
| | | LSKDVVQYEQEIESLEATYHITIMALTIMGVIFLISIIVLVCSCDKNNDQYK |
| | | FHKLLP |
| ARV-MPX-mRNA-1 | 348 | TAATACGACTCACTATAGGGagAGAGATAAACATAAACATAAACGACAAGAA |
| (MPX viral proteins | | ACACATACAAAAGAAACAGAACAGAAAACAGCCACCATGGGAGCCGCTGCTT |
| of M1R, A35R and | | CCATTCAAACAACCGTGAACACCCTGTCTGAAAGAATCAGCAGCAAGCTGGA |
| B6R); DNA sequence | | ACAAGAGGCCAATGCCAGCGCCCAGACCAAGTGCGACATCGAAATCGGCAAT |
| | | TTCTACATCAGACAGAATCACGGTTGTAATATCACCGTGAAAAACATGTGCA |
| | | GCGCCGACGCTGATGCCCAGCTCGACGCCGTCCTGTCTGCTGCTACAGAGAC |
| | | ATATTCTGGCCTGACTCCCGAACAGAAGGCCTACGTCCCCGCCATGTTCACC |
| | | GCCGCCCTGAACATCCAGACASCTGTGAACACCGTTGTGCGAGATTTCGAGA |
| | | ACTACGTGAAGCAGACATGCAATAGCAGCGCCGTGGTCGACAACAAGCTGAA |
| | | GATCCAGAACGTGATCATCGACGAGTGCTACGGCGCCCCTGGCAGCCCCACC |
| | | AACCTGGAGTTCATCAACACCGGCAGCAGCAAGGGCAACTGCGCCATCAAAG |
| | | CCCTCATGCAGCTGACCACCAAGGCCACAACCCAAATCGCCCCTAGACAAGT |
| | | GGCCGGCACCGGCGTTCAATTTTACATGATCGTGATTGGCGTGATCATCCTG |
| | | GCTGCGCTGTTCATGTACTACGCCAAACGGATGCTGTTTACCAGCACCAATG |
| | | ACAAGATCAAGCTGATCCTGGCCAACAAGGAAAACGTGCACTGGACCACATA |
| | | CATGGACACCTTCTTCAGAACCAGCCCTATGATCATTGCCACCACCGATATC |
| | | CAGAACGCCACAAACTTCAGCCTGCTGAAGCAGGCCGGAGATGTCGAGGAAA |
| | | ACCCAGGCCCCATGATGACACCTGAAAATGATGAGGAACAGACCAGCGTTTT |
| | | CAGCGCAACAGTGTACGGCGATAAGATCCAGGGCAAGAACAAGCGGAAGAGA |
| | | GTGATCGGCCTGTGCATCAGAATCAGCATGGTGATCAGCCTGCTGAGCATGA |
| | | TCACCATGAGCGCCTTCCTGASCGTGCGGCAGAACCAGTGCATGAGCGCAAA |
| | | TGAGGCCGCTATCACCGATAGCGCCGTGGCCGTGGCCGCTGCTAGCTCTACC |
| | | CACAGAAAGGTGGCCAGCAGCACAACCCAGTACGACCACAAGGAGTCTTGCA |
| | | ACGGCCTGTACTACCAGGGCTCTTGTTACATCCTGCACAGCGACTATAAGTC |
| | | ATTCGAGGACGCCAAGGCTAACTGCGCCGCCGAGAGCAGCACCCTGCCTAAC |
| | | AAGTCCGACGTGCTGACCACTTGGCTGATCGACTACGTGGAGGACACATGGG |
| | | GCTCCGATGGCAATCCTATCACCAAAACCACATCCGACTATCAGGACAGCGA |
| | | CGTGAGCCAGGAGGTGCGGAAGTACTTCTGCACCGCCACAAACTTCAGCCTG |
| | | CTGAAACAGGCCGGGGACGTGGAAGAGAACCCAGGACCTATGAAGACCATCT |
| | | CTGTGGTGACCCTTCTTTGCGTGCTGCCTGCCGTGGTCTACAGCACCTGCAC |
| | | CGTGCCCACCATGAACAACGCCAAGCTCACATCCACCGAAACAAGCTTCAAC |
| | | GACAAGCAGAAAGTGACCTTCACCTGCGACAGCGGATATCATTCTCTGGACC |
| | | CCAACGCAGTGTGCGAGACAGACAAGTGGAAATACGAGAATCCTTGTAAAAA |
| | | GATGTGTACAGTGTCCGATTACGTGTCCGAGCTGTACGATAAGCCTCTGTAC |
| | | GAGGTGAATTCTACCATGACCCTGAGCTGTAATGGAGAGACGAAGTATTTTA |
| | | GATGCGAGGAAAAGAACGGCAACACAAGCTGGAACGATACAGTCACCTGTCC |
| | | TAACGCTGAATGCCAGCCCCTGCAGCTGGAGCACGGCAGTTGCCAGCCTGTG |
| | | AAAGAGAAGTACAGCTTTGGCGAGTACATGACCATCAACTGCGACGTGGGCT |
| | | ACGAGGTGATTGGCGTCAGCTACATCTCTTGTACCGCCAACAGCTGGAATGT |
| | | GATCCCTAGCTGTCAGCAGAAGTGCGACATCCCCAGCCTGAGCAACGGACTG |
| | | ATCTCCGGCTCTACCTTTAGCATCGGCGGAGTGATCCACCTGAGCTGCAAAA |
| | | GCGGCTTCACCCTGACCGGCTCCCCTTCTAGTACATGCATCGATGGAAAGTG |
| | | GAACCCCATTCTGCCAACCTGTGTGAGGTCCAACGAGGAATTCGACCCTGTG |
| | | GACGACGGCCCTGATGATGAAACCGACCTGTCAAAGCTGTCAAAGGACGTGG |
| | | TGCAGTACGAGCAGGAGATCGAGAGCCTGGAAGCCACATACCACATCATTAT |
| | | CATGGCCCTGACAATCATGGGCGTGATCTTCCTGATCTCTATCATCGTGCTG |
| | | GTGTGCAGCTGTGATAAGAACAACGACCAGTACAAGTTCCACAAGCTGCTGC |
| | | CTTGACGTACGGCTGGAGCCTCGGTGGCCTAGCTTCTTGCCCCTTGGGCCTC |
| | | CCCCCAGCCCCTCCTCCCCTTCCTGCACCCGTACCCCCGTGGTCTTTGAATA |
| | | AAGTCTGAGTGGGCGGCAaaaaaaaaaaaaaaaaaaaaaaaaaaaaagcata |
| | | tgactaaaaaaaaaaaaaaaaaaaaaaaaaaaaaaaaaaaaaaaaaaaaaaa |
| | | aaaaaaaaaaaaaaaaaaaaaaaAAAAGAAGAGCAAGCTT |
| ARV-MPX-mRNA-2 | 349 | MMKMKMMVRIYFVSLSALLFHSYAIDIENEITEFFNKMRDTLPAKDSKWLNP |
| (MPX viral proteins | | VCMFGGTMNDMAALGEPFSAKCPPIEDSLLSHRYKDYVVKWERLEKNRRRQV |
| of IFNbp (B16R), | | SNKRVKHGDLWIANYTSKFSNRRYLCTVTTKNGDCVQGVVRSHVWKPSSCIP |
| F3L and Multiple T | | KTYELGTYDKYGIDLYCGILYAKHYNNITWYKDNKEINIDDFKYSQAGKELI |
| cell Epitopes (MTE); | | IHNPELEDSGRYDCYVHYDDVRIKNDIVVSRCKILTVIPSQDHRFKLILDPK |
| Protein sequence | | INVTIGEPANITCSAVSTSLFVDDVLIEWENPSGWIIGLDFGVYSILTSRGG |
| | | ITEATLYFENVTEEYIGNTYTCRGHNYYFDKILTTTVVLEATNFSLLKQAGD |
| | | VEENPGPMEKREVNKATYDLQRSTMVYSSDDTPPRWSTTMDADTRPTDSDAD |
| | | AIIDDVSREKSMREDNKSFDDVIPVKKIIYWKGVNPVTVINEYCQITRRDWS |
| | | FRIESVGPSNSPTFYACVDIDGRVFDKADGKSKRDAKNNAAKLAVDKALSYV |
| | | IIRFATNFSLLKQAGDVEENPGPSLTILDDNLYKVYNGRRGKIDYYIPYVED |
| | | FVRHCLTEYILWVSHRYMSNLFDIPLLTVYWPCVDAVKDVTITKKNNIDHIV |
| | | WINNSWKFNSERKKTYNDHIVNLLFCLSTEERHIFLDYKKYYFTFLVIAINA |
| | | MFNSYMSNLFDIPLLTVYWPVRHCLTEYILWVSHRYDVSLSAYIIRVTTAED |
| | | NYPSNKNYEITLR |
| ARV-MPX-mRNA-2 | 350 | TAATACGACTCACTATAGGGagAGAGATAAACATAAACATAAACGACAAGAA |
| (Monkeypox (MPX) | | ACACATACAAAAGAAACAGAACAGAAAACAGCCACCATGATGAAGATGAAGA |
| viral proteins of | | TGATGGTCAGAATCTACTTCGTGTCCCTGAGCCTGTTGCTGTTTCACAGCTA |
| IFNbp (B16R), F3L | | CGCTATCGACATCGAGAACGAGATCACCGAGTTCTTCAACAAGATGCGCGAC |
| and Multiple T cell | | ACTCTGCCTGCCAAGGACAGCAAGTGGCTGAACCCTGTGTGCATGTTCGGCG |
| Epitopes (MTE); | | GAACCATGAACGACATGGCCGCTCTGGGAGAACCCTTCAGCGCCAAGTGCCC |
| DNA sequence | | TCCTATCGAGGATTCTCTCCTGAGCCACAGATACAAGGACTACGTGGTCAAG |
| | | TGGGAAAGACTGGAAAAAAACCGGAGAAGGCAGGTGTCCAACAAGCGGGTGA |
| | | AACACGGAGATCTGTGGATCGCCAATTACACCTCCAAGTTCAGCAACAGACG |
| | | GTACCTGTGCACCGTTACAACAAAGAACGGCGATTGCGTGCAAGGCGTGGTG |
| | | CGGTCCCACGTGTGGAAACCTAGCAGCTGCATCCCCAAGACCTACGAGCTGG |
| | | GCACCTATGATAAGTACGGCATAGATCTGTATTGCGGCATCCTGTACGCTAA |
| | | ACACTACAACAACATCACCTGGTACAAGGACAACAAGGAAATCAATATCGAT |
| | | GACTTCAAGTACAGCCAGGCTGGCAAAGAGCTTATTATCCACAACCCTGAGC |
| | | TGGAAGATAGCGGAAGATACGACTGCTACGTGCACTACGACGACGTGCGGAT |
| | | CAAGAATGACATTGTGGTGTCTAGATGCAAGATCCTGACCGTGATCCCTTCT |
| | | CAGGACCACCGGTTCAAACTGATCCTCGATCCTAAGATCAATGTGACCATTG |
| | | GCGAGCCCGCCAACATCACCTGTAGCGCCGTGTCCACCAGCCTGTTCGTGGA |
| | | TGATGTTCTGATCGAGTGGGAGAATCCTAGTGGATGGATCATCGGCCTGGAC |
| | | TTCGGCGTTTACAGCATCCTGACAAGCCGGGGAGGCATCACCGAGGCCACCC |
| | | TGTACTTCGAGAACGTGACCGAGGAATACATCGGGAACACCTACACCTGCAG |
| | | AGGCCACAATTACTACTTCGACAAGACCCTGACCACAACAGTGGTGCTGGAA |
| | | GCCACAAACTTCAGCCTGCTGAAGCAGGCCGGCGACGTTGAGGAAAATCCTG |
| | | CCCCTATGGAAAAGAGAGAGGTGAACAAGGCCCTGTACGACCTGCAGCGGTC |
| | | TACAATGGTGTACAGCAGCGACGATACCCCTCCAAGATGGTCCACCACCATG |
| | | GACGCTGATACAAGACCTACCGACTCCGACGCCGACGCTATCATCGACGACG |
| | | TGAGCAGAGAAAAGTCTATGAGAGAGGATAACAAAAGCTTCGACGACGTGAT |
| | | TCCAGTCAAAAAGATCATCTACTGGAAGGGCGTGAATCCAGTGACCGTGATT |
| | | AACGAGTACTGTCAGATCACAAGAAGAGACTGGAGCTTTAGAATCGAGAGCG |
| | | TGGGCCCTAGCAACTCTCCTACATTCTACGCTTGTGTGGACATCGACGGCAG |
| | | AGTCTTTGATAAGGCCGACGGCAAGAGCAAGCGGGACGCCAAAAACAACGCC |
| | | GCCAAGCTGGCCGTGGACAAGCTGCTGAGCTACGTGATCATTAGATTCGCCA |
| | | CCAACTTCAGCCTGCTGAAACAGGCCGGCGATGTTGAAGAGAACCCCGGCCC |
| | | CAGCCTGACCATCCTGGACGACAACCTGTACAAGGTCTACAACGGCCGGCGG |
| | | GGCAAGATCGACTACTACATCCCCTACGTGGAAGATTTCGTGCGCCACTGTC |
| | | TGACCGAGTACATCCTGTGGGTCTCCCACCGGTACATGAGCAACCTGTTCGA |
| | | CATTCCTCTGCTGACTGTGTACTGGCCTTGTGTGGACGCCGTGAAGGACGTG |
| | | ACCATCACAAAAAAGAACAATATCGATCACATCGTGTGGATCAACAACTCTT |
| | | GGAAGTTTAACAGTGAAAGAAAGAAGACCTACAACGACCACATCGTGAACCT |
| | | GCTGTTCTGCCTGAGCACAGAAGAGAGACACATCTTCCTGGACTATAAGAAG |
| | | TACTACTTCACCTTTCTGGTGATCGCCATCAACGCCATGTTCAACAGCTACA |
| | | TGTCTAACCTGTTTGACATCCCCCTGCTGACAGTGTATTGGCCCGTGAGGCA |
| | | TTGCCTGACGGAATACATCCTGTGGGTGTCTCATAGATACGACGTGAGCCTG |
| | | AGCGCATACATCATCCGGGTGACAACAGCCGAGGACAATTATCCTTCTAATA |
| | | AAAACTATGAGATCACCCTCAGATGACGTACGGCTGGAGCCTCGGTGGCCTA |
| | | GCTTCTTGCCCCTTGGGCCTCCCCCCAGCCCCTCCTCCCCTTCCTGCACCCG |
| | | TACCCCCGTGGTCTTTGAATAAAGTCTGAGTGGGCGGCAaaaaaaaaaaaaa |
| | | aaaaaaaaaaaaaaaagcatatgactaaaaaaaaaaaaaaaaaaaaaaaaaa |
| | | aaaaaaaaaaaaaaaaaaaaaaaaaaaaaaaaaaaaaaaaaaaaAAAAGAAG |
| | | AGCAAGCTT |

TABLE 2
list of T cell epitopes in the constructs
DNA and Protein Sequences in Constructs

| | SEQ | |
|---|---|---|
| | ID | List of T cell epitopes |
| mRNA | NO: | in the mRNA |
| ARV- | 351 | KSYELQTPF |
| COV- | 352 | YELQTPFEI |
| MTE | 353 | NYMPYFFTL |
| (SARS- | 354 | MPYFFTLLL |
| COV-2 | 355 | FLLNKEMYL |
| Multi-T- | 356 | SAFAMMFVK |
| cell- | 357 | MTNRQFHQK |
| Epitopes); | 358 | RQFHQKLLK |
| protein | 359 | SMMGFKMNY |
| Sequence | 360 | TYRRLISMMGFKMNY (MHC 2) |
| | 361 | LPVNVAFEL |
| | 362 | HSYFTSDYY |
| | 363 | LWLLWPVTL |
| | 364 | ASFRLFARTRSMWSF |
| | 365 | SFRLFARTR |
| | 366 | SELVIGAVI |
| | 367 | LPKEITVAT |
| | 368 | SYFIASFRLFARTRS (MHC 2) |
| | 369 | ASFRLFARTRSMWSF (MHC 2) |
| | 370 | NTASWFTAL |
| | 371 | FTALTQHGK |
| | 372 | MARKTLNSL |
| | 373 | LSPRWYFYY |
| | 374 | LSPRWYFYYLGTGPE (MHC 2) |
| | 375 | AQFAPSASAF |
| | 376 | LLNKHIDAYK |
| | 378 | NPLLYDANYFLCWHTNCYDYCIPYNSVTSSI |
| | 379 | RLFARTRSMWSFNPETNILLNVPLHGTILTR |
| | | PLLESELVIGAVILRGHLRIAGHHL |
| | 380 | NSSPDDQIGYY |
| | 381 | RRGPEQTQGNFGDQELIRQGTDYKHWPQIAQ |
| | | FAPSASAFFGM |
| | 383 | SALNHTKKW |
| | 384 | QVNGLTSIKW |
| | 385 | SIKWADNNCY |
| | 386 | YLATALLTL |
| | 387 | TTLPVNVAF |
| | 388 | GFLFLTWICLLQFAYANRNRFLYI |
| | 389 | RNRFLYIIKLIFLWLLWPVTLACFV |
| | 390 | LKKLLEQWNLVIGFLFLTW |
| | 391 | LWPVTLACFVLAAVYRINWI |
| | 392 | LVGLMWLSYFIASFRLFARTRSMWSFNP |
| | 393 | MWSFNPETNILLNVPLHGTIL |
| | 394 | IGAVILRGHLRIAGHHLGRCD |
| | 395 | PKEITVATSRTLSYYKL |
| | 396 | TLSYYKLGASQRVAGDSG |
| | 398 | TTDPSFLGRY |
| | 399 | IEYPIIGDEL |
| | 400 | VYFLQSINF |
| | 401 | NRFLYIIKL |
| | 402 | SELVIGAVIL |
| | 403 | RWYFYYLGTGPEAGL (MHC 2) |
| | 404 | MEVTPSGTWL |
| | 405 | ASWFTALTQHGKED (MHC 2) |
| | 406 | IGYYRRATRRIRGGD (MHC 2) |
| | 407 | GTWLTYTGAIKLDDK (MHC 2) |
| | 409 | RTAPHGHVM |
| | 410 | KSYELQTPF |
| | 411 | KLAKKFDTF |
| | 412 | FLGRYMSAL |
| | 413 | CTNYMPYFF |
| | 414 | YMPYFFTLL |
| | 415 | MPYFFTLLL |
| | 416 | FLLNKEMYL |
| | 417 | TQYNRYLAL |
| | 418 | AMSAFAMMF |
| | 419 | RQFHQKLLK |
| | 420 | SMMGFKMNY |
| | 421 | KMNYQVNGY |
| | 422 | YVMHANYIF |
| | 423 | VMHANYIFW |
| | 424 | IQLSSYSLF |
| | 425 | APFLYLYAL |
| | 426 | YANRNRFLY |
| | 427 | YFIASFRLF |
| | 428 | RLFARTRSM |
| | 429 | ATSRTLSYY |
| | 430 | ITLATCELY |
| | 431 | FPRGQGVPI |
| | 432 | LSPRWYFYY |
| | 433 | SPRWYFYYL |
| | 434 | QPYVFIKRSDARTAP |
| | 435 | QIDGYVMHANYIFWR |
| | 436 | YFLCWHTNCYDYCIPY |
| | 437 | KDCVVLHSYFTSDYYQLY |
| | 438 | YFTSDYYQLYSTQLSTDTGV |
| | 439 | GVEHVTFFIYNKIVDEPEEH |
| | 440 | LRIAGHHLGRCDIKDLPK |
| | 441 | GAVILRGHLRIAGHHLGR |
| | 442 | TSRTLSYYKLGASQRVA |
| | 443 | IGNYKLNTDHSSSSDNIA |
| | 444 | PKEITVATSRTLSYYKL |
| | 445 | LITLATCELYHYQECVR |
| | 446 | DQIGYYRRATRRIR |
| | 447 | LSPRWYFYYLGTGPEAGL |
| | 448 | AFFGMSRIGMEVTPSGTW |
| | 449 | GMEVTPSGTWLTYTGAIK |
| | 450 | LLNKHIDAYKTFPPTEPK |
| | 451 | PNFKDQVILLNKHIDAYK (MHC 2) |

TABLE 3
list of T cell epitopes in the constructs
DNA and Protein Sequences in Constructs

| | SEQ | |
|---|---|---|
| | ID | List of T cell epitopes |
| mRNA | NO: | in the mRNA |
| ARV-HSV-mRNA- | 649 | VYADRLDNRLQLGML |
| 3 (HSV-2 UL19UD | 452 | QASGPHETITALVGA |
| and Multi-T-cell- | 453 | ARHASPFERVRCLLLRS |
| Epitopes); Protein | 454 | EGRKSRRPLTTFGSG |
| sequence | 455 | AAAPAWSRRTLLPEH |
| | 456 | HNLFLWEDQTLLRAT |
| | 457 | RGAFEDRSYPAVFYLLQAA |
| | 458 | KNACPLLIFDRTRKFVLACP |
| | 459 | YADRLDNRLQLGMLIPGAV |
| | 460 | KRFGGHYMESVFQMYTRIA |
| | 461 | ILGILVHLRIRTREASFEE |
| | 462 | ETHFTQYLIYDASPLKGLS |
| | 463 | AMKTSNALCVRGARPFSHF |
| | 464 | PALMLEYFCRCAREESKRV |
| | 465 | DTSMSLADFHGEEFEKLYE |
| | 466 | NAWRQRLAHGRVRWVAECQ |
| | 467 | TQYLIYDASPLKGLSL |
| | 468 | TRMAEYDRVHIYYNHR |
| | 469 | ISNQALLRMACEVRQ |
| | 470 | GNWTGAPDVSALGAQG |
| ARV-HSV-mRNA- | 471 | ITQFILEHRARASCK |
| 2 (HSV-2 | 472 | LPRFIPENQRTVALY |
| glycoprotein D and | 473 | YTSTLLPPELSDTTN |
| glycoprotein B); | 474 | DSGLLDYTEVQRRNQ |
| Protein sequence | 475 | AALLVVAVGLRV |
| | 476 | AALLVVAVGLRVVCAKYALA |
| | 477 | AAPANPGLIIGA |
| | 478 | ALLVVAVGLRVV |
| | 479 | ANPGLIIGALAG |
| | 480 | APAAPANPGLII |
| | 481 | APANPGLIIGAL |
| | 482 | AVGLRVVCAKYA |
| | 483 | AYQQGVTVDSIG |
| | 484 | CRSVLLHAPSEAPQIVRGAS |
| | 485 | DEARKHTYNLTIAWYRMGDN |
| | 486 | DPEDSALLEDPA |
| | 487 | DPSLKMADPNRFRGKNLPVL |
| | 488 | EAPQIVRGASDEARKHTYNL |
| | 489 | EDPEDSALLEDP |
| | 490 | EDSALLEDPAGT |
| | 491 | ELSDTTNATQPELVPEDPED |
| | 492 | ELVPEDPEDSAL |
| | 493 | ENQRTVALYSLKIAGWHGPK |
| | 494 | GLRVVCAKYALA |
| | 495 | GVTVDSIGMLPR |
| | 496 | HAPAAPANPGLI |
| | 497 | HAPAAPANPGLIIGALAGST |
| | 498 | KAYQQGVTVDSI |
| | 499 | KAYQQGVTVDSIGMLPRFTP |
| | 500 | LAALVIGGIAFWVRRRRSVA |
| | 501 | LIIGALAGSTLAALVIGGIA |
| | 502 | LLVVAVGLRVVC |
| | 503 | LVPEDPEDSALL |
| | 504 | LVVAVGLRVVCA |
| | 505 | MGRLTSGVGTAALLVVAVGL |
| | 506 | NPGLIIGALAGS |
| | 507 | PAAPANPGLIIG |
| | 508 | PANPGLIIGALA |
| | 509 | PEDPEDSALLED |
| | 510 | PEDSALLEDPAG |
| | 511 | PELVPEDPEDSA |
| | 512 | PELVPEDPEDSALLEDPAGT |
| | 513 | PGLIIGALAGST |
| | 514 | QGVTVDSIGMLP |
| | 515 | QQGVTVDSIGML |
| | 516 | RASCKYALPLRIPPAACLTS |
| | 517 | RVVCAKYALADPSLKMADPN |
| | 518 | SIGMLPRFTPENQRTVALYS |
| | 519 | TVDSIGMLPRFT |
| | 520 | VAVGLRVVCAKY |
| | 521 | VDSIGMLPRFTP |
| | 522 | VGLRVVCAKYAL |
| | 523 | VPEDPEDSALLE |
| | 524 | VTVDSIGMLPRF |
| | 525 | VVAVGLRVVCAK |
| | 526 | YQQGVTVDSIGM |
| | 527 | NKRVFCAAVGRLA |
| | 528 | IPSIQDVAPHHAPAAPANPG |
| | 529 | LKIAGWHGPKPPYTSTLLPP |
| | 530 | RFRGKNLPVLDQLTDPPGVK |
| | 531 | GTTVNCIVEEVDARS |
| | 532 | HVNDMLGRIAVAWCE |
| | 533 | LDYTEVQRRNQLHDL |
| | 534 | YPYDEFVLATGDFVY |
| | 535 | IAVVFKENIAPY |
| | 536 | YMALVSAMERT |
| | 537 | ADMRIYSEALYHPQL |
| | 538 | ATGDIIYMSPFYGYR |
| | 539 | FVMSPFFGLREGSH |

TABLE 4
Sequences of the constructs
DNA and Protein Sequences in Constructs

| | SEQ ID | |
|---|---|---|
| Protein | NO: | Sequence |
| FCoV SPIKE; Protein | 540 | MILLILALLSTAKSEDAPHGVTLPYFNTSYDNNKFELNFYNFLQTWDIPPN |
| Sequence | | TETILGGYLPYCGNGVNCGWYNFVYGRPVGSNGKYAYINTQNLNIPNVHGV |
| | | YFDVREHNSDGVWDTRDRVGLLTATHGSAHYSTIMVTQDSVEENQPHFAVK |
| | | ICHWKPGNISSYHQFNVEFGDGGQCVFNKRFSLDTVLTINDFYGFQWTDTY |
| | | VDIYLGGTITKVWIANDWSVVESSISYHWNQLNYGYYIQFVNRITFYIYNN |
| | | TGGSNYTHLRLSECHGAYCAGYAKNVFVPIDGKIPESFSFSNWFLLSDKST |
| | | LVQGRILSKQPVFVQCLRPVPLWSNNSAVVYFKNDAFCHNVTTDVLRFNLN |
| | | FSDTDIYIESTKDDQLHFTFEDNTTASIACYSSAN |
| | | VTDFQPANNSVSHIPFGKTAYFCFATFSHSVVSRQFLGILPPIVREFAFGR |
| | | DGSIFVNGYKYFSLPPIKSVNFSISSVEQYGFWTIAYTNYTDVMVDVNGTG |
| | | ITRLFYCDSPLNRIKCQQLKYELPDGFYSASMLVKKDLPKTFVTMPQFYNW |
| | | MNVTLHVVLNDTEKKPDTILAKAPELASLADIHFEVAQSNGSVTNVTSICV |
| | | QTRQLALFYKYTSLQGLYTYSNLVELQNYDCPFSPQQFNNYLQFETLCFDV |
| | | NPAVAGCKWSLVHDTRWRTQFATITVSYKEGSMITTMPSSQLGFQDISVLV |
| | | KDECTDYNIYGFQGTGVIRNTTSRLVAGLYYTSISGDLLAFKNSTTGEIFT |
| | | VVPCDLTAQAAVINDEIVGVITSVNQTDLFEFVNHTSTRRSRIAAVPQAAT |
| | | TYTMPQFYYITKWNNDTSTNCTSVITYSSFAICNTGEIKYVNVTHVEVVVV |
| | | KPVSTGNITIPKNFTVAVQAEYIQIQVKPVAVDCAKYVCNGNRHCLNLLTQ |
| | | YTSACQTIENALNLGARLESLMLNDMITVSYRSLELATVEKFNTTVVGGEK |
| | | LGGFYFDGLRALLPPTIGKRSAVEDLLFNKVVTSGLGTVDDDYKKCSSGTD |
| | | VADLVCAQYYNGIMVLPGVVDGNKMAMYTASLIGGMALGSITSAVAVPFAM |
| | | QVQARLNYVALQTDVLQENQKILANAFNNAIGNITLALGKVSNAITTTSYG |
| | | FNSMALALTKIQSVVNQQGEALSQLTSQLQKNFQAISSSIAEIYNRLEKVE |
| | | ADAQVDRLITGRLAALNAYVSQTLTQYAEVKASRQLAMEKVNECVKSQSDR |
| | | YGFCGNGTHLFSLVNSAPDGLLFFHTVLLPTEWEEVTAWSGICVNDTYAYV |
| | | LKDFEYSIFSYNNTYMVTPRNMFQPRKPHMSDFVQITSCEVTFLNTTYTTF |
| | | QEIVIDYIDINKTISDMLEQYNPNHTIPDLDLQLEIFNQTKINLTAEIDQL |
| | | EQRADNLTTIAHELQQYIDNLNKTLVDLEWLNRIETYVKWPWYVWLLIGLV |
| | | VVFCIPLLLFCCLSTGCCGCFGCLGSCCNSFCSRRQFESYEPIEKVHIH |
| FCoV SPIKE; DNA | 541 | atgatattgctaatactagctctacttagcactgctaagtctgaagatgct |
| Sequence | | cctcatggtgtcaccttaccgtactttaacacttcctatgacaataacaag |
| | | tttgaacttaatttctataatttccttcaaacttgggatataccaccaaat |
| | | acggaaactattcttggtggttacctgccatattgtggaaacggtgtaaat |
| | | tgtgggtggtataattttgtttatggtcgacctgtgggatctaatggtaag |
| | | tatgcatacataaacacgcaaaacctgaacatacctaatgtccatggtgtc |
| | | tacttcgatgtgagagaacataattctgatggcgtgtgggacacgcgagat |
| | | cgcgttggcttattgatagccattcatggctccgcgcattacagtctacta |
| | | atggttttacaagactcggtggaagagaatcagcctcattttgctgttaaa |
| | | atctgccattggaaaccaggtaacataagttcctatcatcaatttaatgtt |
| | | gagtttggagacggaggccaatgtgtgtttaacaagagattctcattggac |
| | | acagtgttgacaactaatgacttttatggcttccagtggactgatacatat |
| | | gtggatatctatttaggtggcactattactaaagtgtggattgccaatgat |
| | | tggagtgttgttgaatctagtatctcctatcactggaaccagcttaattat |
| | | ggatattacatccaatttgtcaaccgcactaccttttacatctataataat |
| | | actggtggttcaaattatacccatttgcgactcagcgagtgccacggagcg |
| | | tattgtgctggttatgctaaaaatgtctttgtgccaattgatggcaaaata |
| | | ccagaaagcttttcttttagtaactggtttctgttatcagacaagtctaca |
| | | ttggtgcaaggccgcattcttagcaaacagccagtgtttgtacaatgtctt |
| | | aggcctgtaccattgtggtctaacaatagtgctgtggtgtattttaaaaat |
| | | gatgctttctgccacaatgttacgacagatgttttgaggttcaatctaaac |
| | | tttagtgatactgatatctacatagagtcaactaaggatgatcaattgcac |
| | | ttcacatttgaagataatacaactgcctctatagcctgttatagcagtgcc |
| | | aacgttactgatttccagcctgcaaacaatagtgtctctcatattccattt |
| | | ggcaagactgcgtatttctgttttgccactttctctcactctgttgtgagc |
| | | agacagtttctgggcatactcccaccaattgttcgagagtttgcatttggc |
| | | agagatggatccatttttgttaatggctataaatattttagtctaccacct |
| | | atcaagagtgtcaatttttccattagctcagttgagcagtatggattttgg |
| | | accatagcttacactaactatacagacgtaatggtggatgttaacggcaca |
| | | ggtattactaggttattctattgtgactcaccactcaatagaattaagtgt |
| | | caacaattgaagtatgagctaccagacggtttttattctgctagtatgctc |
| | | gttaaaaaggacctacctaaaacatttgtcaccatgccacagttttacaac |
| | | tggatgaacgtcacgttacatgttgtattaaatgacacagagaaaaaacct |
| | | gacactattcttgctaaggctcctgagctagcatcacttgcggatatacat |
| | | tttgaagtagctcagtctaatggtagtgtaacgaatgtcactagtatatgt |
| | | gtccaaacaagacagttggctctattttataagtacaccagcttacaaggt |
| | | ttgtacacttattctaacctagtggagttacaaaattatgactgccctttt |
| | | tcaccacaacagtttaataattatctgcagtttgagactttgtgttttgat |
| | | gtgaatccagctgttgcaggttgtaagtggtcgttagtccatgacactagg |
| | | tggcgtacccaattcgccactatcacagtttcttacaaagagggttctatg |
| | | atcactactatgccaagttcacagttgggttttcaagatatctctgttttg |
| | | gtaaaagacgaatgcactgattacaacatttatggatttcagggcacaggt |
| | | gttattagaaataccacctctaggttagtggctggtctctattacacatct |
| | | attagtggcgaccttcttgcttttaaaaatagtactactggggaaattttc |
| | | actgtagtgccatgtgacttaacagctcaagctgcagttattaatgacgaa |
| | | atagttggagttataacatccgttaatcaaactgatctgtttgagtttgta |
| | | aaccacacatcaactcgaagatcgcgcatagcagcagtgccacaagcggca |
| | | actacctatactatgccgcaattttattacataacaaagtggaataatgac |
| | | acctcgactaattgtacatctgtcattacctattcctcctttgctatttgt |
| | | aatactggtgaaattaaatatgttaatgttactcatgttgaagttgtagtt |
| | | gttaaacctgtttcaacaggtaacataactatacctaaaaatttcacggtt |
| | | gcagtacaagccgaatacattcagattcaagtaaagcctgttgctgtggat |
| | | tgtgctaagtatgtctgtaatggtaatagacattgccttaacttgttaaca |
| | | caatacacctcagcttgtcaaacaatagaaaatgccctcaatcttggtgca |
| | | cgccttgaatctttaatgcttaatgatatgattacagtatcatatcgcagt |
| | | ctagagcttgcaactgttgaaaagtttaacactactgttgtaggtggtgaa |
| | | aagcttggtggtttctattttgacggtttgagagccttgctaccacctaca |
| | | attggtaaacggtcagctgttgaagacctattgttcaacaaagtggtgacc |
| | | agcggtcttggcaccgttgacgatgactataaaaagtgttcttctggcact |
| | | gatgttgcagatttagtttgtgcccaatattacaatggcataatggtttta |
| | | cctggtgttgtggatggcaataagatggctatgtataccgcatccttaatt |
| | | ggcggtatggccttgggttctattacatctgctgtagccgtccctttcgct |
| | | atgcaagtgcaggcacggctcaattatgttgcactacaaactgatgtttta |
| | | caggaaaaccagaaaatacttgctaatgctttcaataatgccattggtaac |
| | | attactttagcgcttggaaaagtctccaatgctattacaaccacatcatat |
| | | ggttttaatagtatggccttggcactgactaaaattcagagtgtggttaac |
| | | caacagggtgaggcgttgagtcaacttaccagtcagttacagaaaaatttc |
| | | caggctattagtagttctattgctgaaatctacaatagactggaaaaagtg |
| | | gaagctgatgctcaagttgaccgtctcatcaccgggagattagcagcactt |
| | | aatgcttatgtgtctcaaactttaactcaatatgctgaagtcaaggctagt |
| | | agacaattggcaatggagaaggttaatgagtgcgtgaagtcccagtcggat |
| | | aggtatgggttctgtggaaatggaacacacctattctcacttgtcaattct |
| | | gcacctgatggtttacttttctttcacacagtgttacttcctacagaatgg |
| | | gaagaggtgacggcatggtcaggaatatgtgttaatgacacatatgcatat |
| | | gtgttgaaagactttgaatattctatttttagctataacaatacgtatatg |
| | | gtgactcctcgtaatatgtttcaacctagaaaacctcatatgagtgatttc |
| | | gttcaaattacgagctgtgaagtgacttttcttaacactacatatacgaca |
| | | tttcaggagattgtgattgattatattgatatcaacaagactatctctgat |
| | | atgcttgaacagtataatcctaatcacacaatacctgatttagatttacag |
| | | ctagaaatctttaaccagacaaagttaaacctcactgcagaaatagaccaa |
| | | ttagaacaaagagcagataacctcactaccatcgcacatgaactacagcag |
| | | tacattgacaaccttaataagacccttgttgaccttgaatggctcaatagg |
| | | attgaaacttatgtaaaatggccgtggtatgtgtggctactaattggacta |
| | | gtagttgtcttttgcataccattgttgctgttttgctgtctgagtactggt |
| | | tgttgtgggtgttttggttgtcttggaagttgttgtaattccttttgtagt |
| | | agaagacaatttgaaagttacgaacccattgaaaaggttcacattcattaa |
| FCoV S-WT; Protein | 633 | MILLLILLAVVRSEDAPHGVTLPHFNTSYNNAKFELNFYNFLQTWDIPPNT |
| Sequence | | ETILGGYLPYCGKGDNCGWYNFAYQQHDALNGKYAYINTQNLNIPNVHGVY |
| | | FDVRERYYDDGVWDIADRVGLLIAIHGNSYYSLLMVLQDDVEENKPHVAVK |
| | | ICHWRPGNISTYHQFNVDLGDGGQCVFNQRFSLDTKLTADDFYGFQWTDTY |
| | | VDIYLGGTITKVWVDNDWSIVEASISYHWNRVNYGYYMQFVNRTTYYAYNN |
| | | TGGSNYNHLQ;SECNSDYCAGYAKNVFVPTDGKTPESFSFSNWFLLSDKST |
| | | LVQGRVLSSQPVIVQCLRPVPSWSNNTAMVYFTNDVSCPNVTADVLRFNLN |
| | | FSDTDVYTESTKDDQLYFTFEDNTTASIACYSSANVTDFQPANNSVSHVPF |
| | | GKTAHSYFCFANFSHSLVSRQFLGILPPTVREFAFGRDGSIFVNGYKYFSL |
| | | PPIKSVNFSISSVEQYGFWTIAYTNYTDVMVDVNGTGITRLFYCDSPLNRI |
| | | KCQQLKHELPDGFYSASMLVKKDLPKTFVTMPQFYNWMNVTLHVVLNDTEK |
| | | RADIILAKAPELASLADIHFEIAQANGSVTNVTSLCVQARQLALFYKYTSL |
| | | QGLYTYSNLVELQNYDCPFSPQQFNNYLQFETLCLDVNPAVAGCKWSLVHD |
| | | NKWRTQFATITVSYKEGAMITTMPKAQLGFQDISNLVKDECTDYNIYGFQG |
| | | TGIIRNTTSRLVAGLYYTSISGDLLAFKNSTTGEIFTVVPCDLTAQAAVIN |
| | | DEIVGAITAVNQTDLFEFVNHTQSRRSRRSTLETVQTYTMPQFYYITKWNN |
| | | DTSTNCTSVITYSSFAICNTGEIKYVNVTHVETVDDNIGVIKPISTGNISI |
| | | PKNFTVAVQAEYIQIQVKPVVVDCAKYVCNGNRHCLSLLTQYTSACQTIEN |
| | | ALNLGARLESLMLNDMITVSDRSLELATVEKFNTTALGGEKIGGFYFDGLS |
| | | SLLPPKIGKRSAVEDLLFNKVVTSGLGTVDDDYKKCSAGTDVADLVCAQYY |
| | | NGIMVLPGVVDHNKMAIYTASLIGGMAMGSITSAVAVPFAMQVQARLNYVA |
| | | LQTDVLQENQKILANAFNNAIGNITLALGKVSNAITTISDGFNSMASALTK |
| | | IQSVVNQQGEALSQLTSQLQKNFQAISSSIAEIYNRLEKVEADAQVDRLIT |
| | | GRLAALNAYVSQTLTQYAEVKASRQLAMEKVNECVKSQSDRYGFCGNGTHL |
| | | FSLVNSAPDGLLFFHTVLLPTEWEEVTAWSGICVNDTYAYVLKDFEYSIFS |
| | | YNNTYMVTPRNMFQPRKPQMSDFVQITSCEVTFLNTTYTTFQEIVIDYIDI |
| | | NKTTSDMLEQYNPNYTTPELDLQLEIFNQTKLNLTAETDQLEQRADNLTNI |
| | | AHELQQYIDNLNKTLVDLEWLNRIETYVKWPWYVWLLIGLVVVFCIPLLLF |
| | | CCLSTGCCGCFGCLGSCCHSLCSRRQFESYEPIEKVHIH* |
| FCoV S-WT; DNA | 634 | GAATTCGGCTTTAATACGACTCACTATAAGGACATTAGCTTCTGACACAAC |
| Sequence | | TGTGTTCACTAGCAACCTCAAACAGCCACCatgatactgttacttacactt |
| | | ctggccgttgtcaggtctgaagatgctcctcatggtgttactctacctcac |
| | | ttcaatacgtcttataacaatgccaagtttgaacttaatttttacaatttc |
| | | ttacaaacttgggatataccaccaaacacagaaaccattttgggcggttat |
| | | ctgccatattgtggaaaaggtgataattgtggatggtataattttgcttat |
| | | cagcaacatgatgctctaaatggtaagtacgcttacataaacacacaaaat |
| | | ctgaatataccaaacgttcatggcgtctacttcgacGTGcgagaaaggtat |
| | | tatgatgatggcgtgtgggatatagctgatagagttggtctattgattgcc |
| | | atacatGGAaattcgtattacagtttgcttatggttttacaagatgacgtg |
| | | gaagaaaataagcctcacgttgcagttaaaatctgccattggaggccaggt |
| | | aatataagcacttaccatcaatttaatgttgatttaggagacggtggtcaa |
| | | tgcgtgtttaaccagagattctcattggacaccaaattgacagctgatgat |
| | | ttttatggcttccagtggactgatacatatgtagacatatatttaggtggc |
| | | actattactaaagtgtgggttgacaatgactggagtattgttgaagccagt |
| | | atttcctaccattggaatcgcgttaactatggctactacatgcaatttgtc |
| | | aaccgcaccacgtattatgcctacaataatactggtggttcaaattacaac |
| | | catctgcagttgagcgagtgcaatagtgattattgtgctggttatgctaag |
| | | aacgtctttgtgccaattgatggcaaaataccagaaagcttttcttttagt |
| | | aactggtttctgctatcagataaatccaccttagtgcaagggcgtgttctt |
| | | agtagtcagccggttcttgtacaatgtctcaggcctgtaccatcgtggtct |
| | | aacaatactgctatggtgtattttacaaatgatgtctcttgccctaacgtc |
| | | acggcagacgttctgaggttcaatctaaattttagtgacactgatgtttat |
| | | acggagtcaactaaggatgatcagttgtatttcacattcgaagataataca |
| | | actgcttctatagcctgttatagcagtgctaatgttactgacttccaacca |
| | | gcaaataatagcgtctcacatgttccatttggtaaaactgcgcattcttat |
| | | ttctgttttgccaatttttctcactctcttgtgagtagacagtttctgggc |
| | | atacttccaccaactgttcgagagtttgctttcggcagagatggatccatt |
| | | tttgttaatggttataaatactttagtctaccacctattaagagtgttaat |
| | | ttctccattagctcagttgagcagtatggcttttggaccatagcctacact |
| | | aactacacagatgtaatggtggatgttaatggaacaggtattactaggttg |
| | | ttctattgtgactcacccctcaataggattaagtgccaacaattgaagcat |
| | | gaactaccagatgggttttattctgctagcatgcttgtcaaaaaggatcta |
| | | cctaaaacatttgtaactatgccacaattttataactggatgaatgttacg |
| | | ttacatgtcgtgttgaatgacactgagaagagagctgatattatcttggct |
| | | aaggctccagaactagcctcacttgctgatatacattttgaaatagcccag |
| | | gctaatggcagtgtaactaatgttactagcctatgtgtacaagcaagacag |
| | | ttagctctattttataagtatactagcttacaaggtttgtatacctattct |
| | | aatcttgtggagttacaaaattatgactgtcctttttcgccacagcagttt |
| | | aataattatttgcagtttgaaactttgtgtcttgatgtaaatccagctgtt |
| | | gcaggttgtaagtggtcgttagtccatgataataagtggcgcacacagttc |
| | | gccactatcactgtttcctacaaagaaggtgctatgattacaactatgcca |
| | | aaagcacagctgggcttccaagatatttctaatttggtaaaagatgaatgc |
| | | actgattacaacatatatggatttcagggcacaggcattattagaaatacc |
| | | acctcacgtttagtggctggcctctactacacatctatcagtggcgacctt |
| | | cttgcttttaaaaatagtactactggggaaattttcactgtagtgccatgt |
| | | gatttaacagcacaggcagctgtgattaacgatgaaatagtgggagctata |
| | | acagccgttaatcaaactgatctatttgaatttgtcaatcacacacagtcg |
| | | agaaggtcacgtaggtcaacactagaaacagtacaaacctacactatgccg |
| | | caattttattacataacaaagtggaataatgacacatcgactaattgtacg |
| | | tctgtcattacgtactcctcctttgctatttgtaatactggtgaaattaaa |
| | | tatgttaatgtcactcatgttgaaacggtggatgataatatcggtgttatc |
| | | aaacctatttcaacaggtaacatatctatacctaaaaatttcacagtggca |
| | | gtgcaagctgaatatattcagattcaagtcaaacctgttgttgtagattgt |
| | | gccaagtacgtctgcaatggtaatagacattgccttagcttgctaacacaa |
| | | tacacatcagcttgtcaaacgattgaaaatgcccttaatcttggtgcacgt |
| | | cttgaatctttgatgcttaacgatatgatcacagtgtcagatcgtagtctg |
| | | gaacttgcaaccgttgaaaagtttaataccactgctctaggtggtgaaaaa |
| | | atcggtggtttttactttgatggcttgagtagtctgttaccacctaaaata |
| | | ggtaagaggtctgctgttgaggatttattgtttaataaggtggtaaccagt |
| | | ggtcttggtactgttgatgatgattataaaaagtgctcagccggcactgat |
| | | gtagctgacttagtttgtgcccaatattacaatggcataatggttttacct |
| | | ggtgttgtagaccacaataagatggctatctatactgcctctctaatagga |
| | | ggtatggccatgggctctattacatctgctgtggctgtcccttttgcaatg |
| | | caagtgcaggctaggcttaattatgttgcattgcaaacggatgtcctacag |
| | | gaaaaccagaaaatacttgctaatgcctttaataatgccattggtaacatt |
| | | acactagcacttggaaaagtttccaatgctattacgaccatatcagatggt |
| | | tttaatagtatggcttcagcactgactaagatccagagtgtagttaatcag |
| | | cagggtgaagcgttgagtcaacttaccagtcagttacagaaaaacttccaa |
| | | gctattagtagttccattgctgaaatttacaatagattggaaaaagtggaa |
| | | gctgatgctcaagttgatcgtctcattactggtagattggcagcacttaat |
| | | gcttatgtatcccaaactttaactcagtatgctgaagtcaaggctagtaga |
| | | caacttgcaatggaaaaagttaatgagtgtgtgaaatcacagtcggatagg |
| | | tatggtttctgtggcaatggaacccacttattctctcttgtcaattctgca |
| | | cctgatggtttgcttttctttcacacagtgttacttcctacggagtgggaa |
| | | gaggtgacggcatggtcaggaatatgtgttaacgacacatatgcatatgtg |
| | | ttgaaagattttgaatattctattttcagctacaataacacgtatatggtg |
| | | actcctcgtaatatgttccagcctagaaaacctcagatgagtgatttcgtg |
| | | caaattacgagctgtgaagtgacattcctgaacactacatatacgacattt |
| | | caggagattgtgattgattatattgatatcaacaagactatctctgatatg |
| | | cttgaacaatataaccccaactacacgacacctgaattagatttacagctg |
| | | gaaatcttcaatcagacaaagttaaatctcactgcagaaatagaccaattg |
| | | gaacaaagagctgacaatcttacgaacatagcacacgaactacaacagtac |
| | | attgacaatcttaataagacacttgttgaccttgaatggctcaacaggatt |
| | | gaaacttatgtaaaatggccttggtatgtgtggctacttattggtttagta |
| | | gtcgtcttctgcatacctttgttactgttttgctgtcttagtactggttgc |
| | | tgtgggtgctttggttgtcttggcagttgttgtcactctctttgtagtaga |
| | | cgtcaatttgaaagctatgaacctattgaaaaggttcacattcattaaTGA |
| | | CGTACGGCTGGAGCCTCGGTGGCCTAGCTTCTTGCCCCTTGGGCCTCCCCC |
| | | CAGCCCCTCCTCCCCTTCCTGCACCCGTACCCCCGTGGTCTTTGAATAAAG |
| | | TCTGAGTGGGCGGCAaaaaaaaaaaaaaaaaaaaaaaaaaaaaagcatatg |
| | | actaaaaaaaaaaaaaaaaaaaaaaaaaaaaaaaaaaaaaaaaaaaaaaaa |
| | | aaaaaaaaaaaaaaaaaaaaaaAAAAGAAGAGCAAGCTT |
| FCoV S-2P; Protein | 635 | MILLLTLLAVVRSEDAAKNVFVPIDGKIPESFSFSNWFLLTDKSTLVQGRV |
| Sequence | | LSKQPVFVQCLRPVPTSSNVTAVVHFKNDVFCPNVTADVLRFNLNFSDSNY |
| | | TESNNTDQLYFTFEDNTTASIACYSSANVTDFQPANNSVSHIPFGKTTHSY |
| | | FCFANFSHSVVSRQFLGILPPTVREFAFGRDGSIFVNGYKYFSLPPIKSVN |
| | | FSITPVSNNTFWTIAYTNYTDVMVDVNGTGITRLFYCDSPLNRIKCQQLKH |
| | | ELPDGFYSASMLVKKDLPKTFVTMPQFYNWMNVTLHVVLNDTEKKADIILA |
| | | KAPELASLADIHFEIAQANGSVTNVTSLCVQTRQLALFYKYTSLQGLYTYS |
| | | NLVELQNYDCPFSPQQFNNYLQFETLCFDVNPAVAGCKWSLVHDVKWRTQF |
| | | ATITVSYKDGAMITTMPKAQLGFQDISNLVKDECTDYNIYGFQGTGIIRNT |
| | | TSRLVAGLYYTSASGDLLAFKNSTTGEIFTVVPCDLTAQAAVINDEIVGAI |
| | | TAVNQTDLFEFVGGNGSVTTYTMPQFYYITKWNNDTSTNCTSVITYSSFAI |
| | | CNTGEIKYVNVTHVEIVDDSIGVIKPISTGNITIPKNFTVAVQAEYIQIQV |
| | | KPVVVDCAKYVCNGNRHCLSLLTQYTSACQTIENALNLGARLESLMLNDMI |
| | | TVSDRSLELATVEKFNTTVLGGEKLGGFYFDGLSSLLPPTIGKRSAVEDLL |
| | | FNKVVTSGLGTVDDDYKKCSAGTDVADLVCAQYYNGIMVLPGVVDDNKMAM |
| | | YTASLIGGMAMGSITSAVAVPFAMQVQARLNYVALQTDVLQENQKILANAF |
| | | NNAIGNITLALGKVSNAITTISDGFNTMASALTKIQSVVNQQGEALSQLIS |
| | | QTQKNFQATSSSTAETYNRLEPPFADAQVDRTTTGRTAATNAYVSQTLTQY |
| | | AEVKASRQLAMEKVNECVKSQSDRYGFCGNGTHLESLVNSAPDGLLFFHTV |
| | | LLPTEWEEVTAWSGICVNDTYAYVLKDFEYSIFSYNNTYMVTPRNMFQPRK |
| | | PQMSDFVQITSCEVTFLNTTYSTFQEIVIDYIDINKTIADMLEQYNPNYTT |
| | | PELDLQLEIFNQTKLNLTAEIDQLEQRADNLTTIAHELQQYIDNLNKTLVD |
| | | LEWLNRIETYVKWPWYVWLLIGLVVVFCIPLLLFCCLSTGCCGCFGCLGSC |
| | | CHSLCSRRQFESYEPIEKVHIH* |
| FCoV S-2P; DNA | 636 | GAATTCGGCTTTAATACGACTCACTATAAGGACATTAGCTTCTGACACAAC |
| Sequence | | TGTGTTCACTAGCAACCTCAAACAGCCACCATGATTCTGCTGCTGACTCTG |
| | | TTAGCAGTGGTCAGAAGTGAGGATGCTGCTAAGAATGTGTTTGTCCCTATA |
| | | GATGGCAAGATCCCAGAATCCTTCAGCTTCTCGAACTGGTTCCTGTTGACC |
| | | GACAAGAGCACTTTGGTCCAGGGACGCGTGCTGAGCAAACAACCAGTCTTC |
| | | GTCCAGTGCCTCCGCCCAGTGCCAACAAGCAGCAACGTCACAGCGGTTGTG |
| | | CACTTTAAAAATGATGTCTTCSGTCCAAATGTTACCGCTGATGTGCTTAGA |
| | | TTCAATCTCAACTTTTCTGATTCCAATTACACAGAATCCAACAACACGGAC |
| | | CAGCTCTACTTCACTTTCGAGGACAACACAACCGCTAGTATCGCCTGCTAC |
| | | TCCTCAGCGAATGTTACCGATTTCCAACCAGCCAACAATAGCGTGAGTCAC |
| | | ATCCCTTTTGGGAAGACAACCCACAGCTACTTCTGCTTCGCAAATTTCAGT |
| | | CATTCTGTCGTTAGCCGGCAATTCTTGGGGATCCTGCCGCCTACCGTGCGG |
| | | GAGTTTGCCTTCGGACGGGACGGCTCGATCTTTGTCAACGGCTACAAGTAT |
| | | TTTAGTCTCCCGCCGATCAAATCGGTGAACTTCTCAATTACACCTGTCTCC |
| | | AATAATACCTTCTGGACAATTGCATACACAAATTACACAGATGTGATGGTG |
| | | GATGTTAACGGGACAGGAATTACCAGGCTgTTCTACTGTGACAGTCCTTTG |
| | | AACAGGATAAAGTGCcaGCAGTTGAAGCATGAACTGCCAGATGGGTTCTAT |
| | | TCTGCTTCCATGCTGGTCAAGAAAGATCTCCCAAAAACGTTCGTGACAATG |
| | | CCTCAATTCTACAACTGGATGAACGTAACCCTGCACGTCGTACTAAATGAT |
| | | ACCGAGAAGAAGGCTGATATCATCCTCGCAAAGGCTCCAGAGCTGGCTTCC |
| | | CTGGCAGATATTCACTTCGAGATTGCACAGGCCAATGGCTCTGTGACGAAT |
| | | GTTACCTCGCTGTGTGTGCAGACGAGGCAGCTGGCCCTCTTCTATAAGTAT |
| | | ACCAGCCTGCAAGGCCTGTATACGTATAGTAATTTGGTGGAATTGCAGAAT |
| | | TATGACTGCCCCTTTTCTCCACAGcagTTTAATAACTACCTGCAATTTGAG |
| | | ACCCTCTGCTTTGATGTAAACCCTGCAGTCGCCGGGTGTAAGTGGTCACTG |
| | | GTGCATGACGTTAAGTGGAGAACCCAGTTTGCTACGATAACAGTGTCGTAC |
| | | AAAGACGGAGCTATGATAACTACTATGCCCAAAGCCCAGCTTGGCTTCCAG |
| | | GACATTTCGAACCTTGTGAAGGATGAATGTACCGACTATAATATCTATGGA |
| | | TTCCAGGGGACAGGCATCATCCGGAACACCACCTCCCGACTGGTGGCCGGC |
| | | TTGTATTACACTTCTGCATCTGGCGATTTGCTAGCCTTTAAGAACTCCACT |
| | | ACTGGGGAGATCTTCACCGTCGTTCCCTGTGATCTGACAGCTCAGGCCGCA |
| | | GTTATTAATGATGAGATTGTCGGTGCCATTACCGCCGTGAACCAGACTGAT |
| | | CTCTTTGAATTTGTGGGCGGTAATGGATCAGTGACTACATACACCATGCCT |
| | | CAGTTTTATTATATCACCAAGTGGAACAATGACACTTCAACCAACTGCACC |
| | | TCTGTCATTACATACTCCAGCTTTGCTATCTGCAACACAGGAGAAATCAAG |
| | | TACGTCAATGTAACCCATGTGGAGATCGTCGATGATTCAATCGGAGTCATT |
| | | AAACCTATATCAACTGGAAACATCACAATCCCCAAGAATTTTACTGTCGCA |
| | | GTGCAAGCAGAGTACATTCAGATCCAGGTCAAGCCTGTAGTGGTGGACTGT |
| | | GCCAAATATGTTTGCAATGGAAATAGGCACTGCCTGTCCCTGCTCACTCAG |
| | | TATACTTCAGCCTGTCAAACTATTGAGAATGCACTCAACCTCGGGGCCCGT |
| | | CTTGAGTCACTGATGTTGAATGATATGATCACCGTGTCTGACCGGTCTTTG |
| | | GAACTTGCAACGGTGGAGAAGTTCAACACCACAGTACTTGGAGGAGAAAAA |
| | | CTCGGTGGCTTCTACTTTGACGGGCTGAGTTCTCTATTGCCTCCAACCATC |
| | | GGAAAGAGGAGCGCTGTGGAAGACCTGTTATTCAATAAAGTTGTTACATCT |
| | | GGTCTGGGCACCGTGGACGATGACTACAAAAAATGTTCCGCAGGCACTGAT |
| | | GTCGCTGACCTGGTGTGCGCCCAGTACTATAATGGTATCATGGTTTTACCC |
| | | GGTGTGGTTGACGATAATAAGATGGCCATGTATACGGCCAGCCTGATAGGC |
| | | GGCATGGCAATGGGCTCCATCACGTCAGCCGTTGCGGTGCCCTTCGCCATG |
| | | CAGGTGCAAGCGCGTCTAAACTACGTGGCGCTTCAGACCGACGTGCTGCAG |
| | | GAAAATCAAAAGATACTAGCCAACGCGTTCAACAATGCTATCGGGAATATT |
| | | ACTCTTGCCTTGGGGAAAGTGAGTAATGCCATCACTACCATCTCTGATGGC |
| | | TTCAACACCATGGCTAGTGCCCTCACCAAGATACAGTCCGTAGTGAATCAG |
| | | CAGGGTGAGGCTCTTAGCCAGTTGATCTCCCAGCTGCAGAAGAACTTTCAA |
| | | GCAATTTCTTCTTCCATAGCTGAAATTTACAACCGCCTGGAgCCACCGGAG |
| | | GCCGATGCCCAGGTAGATAGGCTGATTACAGGGCGTCTCGCCGCTCTTAAT |
| | | GCCTATGTCAGCCAGACCTTGACACAGTACGCAGAGGTTAAAGCAAGTCGG |
| | | CAGTTAGCGATGGAGAAAGTTAATGAGTGCGTGAAATCCCAATCCGACCGG |
| | | TATGGCTTTTGTGGCAATGGAACACACCTGTTTTCGTTGGTGAACAGCGCT |
| | | CCTGACGGTCTCCTATTCTTCCACACCGTGTTGCTGCCCACAGAATGGGAG |
| | | GAGGTGACAGCCTGGAGCGGGATTTGTGTCAACGACACCTATGCCTACGTT |
| | | CTCAAAGACTTTGAATACAGCATTTTTAGTTACAATAACACGTACATGGTC |
| | | ACACCCAGAAACATGTTTCAGCCCCGAAAGCCCCAGATGAGCGATTTTGTT |
| | | CAGATAACCTCGTGTGAAGTGACCTTTCTGAATACTACTTACACCACGTTT |
| | | CAGGAGATCGTCATTGACTATATTGACATAAACAAGACTATTGCTGACATG |
| | | CTTGAACAGTACAACCCAAACTATACTACTCCTGAGTTGGACCTTCAGCTG |
| | | GAGATCTTTAACCAAACAAAACTGAACCTGACTGCAGAAATAGATCAGCTC |
| | | GAGCAGAGAGCCGATAACCTAACAACCATAGCTCATGAGCTCCAGCAATAC |
| | | ATCGACAATCTGAACAAGACTCTTGTAGACCTGGAATGGTTGAACAGAATC |
| | | GAAACATACGTAAAATGGCCCTGGTATGTGTGGCTCCTCATCGGCCTCGTC |
| | | GTAGTGTTCTGCATCCCCCTGTTGCTTTTTTGTTGCCTGTCTACTGGTTGC |
| | | TGTGGTTGTTTTGGATGTCTCGGCAGCTGCTGCCACTCCCTGTGCAGCAGA |
| | | CGCCAGTTCGAGAGCTATGAACCCATCGAGAAGGTTCATATTCATTGATAA |
| | | CGTACGGCTGGAGCCTCGGTGGCCTAGCTTCTTGCCCCTTGGGCCTCCCCC |
| | | CAGCCCCTCCTCCCCTTCCTGCACCCGTACCCCCGTGGTCTTTGAATAAAG |
| | | TCTGAGTGGGCGGCAaaaaaaaaaaaaaaaaaaaaaaaaaaaaagcatatg |
| | | actaaaaaaaaaaaaaaaaaaaaaaaaaaaaaaaaaaaaaaaaaaaaaaaa |
| | | aaaaaaaaaaaaaaaaaaaaaaAAAAGAAGAGCAAGCTT |
| FCoV S-2P2CD; | 637 | MILLLTLLAVVRSEDAAKNVFVPIDGKIPESFSFSNWFLLTDKSTLVQGRV |
| Protein Sequence | | LSKQPVFVQCLRPVPTSSNVTAVVHFKNDVFCPNVTADVLRFNLNFSDSNY |
| | | TESNNTDQLYFTFEDNTTASIACYSSANVTDFQPANNSVSHIPFGKTTHSY |
| | | FCFANFSHSVVSRQFLGILPPTVREFAFGRDGSIFVNGYKYFSLPPIKSVN |
| | | FSITPVSNNTFWTTAYTNYTDVMVDVNGTGTTRLFYCDSPLNRIKCRQLKH |
| | | ELPDGFYSASMLVKKDLPKTFVTMPQFYNWMNVTLHVVLNDTEKKADIILA |
| | | KAPELASLADIHFEIAQANGSVINVTSLCVQTRQLALFYKYTSLQGLYTYS |
| | | NLVELQNYDCPFSPQKFNNYLQFETLCFDVNPAVAGCKWSLVHDVKWRTQF |
| | | ATITVSYKDGAMITTMPKAQLGFQDISNLVKDECTDYNIYGFQGTGIIRNT |
| | | TSRLVAGLYYTSASGDLLAFKNSTTGEIFTVVPCDLTAQAAVINDEIVGAI |
| | | TAVNQTDLFEFVGGNGSVTTYTMPQFYYITKWNNDTSTNCTSVITYSSFAI |
| | | CNTGEIKYVNVTHVEIVDDSIGVIKPISTGNITIPKNFTVAVQAEYIQIQV |
| | | KPVVVDCAKYVCNGNRHCLSLLTQYTSACQTIENALNLGARLESLMLNDMI |
| | | TVSDRSLELATVEKFNTTVLGGEKLGGFYFDGLSSLLPPTIGKRSAVEDLL |
| | | FNKVVTSGLGTVDDDYKKCSAGTDVADLVCAQYYNGIMVLPGVVDDNKMAM |
| | | YTASLIGGMAMGSITSAVAVPFAMQVQARLNYVALQTDVLQENQKILANAF |
| | | NNAIGNITLALGKVSNAITTISDGFNTMASALTKIQSVVNQQGEALSQLIS |
| | | QLQKNFQAISSSIAEIYNRLDPPEADAQVDRLITGRLAALNAYVSQTLTQY |
| | | AEVKASRQLAMEKVNECVKSQSDRYGFCGNGTHLFSLVNSAPDGLLFFHTV |
| | | LLPTEWEEVTAWSGICVNDTYAYVLKDFEYSIFSYNNTYMVTPRNMFQPRK |
| | | PQMSDFVQITSCEVTFLNTTYTTFQEIVIDYIDINKTIADMLEQYNPNYTT |
| | | PELDLQLEIFNQTKLNLTAEIDQLEQRADNLTTIAHELQQYIDNLNKTLVD |
| | | LEWLNRIETYVKWPWYVWLLIGLVVVFCIPLLLFCCLSTGCCGCFGCLGSC |
| | | CHSLCSRRQFESYEPIEKVHIH* |
| FCoV S-2P2CD; | 638 | GAATTCGGCTTTAATACGACTCACTATAAGGACATTAGCTTCTGACACAAC |
| DNA Sequence | | TGTGTTCACTAGCAACCTCAAACAGCCACCATGATTCTGCTGCTGACTCTG |
| | | TTAGCAGTGGTCAGAAGTGAGGATGCTGCTAAGAATGTGTTTGTCCCTATA |
| | | GATGGCAAGATCCCAGAATCCTTCAGCTTCTCGAACTGGTTCCTGTTGACC |
| | | GACAAGAGCACTTTGGTCCAGGGACGCGTGCTGAGCAAACAACCAGTCTTC |
| | | GTCCAGTGCCTCCGCCCAGTGCCAACAAGCAGCAACGTCACAGCGGTTGTG |
| | | CACTTTAAAAATGATGTCTTCTGTCCAAATGTTACCGCTGATGTGCTTAGA |
| | | TTCAATCTCAACTTTTCTGATTCCAATTACACAGAATCCAACAACACGGAC |
| | | CAGCTCTACTTCACTTTCGAGGACAACACAACCGCTAGTATCGCCTGCTAC |
| | | TCCTCAGCGAATGTTACCGATTTCCAACCAGCCAACAATAGCGTGAGTCAC |
| | | ATCCCTTTTGGGAAGACAACCCACAGCTACTTCTGCTTCGCAAATTTCAGT |
| | | CATTCTGTCGTTAGCCGGCAATTCTTGGGGATCCTGCCGCCTACCGTGCGG |
| | | GAGTTTGCCTTCGGACGGGACGGCTCGATCTTTGTCAACGGCTACAAGTAT |
| | | TTTAGTCTCCCGCCGATCAAATCGGTGAACTTCTCAATTACACCTGTCTCC |
| | | AATAATACCTTCTGGACAATTGCATACACAAATTACACAGATGTGATGGTG |
| | | GATGTTAACGGGACAGGAATTACCAGGCTGTTCTACTGTGACAGTCCTTTG |
| | | AACAGGATAAAGTGCAGGCAGTTGAAGCATGAACTGCCAGATGGGTTCTAT |
| | | TCTGCTTCCATGCTGGTCAAGAAAGATCTCCCAAAAACGTTCGTGACAATG |
| | | CCTCAATTCTACAACTGGATGAACGTAACCCTGCACGTCGTACTAAATGAT |
| | | ACCGAGAAGAAGGCTGATATCATCCTCGCAAAGGCTCCAGAGCTGGCTTCC |
| | | CTGGCAGATATTCACTTCGAGATTGCACAGGCCAATGGCTCTGTGACGAAT |
| | | GTTACCTCGCTGTGTGTGCAGACGAGGCAGCTGGCCCTCTTCTATAAGTAT |
| | | ACCAGCCTGCAAGGCCTGTATACGTATAGTAATTTGGTGGAATTGCAGAAT |
| | | TATGACTGCCCCTTTTCTCCACAGAAATTTAATAACTACCTGCAATTTGAG |
| | | ACCCTCTGCTTTGATGTAAACCCTGCAGTCGCCGGGTGTAAGTGGTCACTG |
| | | GTGCATGACGTTAAGTGGAGAACCCAGTTTGCTACGATAACAGTGTCGTAC |
| | | AAAGACGGAGCTATGATAACTACTATGCCCAAAGCCCAGCTTGGCTTCCAG |
| | | GACATTTCGAACCTTGTGAAGGATGAATGTACCGACTATAATATCTATGGA |
| | | TTCCAGGGGACAGGCATCATCCGGAACACCACCTCCCGACTGGTGGCCGGC |
| | | TTGTATTACACTTCTGCATCTGGCGATTTGCTAGCCTTTAAGAACTCCACT |
| | | ACTGGGGAGATCTTCACCGTCGTTCCCTGTGATCTGACAGCTCAGGCCGCA |
| | | GTTATTAATGATGAGATTGTCGGTGCCATTACCGCCGTGAACCAGACTGAT |
| | | CTCTTTGAATTTGTGGGCGGTAATGGATCAGTGACTACATACACCATGCCT |
| | | CAGTTTTATTATATCACCAAGTGGAACAATGACACTTCAACCAACTGCACC |
| | | TCTGTCATTACATACTCCAGCTTTGCTATCTGCAACACAGGAGAAATCAAG |
| | | TACGTCAATGTAACCCATGTGGAGATCGTCGATGATTCAATCGGAGTCATT |
| | | AAACCTATATCAACTGGAAACATCACAATCCCCAAGAATTTTACTGTCGCA |
| | | GTGCAAGCAGAGTACATTCAGATCCAGGTCAAGCCTGTAGTGGTGGACTGT |
| | | GCCAAATATGTTTGCAATGGAAATAGGCACTGCCTGTCCCTGCTCACTCAG |
| | | TATACTTCAGCCTGTCAAACTATTGAGAATGCACTCAACCTCGGGGCCCGT |
| | | CTTGAGTCACTGATGTTGAATGATATGATCACCGTGTCTGACCGGTCTTTG |
| | | GAACTTGCAACGGTGGAGAAGTTCAACACCACAGTACTTGGAGGAGAAAAA |
| | | CTCGGTGGCTTCTACTTTGACGGGCTGAGTTCTCTATTGCCTCCAACCATC |
| | | GGAAAGAGGAGCGCTGTGGAAGACCTGTTATTCAATAAAGTTGTTACATCT |
| | | GGTCTGGGCACCGTGGACGATGACTACAAAAAATGTTCCGCAGGCACTGAT |
| | | GTCGCTGACCTGGTGTGCGCCCAGTACTATAATGGTATCATGGTTTTACCC |
| | | GGTGTGGTTGACGATAATAAGATGGCCATGTATACGGCCAGCCTGATAGGC |
| | | GGCATGGCAATGGGCTCCATCACGTCAGCCGTTGCGGTGCCCTTCGCCATG |
| | | CAGGTGCAAGCGCGTCTAAACTACGTGGCGCTTCAGACCGACGTGCTGCAG |
| | | GAWAATCAAAAGATACTAGCCAACGCGTTCAACAATGCTATCGGGAATATT |
| | | ACTCTTGCCTTGGGGAAAGTGAGTAATGCCATCACTACCATCTCTGATGGC |
| | | TTCAACACCATGGCTAGTGCCCTCACCAAGATACAGTCCGTAGTGAATCAG |
| | | CAGGGTGAGGCTCTTAGCCAGTTGATCTCCCAGCTGCAGAAGAACTTTCAA |
| | | GCAATTTCTTCTTCCATAGCTGAAATTTACAACCGCCTGGACCCACCGGAG |
| | | GCCGATGCCCAGGTAGATAGGCTGATTACAGGGCGTCTCGCCGCTCTTAAT |
| | | GCCTATGTCAGCCAGACCTTGACACAGTACGCAGAGGTTAAAGCAAGTCGG |
| | | CAGTTAGCGATGGAGAAAGTTAATGAGTGCGTGAAATCCCAATCCGACCGG |
| | | TATGGCTTTTGTGGCAATGGAACACACCTGTTTTCGTTGGTGAACAGCGCT |
| | | CCTGACGGTCTCCTATTCTTCCACACCGTGTTGCTGCCCACAGAATGGGAG |
| | | GAGGTGACAGCCTGGAGCGGGATTTGTGTCAACGACACCTATGCCTACGTT |
| | | CTCAAAGACTTTGAATACAGCATTTTTAGTTACAATAACACGTACATGGTC |
| | | ACACCCAGAAACATGTTTCAGCCCCGAAAGCCCCAGATGAGCGATTTTGTT |
| | | CAGATAACCTCGTGTGAAGTGACCTTTCTGAATACTACTTACACCACGTTT |
| | | CAGGAGATCGTCATTGACTATATTGACATAAACAAGACTATTGCTGACATG |
| | | CTTGAACAGTACAACCCAAACTATACTACTCCTGAGTTGGACCTTCAGCTG |
| | | GAGATCTTTAACCAAACAAAACTGAACCTGACTGCAGAAATAGATCAGCTC |
| | | GAGCAGAGAGCCGATAACCTAACAACCATAGCTCATGAGCTCCAGCAATAC |
| | | ATCGACAATCTGAACAAGACTCTTGTAGACCTGGAATGGTTGAACAGAATC |
| | | GAAACATACGTAAAATGGCCCTGGTATGTGTGGCTCCTCATCGGCCTCGTC |
| | | GTAGTGTTCTGCATCCCCCTGTTGCTTTTTTGTTGCCTGTCTACTGGTTGC |
| | | TGTGGTTGTTTTGGATGTCTCGGCAGCTGCTGCCACTCCCTGTGCAGCAGA |
| | | CGCCAGTTCGAGAGCTATGAACCCATCGAGAAGGTTCATATTCATTGATAA |
| | | CGTACGGCTGGAGCCTCGGTGGCCTAGCTTCTTGCCCCTTGGGCCTCCCCC |
| | | CAGCCCCTCCTCCCCTTCCTGCACCCGTACCCCCGTGGTCTTTGAATAAAG |
| | | TCTGAGTGGGCGGCAaaaaaaaaaaaaaaaaaaaaaaaaaaaaagcatatg |
| | | actaaaaaaaaaaaaaaaaaaaaaaaaaaaaaaaaaaaaaaaaaaaaaaaa |
| | | aaaaaaaaaaaaaaaaaaaaaaAAAAGAAGAGCAAGCTT |
| FCoV S-4P; Protein | 639 | MILLLTLLAVVRSEDAAKNVFVPIDGKIPESFSFSNWFLLTDKSTLVQGRV |
| Sequence | | LSKQPVFVQCLRPVPTSSNVTAVVHFKNDVFCPNVTADVLRFNLNFSDSNY |
| | | TESNNTDQLYFTFEDNTTASIACYSSANVTDFQPANNSVSHIPFGKTTHSY |
| | | FCFANFSHSVVSRQFLGILPPTVREFAFGRDGSIFVNGYKYFSLPPIKSVN |
| | | FSITPVSNNTFWTIAYTNYTDVMVDVNGTGITRLFYCDSPLNRIKCQQLKH |
| | | ELPDGFYSASMLVKKDLPKTFVTMPQFYNWMNVTLHVVLNDTEKKADIILA |
| | | KAPELASLADIHFEIAQANGSVTNVTSLCVQTRQLALFYKYTSLQGLYTYS |
| | | NLVELQNYDCPFSPQQFNNYLQFETLCFDVNPAVAGCKWSLVHDVKWRTQF |
| | | ATITVSYKDGAMITTMPKAQLGFQDISNLVKDECTDYNIYGFQGTGIIRNT |
| | | TSRLVAGLYYTSASGDLLAFKNSTIGEIFTVVPCDLIAQAAVINDEIVGAI |
| | | TAVNQTDLFEFVGGNGSVTTYTMPQFYYITKWNNDTSTNCTSVITYSSFAI |
| | | CNTGEIKYVNVTHVEIVDDSIGVIKPISTGNITIPKNFTVAVQAEYIQIQV |
| | | KPVVVDCAKYVCNGNRHCLSLLTQYTSACQTIENALNLGARLESLMLNDMI |
| | | TVSDRSLELATVEKFNTTVLGGEKLGGFYFDGLSSLLPPTIGKRSAVEDLL |
| | | FNKVVTSGLGTVDDDYKKCSAGTDVADLVCAQYYNGIMVLPGVVDDNKMAM |
| | | YTASLIGGMAMGSITSAVAVPFAMQVQARLNYVALQTDVLPENQKILANAF |
| | | NNATGNITLALGKGSNAITTISPGFNTMASALTKTQSVVNQQGEALSQLIS |
| | | QLQKNFQAISSSIAEIYNRLEPPEADAQVDRLITGRLAALNAYVSQTLTQY |
| | | AEVKASRQLAMEKVNECVKSQSDRYGFCGNGTHLFSLVNSAPDGLLFFHTV |
| | | LLPTEWEEVTAWSGICVNDTYAYVLKDFEYSIFSYNNTYMVTPRNMFQPRK |
| | | PQMSDFVQITSCEVTFLNTTYSTFQEIVIDYIDINKTIADMLEQYNPNYTT |
| | | PELDLQLEIFNQTKLNLTAEIDQLEQRADNLTTIAHELQQYIDNLNKTLVD |
| | | LEWLNRIETYVKWPWYVWLLIGLVVVFCIPLLLFCCLSTGCCGCFGCLGSC |
| | | CHSLCSRRQFESYEPIEKVHIH* |
| FCoV S-4P; DNA | 640 | GAATTCGGCTTTAATACGACTCACTATAAGGACATTAGCTTCTGACACAAC |
| Sequence | | TGTGTTCACTAGCAACCTCAAACAGCCACCATGATTCTGCTGCTGACTCTG |
| | | TTAGCAGTGGTCAGAAGTGAGGATGCTGCTAAGAATGTGTTTGTCCCTATA |
| | | GATGGCAAGATCCCAGAATCCTTCAGCTTCTCGAACTGGTTCCTGTTGACC |
| | | GACAAGAGCACTTTGGTCCAGGGACGCGTGCTGAGCAAACAACCAGTCTTC |
| | | GTCCAGTGCCTCCGCCCAGTGCCAACAAGCAGCAACGTCACAGCGGTTGTG |
| | | CACTTTAAAAATGATGTCTTCSGTCCAAATGTTACCGCTGATGTGCTTAGA |
| | | TTCAATCTCAACTTTTCTGATTCCAATTACACAGAATCCAACAACACGGAC |
| | | CAGCTCTACTTCACTTTCGAGGACAACACAACCGCTAGTATCGCCTGCTAC |
| | | TCCTCAGCGAATGTTACCGATTTCCAACCAGCCAACAATAGCGTGAGTCAC |
| | | ATCCCTTTTGGGAAGACAACCCACAGCTACTTCTGCTTCGCAAATTTCAGT |
| | | CATTCTGTCGTTAGCCGGCAATTCTTGGGGATCCTGCCGCCTACCGTGCGG |
| | | GAGTTTGCCTTCGGACGGGACGGCTCGATCTTTGTCAACGGCTACAAGTAT |
| | | TTTAGTCTCCCGCCGATCAAATCGGTGAACTTCTCAATTACACCTGTCTCC |
| | | AATAATACCTTCTGGACAATTGCATACACAAATTACACAGATGTGATGGTG |
| | | GATGTTAACGGGACAGGAATTACCAGGCTgTTCTACTGTGACAGTCCTTTG |
| | | AACAGGATAAAGTGCcaGCAGTTGAAGCATGAACTGCCAGATGGGTTCTAT |
| | | TCTGCTTCCATGCTGGTCAAGAAAGATCTCCCAAAAACGTTCGTGACAATG |
| | | CCTCAATTCTACAACTGGATGAACGTAACCCTGCACGTCGTACTAAATGAT |
| | | ACCGAGAAGAAGGCTGATATCATCCTCGCAAAGGCTCCAGAGCTGGCTTCC |
| | | CTGGCAGATATTCACTTCGAGATTGCACAGGCCAATGGCTCTGTGACGAAT |
| | | GTTACCTCGCTGTGTGTGCAGACGAGGCAGCTGGCCCTCTTCTATAAGTAT |
| | | ACCAGCCTGCAAGGCCTGTATACGTATAGTAATTTGGTGGAATTGCAGAAT |
| | | TATGACTGCCCCTTTTCTCCACAGcagTTTAATAACTACCTGCAATTTGAG |
| | | ACCCTCTGCTTTGATGTAAACCCTGCAGTCGCCGGGTGTAAGTGGTCACTG |
| | | GTGCATGACGTTAAGTGGAGAACCCAGTTTGCTACGATAACAGTGTCGTAC |
| | | AAAGACGGAGCTATGATAACTACTATGCCCAAAGCCCAGCTTGGCTTCCAG |
| | | GACATTTCGAACCTTGTGAAGGATGAATGTACCGACTATAATATCTATGGA |
| | | TTCCAGGGGACAGGCATCATCCGGAACACCACCTCCCGACTGGTGGCCGGC |
| | | TTGTATTACACTTCTGCATCTGGCGATTTGCTAGCCTTTAAGAACTCCACT |
| | | ACTGGGGAGATCTTCACCGTCGTTCCCTGTGATCTGACAGCTCAGGCCGCA |
| | | GTTATTAATGATGAGATTGTCGGTGCCATTACCGCCGTGAACCAGACTGAT |
| | | CTCTTTGAATTTGTGGGCGGTAATGGATCAGTGACTACATACACCATGCCT |
| | | CAGTTTTATTATATCACCAAGTGGAACAATGACACTTCAACCAACTGCACC |
| | | TCTGTCATTACATACTCCAGCTTTGCTATCTGCAACACAGGAGAAATCAAG |
| | | TACGTCAATGTAACCCATGTGGAGATCGTCGATGATTCAATCGGAGTCATT |
| | | AAACCTATATCAACTGGAAACATCACAATCCCCAAGAATTTTACTGTCGCA |
| | | GTGCAAGCAGAGTACATTCAGATCCAGGTCAAGCCTGTAGTGGTGGACTGT |
| | | GCCAAATATGTTTGCAATGGAAATAGGCACTGCCTGTCCCTGCTCACTCAG |
| | | TATACTTCAGCCTGTCAAACTATTGAGAATGCACTCAACCTCGGGGCCCGT |
| | | CTTGAGTCACTGATGTTGAATGATATGATCACCGTGTCTGACCGGTCTTTG |
| | | GAACTTGCAACGGTGGAGAAGTTCAACACCACAGTACTTGGAGGAGAAAAA |
| | | CTCGGTGGCTTCTACTTTGACGGGCTGAGTTCTCTATTGCCTCCAACCATC |
| | | GGAAAGAGGAGCGCTGTGGAAGACCTGTTATTCAATAAAGTTGTTACATCT |
| | | GGTCTGGGCACCGTGGACGATGACTACAAAAAATGTTCCGCAGGCACTGAT |
| | | GTCGCTGACCTGGTGTGCGCCCAGTACTATAATGGTATCATGGTTTTACCC |
| | | GGTGTGGTTGACGATAATAAGATGGCCATGTATACGGCCAGCCTGATAGGC |
| | | GGCATGGCAATGGGCTCCATCACGTCAGCCGTTGCGGTGCCCTTCGCCATG |
| | | CAGGTGCAAGCGCGTCTAAACTACGTGGCGCTTCAGACCGACGTGCTGCCG |
| | | GAAAATCAAAAGATACTAGCCAACGCGTTCAACAATGCTATCGGGAATATT |
| | | ACTCTTGCCTTGGGGAAAGGGAGTAATGCCATCACTACCATCTCTccaGGC |
| | | TTCAACACCATGGCTAGTGCCCTCACCAAGATACAGTCCGTAGTGAATCAG |
| | | CAGGGTGAGGCTCTTAGCCAGTTGATCTCCCAGCTGCAGAAGAACTTTCAA |
| | | GCAATTTCTTCTTCCATAGCTGAAATTTACAACCGCCTGGAgCCACCGGAG |
| | | GCCGATGCCCAGGTAGATAGGCTGATTACAGGGCGTCTCGCCGCTCTTAAT |
| | | GCCTATGTCAGCCAGACCTTGACACAGTACGCAGAGGTTAAAGCAAGTCGG |
| | | CAGTTAGCGATGGAGAAAGTTAATGAGTGCGTGAAATCCCAATCCGACCGG |
| | | TATGGCTTTTGTGGCAATGGAACACACCTGTTTTCGTTGGTGAACAGCGCT |
| | | CCTGACGGTCTCCTATTCTTCCACACCGTGTTGCTGCCCACAGAATGGGAG |
| | | GAGGTGACAGCCTGGAGCGGGATTTGTGTCAACGACACCTATGCCTACGTT |
| | | CTCAAAGACTTTGAATACAGCATTTTTAGTTACAATAACACGTACATGGTC |
| | | ACACCCAGAAACATGTTTCAGCCCCGAAAGCCCCAGATGAGCGATTTTGTT |
| | | CAGATAACCTCGTGTGAAGTGACCTTTCTGAATACTACTTACACCACGTTT |
| | | CAGGAGATCGTCATTGACTATATTGACATAAACAAGACTATTGCTGACATG |
| | | CTTGAACAGTACAACCCAAACTATACTACTCCTGAGTTGGACCTTCAGCTG |
| | | GAGATCTTTAACCAAACAAAACTGAACCTGACTGCAGAAATAGATCAGCTC |
| | | GAGCAGAGAGCCGATAACCTAACAACCATAGCTCATGAGCTCCAGCAATAC |
| | | ATCGACAATCTGAACAAGACTCTTGTAGACCTGGAATGGTTGAACAGAATC |
| | | GAAACATACGTAAAATGGCCCTGGTATGTGTGGCTCCTCATCGGCCTCGTC |
| | | GTAGTGTTCTGCATCCCCCTGTTGCTTTTTTGTTGCCTGTCTACTGGTTGC |
| | | TGTGGTTGTTTTGGATGTCTCGGCAGCTGCTGCCACTCCCTGTGCAGCAGA |
| | | CGCCAGTTCGAGAGCTATGAACCCATCGAGAAGGTTCATATTCATTGATAA |
| | | CGTACGGCTGGAGCCTCGGTGGCCTAGCTTCTTGCCCCTTGGGCCTCCCCC |
| | | CAGCCCCTCCTCCCCTTCCTGCACCCGTACCCCCGTGGTCTTTGAATAAAG |
| | | TCTGAGTGGGCGGCAaaaaaaaaaaaaaaaaaaaaaaaaaaaaagcatatg |
| | | actaaaaaaaaaaaaaaaaaaaaaaaaaaaaaaaaaaaaaaaaaaaaaaaa |
| | | aaaaaaaaaaaaaaaaaaaaaaAAAAGAAGAGCAAGCTT |
| FCoV S-2P-HA; | 641 | MILLLTLLAVVRSEDAAKNVFVPIDGKIPESFSFSNWFLLTDKSTLVQGRV |
| Protein Sequence | | LSKQPVFVQCLRPVPTSSNVTAVVHFKNDVFCPNVTADVLRFNLNFSDSNY |
| | | TESNNTDQLYFTFEDNTTASIACYSSANVTDFQPANNSVSHIPFGKTTHSY |
| | | FCFANFSHSVVSRQFLGILPPTVREFAFGRDGSIFVNGYKYFSLPPIKSVN |
| | | FSITPVSNNTFWTIAYTNYTDVMVDVNGTGITRLFYCDSPLNRIKCQQLKH |
| | | ELPDGFYSASMLVKKDLPKTFVTMPQFYNWMNVTLHVVLNDTEKKADIILA |
| | | KAPELASLADIHFEIAQANGSVTNVTSLCVQTRQLALFYKYTSLQGLYTYS |
| | | NLVELQNYDCPFSPQQFNNYLQFETLCFDVNPAVAGCKWSLVHDVKWRTQF |
| | | ATITVSYKDGAMITTMPKAQLGFQDISNLVKDECTDYNIYGFQGTGIIRNT |
| | | TSRLVAGLYYTSASGDLLAFKNSTTGEIFTVVPCDLTAQAAVINDEIVGAI |
| | | TAVNQTDLFEFVGGNGSVTTYTMPQFYYITKWNNDTSTNCTSVITYSSFAI |
| | | CNTGEIKYVNVTHVEIVDDSIGVIKPISTGNITIPKNFTVAVQAEYIQIQV |
| | | KPVVVDCAKYVCNGNRHCLSLLTQYTSACQTIENALNLGARLESLMLNDMI |
| | | TVSDRSLELATVEKFNTTVLGGEKLGGFYFDGLSSLLPPTIGKRSAVEDLL |
| | | FNKVVTSGLGTVDDDYKKCSAGTDVADLVCAQYYNGIMVLPGVVDDNKMAM |
| | | YTASLIGGMAMGSITSAVAVPFAMQVQARLNYVALQTDVLQENQKILANAF |
| | | NNAIGNITLALGKVSNAITTISDGFNTMASALTKIQSVVNQQGEALSQLIS |
| | | QLQKNFQAISSSIAEIYNRLEPPEADAQVDRLITGRLAALNAYVSQTLTQY |
| | | AEVKASRQLAMEKVNECVKSQSDRYGFCGNGTHLFSLVNSAPDGLLFFHTV |
| | | LLPTEWEEVTAWSGICVNDTYAYVLKDFEYSIFSYNNTYMVTPRNMFQPRK |
| | | PQMSDFVQITSCEVTFLNTTYTTFQEIVIDYIDINKTIADMLEQYNPNYTT |
| | | PELDLQLEIFNQTKLNLTAEIDQLEQRADNLTTIAHELQQYIDNLNKTLVD |
| | | LEWLNRIETYVKWPWYVWLLIGLVVVFCIPLLLFCCLSTGCCGCFGCLGSC |
| | | CHSLCSRRQFESYEPIEKVHIHDIYPYDVPDYA* |
| FCoV S-2P-HA; | 642 | GAATTCGGCTTTAATACGACTCACTATAAGGACATTAGCTTCTGACACAAC |
| DNA Sequence | | TGTGTTCACTAGCAACCTCAAACAGCCACCATGATTCTGCTGCTGACTCTG |
| | | TTAGCAGTGGTCAGAAGTGAGGATGCTGCTAAGAATGTGTTTGTCCCTATA |
| | | GATGGCAAGATCCCAGAATCCTTCAGCTTCTCGAACTGGTTCCTGTTGACC |
| | | GACAAGAGCACTTTGGTCCAGGGACGCGTGCTGAGCAAACAACCAGTCTTC |
| | | GTCCAGTGCCTCCGCCCAGTGCCAACAAGCAGCAACGTCACAGCGGTTGTG |
| | | CACTTTAAAAATGATGTCTTCTGTCCAAATGTTACCGCTGATGTGCTTAGA |
| | | TTCAATCTCAACTTTTCTGATTCCAATTACACAGAATCCAACAACACGGAC |
| | | CAGCTCTACTTCACTTTCGAGGACAACACAACCGCTAGTATCGCCTGCTAC |
| | | TCCTCAGCGAATGTTACCGATTTCCAACCAGCCAACAATAGCGTGAGTCAC |
| | | ATCCCTTTTGGGAAGACAACCCACAGCTACTTCTGCTTCGCAAATTTCAGT |
| | | CATTCTGTCGTTAGCCGGCAATTCTTGGGGATCCTGCCGCCTACCGTGCGG |
| | | GAGTTTGCCTTCGGACGGGACGGCTCGATCTTTGTCAACGGCTACAAGTAT |
| | | TTTAGTCTCCCGCCGATCAAATCGGTGAACTTCTCAATTACACCTGTCTCC |
| | | AATAATACCTTCTGGACAATTGCATACACAAATTACACAGATGTGATGGTG |
| | | GATGTTAACGGGACAGGAATTACCAGGCTgTTCTACTGTGACAGTCCTTTG |
| | | AACAGGATAAAGTGCcaGCAGTTGAAGCATGAACTGCCAGATGGGTTCTAT |
| | | TCTGCTTCCATGCTGGTCAAGAAAGATCTCCCAAAAACGTTCGTGACAATG |
| | | CCTCAATTCTACAACTGGATGAACGTAACCCTGCACGTCGTACTAAATGAT |
| | | ACCGAGAAGAAGGCTGATATCATCCTCGCAAAGGCTCCAGAGCTGGCTTCC |
| | | CTGGCAGATATTCACTTCGAGATTGCACAGGCCAATGGCTCTGTGACGAAT |
| | | GTTACCTCGCTGTGTGTGCAGACGAGGCAGCTGGCCCTCTTCTATAAGTAT |
| | | ACCAGCCTGCAAGGCCTGTATACGTATAGTAATTTGGTGGAATTGCAGAAT |
| | | TATGACTGCCCCTTTTCTCCACAGcaqTTTAATAACTACCTGCAATTTGAG |
| | | ACCCTCTGCTTTGATGTAAACCCTGCAGTCGCCGGGTGTAAGTGGTCACTG |
| | | GTGCATGACGTTAAGTGGAGAACCCAGTTTGCTACGATAACAGTGTCGTAC |
| | | AAAGACGGAGCTATGATAACTACTATGCCCAAAGCCCAGCTTGGCTTCCAG |
| | | GACATTTCGAACCTTGTGAAGGATGAATGTACCGACTATAATATCTATGGA |
| | | TTCCAGGGGACAGGCATCATCCGGAACACCACCTCCCGACTGGTGGCCGGC |
| | | TTGTATTACACTTCTGCATCTGGCGATTTGCTAGCCTTTAAGAACTCCACT |
| | | ACTGGGGAGATCTTCACCGTCGTTCCCTGTGATCTGACAGCTCAGGCCGCA |
| | | GTTATTAATGATGAGATTGTCGGTGCCATTACCGCCGTGAACCAGACTGAT |
| | | CTCTTTGAATTTGTGGGCGGTAATGGATCAGTGACTACATACACCATGCCT |
| | | CAGTTTTATTATATCACCAAGTGGAACAATGACACTTCAACCAACTGCACC |
| | | TCTGTCATTACATACTCCAGCTTTGCTATCTGCAACACAGGAGAAATCAAG |
| | | TACGTCAATGTAACCCATGTGGAGATCGTCGATGATTCAATCGGAGTCATT |
| | | AAACCTATATCAACTGGAAACATCACAATCCCCAAGAATTTTACTGTCGCA |
| | | GTGCAAGCAGAGTACATTCAGATCCAGGTCAAGCCTGTAGTGGTGGACTGT |
| | | GCCAAATATGTTTGCAATGGAAATAGGCACTGCCTGTCCCTGCTCACTCAG |
| | | TATACTTCAGCCTGTCAAACTATTGAGAATGCACTCAACCTCGGGGCCCGT |
| | | CTTGAGTCACTGATGTTGAATGATATGATCACCGTGTCTGACCGGTCTTTG |
| | | GAACTTGCAACGGTGGAGAAGTTCAACACCACAGTACTTGGAGGAGAAAAA |
| | | CTCGGTGGCTTCTACTTTGACGGGCTGAGTTCTCTATTGCCTCCAACCATC |
| | | GGAAAGAGGAGCGCTGTGGAAGACCTGTTATTCAATAAAGTTGTTACATCT |
| | | GGTCTGGGCACCGTGGACGATGACTACAAAAAATGTTCCGCAGGCACTGAT |
| | | GTCGCTGACCTGGTGTGCGCCCAGTACTATAATGGTATCATGGTTTTACCC |
| | | GGTGTGGTTGACGATAATAAGATGGCCATGTATACGGCCAGCCTGATAGGC |
| | | GGCATGGCAATGGGCTCCATCACGTCAGCCGTTGCGGTGCCCTTCGCCATG |
| | | CAGGTGCAAGCGCGTCTAAACTACGTGGCGCTTCAGACCGACGTGCTGCAG |
| | | GAAAATCAAAAGATACTAGCCAACGCGTTCAACAATGCTATCGGGAATATT |
| | | ACTCTTGCCTTGGGGAAAGTGAGTAATGCCATCACTACCATCTCTGATGGC |
| | | TTCAACACCATGGCTAGTGCCCTCACCAAGATACAGTCCGTAGTGAATCAG |
| | | CAGGGTGAGGCTCTTAGCCAGTTGATCTCCCAGCTGCAGAAGAACTTTCAA |
| | | GCAATTTCTTCTTCCATAGCTGAAATTTACAACCGCCTGGAgCCACCGGAG |
| | | GCCGATGCCCAGGTAGATAGGCTGATTACAGGGCGTCTCGCCGCTCTTAAT |
| | | GCCTATGTCAGCCAGACCTTGACACAGTACGCAGAGGTTAAAGCAAGTCGG |
| | | CAGTTAGCGATGGAGAAAGTTAATGAGTGCGTGAAATCCCAATCCGACCGG |
| | | TATGGCTTTTGTGGCAATGGAACACACCTGTTTTCGTTGGTGAACAGCGCT |
| | | CCTGACGGTCTCCTATTCTTCCACACCGTGTTGCTGCCCACAGAATGGGAG |
| | | GAGGTGACAGCCTGGAGCGGGATTTGTGTCAACGACACCTATGCCTACGTT |
| | | CTCAAAGACTTTGAATACAGCATTTTTAGTTACAATAACACGTACATGGTC |
| | | ACACCCAGAAACATGTTTCAGCCCCGAAAGCCCCAGATGAGCGATTTTGTT |
| | | CAGATAACCTCGTGTGAAGTGACCTTTCTGAATACTACTTACACCACGTTT |
| | | CAGGAGATCGTCATTGACTATATTGACATAAACAAGACTATTGCTGACATG |
| | | CTTGAACAGTACAACCCAAACTATACTACTCCTGAGTTGGACCTTCAGCTG |
| | | GAGATCTTTAACCAAACAAAACTGAACCTGACTGCAGAAATAGATCAGCTC |
| | | GAGCAGAGAGCCGATAACCTAACAACCATAGCTCATGAGCTCCAGCAATAC |
| | | ATCGACAATCTGAACAAGACTCTTGTAGACCTGGAATGGTTGAACAGAATC |
| | | GAAACATACGTAAAATGGCCCTGGTATGTGTGGCTCCTCATCGGCCTCGTC |
| | | GTAGTGTTCTGCATCCCCCTGTTGCTTTTTTGTTGCCTGTCTACTGGTTGC |
| | | TGTGGTTGTTTTGGATGTCTCGGCAGCTGCTGCCACTCCCTGTGCAGCAGA |
| | | CGCCAGTTCGAGAGCTATGAACCCATCGAGAAGGTTCATATTCATGATATC |
| | | TACCCATACGACGTACCAGATTACGCTTGACGTACGGCTGGAGCCTCGGTG |
| | | GCCTAGCTTCTTGCCCCTTGGGCCTCCCCCCAGCCCCTCCTCCCCTTCCTG |
| | | CACCCGTACCCCCGTGGTCTTTGAATAAAGTCTGAGTGGGCGGCAaaaaaa |
| | | aaaaaaaaaaaaaaaaaaaaaaagcatatgactaaaaaaaaaaaaaaaaaa |
| | | aaaaaaaaaaaaaaaaaaaaaaaaaaaaaaaaaaaaaaaaaaaaaaaaaaa |
| | | aAAAAGAAGAGCAAGCTT |
| FCoV S-4P-HA; | 643 | MILLLTLLAVVRSEDAAKNVFVPIDGKIPESFSFSNWELLTDKSTLVQGRV |
| Protein Sequence | | LSKQPVFVQCLRPVPTSSNVTAVVHFKNDVFCPNVTADVLRENLNFSDSNY |
| | | TESNNTDQLYFTFEDNTTASIACYSSANVTDFQPANNSVSHIPFGKTTHSY |
| | | FCFANFSHSVVSRQFLGILPPTVREFAFGRDGSIFVNGYKYFSLPPIKSVN |
| | | FSITPVSNNTFWTIAYTNYTDVMVDVNGTGITRLFYCDSPLNRIKCQQLKH |
| | | ELPDGFYSASMLVKKDLPKTFVTMPQFYNWMNVTLHVVLNDTEKKADIILA |
| | | KAPELASLADIHFEIAQANGSVTNVTSLCVQTRQLALFYKYTSLQGLYTYS |
| | | NLVELQNYDCPFSPQQFNNYLQFETLCFDVNPAVAGCKWSLVHDVKWRTQF |
| | | ATITVSYKDGAMITTMPKAQLGFQDISNLVKDECTDYNIYGFQGTGIIRNT |
| | | TSRLVAGLYYTSASGDLLAFKNSTTGEIFTVVPCDLTAQAAVINDEIVGAI |
| | | TAVNQTDLFEFVGGNGSVTTYTMPQFYYITKWNNDTSTNCTSVITYSSFAI |
| | | CNTGEIKYVNVTHVEIVDDSIGVIKPISTGNITIPKNFTVAVQAEYIQIQV |
| | | KPVVVDCAKYVCNGNRHCLSLLTQYTSACQTIENALNLGARLESLMLNDMI |
| | | TVSDRSLELATVEKFNTTVLGGEKLGGFYFDGLSSLLPPTIGKRSAVEDLL |
| | | FNKVVTSGTGTVDDDYKKCSAGTDVADLVCAQYYNGTMVLPGVVDDNKMAM |
| | | YTASLIGGMAMGSITSAVAVPFAMQVQARLNYVALQTDVLPENQKILANAF |
| | | NNAIGNITLALGKGSNAITTISPGFNTMASALTKIQSVVNQQGEALSQLIS |
| | | QLQKNFQAISSSIAEIYNRLEPPEADAQVDRLITGRLAALNAYVSQTLTQY |
| | | AEVKASRQLAMEKVNECVKSQSDRYGFCGNGTHLFSLVNSAPDGLLFFHTV |
| | | LLPTEWEEVTAWSGICVNDTYAYVLKDFEYSIFSYNNTYMVTPRNMFQPRK |
| | | PQMSDFVQITSCEVTFLNTTYTTFQEIVIDYIDINKTIADMLEQYNPNYTT |
| | | PELDLQLEIFNQTKLNLTAEIDQLEQRADNLTTIAHELQQYIDNLNKTLVD |
| | | LEWLNRIETYVKWPWYVWLLIGLVVVFCIPLLLFCCLSTGCCGCFGCLGSC |
| | | CHSLCSRRQFESYEPIEKVHIHDIYPYDVPDYA* |
| FCoV S-4P-HA; | 644 | GAATTCGGCTTTAATACGACTCACTATAAGGACATTAGCTTCTGACACAAC |
| DNA Sequence | | TGTGTTCACTAGCAACCTCAAACAGCCACCATGATTCTGCTGCTGACTCTG |
| | | TTAGCAGTGGTCAGAAGTGAGGATGCTGCTAAGAATGTGTTTGTCCCTATA |
| | | GATGGCAAGATCCCAGAATCCTTCAGCTTCTCGAACTGGTTCCTGTTGACC |
| | | GACAAGAGCACTTTGGTCCAGGGACGCGTGCTGAGCAAACAACCAGTCTTC |
| | | GTCCAGTGCCTCCGCCCAGTGCCAACAAGCAGCAACGTCACAGCGGTTGTG |
| | | CACTTTAAAAATGATGTCTTCTGTCCAAATGTTACCGCTGATGTGCTTAGA |
| | | TTCAATCTCAACTTTTCTGATTCCAATTACACAGAATCCAACAACACGGAC |
| | | CAGCTCTACTTCACTTTCGAGGACAACACAACCGCTAGTATCGCCTGCTAC |
| | | TCCTCAGCGAATGTTACCGATTTCCAACCAGCCAACAATAGCGTGAGTCAC |
| | | ATCCCTTTTGGGAAGACAACCCACAGCTACTTCTGCTTCGCAAATTTCAGT |
| | | CATTCTGTCGTTAGCCGGCAATTCTTGGGGATCCTGCCGCCTACCGTGCGG |
| | | GAGTTTGCCTTCGGACGGGACGGCTCGATCTTTGTCAACGGCTACAAGTAT |
| | | TTTAGTCTCCCGCCGATCAAATCGGTGAACTTCTCAATTACACCTGTCTCC |
| | | AATAATACCTTCTGGACAATTGCATACACAAATTACACAGATGTGATGGTG |
| | | GATGTTAACGGGACAGGAATTACCAGGCTgTTCTACTGTGACAGTCCTTTG |
| | | AACAGGATAAAGTGCcaGCAGTTGAAGCATGAACTGCCAGATGGGTTCTAT |
| | | TCTGCTTCCATGCTGGTCAAGAAAGATCTCCCAAAAACGTTCGTGACAATG |
| | | CCTCAATTCTACAACTGGATGAACGTAACCCTGCACGTCGTACTAAATGAT |
| | | ACCGAGAAGAAGGCTGATATCATCCTCGCAAAGGCTCCAGAGCTGGCTTCC |
| | | CTGGCAGATATTCACTTCGAGATTGCACAGGCCAATGGCTCTGTGACGAAT |
| | | GTTACCTCGCTGTGTGTGCAGACGAGGCAGCTGGCCCTCTTCTATAAGTAT |
| | | ACCAGCCTGCAAGGCCTGTATACGTATAGTAATTTGGTGGAATTGCAGAAT |
| | | TATGACTGCCCCTTTTCTCCACAGcagTTTAATAACTACCTGCAATTTGAG |
| | | ACCCTCTGCTTTGATGTAAACCCTGCAGTCGCCGGGTGTAAGTGGTCACTG |
| | | GTGCATGACGTTAAGTGGAGAACCCAGTTTGCTACGATAACAGTGTCGTAC |
| | | AAAGACGGAGCTATGATAACTACTATGCCCAAAGCCCAGCTTGGCTTCCAG |
| | | GACATTTCGAACCTTGTGAAGGATGAATGTACCGACTATAATATCTATGGA |
| | | TTCCAGGGGACAGGCATCATCCGGAACACCACCTCCCGACTGGTGGCCGGC |
| | | TTGTATTACACTTCTGCATCTGGCGATTTGCTAGCCTTTAAGAACTCCACT |
| | | ACTGGGGAGATCTTCACCGTCGTTCCCTGTGATCTGACAGCTCAGGCCGCA |
| | | GTTATTAATGATGAGATTGTCGGTGCCATTACCGCCGTGAACCAGACTGAT |
| | | CTCTTTGAATTTGTGGGCGGTAATGGATCAGTGACTACATACACCATGCCT |
| | | CAGTTTTATTATATCACCAAGTGGAACAATGACACTTCAACCAACTGCACC |
| | | TCTGTCATTACATACTCCAGCTTTGCTATCTGCAACACAGGAGAAATCAAG |
| | | TACGTCAATGTAACCCATGTGGAGATCGTCGATGATTCAATCGGAGTCATT |
| | | AAACCTATATCAACTGGAAACATCACAATCCCCAAGAATTTTACTGTCGCA |
| | | GTGCAAGCAGAGTACATTCAGATCCAGGTCAAGCCTGTAGTGGTGGACTGT |
| | | GCCAAATATGTTTGCAATGGAAATAGGCACTGCCTGTCCCTGCTCACTCAG |
| | | TATACTTCAGCCTGTCAAACTATTGAGAATGCACTCAACCTCGGGGCCCGT |
| | | CTTGAGTCACTGATGTTGAATGATATGATCACCGTGTCTGACCGGTCTTTG |
| | | GAACTTGCAACGGTGGAGAAGTTCAACACCACAGTACTTGGAGGAGAAAAA |
| | | CTCGGTGGCTTCTACTTTGACGGGCTGAGTTCTCTATTGCCTCCAACCATC |
| | | GGAAAGAGGAGCGCTGTGGAAGACCTGTTATTCAATAAAGTTGTTACATCT |
| | | GGTCTGGGCACCGTGGACGATGACTACAAAAAATGTTCCGCAGGCACTGAT |
| | | GTCGCTGACCTGGTGTGCGCCCAGTACTATAATGGTATCATGGTTTTACCC |
| | | GGTGTGGTTGACGATAATAAGATGGCCATGTATACGGCCAGCCTGATAGGC |
| | | GGCATGGCAATGGGCTCCATCACGTCAGCCGTTGCGGTGCCCTTCGCCATG |
| | | CAGGTGCAAGCGCGTCTAAACTACGTGGCGCTTCAGACCGACGTGCTGCCG |
| | | GAAAATCAAAAGATACTAGCCAACGCGTTCAACAATGCTATCGGGAATATT |
| | | ACTCTTGCCTTGGGGAAAGGGAGTAATGCCATCACTACCATCTCTccaGGC |
| | | TTCAACACCATGGCTAGTGCCCTCACCAAGATACAGTCCGTAGTGAATCAG |
| | | CAGGGTGAGGCTCTTAGCCAGTTGATCTCCCAGCTGCAGAAGAACTTTCAA |
| | | GCAATTTCTTCTTCCATAGCTGAAATTTACAACCGCCTGGAgCCACCGGAG |
| | | GCCGATGCCCAGGTAGATAGGCTGATTACAGGGCGTCTCGCCGCTCTTAAT |
| | | GCCTATGTCAGCCAGACCTTGACACAGTACGCAGAGGTTAAAGCAAGTCGG |
| | | CAGTTAGCGATGGAGAAAGTTAATGAGTGCGTGAAATCCCAATCCGACCGG |
| | | TATGGCTTTTGTGGCAATGGAACACACCTGTTTTCGTTGGTGAACAGCGCT |
| | | CCTGACGGTCTCCTATTCTTCCACACCGTGTTGCTGCCCACAGAATGGGAG |
| | | GAGGTGACAGCCTGGAGCGGGATTTGTGTCAACGACACCTATGCCTACGTT |
| | | CTCAAAGACTTTGAATACAGCATTTTTAGTTACAATAACACGTACATGGTC |
| | | ACACCCAGAAACATGTTTCAGCCCCGAAAGCCCCAGATGAGCGATTTTGTT |
| | | CAGATAACCTCGTGTGAAGTGACCTTTCTGAATACTACTTACACCACGTTT |
| | | CAGGAGATCGTCATTGACTATATTGACATAAACAAGACTATTGCTGACATG |
| | | CTTGAACAGTACAACCCAAACTATACTACTCCTGAGTTGGACCTTCAGCTG |
| | | GAGATCTTTAACCAAACAAAACTGAACCTGACTGCAGAAATAGATCAGCTC |
| | | GAGCAGAGAGCCGATAACCTAACAACCATAGCTCATGAGCTCCAGCAATAC |
| | | ATCGACAATCTGAACAAGACTCTTGTAGACCTGGAATGGTTGAACAGAATC |
| | | GAAACATACGTAAAATGGCCCTGGTATGTGTGGCTCCTCATCGGCCTCGTC |
| | | GTAGTGTTCTGCATCCCCCTGTTGCTTTTTTGTTGCCTGTCTACTGGTTGC |
| | | TGTGGTTGTTTTGGATGTCTCGGCAGCTGCTGCCACTCCCTGTGCAGCAGA |
| | | CGCCAGTTCGAGAGCTATGAACCCATCGAGAAGGTTCATATTCATGATATC |
| | | TACCCATACGACGTACCAGATTACGCTTGACGTACGGCTGGAGCCTCGGTG |
| | | GCCTAGCTTCTTGCCCCTTGGGCCTCCCCCCAGCCCCTCCTCCCCTTCCTG |
| | | CACCCGTACCCCCGTGGTCTTTGAATAAAGTCTGAGTGGGCGGCAaaaaaa |
| | | aaaaaaaaaaaaaaaaaaaaaaagcatatgactaaaaaaaaaaaaaaaaaa |
| | | aaaaaaaaaaaaaaaaaaaaaaaaaaaaaaaaaaaaaaaaaaaaaaaaaaa |
| | | aAAAAGAAGAGCAAGCTT |
| FCoV S-2P2CD-HA; | 645 | MILLLTLLAVVRSEDAAKNVFVPIDGKIPESFSFSNWFLLTDKSTLVQGRV |
| Protein Sequence | | LSKQPVFVQCLRPVPTSSNVTAVVHFKNDVFCPNVTADVLRFNLNFSDSNY |
| | | TESNNTDQLYFTFEDNTTASIACYSSANVTDFQPANNSVSHIPFGKTTHSY |
| | | FCFANFSHSVVSRQFLGILPPTVREFAFGRDGSIFVNGYKYFSLPPIKSVN |
| | | FSITPVSNNTFWTIAYTNYTDVMVDVNGTGITRLFYCDSPLNRIKCRQLKH |
| | | ELPDGFYSASMLVKKDLPKTFVTMPQFYNWMNVTLHVVLNDTEKKADIILA |
| | | KAPELASLADIHFEIAQANGSVTNVTSLCVQTRQLALFYKYTSLQGLYTYS |
| | | NLVELQNYDCPFSPQKFNNYLQFETLCFDVNPAVAGCKWSLVHDVKWRTQF |
| | | ATITVSYKDGAMITTMPKAQLGFQDISNLVKDECTDYNIYGFQGTGIIRNT |
| | | TSRLVAGLYYTSASGDLLAFKNSTTGEIFTVVPCDLTAQAAVINDEIVGAI |
| | | TAVNQTDLFEFVGGNGSVTTYTMPQFYYITKWNNDTSTNCTSVITYSSFAI |
| | | CNTGEIKYVNVTHVEIVDDSIGVIKPISTGNITIPKNFTVAVQAEYIQIQV |
| | | KPVVVDCAKYVCNGNRHCLSLLTQYTSACQTIENALNLGARLESLMLNDMI |
| | | TVSDRSLELATVEKFNTTVLGGEKLGGFYFDGLSSLLPPTIGKRSAVEDLL |
| | | FNKVVTSGLGTVDDDYKKCSAGTDVADLVCAQYYNGIMVLPGVVDDNKMAM |
| | | YTASLIGGMAMGSITSAVAVPFAMQVQARLNYVALQTDVLQENQKILANAF |
| | | NNAIGNITLALGKVSNAITTISDGFNTMASALTKIQSVVNQQGEALSQLIS |
| | | QLQKNFQAISSSIAEIYNRLDPPEADAQVDRLITGRLAALNAYVSQTLTQY |
| | | AEVKASRQLAMEKVNECVKSQSDRYGFCGNGTHLFSLVNSAPDGLLFFHTV |
| | | LLPTEWEEVTAWSGICVNDTYAYVLKDFEYSIFSYNNTYMVTPRNMFQPRK |
| | | PQMSDFVQITSCEVTFLNTTYTTFQEIVIDYIDINKTIADMLEQYNPNYTT |
| | | PELDLQLEIFNQTKLNLTAEIDQLEQRADNLTTIAHELQQYIDNLNKTLVD |
| | | LEWLNRIETYVKWPWYVWLLIGLVVVFCIPLLLFCCLSTGCCGCFGCLGSC |
| | | CHSLCSRRQFESYEPIEKVHIHDIYPYDVPDYA* |
| FCoV S-2P2CD-HA; | 646 | GAATTCGGCTTTAATACGACTCACTATAAGGACATTAGCTTCTGACACAAC |
| DNA Sequence | | TGTGTTCACTAGCAACCTCAAACAGCCACCATGATTCTGCTGCTGACTCTG |
| | | TTAGCAGTGGTCAGAAGTGAGGATGCTGCTAAGAATGTGTTTGTCCCTATA |
| | | GATGGCAAGATCCCAGAATCCTTCAGCTTCTCGAACTGGTTCCTGTTGACC |
| | | GACAAGAGCACTTTGGTCCAGGGACGCGTGCTGAGCAAACAACCAGTCTTC |
| | | GTCCAGTGCCTCCGCCCAGTGCCAACAAGCAGCAACGTCACAGCGGTTGTG |
| | | CACTTTAAAAATGATGTCTTCSGTCCAAATGTTACCGCTGATGTGCTTAGA |
| | | TTCAATCTCAACTTTTCTGATTCCAATTACACAGAATCCAACAACACGGAC |
| | | CAGCTCTACTTCACTTTCGAGGACAACACAACCGCTAGTATCGCCTGCTAC |
| | | TCCTCAGCGAATGTTACCGATTTCCAACCAGCCAACAATAGCGTGAGTCAC |
| | | ATCCCTTTTGGGAAGACAACCCACAGCTACTTCTGCTTCGCAAATTTCAGT |
| | | CATTCTGTCGTTAGCCGGCAATTCTTGGGGATCCTGCCGCCTACCGTGCGG |
| | | GAGTTTGCCTTCGGACGGGACGGCTCGATCTTTGTCAACGGCTACAAGTAT |
| | | TTTAGTCTCCCGCCGATCAAASCGGTGAACTTCTCAATTACACCTGTCTCC |
| | | AATAATACCTTCTGGACAATTGCATACACAAATTACACAGATGTGATGGTG |
| | | GATGTTAACGGGACAGGAATTACCAGGCTGTTCTACTGTGACAGTCCTTTG |
| | | AACAGGATAAAGTGCAGGCAGTTGAAGCATGAACTGCCAGATGGGTTCTAT |
| | | TCTGCTTCCATGCTGGTCAAGAAAGATCTCCCAAAAACGTTCGTGACAATG |
| | | CCTCAATTCTACAACTGGATGAACGTAACCCTGCACGTCGTACTAAATGAT |
| | | ACCGAGAAGAAGGCTGATATCATCCTCGCAAAGGCTCCAGAGCTGGCTTCC |
| | | CTGGCAGATATTCACTTCGAGATTGCACAGGCCAATGGCTCTGTGACGAAT |
| | | GTTACCTCGCTGTGTGTGCAGACGAGGCAGCTGGCCCTCTTCTATAAGTAT |
| | | ACCAGCCTGCAAGGCCTGTATACGTATAGTAATTTGGTGGAATTGCAGAAT |
| | | TATGACTGCCCCTTTTCTCCACAGAAATTTAATAACTACCTGCAATTTGAG |
| | | ACCCTCTGCTTTGATGTAAACCCTGCAGTCGCCGGGTGTAAGTGGTCACTG |
| | | GTGCATGACGTTAAGTGGAGAACCCAGTTTGCTACGATAACAGTGTCGTAC |
| | | AAAGACGGAGCTATGATAACTACTATGCCCAAAGCCCAGCTTGGCTTCCAG |
| | | GACATTTCGAACCTTGTGAAGGATGAATGTACCGACTATAATATCTATGGA |
| | | TTCCAGGGGACAGGCATCATCCGGAACACCACCTCCCGACTGGTGGCCGGC |
| | | TTGTATTACACTTCTGCATCTGGCGATTTGCTAGCCTTTAAGAACTCCACT |
| | | ACTGGGGAGATCTTCACCGTCGTTCCCTGTGATCTGACAGCTCAGGCCGCA |
| | | GTTATTAATGATGAGATTGTCGGTGCCATTACCGCCGTGAACCAGACTGAT |
| | | CTCTTTGAATTTGTGGGCGGTAATGGATCAGTGACTACATACACCATGCCT |
| | | CAGTTTTATTATATCACCAAGTGGAACAATGACACTTCAACCAACTGCACC |
| | | TCTGTCATTACATACTCCAGCTTTGCTATCTGCAACACAGGAGAAATCAAG |
| | | TACGTCAATGTAACCCATGTGGAGATCGTCGATGATTCAATCGGAGTCATT |
| | | AAACCTATATCAACTGGAAACATCACAATCCCCAAGAATTTTACTGTCGCA |
| | | GTGCAAGCAGAGTACATTCAGATCCAGGTCAAGCCTGTAGTGGTGGACTGT |
| | | GCCAAATATGTTTGCAATGGAAATAGGCACTGCCTGTCCCTGCTCACTCAG |
| | | TATACTTCAGCCTGTCAAACTATTGAGAATGCACTCAACCTCGGGGCCCGT |
| | | CTTGAGTCACTGATGTTGAATGATATGATCACCGTGTCTGACCGGTCTTTG |
| | | GAACTTGCAACGGTGGAGAAGTTCAACACCACAGTACTTGGAGGAGAAAAA |
| | | CTCGGTGGCTTCTACTTTGACGGGCTGAGTTCTCTATTGCCTCCAACCATC |
| | | GGAAAGAGGAGCGCTGTGGAAGACCTGTTATTCAATAAAGTTGTTACATCT |
| | | GGTCTGGGCACCGTGGACGATGACTACAAAAAATGTTCCGCAGGCACTGAT |
| | | GTCGCTGACCTGGTGTGCGCCCAGTACTATAATGGTATCATGGTTTTACCC |
| | | GGTGTGGTTGACGATAATAAGATGGCCATGTATACGGCCAGCCTGATAGGC |
| | | GGCATGGCAATGGGCTCCATCACGTCAGCCGTTGCGGTGCCCTTCGCCATG |
| | | CAGGTGCAAGCGCGTCTAAACTACGTGGCGCTTCAGACCGACGTGCTGCAG |
| | | GAAAATCAAAAGATACTAGCCAACGCGTTCAACAATGCTATCGGGAATATT |
| | | ACTCTTGCCTTGGGGAAAGTGAGTAATGCCATCACTACCATCTCTGATGGC |
| | | TTCAACACCATGGCTAGTGCCCTCACCAAGATACAGTCCGTAGTGAATCAG |
| | | CAGGGTGAGGCTCTTAGCCAGTTGATCTCCCAGCTGCAGAAGAACTTTCAA |
| | | GCAATTTCTTCTTCCATAGCTGAAATTTACAACCGCCTGGACCCACCGGAG |
| | | GCCGATGCCCAGGTAGATAGGCTGATTACAGGGCGTCTCGCCGCTCTTAAT |
| | | GCCTATGTCAGCCAGACCTTGACACAGTACGCAGAGGTTAAAGCAAGTCGG |
| | | CAGTTAGCGATGGAGAAAGTTAATGAGTGCGTGAAATCCCAATCCGACCGG |
| | | TATGGCTTTTGTGGCAATGGAACACACCTGTTTTCGTTGGTGAACAGCGCT |
| | | CCTGACGGTCTCCTATTCTTCCACACCGTGTTGCTGCCCACAGAATGGGAG |
| | | GAGGTGACAGCCTGGAGCGGGATTTGTGTCAACGACACCTATGCCTACGTT |
| | | CTCAAAGACTTTGAATACAGCATTTTTAGTTACAATAACACGTACATGGTC |
| | | ACACCCAGAAACATGTTTCAGCCCCGAAAGCCCCAGATGAGCGATTTTGTT |
| | | CAGATAACCTCGTGTGAAGTGACCTTTCTGAATACTACTTACACCACGTTT |
| | | CAGGAGATCGTCATTGACTATATTGACATAAACAAGACTATTGCTGACATG |
| | | CTTGAACAGTACAACCCAAACTATACTACTCCTGAGTTGGACCTTCAGCTG |
| | | GAGATCTTTAACCAAACAAAACTGAACCTGACTGCAGAAATAGATCAGCTC |
| | | GAGCAGAGAGCCGATAACCTAACAACCATAGCTCATGAGCTCCAGCAATAC |
| | | ATCGACAATCTGAACAAGACTCTTGTAGACCTGGAATGGTTGAACAGAATC |
| | | GAAACATACGTAAAATGGCCCTGGTATGTGTGGCTCCTCATCGGCCTCGTC |
| | | GTAGTGTTCTGCATCCCCCTGTTGCTTTTTTGTTGCCTGTCTACTGGTTGC |
| | | TGTGGTTGTTTTGGATGTCTCGGCAGCTGCTGCCACTCCCTGTGCAGCAGA |
| | | CGCCAGTTCGAGAGCTATGAACCCATCGAGAAGGTTCATATTCATGATATC |
| | | TACCCATACGACGIACCAGATTACGCTTGACGTACGGCTGGAGCCTCGGTG |
| | | GCCTAGCTICTTGCCCCTTGGGCCTCCCCCCAGCCCCTCCTCCCCTTCCTG |
| | | CACCCGTACCCCCGTGGTCTTIGAATAAAGTCTGAGTGGGCGGCAaaaaaa |
| | | aaaaaaaaaaaaaaaaaaaaaaagcatatgactaaaaaaaaaaaaaaaaaa |
| | | aaaaaaaaaaaaaaaaaaaaaaaaaaaaaaaaaaaaaaaaaaaaaaaaaaa |
| | | aAAAAGAAGAGCAAGCTT |
| FCoV II-RBD-MN; | 647 | MILLILALLSTARSEDATHTAVNITIDLGMKRSGYGQPIASTLSNITLPMQ |
| Protein Sequence | | DNNTDVYCIRSNQFSVYVHSTCKSSLWDNIFNQDCTDVLEATAVIKTGTCP |
| | | FSFDKLNNYLTFNKFCLSLSPVGANCKFDVAARTRTNEQVVRSLYVIYEEG |
| | | DNIVGVPSDNSGGSGGSGGSGGSGGSGYIPEAPRDGQAYVRKDGEWVLLST |
| | | FLATNFSLLKQAGDVEENPGPKYILLILACIIACVYGERYCAMQGASTSCI |
| | | NGTENSCQTCFERGDLIWHLANWNFSWSVILIVFITVLQYGRPQFSWLVYG |
| | | IKMLIMWLLWPIVLALTIFNAYSEYQVSRYVMFGFSVAGAVVTFALWMMYF |
| | | VRSIQLYRRTKSWWSFNPETNAILCVNALGRSYVLPLDGTPTGVTLTLLSG |
| | | NLYAEGFKMAGGLTIEHLPKYVMIATPSRTIVYTLVGKQLKATTATGWAYY |
| | | VKSKAGDYSTEARTDNLSEHEKLLHMVATNFSLLKQAGDVEENPGPMATQG |
| | | QRVNWGDEPSKRRGRSNSRGRKNNDIPLSFYNPITLESGSKFWNVCPRDFV |
| | | PKGTGNKDQQTGYWNRQERYRTVKGQRKELPERWFFYFLGTGPHADAKFKD |
| | | KIDGVFWVARDGAMNKPTTLGTRGTNNESKPLKFDGKIPPQFQLEVNRSRN |
| | | NSRSGSQSRSVSRNRSQSRGRQQSNNQNNNVEDTIVAVLQKLGVTDKQRSR |
| | | SKSRDRSDSKSRDTTPKNANKHTWKKTAGKGDVTNFYGARSASANFGDSDL |
| | | VANGNAAKCYPQIAECVPSVSSMLFGSQWSAFEAGDQVKVTLTHTYYLPKD |
| | | DAKTSQFLEQIDAYKRPSQVAKDQRQRKSRSKSADKKPEELSVILVEAYTD |
| | | VFDDTQVEMIDEVTN* |
| FCoV II-RBD-MN; | 648 | GAATTCGGCTTTAATACGACTCACTATAAGGACATTAGCTTCTGACACAAC |
| DNA Sequence | | TGTGTTCACTAGCAACCTCAAACAGCCACCatgatactgttaatattagcg |
| | | cttcttagtaccgctaggtctgaagatgctACGCACACAGCTGTGAATATC |
| | | ACCATCGACCTAGGAATGAAGCGTAGTGGCTATGGGCAGCCCATTGCATCT |
| | | ACACTGAGCAACATAACGTTGCCAATGCAAGACAATAACACAGACGTTTAT |
| | | TGCATCAGATCTAACCAGTTCTCAGTGTATGTGCATTCAACCTGTAAATCT |
| | | TCCCTGTGGGACAATATATTCAATCAGGACTGCACCGATGTTCTGGAAGCC |
| | | ACAGCCGTCATCAAAACAGGAACCTGCCCATTTTCGTTTGACAAACTGAAT |
| | | AACTACCTGACCTTTAATAAGTTCTGTCTCAGTCTgTCGCCGGTAGGAGCT |
| | | AACTGCAAATTCGACGTGGCAGCGAGAACAAGAACTAATGAACAGGTGGTG |
| | | CGGAGCCTGTATGTTATTTATGAAGAGGGGGATAACATCGTAGGGGTCCCT |
| | | AGTGATAATAGCGGAGGCTCGGGCGGGTCTGGTGGGTCTGGGGGTTCAGGG |
| | | GGCTCCGGCTACATCCCTGAAGCTCCGAGAGACGGCCAGGCCTACGTCAGA |
| | | AAAGATGGAGAGTGGGTGCTCCTATCCACCTTCCTGGCTACTAACTTCAGC |
| | | CTGCTGAAGCAGGCTGGAGACGTGGAGGAGAACCCTGGACCTAAGTACATT |
| | | CTCCTgATACTGGCCTGCATCATCGCGTGTGTGTACGGAGAACGCTACTGT |
| | | GCAATGCAGGGAGCATCAACTAGCTGTATCAACGGCACAGAGAACAGCTGC |
| | | CAGACCTGTTTCGAAAGAGGAGACCTCATTTGGCACCTGGCCAACTGGAAT |
| | | TTCAGTTGGTCCGTAATCCTCATAGTCTTCATCACCGTGCTTCAGTATGGA |
| | | AGACCACAGTTCTCCTGGCTgGTTTATGGTATCAAGATGCTGATTATGTGG |
| | | CTGCTCTGGCCCATCGTGCTGGCACTCACCATCTTCAACGCCTATTCCGAG |
| | | TACCAGGTATCCCGGTACGTGATGTTTGGGTTTTCAGTGGCGGGAGCGGTT |
| | | GTCACTTTTGCCCTGTGGATGATGTACTTTGTGCGCAGTATACAGCTCTAC |
| | | AGGAGGACCAAATCTTGGTGGTCATTTAACCCTGAGACGAACGCTATCCTC |
| | | TGCGTGAATGCCCTTGGCAGGTCCTACGTTCTCCCCCTGGACGGTACACCC |
| | | ACTGGAGTCACACTGACTTTGCTCTCCGGCAACTTGTATGCAGAAGGCTTC |
| | | AAAATGGCAGGCGGCCTCACCATAGAGCACTTACCGAAATATGTCATGATT |
| | | GCCACACCTAGCAGGACAATTGTCTACACCTTAGTGGGTAAGCAGCTGAAG |
| | | GCCACGACAGCTACAGGCTGGGCCTACTATGTGAAATCCAAAGCTGGAGAC |
| | | TACAGCACTGAGGCGCGGACTGACAACCTATCTGAACATGAGAAGCTGCTG |
| | | CACATGGTGGCTACTAACTTCAGCCTGCTGAAGCAGGCTGGAGACGTGGAG |
| | | GAGAACCCTGGACCTATGGCCACCCAAGGACAGAGAGTAAACTGGGGCGAT |
| | | GAACCATCCAAACGGAGGGGCCGGTCCAATAGTCGAGGCCGCAAGAACAAT |
| | | GATATTCCGCTGAGCTTCTACAACCCCATAACATTGGAATCAGGAAGCAAG |
| | | TTTTGGAACGTCTGTCCCAGAGATTTTGTCCCAAAGGGCATTGGCAACAAG |
| | | GACCAGCAAATTGGCTACTGGAATAGGCAGGAGAGATACCGCATCGTGAAG |
| | | GGGCAAAGAAAGGAACTGCCAGAGCGCTGGTTCTTCTATTTCCTTGGTACG |
| | | GGCCCACATGCTGATGCCAAATTCAAGGATAAGATCGACGGCGTGTTCTGG |
| | | GTTGCACGGGATGGAGCTATGAACAAACCTACCACACTGGGGACAAGGGGC |
| | | ACCAACAATGAGTCCAAGCCTCTGAAGTTTGATGGGAAAATTCCTCCCCAG |
| | | TTCCAGCTGGAGGTCAACCGAAGCAGGAATAATTCCAGATCTGGGTCCCAA |
| | | TCACGATCTGTAAGTAGGAACCGCTCTCAATCACGGGGGGGGCAGCAGAGC |
| | | AACAACCAGAACAATAATGTGGAGGATACAATTGTTGCAGTCCTGCAGAAG |
| | | CTAGGAGTAACAGATAAACAACGTTCCCGCTCAAAGTCCAGGGACCGGTCG |
| | | GATTCTAAGAGCCGAGACACTACCCCCAAGAATGCAAATAAACACACCTGG |
| | | AAGAAAACTGCAGGCAAGGGTGATGTCACTAACTTCTATGGAGCCCGTAGT |
| | | GCCTCTGCCAATTTcGGGGACTCAGACCTGGTGGCTAATGGAAATGCTGCT |
| | | AAGTGCTACCCTCAGATTGCTGAATGTGTGCCTAGTGTTAGCTCGATGCTg |
| | | TTCGGATCGCAGTGGAGCGCAGAGGAGGCAGGGGATCAAGTGAAAGTAACT |
| | | CTGACTCATACTTACTATCTGCCCAAAGATGATGCTAAGACCTCCCAATTC |
| | | CTGGAGCAGATCGATGCATACAAGCGACCTTCTCAAGTCGCTAAAGATCAG |
| | | CGACAGAGGAAATCTCGCTCCAAGTCAGCTGACAAGAAGCCAGAAGAACTG |
| | | AGCGTGACTCTAGTTGAGGCCTATACTGATGTCTTTGATGACACCCAGGTG |
| | | GAGATGATTGATGAGGTGACCAACTGATAACGTACGGCTGGAGCCTCGGTG |
| | | GCCTAGCTTCTTGCCCCTTGGGCCTCCCCCCAGCCCCTCCTCCCCTTCCTG |
| | | CACCCGTACCCCCGTGGTCTTTGAATAAAGTCTGAGTGGGCGGCAaaaaaa |
| | | aaaaaaaaaaaaaaaaaaaaaaagcatatgactaaaaaaaaaaaaaaaaaa |
| | | aaaaaaaaaaaaaaaaaaaaaaaaaaaaaaaaaaaaaaaaaaaaaaaaaaa |
| | | aAAAAGAAGAGCAAGCTT |

TABLE 5
list of T cell epitopes in the constructs

| | | |
|---|---|---|
| FCoV; T cell | 542 | IDGVFWVAK |
| Epitope sequences | 543 | VIFLVLLGF |
| | 544 | GLFLNTLSF |
| | 545 | VYSIKMLIM |
| | 546 | YMFVNDLTL |
| | 547 | IVLPARHAY |
| | 548 | SHGSIYVTL |
| | 549 | FIYLAYFWY |
| | 550 | WILKSRLIL |
| | 551 | QYGRPQFSW |
| | 552 | AYAKLQLDI |
| | 553 | TPTGITLTL |
| | 554 | EHPVLTWEL |
| | 555 | PMLIAFGYY |
| | 556 | LIGPMLIAF |
| | 557 | ILACIIACV |
| | 558 | VVLLVCVFL |
| | 559 | ELNSIAFAV |
| | 560 | VVSANIKSF |
| | 561 | TDNLSEHEK |
| | 562 | LFLNTLSFI |
| | 563 | TPRNANKHT |
| | 564 | GKLLVCIGF |
| | 565 | PEELSVTLV |
| | 566 | LFVLFLALY |
| | 567 | LVLLGFSCY |
| | 568 | YQVSRYVMF |
| | 569 | GFSVAGAVL |
| | 570 | GVPDSSLRV |
| | 571 | LLIIVLILF |
| | 572 | FVHDRAAPF |
| | 573 | MLIAFGYYI |
| | 574 | VVLRVIFLV |
| | 575 | YIFSQEAVV |
| | 576 | VLLGFSCYT |
| | 577 | AFAVTLKVL |
| | 578 | LYRRTKSWW |
| | 579 | KVCVGVLMF |
| | 580 | FILYNTTTL |
| | 581 | IALLNVIKL |
| | 582 | YMFVNDLTL |
| | 583 | TLVEAYTDV |
| | 584 | VLAETRLLV |
| | 585 | PIVLALTIF |
| | 586 | MPNFSWILK |
| | 587 | AYFWYVNSR |
| | 588 | KSRERADSK |
| | 589 | VTLYGGINY |
| | 590 | YVMIATPSR |
| | 591 | IYVTLYGGI |
| | 592 | MFPRAFTII |
| | 593 | FWNVCPRDF |
| | 594 | VYDFCAKNW |
| | 595 | FALWMMYFV |
| | 596 | FEFILYNTT |
| | 597 | VEDTIVAVL |
| | 598 | RPQFSWLVY |
| | 599 | TFALWMMYF |
| | 600 | AMQQTGMQC |
| | 601 | VTEQSSRSK |
| | 602 | VYGERYCAM |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

We claim:

1. A composition comprising:
a nucleic acid sequence encoding an infection agent antigenic polypeptide, and
a nucleic acid sequence encoding at least one universal T-cell epitope.

2. A composition comprising:
a nucleic acid sequence encoding an infection agent antigenic polypeptide and at least one universal T-cell epitope.

3. The composition of any one of claims 1-2, wherein the at least one universal T-cell epitope is as set forth in Tables 1-5.

4. The composition of any one of claims 1-3, wherein the infection agent is a virus.

5. The composition of claim 4, wherein the virus is a human Metapneumovirus (hMPV), parainfluenza viruses (PIV), respiratory syncytial virus (RSV), measles virus (MeV), influenza virus, herpes simplex virus 1 (HSV1), herpes simplex virus 2 (HSV2), poxvirus, feline coronavirus (FCoV) or a coronavirus, or any combination thereof.

6. The composition of any one of claims 1-5, wherein the nucleic acid sequence encodes at least one human Metapneumovirus (hMPV) antigenic polypeptide, parainfluenza viruses (PIV) antigenic polypeptide, respiratory syncytial virus (RSV) antigenic polypeptide, measles virus (MeV) antigenic polypeptide, influenza virus antigenic polypeptide, herpes simplex virus 1 (HSV1) antigenic polypeptide, herpes simplex virus 2 (HSV2) antigenic polypeptide, poxvirus antigenic polypeptide, feline coronavirus (FCOV) antigenic polypeptide, or a coronavirus antigenic polypeptide, or any combination thereof.

7. The composition of any one of claims 1-5, wherein the nucleic acid sequence encodes at least one human Metapneumovirus (hMPV) antigenic polypeptide, parainfluenza viruses (PIV) antigenic polypeptide, respiratory syncytial virus (RSV) antigenic polypeptide, measles virus (MeV) antigenic polypeptide, influenza virus antigenic polypeptide, poxvirus antigenic polypeptide, feline coronavirus (FCOV) antigenic polypeptide, or a coronavirus antigenic polypeptide, or any combination thereof.

8. The composition of any one of claims 5-7, wherein the coronavirus is a human coronavirus.

9. The composition of any one of claims 5-7, wherein the coronavirus is selected from HCoV-229E, HCoV-OC43, HCoV-HKU1, HCOV-NL, HCOV-NH, HCoV-NL63, SARS-COV, SARS-COV-2, and MERS-COV.

10. The composition of any one of claims 5-8, wherein the coronavirus is SARS-COV-2.

11. The composition of any one of claims 5-7, wherein the poxvirus is monkeypox.

12. The composition of any one of claims 1-5, wherein the nucleic acid sequence encodes at least one human Metapneumovirus (hMPV) antigenic polypeptide, parainfluenza viruses (PIV) antigenic polypeptide, respiratory syncytial virus (RSV) antigenic polypeptide, measles virus (MeV) antigenic polypeptide, influenza virus antigenic polypeptide, herpes simplex virus 1 (HSV1) antigenic polypeptide, herpes simplex virus 2 (HSV2) antigenic polypeptide, poxvirus antigenic polypeptide, feline coronavirus (FCOV) antigenic polypeptide, or any combination thereof.

13. A composition comprising:
a nucleic acid sequence encoding a feline coronavirus (FCOV) antigenic polypeptide, and
a nucleic acid sequence encoding at least one universal T-cell epitope.

14. A composition comprising:
a nucleic acid sequence encoding a feline coronavirus (FCoV) antigenic polypeptide and at least one universal T-cell epitope.

15. A composition comprising:
a nucleic acid sequence encoding a coronavirus antigenic polypeptide, and
a nucleic acid sequence encoding at least one universal T-cell epitope.

16. A composition comprising:
a nucleic acid sequence encoding a coronavirus antigenic polypeptide and at least one universal T-cell epitope.

17. A composition comprising:
a nucleic acid sequence encoding a herpes simplex virus (HSV-1 and/or HSV-2) antigenic polypeptide, and
a nucleic acid sequence encoding at least one universal T-cell epitope.

18. A composition comprising:
a nucleic acid sequence encoding a herpes simplex virus (HSV-1 and/or HSV-2) antigenic polypeptide and at least one universal T-cell epitope.

19. The composition of any one of claims 13-18, wherein the at least one universal T-cell epitope is as set forth in Tables 1-5.

20. The composition of any one of claims 1-19, wherein the nucleic acid sequence encoding the infection agent antigenic polypeptide elicits an antibody response in a subject.

21. The composition of any one of claims 1-20, wherein the nucleic acid sequence encoding the infection agent antigenic polypeptide elicits a broad T-cell response in a subject.

22. The composition of any one of claims 1-21, wherein the nucleic acid sequence encoding the infection agent antigenic polypeptide elicits both an antibody response and a T-cell response in a subject.

23. The composition of any one of claims 1-22, wherein the nucleic acid sequence is a DNA, RNA, any combination thereof (e.g., plasmid DNA, minicircle DNA, minimalistic, immunologically defined gene expression (MIDGE) and Doggybone, messenger RNA (mRNA), circle (cirRNA), self-amplifying-RNA (saRNA, also refer as SAM), or DNA launched SAM (DLSAM)).

24. A nanoparticle comprising:
a nucleic acid sequence encoding an infection agent antigenic polypeptide, and
a nucleic acid sequence encoding at least one universal T-cell epitope.

25. A nanoparticle comprising:
a nucleic acid sequence encoding an infection agent antigenic polypeptide and at least one universal T-cell epitope.

26. The nanoparticle of any one of claims 24-25, wherein the at least one universal T-cell epitope is as set forth in Tables 1-5.

27. The nanoparticle of any one of claims 24-26, wherein the infection agent is a virus.

28. The nanoparticle of claim 27, wherein the virus is a human Metapneumovirus (hMPV), parainfluenza viruses (PIV), respiratory syncytial virus (RSV), measles virus (MeV), influenza virus, herpes simplex virus 1 (HSV1), herpes simplex virus 2 (HSV2), poxvirus, feline coronavirus (FCoV) or a coronavirus, or any combination thereof.

29. The nanoparticle of any one of claims 24-28, wherein the mRNA encodes at least one human Metapneumovirus (hMPV) antigenic polypeptide, parainfluenza viruses (PIV) antigenic polypeptide, respiratory syncytial virus (RSV) antigenic polypeptide, measles virus (MeV) antigenic polypeptide, influenza virus antigenic polypeptide, herpes simplex virus 1 (HSV1) antigenic polypeptide, herpes simplex virus 2 (HSV2) antigenic polypeptide, poxvirus antigenic polypeptide, feline coronavirus (FCOV) antigenic polypeptide, or a coronavirus antigenic polypeptide, or any combination thereof.

30. The nanoparticle of any one of claims 24-28, wherein the nucleic acid sequence encodes at least one human Metapneumovirus (hMPV) antigenic polypeptide, parainfluenza viruses (PIV) antigenic polypeptide, respiratory syncytial virus (RSV) antigenic polypeptide, measles virus (MeV) antigenic polypeptide, influenza virus antigenic polypeptide, poxvirus antigenic polypeptide, feline coronavirus (FCOV) antigenic polypeptide, or a coronavirus antigenic polypeptide, or any combination thereof.

31. The nanoparticle of any one of claims 28-30, wherein the coronavirus is a human coronavirus.

32. The nanoparticle of any one of claims 28-31, wherein the coronavirus is selected from HCoV-229E, HcoV-OC43, HcoV-HKU1, HcoV-NL, HcoV-NH, HcoV-NL63, SARS-CoV, SARS-COV-2, and MERS-COV.

33. The nanoparticle of any one of claims 28-32, wherein the coronavirus is SARS-COV-2.

34. The nanoparticle of any one of claims 28-30, wherein the poxvirus is monkeypox.

35. The nanoparticle of any one of claims 24-27, wherein the nucleic acid sequence encodes at least one human Metapneumovirus (hMPV) antigenic polypeptide, parainfluenza viruses (PIV) antigenic polypeptide, respiratory syncytial virus (RSV) antigenic polypeptide, measles virus (MeV) antigenic polypeptide, influenza virus antigenic polypeptide, herpes simplex virus 1 (HSV1) antigenic polypeptide, herpes simplex virus 2 (HSV2) antigenic polypeptide, poxvirus antigenic polypeptide, feline coronavirus (FCOV) antigenic polypeptide, or any combination thereof.

36. A nanoparticle comprising:
a nucleic acid sequence encoding a feline coronavirus (FCOV) antigenic polypeptide, and
a nucleic acid sequence encoding at least one universal T-cell epitope.

37. A nanoparticle comprising:
a nucleic acid sequence encoding a feline coronavirus (FCOV) antigenic polypeptide and at least one universal T-cell epitope.

38. A nanoparticle comprising:
a nucleic acid sequence encoding a coronavirus antigenic polypeptide, and
a nucleic acid sequence encoding at least one universal T-cell epitope.

39. A nanoparticle comprising:
a nucleic acid sequence encoding a coronavirus antigenic polypeptide and at least one universal T-cell epitope.

40. A nanoparticle comprising:
a nucleic acid sequence encoding a herpes simplex virus (HSV-1 and/or HSV-2) antigenic polypeptide, and
a nucleic acid sequence encoding at least one universal T-cell epitope.

41. A nanoparticle comprising:
a nucleic acid sequence encoding a herpes simplex virus (HSV-1 and/or HSV-2) antigenic polypeptide and at least one universal T-cell epitope.

42. The nanoparticle of any one of claims 36-41, wherein the at least one universal T-cell epitope is as set forth in Tables 1-5.

43. The nanoparticle of any one of claims 24-42, wherein the nucleic acid sequence encoding the infection agent antigenic polypeptide elicits an antibody response in a subject.

44. The nanoparticle of any one of claims 24-43, wherein the nucleic acid sequence encoding the infection agent antigenic polypeptide elicits a T-cell response in a subject.

45. The nanoparticle of any one of claims 24-44, wherein the nucleic acid sequence encoding the infection agent antigenic polypeptide elicits both an antibody response and a T-cell response in a subject.

46. The nanoparticle of any one of claims 24-45, wherein the nucleic acid sequence is DNA, RNA, any combination thereof (e.g., plasmid DNA, minicircle DNA, minimalistic, immunologically defined gene expression (MIDGE) and Doggybone, messenger RNA (mRNA), circle (cirRNA), self-amplifying-RNA (saRNA, also refer as SAM), or DNA launched SAM (DLSAM)).

47. A vaccine comprising:
a nucleic acid sequence encoding an infection agent antigenic polypeptide, and
a nucleic acid sequence encoding at least one universal T-cell epitope.

48. A vaccine comprising:
a nucleic acid sequence encoding an infection agent antigenic polypeptide and at least one universal T-cell epitope.

49. The vaccine of any one of claims 47-48, wherein the at least one universal T-cell epitope is as set forth in Tables 1-5.

50. The vaccine of any one of claims 47-48, wherein the infection agent is a virus.

51. The vaccine of claim 50, wherein the virus is a human Metapneumovirus (hMPV), parainfluenza viruses (PIV), respiratory syncytial virus (RSV), measles virus (MeV), influenza virus, herpes simplex virus 1 (HSV1), herpes simplex virus 2 (HSV2), poxvirus, feline coronavirus (FCoV) or a coronavirus, or any combination thereof.

52. The vaccine of any one of claims 47-50, wherein the nucleic acid sequence encodes at least one human Metapneumovirus (hMPV) antigenic polypeptide, parainfluenza viruses (PIV) antigenic polypeptide, respiratory syncytial virus (RSV) antigenic polypeptide, measles virus (MeV) antigenic polypeptide, influenza virus antigenic polypeptide, herpes simplex virus 1 (HSV1) antigenic polypeptide, herpes simplex virus 2 (HSV2) antigenic polypeptide, poxvirus antigenic polypeptide, feline coronavirus (FCOV) antigenic polypeptide, or a coronavirus antigenic polypeptide, or any combination thereof.

53. The vaccine of any one of claims 47-50, wherein the nucleic acid sequence encodes at least one human Metapneumovirus (hMPV) antigenic polypeptide, parainfluenza viruses (PIV) antigenic polypeptide, respiratory syncytial virus (RSV) antigenic polypeptide, measles virus (MeV) antigenic polypeptide, influenza virus antigenic polypeptide, poxvirus antigenic polypeptide, feline coronavirus (FCoV) antigenic polypeptide, or a coronavirus antigenic polypeptide, or any combination thereof.

54. The vaccine of any one of claims 51-53, wherein the coronavirus is a human coronavirus.

55. The vaccine of any one of claims 51-54, wherein the coronavirus is selected from HCoV-229E, HCoV-OC43, HCoV-HKU1, HCOV-NL, HCOV-NH, HCOV-NL63, SARS-COV, SARS-COV-2, and MERS-COV.

56. The vaccine of any one of claims 51-55, wherein the coronavirus is SARS-COV-2.

57. The vaccine of any one of claims 51-53, wherein the poxvirus is monkeypox.

58. The vaccine of any one of claims 47-50, wherein the nucleic acid sequence encodes at least one human Metapneumovirus (hMPV) antigenic polypeptide, parainfluenza viruses (PIV) antigenic polypeptide, respiratory syncytial virus (RSV) antigenic polypeptide, measles virus (MeV) antigenic polypeptide, influenza virus antigenic polypeptide, herpes simplex virus 1 (HSV1) antigenic polypeptide, herpes simplex virus 2 (HSV2) antigenic polypeptide, poxvirus antigenic polypeptide, feline coronavirus (FCOV) antigenic polypeptide, or any combination thereof.

59. A vaccine comprising:
a nucleic acid sequence encoding a feline coronavirus (FCOV) antigenic polypeptide, and
a nucleic acid sequence encoding at least one universal T-cell epitope.

60. A vaccine comprising:
a nucleic acid sequence encoding a feline coronavirus (FCOV) antigenic polypeptide and
at least one universal T-cell epitope.

61. A vaccine comprising:
a nucleic acid sequence encoding a coronavirus antigenic polypeptide, and
a nucleic acid sequence encoding at least one universal T-cell epitope.

62. A vaccine comprising:
a nucleic acid sequence encoding a coronavirus antigenic polypeptide and at least one universal T-cell epitope.

63. A vaccine comprising:
a nucleic acid sequence encoding a herpes simplex virus (HSV-1 and/or HSV-2) antigenic polypeptide, and
a nucleic acid sequence encoding at least one universal T-cell epitope.

64. A vaccine comprising:
a nucleic acid sequence encoding a herpes simplex virus (HSV-1 and/or HSV-2) antigenic polypeptide and at least one universal T-cell epitope.

65. The vaccine of any one of claims 59-64, wherein the at least one universal T-cell epitope is as set forth in Tables 1-5.

66. The vaccine of any one of claims 47-65, wherein the nucleic acid sequence encoding the infection agent antigenic polypeptide elicits an antibody response in a subject.

67. The vaccine of any one of claims 47-66, wherein the nucleic acid sequence encoding the infection agent antigenic polypeptide elicits a T-cell response in a subject.

68. The vaccine of any one of claims 47-67, wherein the nucleic acid sequence encoding the infection agent antigenic polypeptide elicits both an antibody response and a T-cell response in a subject.

69. The vaccine of any one of claims 47-68, wherein the nucleic acid sequence is a DNA, RNA, any combination thereof (e.g., plasmid DNA, minicircle DNA, minimalistic, immunologically defined gene expression (MIDGE) and Doggybone, messenger RNA (mRNA), circle (cirRNA), self-amplifying-RNA (saRNA, also refer as SAM), or DNA launched SAM (DLSAM)).

70. The vaccine of any one of claims 47-69, wherein the universal T-cell epitope provides broader protection against infection agent variants.

71. A method of treating, inhibiting, reducing, ameliorating, and/or preventing an infection caused by an infection agent in a subject comprising administering to the subject a composition of any one of claims 1-23, a nanoparticle of any one of claims 24-46, or vaccine of any one of claims 47-70.

72. A method of activating T cells, B cells, or any combination thereof comprising contacting a T cells, B cells, or any combination thereof with a composition of any one of claims 1-23, a nanoparticle of any one of claims 24-46, or vaccine of any one of claims 47-70.

73. A method of stimulating the proliferation of T cells, B cells, or any combination comprising contacting a T cells, B cells, or any combination thereof with a composition of any one of claims 1-23, a nanoparticle of any one of claims 24-46, or vaccine of any one of claims 47-70.

74. A method of eliciting an immune response in a subject to an infection agent comprising administering to the subject a composition of any one of claims 1-23, a nanoparticle of any one of claims 24-46, or vaccine of any one of claims 47-70.

75. The method of claim 74, wherein the immune response comprises a cellular immune response, humoral immune response, or any combination thereof.

76. A method of enhancing an immune response generated by a nucleic acid vaccine, the method comprising:
introducing a nucleic acid sequence encoding at least one universal T-cell epitope into the nucleic acid vaccine.

77. A method of enhancing an immune response generated by a nucleic acid vaccine, the method comprising:
introducing a nucleic acid sequence encoding at least one universal T-cell epitope as set forth in Tables 1-5 into the nucleic acid vaccine.

78. The method of claim 77, wherein introduction of the nucleic acid sequence encoding at least one universal T-cell epitope broadens immune response against an infection agent variant.

79. The method of any one of claims 76-78, wherein the nucleic acid sequence is a DNA, RNA, any combination thereof (e.g., plasmid DNA, minicircle DNA, minimalistic, immunologically defined gene expression (MIDGE) and Doggybone, messenger RNA (mRNA), circle (cirRNA), self-amplifying-RNA (saRNA, also refer as SAM), or DNA launched SAM (DLSAM)).

80. The method of any one of claims 71-79, wherein the infection agent is a virus.

81. The method of claim 80, wherein the virus comprises a human Metapneumovirus (hMPV), parainfluenza viruses (PIV), respiratory syncytial virus (RSV), measles virus (MeV), influenza virus, herpes simplex virus 1 (HSV1), herpes simplex virus 2 (HSV2), poxvirus, feline coronavirus (FCoV) or a coronavirus, or any combination thereof.

82. The method of any one of claims 71-81, wherein the mRNA encodes at least one human Metapneumovirus (hMPV) antigenic polypeptide, parainfluenza viruses (PIV) antigenic polypeptide, respiratory syncytial virus (RSV) antigenic polypeptide, measles virus (MeV) antigenic polypeptide, influenza virus antigenic polypeptide, herpes simplex virus 1 (HSV1) antigenic polypeptide, herpes simplex virus 2 (HSV2) antigenic polypeptide, poxvirus antigenic polypeptide, feline coronavirus (FCOV) antigenic polypeptide, or a coronavirus antigenic polypeptide, or any combination thereof.

83. The method of any one of claims 71-81, wherein the nucleic acid sequence encodes at least one human Metapneumovirus (hMPV) antigenic polypeptide, parainfluenza viruses (PIV) antigenic polypeptide, respiratory syncytial virus (RSV) antigenic polypeptide, measles virus (MeV) antigenic polypeptide, influenza virus antigenic polypeptide, poxvirus antigenic polypeptide, feline coronavirus (FCOV) antigenic polypeptide, or a coronavirus antigenic polypeptide, or any combination thereof.

84. The method of any one of claims 81-83, wherein the coronavirus is a human coronavirus.

85. The method of any one of claims 81-84, wherein the coronavirus is selected from HCoV-229E, HCoV-OC43, HCoV-HKU1, HCOV-NL, HCOV-NH, HCOV-NL63, SARS-COV, SARS-COV-2, and MERS-COV.

86. The method of any one of claims 81-85, wherein the coronavirus is SARS-COV-2.

87. The method of any one of claims 81-83, wherein the poxvirus is monkeypox.

88. The method of any one of claims 71-81, wherein the nucleic acid sequence encodes at least one human Metapneumovirus (hMPV) antigenic polypeptide, parainfluenza viruses (PIV) antigenic polypeptide, respiratory syncytial virus (RSV) antigenic polypeptide, measles virus (MeV) antigenic polypeptide, influenza virus antigenic polypeptide, herpes simplex virus 1 (HSV1) antigenic polypeptide, herpes simplex virus 2 (HSV2) antigenic polypeptide, poxvirus antigenic polypeptide, feline coronavirus (FCOV) antigenic polypeptide, or any combination thereof.
